(19) United States
(12) Patent Application Publication
(10) Pub. No.: US 2026/0226425 A1
(43) Pub. Date: Aug. 6, 2026

(54) COLD-ADAPTED, LIVE ATTENUATED SARS-COV-2 VACCINE

(71) Applicants: The University of Tokyo, Tokyo (JP); Wisconsin Alumni Research Foundation, Madison, WI (US)

(72) Inventors: Yoshihiro Kawaoka, Middleton, WI (US); Seiya Yamayoshi, Kanagawa (JP); Satoshi Kamakura, Bunkyo-ku (JP)

(21) Appl. No.: 19/147,944

(22) PCT Filed: Mar. 21, 2024
(86) PCT No.: PCT/US2024/020952

§ 371 Date: Jul. 14, 2025

Related U.S. Application Data
(60) Provisional application No. 63/453,645, filed on Mar. 21, 2023.
(60) Provisional application No. 63/579,686, filed on Aug. 30, 2023.
(60) Provisional application No. 63/557,284, filed on Feb. 23, 2024.

(51) Int. Cl. C12N 7/00; A61K 39/00; A61K 39/215; A61P 31/14; C07K 14/005

(52) U.S. Cl. CPC C12N 7/00; A61K 39/215; A61P 31/14; C07K 14/005; A61K 2039/5254; A61K 2039/543; C12N 2770/20022; C12N 2770/20034

(57) ABSTRACT

An isolated nucleic acid comprising a recombinant coronavirus genome having a genetic modification that provides cold-adapted growth relative to a parent virus that is not cold-adapted and a vaccine comprising the recombinant genome and methods of using the vaccine are provided.

### CROSS-REFERENCE TO RELATED APPLICATIONS

This application claims the benefit of the filing date of U.S. Provisional Application No. 63/453,645, filed on Mar. 21, 2023, and U.S. Provisional Application No. 63/579,686, filed on Aug. 30, 2023, and U.S. Provisional Application No. 63/557,284, filed on Feb. 23, 2024, the disclosures of each of which are incorporated by reference herein.

### INCORPORATION BY REFERENCE OF SEQUENCE LISTING

This application contains a Sequence Listing which has been submitted electronically in ST26 format and is hereby incorporated by reference in its entirety. Said ST26 file, created on Mar. 19, 2024, is named “800128WO1.xml” and is 291,583 bytes in size.

### BACKGROUND

Coronaviruses are single positive stranded RNA viruses that have emerged occasionally from zoonotic sources to infect human populations. Most of the infections in humans cause mild respiratory symptoms, though some recent coronavirus infections in the last decade have resulted in severe morbidity and mortality. These include the severe acute respiratory syndrome coronavirus (SARS-CoV), middle east respiratory syndrome coronavirus (MERS-CoV) and the currently ongoing pandemic of SARS-CoV-2. Infection with these viruses can lead to acute respiratory distress resulting in a high mortality rate. SARS-CoV originated in 2002 in South China and its global spread led to 8096 cases and 774 deaths. The first case of MERS-CoV emerged in 2012 in Saudi Arabia and since then a total of 2494 cases and 858 associated deaths have been reported. 2019 SARS CoV-2 emerged in Wuhan, China at the end of December 2019 and by March 8th 2020 had resulted in 118,096 cases including 4262 deaths globally. The rapid spread of 2019 SARS-CoV2 resulted in the World Health Organization declaring a global pandemic of international concern.

At least 7 different SARS-CoV-2 vaccines are being used worldwide including the Pfizer-BioNTech COVID-19 vaccine (BNT162b2) and two versions of the AstraZeneca/Oxford COVID-19 vaccine (AZD1222).

There are many hurdles to overcome in the development of an effective vaccine for CoVs. Firstly, immunity, whether it is natural or artificial, does not necessarily prevent subsequent infection. Secondly, the propensity of the viruses to recombine may pose a problem. Additionally, vaccination with a viral S-protein has been shown to lead to enhanced disease in the case of FIPV (feline infectious peritonitis virus), a highly virulent strain of feline CoV. This enhanced pathogenicity of the disease is caused by non-neutralizing antibodies that facilitate viral entry into host cells in a process called antibody-dependent enhancement (ADE). After primary infection of one strain of a virus, neutralizing antibodies are produced against the same strain of the virus. However, if a different strain infects the host in a secondary infection, non-neutralizing antibodies produced during the first infection, which do not neutralize the virus, instead, bind to the virus and then bind to the IgG Fe receptors on immune cells and mediate viral entry into these cells.

### SUMMARY

The present disclosure relates to cold-adapted coronaviruses, e.g., cold-adapted SARS-CoV-2 viruses. Cold-adapted vaccines trigger immune responses similar to those elicited upon natural infection and have a long history in the control of viral infections in humans. The process of cold-adaptation of a virus at lower temperatures (e.g., 25° C.) results in an attenuated virus characterized with a compromised ability to grow efficiently at higher temperatures (e.g., 37-40° C.).

A “cold-adapted coronavirus” within the scope of the disclosure includes a virus that is obtained by sequential propagation at progressively lower temperatures, by introduction of an alteration into one or more regions of the viral genome that yields a cold-adapted virus, or is isolated from a sample, e.g., physiological sample, that is at a lower than physiological host temperature. A “cold-adapted virus” produces viral yields that are up to about 100-times, 200-times, 300 times, 400-times, 500-times, 600-times, 700-times, 800-time, 900-times, 1000-times, or more, greater at lower temperatures, e.g., less than 33° C. and greater than 22° C. or less than 31° C. and greater than 24° C., than wild-type virus.

For example, a coronavirus isolate was passaged 15 times at 37° C. and then passaged progressively lower temperatures from 35° C. to 25° C. with the last 3 passages at 25° C. To demonstrate attenuation, golden Syrian hamsters were infected with the parent virus (before passaging) and the cold-adapted virus after 15 passages. Syrian hamsters are highly susceptible to SARS-CoV-2 infection and present with pathological phenotypes similar to those of infected humans; therefore, Syrian hamsters are a model to evaluate the attenuation of cold-adapted SARS-CoV-2. Animals were infected by intranasal inoculation with 1000 plaque-forming units of virus. While infection with the parental virus caused 10% body weight loss, this reduction in body weight was not observed with the cold-adapted virus. Four days after infection, the amount of virus in the lungs was determined. There was a significant reduction (about 1000-fold) in the amount of virus in the lungs of animals infected with the cold-adapted virus compared to the lungs of animals infected with the parent virus. Given the attenuation in hamsters, cold-adapted versions of a corona virus such as SARS-CoV-2 may be used as a vaccine candidate.

The disclosure thus provides for isolated cold-adapted coronaviruses, e.g., cold-adapted SARS-CoV-2 viruses, that have one or more mutations relative to a parental virus that is not cold-adapted, e.g., the replication efficiency of the parental virus is restricted under a low temperature, thereby resulting in decreased viral growth, as well as associated methods of making and using such viruses.

Also provided herein is a method of treating, inhibiting or preventing a coronavirus infection, e.g., a SARS-CoV-2 infection, in a subject in need thereof, comprising administering to the subject a pharmaceutical composition described herein.

The disclosure thus provides an isolated cold-adapted coronavirus wherein the the genome of the coronavirus is a mutant genome where expression of coronavirus S, E, M, N, ORF1, e.g., ORF 1a, ORF3, e.g., ORF3a, ORF6, ORF7, and/or ORF8, comprises one or more mutations in one or more open reading frames. The one or more mutations include but are not limited to one or more nucleotide deletion(s), substitution(s), insertion(s), or any combination thereof. In embodiments, the one or more nucleotide deletions result in an amino acid deletion in one of the open reading frames. In embodiments, the one or more mutations include a substitution of one or more nucleotides. In embodiments, the one or more substitutions result in one or more amino acid substitutions in polypeptides encoded by the one of the open reading frames. In embodiments, the one or more mutations include an insertion of one or more nucleotides.

In embodiments, the open reading frame encodes a non-structural protein. In embodiments, the mutation is in an open reading frame for Nsp1, Nsp2, Nsp3, Nsp6, Nsp13, Nsp14, Nsp15, S, ORF6, ORF7b-ORF8, or any combination thereof. In embodiments, the one or more mutations include a deletion of a codon for the amino acid at position 85 in Nsp1. In one embodiment, the one or more mutations include a nucleotide substitution in the gene for Nsp2 at position 1341 or 1495, or both. In one embodiment, the one or more mutations include an amino acid substitution in Nsp3 at position 272 or 528, or both. In one embodiment, the amino acid at position 272 is not Y. In one embodiment, the amino acid at position 272 is N, D, E or Q. In one embodiment, the amino acid at position 528 is not L. In one embodiment, the amino acid at position 528 is F, W or Y. In one embodiment, the one or more mutations include an amino acid substitution in Nsp6 at position 260. In one embodiment, the amino acid at position 260 is not L. In one embodiment, the amino acid at position 260 is F, W or Y. In one embodiment, the one or more mutations include a nucleotide substitution in the gene for Nsp13 at position 16575. In one embodiment, the one or more mutations include an amino acid substitution in Nsp14 at position 360. In one embodiment, the amino acid at position 360 is not A. In one embodiment, the amino acid at position 360 is S, T, L, I or M. In one embodiment, the one or more mutations include an amino acid substitution in Nsp15 at position 233. In one embodiment, the amino acid at position 233 is not E. In one embodiment, the amino acid at position 233 is A, I, L, G, S, or T. In one embodiment, the one or more mutations include an amino acid substitution in S at position 64, 235, 1094, or a combination thereof. In one embodiment, the amino acid at position 64 is not W, position 253 is not D, or position 1094 is not V. In one embodiment, the amino acid at position 64 is R, H or K; position 253 is G, A, L, I, T or S; or position 1094 is F, Y or W. In one embodiment, the one or more mutations include a deletion of the C-terminus of polypeptide encoded by ORF6. In one embodiment, the one or more mutations include a deletion of ORF7b and/or ORF8.

Also provided is a method of making a cold-adapted coronavirus comprising modifying the genome thereof to include one or more mutations in one or more open reading frames. In one embodiment, the coronavirus is SARS-CoV-2. In one embodiment, the one or more mutations include a deletion of one or more nucleotides. In one embodiment, the one or more nucleotide deletions result in an amino acid deletion in one of the open reading frames. In one embodiment, the one or more mutations include a substitution of one or more nucleotides. In one embodiment, the one or more substitutions result in one or more amino acid substitutions in a polypeptide encoded by one of the open reading frames. In one embodiment, the one or more mutations include an insertion of one or more nucleotides. In one embodiment, the open reading frame encodes a non-structural protein. In one embodiment, in the mutation is in an open reading frame for Nsp1, Nsp2, Nsp3, Nsp6, Nsp13, Nsp14, Nsp15, S, ORF6, ORF7b-ORF8, or any combination thereof. In one embodiment, the one or more mutations include a deletion of a codon for the amino acid at position 85 in Nsp1. In one embodiment, the one or more mutations include a nucleotide substitution in the gene for Nsp2 at position 1341 or 1495, or both. In one embodiment, the one or more mutations include an amino acid substitution in Nsp3 at position 272 or 528, or both. In one embodiment, the amino acid at position 272 is not Y. In one embodiment, the amino acid at position 272 is N, D, E or Q. In one embodiment, the amino acid at position 528 is not L. In one embodiment, the amino acid at position 528 is F, W or Y. In one embodiment, the one or more mutations include an amino acid substitution in Nsp6 at position 260. In one embodiment, the amino acid at position 260 is not L. In one embodiment, the amino acid at position 260 is F, W or Y. In one embodiment, the one or more mutations include a nucleotide substitution in the gene for Nsp13 at position 16575. In one embodiment, the one or more mutations include an amino acid substitution in Nsp14 at position 360. In one embodiment, the amino acid at position 360 is not A. In one embodiment, the amino acid at position 360 is S, T, L, I or M. In one embodiment, the one or more mutations include an amino acid substitution in Nsp15 at position 233. In one embodiment, the amino acid at position 233 is not E. In one embodiment, the amino acid at position 233 is A, I, L, G, S, or T. In one embodiment, the one or more mutations include an amino acid substitution in S at position 64, 253, 1094, or a combination thereof. In one embodiment, the amino acid at position 64 is not W, position 253 is not D, or position 1094 is not V. In one embodiment, the amino acid at position 64 is R, H or K; position 253 is G, A, L, I, T or S; or position 1094 is F, Y or W. In one embodiment, the one or more mutations include a deletion of the C-terminus of polypeptide encoded by ORF6. In one embodiment, the one or more mutations include a deletion of ORF7b and/or ORF8.

Further provided is a method to immunize an animal, comprising administering to the animal a composition comprising the cold-adapted coronavirus. In one embodiment, the animal is a mammal. In one embodiment, the mammal is a human. In one embodiment, the composition is injected. In one embodiment, the composition is systemically administered. In one embodiment, the composition is intranasally administered. In one embodiment, the composition is subcutaneously administered. In one embodiment, the composition is intramuscularly administered.

The disclosure also provides a pharmaceutical composition comprising the cold-adapted coronavirus. In one embodiment, the composition further comprises a pharmaceutically acceptable carrier. In one embodiment, the composition further comprises a different immunogen. In one embodiment, the composition further comprises a different virus, e.g., the different virus is a not a coronavirus. In one embodiment, the different virus is an influenza virus. In some embodiments, the pharmaceutical composition having a cold-adapted coronavirus is administered in addition to one or more therapeutics for the coronavirus, e.g., a different coronavirus vaccine, or for a different virus, e.g., influenza virus. In some embodiments, the pharmaceutical composition having a cold-adapted coronavirus disclosed herein is administered 1-10 weeks before or after a first administration of a coronavirus vaccine. In some embodiments, the pharmaceutical composition is administered 1-6 weeks, 1-6 months or 1-2 years or later after a first administration of a coronavirus vaccine. In some embodiments, the pharmaceutical composition is administered on the same day or simultaneously with an administration of another pharmaceutical composition. In some embodiments, the pharmaceutical composition is co-formulated with a different coronavirus immunogenic composition, e.g., a different SARS-CoV-2 immunogenic composition, or a different viral immunogenic composition. In some embodiments, the pharmaceutical composition having a cold-adapted SARS-CoV-2 is administered before an administration of a different SARS-CoV-2 pharmaceutical composition, such as 2-10 weeks before an administration of the SARS-CoV-2 spike protein pharmaceutical composition.

In some embodiments, the pharmaceutical composition is administered prophylactically. In some embodiments, the pharmaceutical composition is administered once every 1, 2, 3, 4, 5, 6 or more months, e.g., every 7-14, 14-21, 21-28, or 28-35 months.

Also provided herein is a use of a composition described herein for preparing a therapeutic for treating, inhibiting or preventing a respiratory viral infection caused by a coronavirus including a SARS CoV-2.

Also provided herein is a composition described herein or a pharmaceutical composition described herein for use as a medicament.

Also provided herein is a composition described herein or a pharmaceutical composition described herein for use in the treatment or prevention of a respiratory viral infection caused by a coronavirus such as a SARS CoV-2.

### BRIEF DESCRIPTION OF THE FIGURES

FIG. 1. Generation of a cold-adapted coronavirus.

FIG. 2. Propagation of an exemplary seed (vaccine) virus in Vero cells.

FIG. 3. Pathogenicity and immunogenicity (attenuation) of an exemplary seed (vaccine) virus in hamsters.

FIG. 4. No body weight loss after infection with an exemplary cold-adapted coronavirus.

FIG. 5. Respiratory function test based on WBP (whole-body plethysmography) Penh: Index that combines the box pressure signals from both inspiration (PIF) and expiration (PEF), with the timing comparison of early and late expiration (PAU). Penh=PAU*(PEF/PIF). Rpef: Ratio of time required to reach the maximum expiratory flow (PEF) relative to time of expiration (Te).

FIG. 6. Virus titers in the respiratory organs of infected hamsters (3 dpi).

FIG. 7. CT images of infected hamster lungs confirming attenuation of the vaccine virus in hamsters

FIG. 8. Table of exemplary cold-adapted coronavirus induces neutralizing antibodies against wild-type, Beta, Gamma, Delta and BA.1.

FIG. 9. Experimental design of immunization with an exemplary cold-adapted coronavirus and challenge of immunized hamsters with the wild-type virus.

FIG. 10. The body weight change of immunized hamsters upon challenge with the wild-type virus was similar between animals previously immunized with either the wild-type or the exemplary cold-adapted coronavirus.

FIG. 11. Respiratory functions of hamsters challenged with the wild-type virus were similar between animals previous immunized with either the wild-type or the exemplary cold-adapted coronavirus. Penh: Index that combines the box pressure signals from both inspiration (PIF) and expiration (PEF), with the timing comparison of early and late expiration (PAU). Penh=PAU*(PEF/PIF). Rpef: Ratio of time required to reach the maximum expiratory flow (PEF) relative to time of expiration (Te).

FIG. 12. The levels of virus titer reduction in respiratory organs were similar between animals previous immunized with either the wild-type or the exemplary cold-adapted coronavirus.

FIG. 13. Micro-CT analysis in the lungs of cold-adapted coronavirus-immunized hamsters showing no lung lesions in hamsters previously immunized with either the wild-type or the exemplary cold-adapted coronavirus upon challenge with the wild-type virus.

FIG. 14. Experimental design of immunization with the exemplary cold-adapted coronavirus and challenge of immunized hamsters with the Delta variant.

FIG. 15. The body weight change of immunized hamsters upon challenge with the Delta variant was similar between animals previously immunized with either the wild-type or the exemplary cold-adapted coronavirus.

FIG. 16. Respiratory functions of hamsters challenged with the Delta variant were similar between animals previous immunized with either the wild-type or the exemplary cold-adapted coronavirus.

FIG. 17. The levels of virus titer reduction in respiratory organs were similar between animals previous immunized with either the wild-type or the exemplary cold-adapted coronavirus.

FIG. 18. Micro-CT analysis in the lungs of cold-adapted coronavirus-immunized hamsters showing no lung lesions in hamsters previously immunized with either the wild-type or the exemplary cold-adapted coronavirus upon challenge with the Delta variant.

FIG. 19. Attenuation stability test in hamsters showing in vivo stability of the attenuated cold-adapted coronavirus that was passaged in the nasal turbinates 3 times or isolated from the lungs of hamsters 3-5 days after infection and then tested for cold adaptation, temperature sensitivity, and pathogenicity.

FIG. 20. Attenuation stability test in hamsters showing the attenuated cold-adapted coronavirus in the nasal turbinate did not change during the 3 passages.

FIG. 21. The cold-adapted coronavirus passaged 3 times in nasal turbinate remained similar to the original cold-adapted coronavirus.

FIG. 22. Pathogenicity of the cold-adapted coronavirus passaged in nasal turbinates.

FIGS. 23A-23D. Pathogenicity of passaged cold-adapted coronaviruses. Hamsters lost about 5% of body weight in the wild-type group and gained weight in remaining groups (FIG. 23A). Respiratory function declined in the wild-type group and remained substantially steady in remaining groups (FIG. 23B). CT scans of lungs of each group are shown in FIG. 23C. Virus titer in nasal turbinates (NT) and lungs is shown in FIG. 23D.

FIG. 24. Attenuation stability test in hamsters showing cold-adapted coronavirus was detected in the lungs of hamsters at 5 days post-infection.

FIG. 25. Seed cold-adapted coronaviruses isolated from hamster lung at 5 days post-infection showing similar growth kinetics at 25, 32, 37, and 39° C. to those of the cold-adapted coronavirus not passaged through hamster lungs.

FIG. 26. Pathogenicity of cold-adapted coronaviruses isolated from hamster lung.

FIGS. 27A-D. Body weight change (FIG. 27A), respiratory function for Penh and Rpef (FIG. 27b), micro-CT analysis (FIG. 27C), and virus titers in the nasal turbinates and lungs of the hamsters (FIG. 27D) showed that the two isolated viruses maintained the attenuated phenotype in the hamsters.

FIGS. 28A-D. Experimental design to test attenuation stability in Vero cells (FIG. 28A). Table of virus titers of cold-adapted coronavirus passaged through Vero cells at different temperatures (FIG. 28B). Experimental design to test the pathogenicity of the 3 passaged viruses by intranasal inoculation of hamsters with 10³ PFU of passaged viruses (FIG. 28C). Body weight change, respiratory function for Penh and Rpef, micro-CT analysis, and virus titers in the nasal turbinates and lungs of the hamsters (FIG. 28D).

FIG. 29. Virus titer of recombinant viruses possessing a single amino acid mutation in Vero cells.

FIG. 30. Pathogenicity of single amino acid mutants in hamster. These viruses were generated by reverse genetics using the BAC system.

FIG. 31. Graph showing ORF1ab-Y1090N and S-V1094F mutants suppressed the body weight loss of infected hamsters.

FIG. 32. Graphs showing respiratory function of ORF1ab-M85del and ORF1ab-Y1090N mutations in cold-adapted coronavirus contributed to attenuation in hamsters.

FIG. 33. Micro-CT images showing that ORF1ab-M85del and ORF1ab-Y1090N mutations in cold-adapted coronavirus caused attenuation in hamsters.

FIG. 34. Exemplary parental virus genomic sequence (SEQ ID NO:1).

FIG. 35. Genomic structure of an exemplary coronavirus (SEQ ID NO:2).

FIG. 36. Exemplary SARS CoV-2 genome (Delta variant) for modification (SEQ ID NO:3).

FIG. 37. Exemplary SARS CoV-2 genome (Omicron variant) for modification (SEQ ID NO:4).

FIG. 38. Experimental design to test the attenuation of the cold-adapted coronavirus and induction of antibodies and cellular immunity in human ACE2-transgenic mice.

FIG. 39. Graphs of AC70 and K18 mice body weight vs. days post infection with cold-adapted coronavirus.

FIG. 40. Graphs of cold-adapted coronavirus titer in nasal turbinates and lungs of AC70 or K18 mice.

FIG. 41. Anti-RBD and Anti-S ELISA titer in K18 mice.

FIG. 42. Th1/Th2 balance in K18-tg mice.

FIG. 43. Experimental design to assess cellular immunity against the S protein in K18-tg mice.

FIG. 44. Graph showing IFNγ- and TNFα-positive CD4 and CD8 T cells were detected in the splenocytes of K18-mice infected with the vaccine cold-adapted coronavirus.

FIG. 45. ELISPOT showed that cellular immunity against the spike protein was induced in K18-mice after vaccine cold-adapted coronavirus infection.

FIG. 46. Experimental design for a challenge test of cold-adapted coronavirus immunized hamsters with omicron variant BA.1,

FIG. 47. Graph of body weight loss of cold-adapted coronavirus-immunized hamsters challenged with Wuhan-like, Delta, and BA.1 virus.

FIG. 48. Respiratory function test based on WBP.

FIG. 49. Virus titers in the lungs and nasal turbinates of cold-adapted coronavirus-immunized hamsters challenged with Wuhan-like, Delta, and BA.1 virus.

FIG. 50. Micro-CT images of challenged immunized hamster lungs.

FIG. 51. Molecular Basis for the attenuation of recombinant vaccine cold-adapted coronaviruses.

FIG. 52. Experimental design for growing recombinant viruses in Vero cells at 25, 32, and 37° C.

FIG. 53. Graph of virus titer of recombinant viruses in Vero cells.

FIG. 54. Graph of virus titer of reverse engineered wild type and cold-adapted coronavirus in Vero cells.

FIG. 55. Graph of virus titer of nsp-M85ins and nsp3-Y272N recombinant viruses in Vero cells.

FIG. 56. Graph of virus titer of nsp6-F260L and S-F1094V recombinant viruses in Vero cells.

FIG. 57. Experimental design to test the pathogenicity of the recombinant viruses.

FIG. 58. Body weight changes in hamsters.

FIG. 59. Respiratory function in hamsters.

FIG. 60. Recombinant virus titer in nasal turbinates and lung of hamsters.

FIGS. 61A-B. Mutations introduced into wild-type SARS-Cov-2/UT-HPCo-038/human/2020/Tokyo (SEQ ID NO:78) (FIG. 61A). Mutations introduced into vaccine cold-adapted coronavirus (FIG. 61B).

FIGS. 62A-B. Schematic of genome of vaccine cold-adapted coronavirus and recombinant vaccine cold-adapted coronavirus with the S gene replaced with the S gene of XBB.1.5 virus (FIG. 61A). Sequence of SARS CoV-2 virus, “EG.5.1” (FIG. 61B; SEQ ID NO: 6).

FIG. 63. Experimental design for testing virus titers of vaccine cold-adapted coronaviruses possessing the S gene of XBB.1.5 harboring the V1094F substitution or the W64R, D253G, and V1094F substitutions.

FIG. 64. Graph showing vaccine cold-adapted coronaviruses possessing the XBB.1.5 S gene showed similar growth kinetics to those of the vaccine cold-adapted coronavirus (LAV).

FIG. 65. Experimental design to test the pathogenicity of the three viruses possessing the XBB.1.5 S gene.

FIG. 66. Graph of body weight change of hamsters infected with the three viruses possessing the XBB.1.5 S gene.

FIG. 67. Graph of respiratory function of hamsters infected with the three viruses possessing the XBB.1.5 S gene.

FIG. 68. Graph of virus titers in nasal turbinates and lung of hamsters infected with the three viruses possessing the XBB.1.5 S gene.

FIGS. 69A-B. Experimental design to test boost immunization with vaccine cold-adapted coronavirus in hamsters that received mRNA vaccination once (FIG. 69A) or twice (FIG. 69B).

FIG. 70. Graph of antibody titers after the boost immunization with the vaccine cold-adapted coronavirus.

FIG. 71. Experimental design to test vaccine cold-adapted coronavirus attenuation in non-human primates (macaques).

FIG. 72. Graph of body weight of macaques infected with vaccine cold-adapted coronavirus or wild-type virus.

FIG. 73. Graphs of vaccine see cold-adapted coronavirus virus loads in the nasal, oral, and rectal swabs of macaques.

FIG. 74. Graph showing vaccine cold-adapted coronavirus RNA was not detected in any organ tested, whereas wild-type virus RNA was detected in the olfactory bulb, turbinates, ileums, and trachea of macaques.

FIG. 75. Experiment design to show vaccine cold-adapted coronavirus efficacy in macaques.

FIG. 76. Graph of body weight change of immunized macaques after challenge infection.

FIG. 77. Graphs of viral loads in the nasal, oral, and rectal swabs after challenge infection.

FIG. 78. Graph of virus titers in organs after challenge infection.

FIG. 79. Exemplary parental virus genomic sequence Wuhan-Hu-1 (SEQ ID NO: 5).

FIG. 80. Exemplary parental virus genomic sequence SARS-Cov-2/UT-HPCo-038/human/2020/Tokyo (SEQ ID NO:78).

### DETAILED DESCRIPTION

Currently, vaccines against SARS-CoV-2 are only directed against the spike protein. A cold-adapted, live attenuated coronavirus vaccine may elicit a different or enhanced immune response against the virus, resulting in superior protection with one immunization.

### Definitions

The term “isolated” when used in relation to a nucleic acid, peptide, polypeptide or virus refers to a nucleic acid sequence, peptide, polypeptide or virus that is identified and separated from at least one contaminant nucleic acid, polypeptide or other biological component with which it is ordinarily associated in its natural source, e.g., so that it is not associated with in vivo substances, or is substantially purified from in vitro substances. Isolated nucleic acid, peptide, polypeptide or virus is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded).

As used herein, “substantially purified” means the object species is the predominant species, e.g., on a molar basis it is more abundant than any other individual species in a composition, and preferably is at least about 80% of the species present, and optionally 90% or greater, e.g., 95%, 98%, 99% or more, of the species present in the composition.

As used herein, “substantially free” means below the level of detection for a particular infectious agent using standard detection methods for that agent.

A “recombinant” virus is one which has been manipulated in vitro, e.g., using recombinant DNA techniques, to introduce changes to the viral genome. Viruses can be prepared by recombinant or nonrecombinant techniques.

As used herein, the term “recombinant nucleic acid” or “recombinant DNA sequence or segment” refers to a nucleic acid, e.g., to DNA, that has been derived or isolated from a source, that may be subsequently chemically altered in vitro, so that its sequence is not naturally occurring, or corresponds to naturally occurring sequences that are not positioned as they would be positioned in the native genome. An example of DNA “derived” from a source, would be a DNA sequence that is identified as a useful fragment, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the disclosure, by the methodology of genetic engineering.

The term “heterologous” as it relates to nucleic acid sequences such as gene sequences encoding a protein and control sequences, denotes sequences that are not normally joined together, and/or are not normally associated with a particular cell, e.g., are from different sources (for instance, sequences from a virus are heterologous to sequences in the genome of an uninfected cell). Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature, i.e., a heterologous promoter. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this disclosure.

By “DNA” is meant a polymeric form of deoxyribonucleotides (adenine, guanine, thymine, or cytosine) in double-stranded or single-stranded form found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the nontranscribed strand of DNA (i.e., the strand having the sequence complementary to the mRNA). The term captures molecules that include the four bases adenine, guanine, thymine, or cytosine, as well as molecules that include base analogues which are known in the art.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

A “gene,” “polynucleotide,” “coding region,” “sequence,” “segment,” “fragment” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “control elements” refers collectively to promoter regions, polyadenylation signals, transcription termination sequences, upstream regulatory domains, origins of replication, internal ribosome entry sites (“IRES”), enhancers, splice junctions, and the like, which collectively provide for the replication, transcription, post-transcriptional processing and translation of a coding sequence in a recipient cell. Not all of these control elements need always be present so long as the selected coding sequence is capable of being replicated, transcribed and translated in an appropriate host cell.

The term “promoter” is used herein in its ordinary sense to refer to a nucleotide region comprising a DNA regulatory sequence, wherein the regulatory sequence is derived from a gene which is capable of binding RNA polymerase and initiating transcription of a downstream (3′ direction) coding sequence. By “enhancer” is meant a nucleic acid sequence that, when positioned proximate to a promoter, confers increased transcription activity relative to the transcription activity resulting from the promoter in the absence of the enhancer domain.

By “operably linked” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. “Operably linked” with reference to peptide and/or polypeptide molecules is meant that two or more peptide and/or polypeptide molecules are connected in such a way as to yield a single polypeptide chain, i.e., a fusion polypeptide, having at least one property of each peptide and/or polypeptide component of the fusion. The fusion polypeptide may be chimeric, i.e., composed of heterologous molecules.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less or 2 bases or less. When using oligonucleotides as probes or treatments, the sequence homology between the target nucleic acid and the oligonucleotide sequence is generally not less than 17 target base matches out of 20 possible oligonucleotide base pair matches (85%); e.g., not less than 9 matches out of 10 possible base pair matches (90%), or not less than 19 matches out of 20 possible base pair matches (95%). The correspondence between one sequence and to another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions which form stable duplexes between homologous regions, followed by digestion with single strand-specific nuclease(s), and size determination of the digested fragments. Two DNA, or two polypeptide, sequences are “substantially homologous” to each other when at least about 80%, e.g., at least about 90%, such as at least about 95% of the nucleotides, or amino acids, respectively match over a defined length of the molecules, as determined using the methods above.

The term “selectively hybridize” means to detectably and specifically bind. Polynucleotides, oligonucleotides and fragments of the disclosure selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the disclosure and a nucleic acid sequence of interest is at least 65%, and more typically with increasing homologies of at least about 70%, about 90%, about 95%, about 98%, and 100%.

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less or 2 or less. Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof may be homologous if their amino acids are greater than or equal to 50% identical when optimally aligned using the ALIGN program.

The term “corresponds to” is used herein to mean that a polynucleotide sequence is homologous (e.g., is identical, not strictly evolutionarily related) to all or a portion of a reference polynucleotide sequence that encodes a polypeptide or its complement, or that a polypeptide sequence is identical in sequence or function to a reference polypeptide sequence. For illustration, the nucleotide sequence “TATAC” corresponds to a reference sequence “TATAC” and is complementary to a reference sequence “GTATA”.

The following terms are used to describe the sequence relationships between two or more polynucleotides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing, or may comprise a complete cDNA or gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window”, as used herein, refers to a conceptual segment of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by using local homology algorithms or by a search for similarity method, by computerized implementations of these algorithms (GAP, BESTFIT, FASTA, and TFASTA Genetics Software Package or by inspection, and the best alignment (i.e., resulting in the highest percentage of homology over the comparison window) generated by the various methods is selected.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, e.g., at least 90 to 95 percent sequence identity, or at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

As applied to polypeptides, the term “substantial identity” means that two peptide sequences, when optimally aligned, such as by the programs GAP or BESTFIT using default gap weights, share at least about 80% sequence identity, at least about 90% sequence identity, at least about 95% percent sequence identity, or at least about 99% sequence identity.

By “mammal” is meant any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats, rabbits and guinea pigs, and the like.

By “derived from” is meant that a nucleic acid molecule was either made or designed from a parent nucleic acid molecule, the derivative retaining substantially the same functional features of the parent nucleic acid molecule, e.g., encoding a gene product with substantially the same activity as the gene product encoded by the parent nucleic acid molecule from which it was made or designed.

By “expression construct” or “expression cassette” is meant a nucleic acid molecule that is capable of directing transcription. An expression construct includes, at the least, a promoter. Additional elements, such as an enhancer, and/or a transcription termination signal, may also be included.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature.

The term “wild-type” or “native” refers to a gene or gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “recombinant protein” or “recombinant polypeptide” as used herein refers to a protein molecule expressed from a recombinant DNA molecule. In contrast, the term “native protein” is used herein to indicate a protein isolated from a naturally occurring (i.e., a nonrecombinant) source. Molecular biological techniques may be used to produce a recombinant form of a protein with identical properties as compared to the native form of the protein.

Methods of alignment of sequences for comparison are well known in the art. Thus, the determination of percent identity between any two sequences can be accomplished using a mathematical algorithm.

Computer implementations of these mathematical algorithms can be utilized for comparison of sequences to determine sequence identity. Alignments using these programs can be performed using the default parameters. Software for performing BLAST analyses is publicly available through the National Center for Biotechnology Information (http://www.ncbi.nlm.nih.gov/). The algorithm may involve first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm may also perform a statistical analysis of the similarity between two sequences. One measure of similarity provided by the BLAST algorithm may be the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

The BLASTN program (for nucleotide sequences) may use as defaults a wordlength (W) of 11, an expectation (E) of 10, a cutoff of 100, M=5, N=−4, and a comparison of both strands. For amino acid sequences, the BLASTP program may use as defaults a wordlength (W) of 3, an expectation (E) of 10, and the BLOSUM62 scoring matrix. See ncbi.nlm.nih.gov. Alignment may also be performed manually by inspection.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Methods of identifying nucleotide and amino acid conservative substitutions which do not eliminate peptide function are well-known in the art.

A “vector” or “construct” (sometimes referred to as gene delivery or gene transfer “vehicle”) refers to a macromolecule or complex of molecules comprising a polynucleotide or virus to be delivered to a host cell, either in vitro or in vivo. The polynucleotide or virus to be delivered may comprise a coding sequence of interest for gene therapy. Vectors include, for example, viral vectors (such as coronavirus, filovirus, adenovirus, adeno-associated virus (AAV), lentivirus, herpesvirus and retrovirus vectors), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

A “recombinant viral vector” refers to a viral vector comprising one or more modifications, including deletions, insertions, substitutions, and/or heterologous genes or sequences. Since many viral vectors exhibit size constraints associated with packaging, the heterologous genes or sequences are typically introduced by replacing one or more portions of the viral genome. Such viruses may become replication-defective or replication-incompetent, e.g., requiring the deleted function(s) to be provided in trans during viral replication and encapsidation (by using, e.g., a helper virus or a packaging cell line carrying genes for replication and/or encapsidation). Modified viral vectors in which a polynucleotide to be delivered is carried on the outside of the viral particle have also been described.

As used herein, “substantially pure” means an object species is the predominant species present (i.e., on a molar basis it is more abundant than any other individual species in the composition), and optionally a substantially purified fraction is a composition wherein the object species comprises at least about 50 percent (on a molar basis) of all macromolecular species present. Generally, a substantially pure composition will comprise more than about 80 percent of all macromolecular species present in the composition, more than about 85%, about 90%, about 95%, and about 99%. For example, the object species is purified to essential homogeneity (contaminant species cannot be detected in the composition by conventional detection methods) wherein the composition consists essentially of a single macromolecular species.

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The terms “effective amount” or “therapeutically effective amount” or “therapeutic effect” refer to an amount of a therapeutic effective to “treat” a disease or disorder in a subject or mammal. The therapeutically effective amount has a therapeutic effect and as such casn prevent the development of a disease or disorder; slow down the development of a disease or disorder; slow down the progression of a disease or disorder; relieve to some extent one or more of the symptoms associated with a disease or disorder; reduce morbidity and mortality; improve quality of life; or a combination of such effects.

The terms “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent or slow the development of a targeted pathologic condition or disorder. Thus, those in need of treatment include those already with the disorder; those prone to have the disorder; and those in whom the disorder is to be prevented.

As used in the present disclosure and embodiments, the singular forms “a”, “an” and “the” include plural forms unless the context clearly dictates otherwise.

The term “therapeutic” refers a composition that is used to treat, inhibit or prevent a disease or a condition, such as viral infection, e.g., coronaviral infection. For example, a therapeutic may be vaccine. A therapeutic may be a drug, e.g., a small molecule drug. A therapeutic may be administered to a subject in need thereof, to prevent a disease or an infection, or to reduce or ameliorate one or more symptoms associated with a disease. A therapeutic may also be considered to treat at least a symptom of the disease.

“Transfected,” “transformed” or “transgenic” is used herein to include any host cell or cell line, which has been altered or augmented by the presence of at least one recombinant DNA sequence. The host cells of the present disclosure are typically produced by transfection with a DNA sequence in a plasmid expression vector, as an isolated linear DNA sequence, or infection with a recombinant viral vector.

The basic organization of the coronavirus genome is shared with other members of the Nidovirus order (the torovirus genus, also in the family Coronaviridae, and members of the family Arteriviridae) in that the nonstructural proteins involved in proteolytic processing, genome replication, and subgenomic mRNA synthesis (transcription) (an estimated 14-16 end products for coronaviruses) are encoded within the 50-proximal two-thirds of the genome on gene 1 and the (mostly) structural proteins are encoded within the 30-proximal one-third of the genome (8-9 genes for coronaviruses). Genes for the major structural proteins in all coronaviruses occur in 5′ to 3′ order as S (spike), E (envelope), M, and N. The genome sizes range from 27,317 nt for HCoV-229E to 31,357 nt for MHV-A59.

In general, coronavirus 5′ UTRs range in length from 209 to 528 nt and contain a similarly positioned short, AUG-initiated open reading frame (ORF) relative to the 5′ end. The short AUG-initiated ORFs (except for HCoV-229E) begin in a suboptimal Kozak context for translation and potentially encode peptides of 3-11 amino acids. The 3′ UTRs range from 288 to 506 nt and possess an octameric sequence of GGAAGAGC beginning at base 73 to 80 upstream from the poly(A) tail, and 30-terminal poly(A) tail.

In general, the coronaviruses have a large gene 1 (separated into ORFs 1a and 1b and extending over approximately two-thirds of the genome) encoding nonstructural proteins involved in proteolytic processing of the gene 1 polyprotein products, virus genome replication, and sgmRNA synthesis (transcription). Gene 1 is translated as ORFs 1a and 1ab, with 1ab resulting from a pseudoknot-induced −1 ribosomal frame shifting event at a slippery sequence of UUUAAAC at the ORF 1a/1b junction.

Within the 3′-proximal one-third of the genome, besides ORFs encoding the structural proteins, a variable number of other ORFs appearing to be virus- or group-specific, many apparently encoding nonstructural proteins, are also found here. These (and their potential products) include ORF 3a (7.7-kDa protein), ORF 3b (27.7-kDa protein), and ORF 7 [0.7-kDa hydrophobic protein (HP)] in TGEV; ORF 3 (25.3-kDa protein) in PEDV; ORF 4a (15.3-kDa protein) and ORF 4b (10.2-kDa protein) in HCoV-229E; ORF 2a (32-kDa protein), ORF 2b [65-kDa complete or 34.6-kDa truncated hemagglutininesterase (HE) protein, depending on the strain], ORF 4 (17.8-kDa protein), ORF 5a (13.1-kDa protein), and an ORF internal to gene 7 [23-kDa internal (I) protein] in MHV; ORF 2a (32-kDa protein), ORF 2b (65-kDa HE protein), ORF 4a (4.9-kDa protein), ORF 4b (4.8-kDa protein), ORF 5 (12.7-kDa protein), and an ORF internal to gene 7 (23-kDa I protein) in BCoV; and ORF 3a (6.7-kDa protein), ORF 3b (7.4-kDa protein), ORF 5a (7.5-kDa protein), and ORF 5b (9.5-kDa protein) in IBV. Some of these, such as ORFs 3a and 3b in TGEV and ORFs 2a, 2b (HE), 4, 5a, and I in MHV, have been shown to be nonessential for replication in cell culture.

Coronaviruses (order Nidovirales, family Coronaviridae, and subfamily Orthocoronavirinae) are spherical (125 nm diameter), and enveloped with club-shaped spikes on the surface giving the appearance of a solar corona. Within the helically symmetrical nucleocapsid is the large positive sense, single stranded RNA. With genome sizes ranging from 26 to 32 kilobases (kb) in length, CoVs have the largest genome for RNA viruses. Of the four coronavirus genera (α, β, γ, δ), human coronaviruses (HCoVs) are classified under α-CoV and β-CoV. SARS-CoV-2 is a β-CoV.

Replication of coronaviruses begins with attachment and entry. Attachment of the virus to the host cell is initiated by interactions between the S protein and its specific receptor. Following receptor binding, the virus enters host cell cytosol via cleavage of S protein by a protease enzyme, followed by fusion of the viral and cellular membranes. The next step is the translation of the replicase gene from the virion genomic RNA and then translation and assembly of the viral replicase complexes. Following replication and subgenomic RNA synthesis, encapsidation occurs resulting in the formation of the mature virus. Following assembly, virions are transported to the cell surface in vesicles and released by exocytosis.

For SARS-Cov-2, next-generation sequencing also shows 79% homology to SARS-CoV and 50% to MERS-CoV. Phylogenetic analysis has placed SARS-CoV-2 under the subgenus Sarbecovirus of the genus Betacoronavirus.

The organization of the coronavirus genome is 5′-leader-UTR-replicase-S (Spike)-E (Envelope)-M (Membrane)-N(Nucleocapsid)-3′UTR-poly(A) tail with accessory genes interspersed within the structural genes at the 3′ end of the genome.

The four structural proteins are required by most CoVs to produce a structurally complete viral particle, suggesting that some CoVs may encode additional proteins with overlapping compensatory functions. While each of the major protein plays a primary role in the structure of the virus particle, they are also involved in other aspects of the replication cycle.

The S protein (about 150 kDa) mediates attachment of the virus to the host cell surface receptors resulting in fusion and subsequent viral entry. In some CoVs, the S protein also mediate cell-cell fusion between infected and adjacent, uninfected cells resulting in formation of multinucleated giant cells, a strategy that allows direct viral spread between cells while avoiding virus-neutralizing antibodies.

The S protein utilizes an N-terminal signal sequence to gain access into the endoplasmic reticulum (ER), and is heavily N-linked glycosylated. Homotrimers of the virus-encoded S protein make up the distinctive spike-like structure. This trimeric S glycoprotein is a class I fusion protein that mediates attachment to the host receptor. In most coronaviruses, S is cleaved by a host cell furin-like protease into two separate polypeptides, namely S1 and S2. S1 makes up the large receptor-binding domain of the S protein and S2 forms the stalk of the spike.

The M protein (about 25-30 kDa) with three transmembrane domains is the most abundant structural protein and defines the shape of the viral envelope. It has a small N-terminal glycosylated ectodomain and a much larger C-terminal endodomain that extends 6-8 nm into the viral particle. Studies have shown that the M protein exists as a dimer, and may adopt two different conformations allowing it to promote membrane curvature as well as bind to the nucleocapsid. Interaction of S with M protein is necessary for retention of S in the ER-Golgi intermediate compartment (ERGIC)/Golgi complex and its incorporation into new virions, but is not required for the assembly process.44 Binding of M to N protein stabilises the nucleocapsid (N protein-RNA complex), as well as the internal core of virions, and, ultimately, helps complete the viral assembly. Together, M and E proteins make up the viral envelope and their interaction is sufficient for the production and release of virus-like particles (VLPs).

The E protein (about 8-12 kDa) is the smallest of the major structural proteins. This transmembrane protein has a N-terminal ectodomain and a C-terminal endodomain with ion channel activity. During the replication cycle, E is abundantly expressed inside the infected cell, but only a small portion is incorporated into the virus envelope. The majority of the protein participates in viral assembly and budding. Recombinant CoVs without E have been shown to exhibit significantly reduced viral titres, crippled viral maturation, or yield incompetent progeny, thereby demonstrating the importance of E protein in virus production and maturation.

The N protein is the only one that binds to the RNA genome. The protein is composed of two separate domains, an N-terminal domain (NTD) and a C-terminal domain (CTD). It has been suggested that optimal RNA binding requires contribution from both these domains. It is also involved in viral assembly and budding, resulting in complete virion formation.

The SARS-CoV-2 genome is similar to that of typical CoVs and contains at least ten open reading frames (ORFs). The 5′-terminal two-thirds of the genome ORF1a/b encodes two large polyproteins, which form the viral replicase transcriptase complex. The other ORFs of SARSCoV-2 on the one-third of the genome encode the same four main structural proteins: spike(S), envelope (E), nucleocapsid (N) and membrane (M) proteins, as well as several accessory proteins with unknown functions which do not participate in viral replication.

Attachment of the virus to the host cell is initiated by interactions between the S protein and its receptor. The site of receptor binding domains (RBD) within the S1 region of a coronavirus S protein varies for each coronavirus. MHV have the RBD at the N-terminus, whereas SARS-CoV have the RBD at the C-terminus. The S-protein/receptor interaction is the primary determinant to infect a host species and also controls viral tissue tropism. Many coronaviruses utilize peptidases as their cellular receptor however, SARS-CoV and HCoV-NL63 use angiotensin-converting enzyme 2 (ACE2) as their receptor.

Following receptor binding, the virus enters host cell cytosol via acid-dependent proteolytic cleavage of S protein by a cathepsin, TMPRRS2 or another protease, followed by fusion of the viral and cellular membranes. S protein cleavage occurs at two sites within the S2 portion of the protein, with the first cleavage for separating the RBD and fusion domains of the S protein, and the second to expose the fusion peptide (cleavage at S2′). Cleavage at S2′ exposes a fusion peptide that inserts into the membrane, followed by the joining of two heptad repeats in S2 forming an antiparallel six-helix bundle. The formation of this bundle results in fusion and ultimate release of the viral genome into the cytoplasm.

The next step in the coronavirus lifecycle is the translation of the replicase gene from the virion genomic RNA. Coronaviruses encode either two or three proteases that cleave the replicase polyproteins. Next, many of the nonstructural proteins (nsps) assemble into the replicase-transcriptase complex (RTC) to create an environment suitable for RNA synthesis, and ultimately are responsible for RNA replication and transcription of the sub-genomic RNAs. The nsps also contain other enzyme domains and functions.

Viral RNA synthesis follows the translation and assembly of the viral replicase complexes. Viral RNA synthesis produces both genomic and sub-genomic RNAs. Sub-genomic RNAs serve as mRNAs for the structural and accessory genes which reside downstream of the replicase polyproteins. All positive-sense sub-genomic RNAs are 3′ co-terminal with the full-length viral genome and thus form a set of nested RNAs, a distinctive property of the order Nidovirales. Both genomic and sub-genomic RNAs are produced through negative-strand intermediates. These negative-strand intermediates are only about 1% as abundant as their positive-sense counterparts and contain both poly-uridylate and anti-leader sequences.

Coronaviruses are also known for their ability to recombine using both homologous and non-homologous recombination. The ability of these viruses to recombine is tied to the strand switching ability of the RNA-dependent RNA polymerase (RdRp). It is likely that recombination plays a significant role in viral evolution and is the basis for targeted RNA recombination, a reverse genetics tool used to engineer viral recombinants at the 3′ end of the genome.

Following replication and subgenomic RNA synthesis, the S, E, and M proteins are translated and inserted into the endoplasmic reticulum (ER). These proteins move along the secretory pathway into the endoplasmic reticulum-Golgi intermediate compartment (ERGIC). In the compartment, the viral genomes that are encapsidated by the N protein, will bud into the membrane resulting in formation of the mature virus.

The M protein directs most of the protein-protein interactions required for coronaviruses assembly. However, virus-like particles (VLPs) can only be formed when M protein is expressed along with E protein, suggesting the need for these two proteins to produce coronavirus envelope. Additional roles of the E protein include inducing membrane curvature and preventing M protein aggregation. Following assembly, virions are transported to the cell surface in vesicles and released by exocytosis.

Some subsets of b-CoVs also comprise a fifth structural protein, hemagglutinin-esterase (HE), which enhances S protein-mediated cell entry and viral spread through the mucosa via its acetyl-esterase activity. Homo-trimers of the S glycoprotein make up the distinctive spike structure on the surface of the virus. These trimers are a class I fusion protein, mediating virus attachment to the host receptor by interaction of the S protein and its receptor. In most CoVs, S is cleaved by host cell protease into two separate polypeptides-S1 and S2. S1 contains the receptor-binding domain (RBD) of the S protein (the exact positioning of the RBD varies depending on the viral strain), while S2 forms the stem of the spike molecule.

The S domain, which comprises S1 and S2 domains, responsible for receptor binding and cell membrane fusion respectively.

### Methods

In one embodiment, coronavirus is serially passaged in cells at progressively lower temperatures. The cells may be infected and then cultured for 1, 2, 3, 4 or more days at one temperature, e.g., 35-37° C., supernatant is collected then used to infect fresh cells at a lower temperature, e.g., from 0.5 to 2 degrees lower such as from 33° C. to 34° C. and cultured for 1, 2, 3, 4 or more days at that temperature. That is repeated until a specific temperature or temperature range is reached, e.g., 25-27° C.

In one embodiment, prior to infection with coronavirus, the cells are cold adapted. In one embodiment, the cells are mammalian cells such as Vero cells, HT-29 cells, CaCO-2 cells, HeLa cells, HEP-2 cells, HCT-8 cells, HL-60 cells, A549 cells, VeroE6-TMPRSS2 cells, or VeroE6-TMPRSS2-ACE2.

There is also provided a vector comprising a nucleic acid molecule of the invention having a coronavirus genome from a cold-adapted coronavirus or a portion thereof.

Optionally a vector of the disclosure comprises a nucleic acid molecule comprising a coronavirus genome comprising a nucleic acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% nucleic acid sequence identity over its entire length to one of SEQ ID Nos. 1-4, and comprises one or more mutations relative to one of SEQ ID Nos. 1-4, that is/are disclosed herein.

Viral vaccine vectors may use live viruses to deliver nucleic acid (for example, DNA or RNA) into human or non-human animal cells. The nucleic acid contained in the virus encodes one or more antigens that, once expressed in the infected human or non-human animal cells, elicit an immune response. Both humoral and cell-mediated immune responses can be induced by viral vaccine vectors. Viral vaccine vectors combine many of the positive qualities of nucleic acid vaccines with those of live attenuated vaccines. Like nucleic acid vaccines, viral vaccine vectors carry nucleic acid into a host cell for production of antigenic proteins that can be tailored to stimulate a range of immune responses, including antibody, T helper cell (CD4+ T cell), and cytotoxic T lymphocyte (CTL, CD8+ T cell) mediated immunity. Viral vaccine vectors, unlike nucleic acid vaccines, also have the potential to actively invade host cells and replicate, much like a live attenuated vaccine, further activating the immune system like an adjuvant. A viral vaccine vector therefore generally comprises a live attenuated virus that is genetically engineered to carry nucleic acid (for example, DNA or RNA) encoding protein antigens. Although viral vaccine vectors may produce stronger immune responses than nucleic acid vaccines, for some diseases viral vectors are used in combination with other vaccine technologies in a strategy called heterologous prime-boost. In this system, one vaccine is given as a priming step, followed by vaccination using an alternative vaccine as a booster. The heterologous prime-boost strategy aims to provide a stronger overall immune response. Viral vaccine vectors may be used as both prime and boost vaccines as part of this strategy.

There is also provided a method of inducing an immune response to a coronavirus in a subject, which comprises administering to the subject an effective amount of a cold-adapted coronavirus or a portion thereof, a nucleic acid having a nucleotide sequence of a cold-adapted coronavirus or a portion thereof, a vector having a nucleotide sequence of a cold-adapted coronavirus or a portion thereof, or a pharmaceutical composition having a cold-adapted coronavirus or a portion thereof.

There is also provided a method of immunizing a subject against a cold-adapted coronavirus or a portion thereof, which comprises administering to the subject an effective amount of a cold-adapted coronavirus, a nucleic acid a nucleotide sequence of a cold-adapted coronavirus or a portion thereof, a vector having a nucleotide sequence of a cold-adapted coronavirus or a portion thereof, or a pharmaceutical composition having a cold-adapted coronavirus or a portion thereof.

There is further provided a cold-adapted coronavirus, a nucleotide sequence of a cold-adapted coronavirus or a portion thereof, a vector having a nucleotide sequence of a cold-adapted coronavirus or a portion thereof, or a pharmaceutical composition having a cold-adapted coronavirus or a portion thereof, for use as a medicament.

There is further provided a cold-adapted coronavirus or a portion thereof, a nucleotide sequence of a cold-adapted coronavirus or a portion thereof, a vector having a nucleotide sequence of a cold-adapted coronavirus or a portion thereof, or a pharmaceutical composition having a cold-adapted coronavirus or a portion thereof, for use in the prevention, treatment, or amelioration of a coronavirus infection.

There is also provided use of a cold-adapted coronavirus or a portion thereof, a nucleotide sequence of a cold-adapted coronavirus or a portion thereof, a vector having a nucleotide sequence of a cold-adapted coronavirus or a portion thereof, or a pharmaceutical composition having a cold-adapted coronavirus or a portion thereof, in the manufacture of a medicament for the prevention, treatment, or amelioration of a coronavirus infection.

### Administration

Any suitable route of administration may be used. Methods of administration include, but are not limited to, intradermal, intramuscular, intraperitoneal, parenteral, intravenous, subcutaneous, vaginal, rectal, intranasal, inhalation or oral. Parenteral administration, such as subcutaneous, intravenous or intramuscular administration, is generally achieved by injection. Injectables can be prepared in conventional forms, either as liquid solutions or suspensions, solid forms suitable for solution or suspension in liquid prior to injection, or as emulsions.

Injection solutions and suspensions can be prepared from sterile powders, granules, and tablets of the kind previously described. Administration can be systemic or local.

Compositions may be administered in any suitable manner, such as with pharmaceutically acceptable carriers. Pharmaceutically acceptable carriers are determined in part by the particular composition being administered, as well as by the particular method used to administer the composition. Preparations for parenteral administration include sterile aqueous or nonaqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Administration can be accomplished by single or multiple doses. The dose administered to a subject in the context of the present disclosure should be sufficient to induce a beneficial therapeutic response in a subject over time, or to inhibit or prevent infection. The dose required will vary from subject to subject depending on the species, age, weight and general condition of the subject, the severity of the infection being treated, the particular composition being used and its mode of administration. An appropriate dose can be determined by one of ordinary skill in the art using only routine experimentation.

Pharmaceutically acceptable carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, and magnesium carbonate. Any of the common pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives and the like. Other media that can be used with the compositions and methods provided herein are normal saline and sesame oil.

In some embodiments, the compositions comprise a pharmaceutically acceptable carrier and/or an adjuvant. For example, the adjuvant can be alum, Freund's complete adjuvant, a biological adjuvant or immunostimulatory oligonucleotides (such as CpG oligonucleotides).

The pharmaceutically acceptable carriers (vehicles) useful in this disclosure are conventional. Remington's Pharmaceutical Sciences, by E. W. Martin, Mack Publishing Co., Easton, PA, 15th Edition (1975), describes compositions and formulations suitable for pharmaceutical delivery of one or more therapeutic compositions, such as one or more vaccines, and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Optionally a composition is administered intramuscularly.

Optionally a composition is administered intramuscularly, intradermally, subcutaneously by needle or by gene gun, or electroporation.

A vaccine of the disclosure includes an isolated cold-adapted virus of the disclosure, and optionally one or more other isolated viruses including influenza viruses, one or more immunogenic proteins or glycoproteins of one or more isolated viruses or one or more other pathogens, e.g., an immunogenic protein from one or more bacteria, influenza viruses, yeast or fungi, or isolated nucleic acid encoding one or more viral proteins (e.g., DNA vaccines) including one or more immunogenic proteins of the isolated cold-adapted coronavirus of the disclosure. In one embodiment, the cold-adapted coronavirus of the disclosure may be a vaccine vectors for other pathogens.

A complete virion vaccine may be concentrated by ultrafiltration and then purified by zonal centrifugation or by chromatography. Viruses other than the virus of the disclosure, such as those included in a multivalent vaccine, may be inactivated before or after purification using formalin or beta-propiolactone, for instance.

A subunit vaccine comprises purified glycoproteins. Such a vaccine may be prepared as follows: using viral suspensions fragmented by treatment with detergent, the surface antigens are purified, by ultracentrifugation for example. The detergent used may be cationic detergent for example, such as hexadecyl trimethyl ammonium bromide, an anionic detergent such as ammonium deoxycholate; or a nonionic detergent such as that commercialized under the name TRITON X100. The hemagglutinin may also be isolated after treatment of the virions with a protease such as bromelin, and then purified. A subunit vaccine may be combined with an attenuated virus of the disclosure in a multivalent vaccine.

A split vaccine comprises virions which have been subjected to treatment with agents that dissolve lipids. A split vaccine can be prepared as follows: an aqueous suspension of the purified virus obtained as above, inactivated or not, is treated, under stirring, by lipid solvents such as ethyl ether or chloroform, associated with detergents. The dissolution of the viral envelope lipids results in fragmentation of the viral particles. The aqueous phase is recuperated containing the split vaccine, constituted mainly of hemagglutinin and neuraminidase with their original lipid environment removed, and the core or its degradation products. Then the residual infectious particles are inactivated if this has not already been done. A split vaccine may be combined with an attenuated virus of the disclosure in a multivalent vaccine.

Inactivated virus vaccines are provided by inactivating replicated virus using known methods, such as, but not limited to, formalin or β-propiolactone treatment. Inactivated vaccine types that can be used in the disclosure can include whole-virus (WV) vaccines or subvirion (SV) (split) vaccines. The WV vaccine contains intact, inactivated virus, while the SV vaccine contains purified virus disrupted with detergents that solubilize the lipid-containing viral envelope, followed by chemical inactivation of residual virus. An inactivated virus vaccine may be combined with an attenuated virus of the disclosure in a multivalent vaccine.

Live, attenuated virus vaccines, such as those including a cold-adapted coronavirus of the disclosure can be used for preventing, inhibiting or treating coronavirus infection. Attenuation may be achieved in a single step by transfer of attenuated genes from an attenuated donor virus to a recipient virus according to known methods. The attenuated genes are derived from an attenuated parent.

A cold adapted (ca) donor virus can be used for attenuated vaccine production. Live, attenuated virus vaccines can be generated that are: (a) infectious, (b) attenuated for seronegative mammals and immunologically primed mammals, (c) immunogenic and/or (d) genetically stable.

One or more attenuating mutations can be introduced into virus genes by site-directed mutagenesis to rescue infectious viruses bearing these mutant genes. Attenuating mutations can be introduced into non-coding regions of the genome, as well as into coding regions. Thus, new dviruses can also be generated bearing attenuating mutations introduced by site-directed mutagenesis, and such new viruses can be used in the production of live attenuated vaccine candidates. Similarly, other known and suitable attenuated strains can be employed with one or more of the mutations disclosed herein to obtain attenuated vaccines suitable for use in the vaccination of mammals.

In one embodiment, such attenuated viruses maintain the genes from the virus that encode antigenic determinants substantially similar to those of the original clinical isolates. This is because the purpose of the attenuated vaccine is to provide substantially the same antigenicity as the original clinical isolate of the virus, while at the same time lacking pathogenicity to the degree that the vaccine causes minimal chance of inducing a serious disease condition in the vaccinated mammal.

The viruses in a multivalent vaccine can thus be attenuated or inactivated, formulated and administered, according to known methods, as a vaccine to induce an immune response in an animal, e.g., a mammal. Methods are well-known in the art for determining whether such attenuated or inactivated vaccines have maintained similar antigenicity to that of the clinical isolate or high growth strain derived therefrom.

In one embodiment, a vaccine comprises a dose of the cold-adapted virus that is 10² to 10⁵ TCID₅₀. In one embodiment, a vaccine comprises a dose of the cold-adapted virus that is 10³ to 10⁶ TCID₅₀. In one embodiment, a vaccine comprises a dose of the cold-adapted virus that is 10⁴ to 10⁷ TCID₅₀. In one embodiment, a vaccine comprises a dose of the cold-adapted virus that is 10⁵ to 10⁸ TCID₅₀.

### Pharmaceutical Compositions

Pharmaceutical compositions of the present disclosure, suitable for inoculation, e.g., nasal, parenteral or oral administration, comprise one or more virus isolates, e.g., one or more cold-adapted SARS-CoV-2 viruses, a subunit thereof, isolated protein(s) thereof, and/or isolated nucleic acid encoding one or more proteins thereof, optionally further comprising sterile aqueous or non-aqueous solutions, suspensions, and emulsions. The compositions can further comprise auxiliary agents or excipients, as known in the art. The composition of the disclosure is generally presented in the form of individual doses (unit doses).

Conventional vaccines generally contain about 0.1 to 200 μg, e.g., 1 to 5 ug, 5 to 20 μg, 10 to 30 μg or 30 to 100 μg, of S protein from each of the strains entering into the composition. The vaccine forming the main constituent of the vaccine composition of the disclosure may comprise a single cold-adapted SARS-CoV-2 virus, or a combination of viruses, for example, at least two or three different viruses, including one or more other coronaviruses.

Preparations for parenteral administration include sterile aqueous or non-aqueous solutions, suspensions, and/or emulsions, which may contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents.

When a composition of the present disclosure is used for administration to an individual, it can further comprise salts, buffers, adjuvants, or other substances which are desirable for improving the efficacy of the composition. For vaccines, adjuvants, substances which can augment a specific immune response, can be used. Normally, the adjuvant and the composition are mixed prior to presentation to the immune system, or presented separately, but into the same site of the organism being immunized.

Heterogeneity in a vaccine may be provided by mixing viruses for at least two virus strains, such as 2-20 strains or any range or value therein. Vaccines can be provided for variations in a single strain of virus, using techniques known in the art.

A pharmaceutical composition according to the present disclosure may further or additionally comprise at least one chemotherapeutic compound, for example, for gene therapy, immunosuppressants, anti-inflammatory agents or immune enhancers, and for vaccines, chemotherapeutics including, but not limited to, gamma globulin, amantadine, guanidine, hydroxybenzimidazole, interferon-α, interferon-β, interferon-γ, tumor necrosis factor-alpha, thiosemicarbarzones, methisazone, rifampin, ribavirin, a pyrimidine analog, a purine analog, foscarnet, phosphonoacetic acid, acyclovir, dideoxynucleosides, a protease inhibitor, or ganciclovir.

The composition can also contain variable but small quantities of endotoxin-free formaldehyde, and preservatives, which have been found safe and not contributing to undesirable effects in the organism to which the composition is administered.

The administration of the cold-adapted SARS-CoV-2 virus containing composition (or the antisera that it elicits) may be for either a “prophylactic” or “therapeutic” purpose. When provided prophylactically, the compositions of the disclosure which are vaccines are provided before any symptom or clinical sign of a pathogen infection becomes manifest. The prophylactic administration of the composition serves to prevent or attenuate any subsequent infection.

When provided therapeutically, a viral vaccine is provided upon the detection of a symptom or clinical sign of actual infection. The therapeutic administration of the compound(s) serves to attenuate any actual infection.

Thus, a vaccine composition of the present disclosure may be provided either before the onset of infection (so as to prevent or attenuate an anticipated infection) or after the initiation of an actual infection.

A composition is said to be “pharmacologically acceptable” if its administration can be tolerated by a recipient mammal. Such an agent is said to be administered in a “therapeutically effective amount” if the amount administered is physiologically significant. A composition of the present disclosure is physiologically significant if its presence results in a detectable change in the physiology of a recipient patient, e.g., enhances at least one primary or secondary humoral or cellular immune response against at least one strain of an infectious coronavirus.

The “protection” provided need not be absolute, i.e., the coronavirus infection need not be totally prevented or eradicated, if there is a statistically significant improvement compared with a control population or set of mammals. Protection may be limited to mitigating the severity or rapidity of onset of symptoms or clinical signs of the virus infection.

A composition comprising a cold-adapted SARS-CoV-2 virus may confer resistance to one or more pathogens, e.g., one or more SARS-CoV-2 strains, by either passive immunization or active immunization. In active immunization, an attenuated live vaccine composition is administered prophylactically to a host (e.g., a mammal), and the host's immune response to the administration protects against infection and/or disease. For passive immunization, the elicited antisera can be recovered and administered to a recipient suspected of having an infection caused by at least one virus strain.

In one embodiment, the vaccine is provided to a mammalian female (at or prior to pregnancy or parturition), under conditions of time and amount sufficient to cause the production of an immune response which serves to protect both the female and the fetus or newborn (via passive incorporation of the antibodies across the placenta or in the mother's milk).

The present disclosure thus includes methods for preventing or attenuating a disorder or disease, e.g., an infection by at least one strain of pathogen. As used herein, a vaccine is said to prevent or attenuate a disease if its administration results either in the total or partial attenuation (i.e., suppression) of a clinical sign or condition of the disease, or in the total or partial immunity of the individual to the disease.

A composition having at least one cold-adapted SARS-CoV-2 virus of the present disclosure, including one which is attenuated and one or more other isolated viruses, one or more isolated viral proteins thereof, one or more isolated nucleic acid molecules encoding one or more viral proteins thereof, or a combination thereof, may be administered by any means that achieve the intended purposes.

For example, administration of such a composition may be by various parenteral routes such as subcutaneous, intravenous, intradermal, intramuscular, intraperitoneal, intranasal, oral or transdermal routes. Parenteral administration can be accomplished by bolus injection or by gradual perfusion over time.

A typical regimen for preventing, suppressing, or treating a virus related pathology, comprises administration of an effective amount of a vaccine composition as described herein, administered as a single treatment, or repeated as enhancing or booster dosages, over a period up to and including between one week and about 24 months, or any range or value therein.

According to the present disclosure, an “effective amount” of a composition is one that is sufficient to achieve a desired effect. It is understood that the effective dosage may be dependent upon the species, age, sex, health, and weight of the recipient, kind of concurrent treatment, if any, frequency of treatment, and the nature of the effect wanted. The ranges of effective doses provided below are not intended to limit the disclosure and represent dose ranges.

The dosage of a live, attenuated or killed virus vaccine for an animal such as a mammalian adult organism may be from about 10²-10¹⁵, e.g., 10³-10¹², plaque forming units (PFU)/kg, or any range or value therein. The dose of inactivated vaccine may range from about 0.1 to 1000, e.g., 2.5 to 10 μg, of S protein. However, the dosage should be a safe and effective amount as determined by conventional methods, using existing vaccines as a starting point.

The dosage of immunoreactive S in each dose of replicated virus vaccine may be standardized to contain a suitable amount, e.g., 30 to 100 μg or any range or value therein, or the amount recommended by government agencies or recognized professional organizations.

The dosage of immunoreactive S in each dose of virus vaccine can be standardized to contain a suitable amount, e.g., 1-50 μg or any range or value therein, or the amount recommended by the U.S. Public Health Service (PHS), which is usually 15 μg per component for older children (greater than or equal to 3 years of age), and 7.5 μg per component for children less than 3 years of age. The quantity of NA can also be standardized, however, this glycoprotein can be labile during the processor purification and storage. Each 0.5-ml dose of vaccine may contains approximately 1-50 billion virus particles, for example 10 billion particles.

### Exemplary SARS-CoV-2 Genomes and Encoded Products

An exemplary cold-adapted coronavirus has one or more alterations in one or more of Nsp1, Nsp2, Nsp3, Nsp4, Nsp5, Nsp6, Nsp7, Nsp8, Nsp9, Nsp10, Nsp11, Nsp12, Nsp13, Nsp14, Nsp15, Nsp16, S, E, M, N, or any combination thereof relative to a:

The Nsp1 amino acid sequence is provided below as SEQ ID NO:20:

MESLVPGFNEKTHVQLSLPVLQVRDVLVRGFGDSVEEVLSEARQHLKD
GTCGLVEVEKGVLPQLEQPYVFIKRSDARTAPHGHVMVELVAELEGIQ
YGRSGETLGVLVPHVGEIPVAYRKVLLRKNGNKGAGGHSYGADLKSFD
LGDELGTDPYEDFQENWNTKHSSGVTRELMRELNGG

Nsp2 amino acid sequence is provided below as SEQ ID NO:21:

AYTRYVDNNFCGPDGYPLECIKDLLARAGKASCTLSEQLDFIDTK
RGVYCCREHEHEIAWYTERSEKSYELQTPFEIKLAKKFDTFNGECPN
FVFPLNSIIKTIQPRVEKKKLDGFMGRIRSVYPVASPNECNQMCLST
LMKCDHCGETSWQTGDFVKATCEFCGTENLTKEGATTCGYLPQNAVV
KIYCPACHNSEVGPEHSLAEYHNESGLKTILRKGGRTIAFGGCVFSY
VGCHNKCAYWVPRASANIGCNHTGVVGEGSEGLNDNLLEILQKEKVN
INIVGDFKLNEEIAIILASFSASTSAFVETVKGLDYKAFKQIVESCG
NFKVTKGKAKKGAWNIGEQKSILSPLYAFASEAARVVRSIFSRTLET
AQNSVRVLQKAAITILDGISQYSLRLIDAMMFTSDLATNNLVVMAYI
TGGVVQLTSQWLTNIFGTVYEKLKPVLDWLEEKFKEGVEFLRDGWEI
VKFISTCACEIVGGQIVTCAKEIKESVQTFFKLVNKFLALCADSIII
GGAKLKALNLGETFVTHSKGLYRKCVKSREETGLLMPLKAPKEIIFL
EGETLPTEVLTEEVVLKTGDLQPLEQPTSEAVEAPLVGTPVCINGLM
LLEIKDTEKYCALAPNMMVTNNTFTLKGG

Nsp3 amino acid sequence is provided below as SEQ ID NO:22:

APTKVTFGDDTVIEVQGYKSVNITFELDERIDKVLNEKCSAYTVELGTEV
NEFACVVADAVIKTLQPVSELLTPLGIDLDEWSMATYYLFDESGEFKLAS
HMYCSFYPPDEDEEEGDCEEEEFEPSTQYEYGTEDDYQGKPLEFGATSA
ALQPEEEQEEDWLDDDSQQTVGQQDGSEDNQTTTIQTIVEVQPQLEMEL
TPVVQTIEVNSFSGYLKLTDNVYIKNADIVEEAKKVKPTVVVNAANVYL
KHGGGVAGALNKATNNAMQVESDDYIATNGPLKVGGSCVLSGHNLAK
HCLHVVGPNVNKGEDIQLLKSAYENFNQHEVLLAPLLSAGIFGADPIHSL
RVCVDTVRTNVYLAVFDKNLYDKLVSSFLEMKSEKQVEQKIAEIPKEEV
KPFITESKPSVEQRKQDDKKIKACVEEVTTTLEETKFLTENLLLYIDING
NLHPDSATLVSDIDITFLKKDAPYIVGDVVQEGVLTAVVIPTKKAGGTTE
MLAKALRKVPTDNYITTYPGQGLNGYTVEEAKTVLKKCKSAFYILPSIIP
NEKQEILGTVSWNLREMLAHAEETRKLMPVCVETKAIVSTIQRKYKGIKI
QEGVVDYGARFYFYTSKTTVASLINTLNDLNETLVTMPLGYVTHGLNLEE
AARYMRSLKVPATVSVSSPDAVTAYNGYLTSSSKTPEEHFIETISLAGSY
KDWSYSGQSTQLGIEFLKRGDKSVYYT
SNPTTFHLDGEVITFDNLKTLLSLREVRTIKVFTTVDNINLHTQVVDMSM
TYGQQFGPTYLDGADVTKIKPHNSHEGKTFYVLPNDDTLRVEAFEYYHT
TDPSFLGRYMSALNHTKKWKYPQVNGLTSIKWADNNCYLATALLTLQQ
IELKFNPPALQDAYYRARAGEAANFCALILAYCNKTVGELGDVRETMSY
LFQHANLDSCKRVLNVVCKTCGQQQTTLKGVEAVMYMGTLSYEQFKK
GVQIPCTCGKQATKYLVQQESPFVMMSAPPAQYELKHGTFTCASEYTG
NYQCGHYKHITSKETLYCIDGALLTKSSEYKGPITDVFYKENSYTTTIKP
VTYKLDGVVCTEIDPKLDNYYKKDNSYFTEQPIDLVPNQPYPNASFDNF
KFVCDNIKFADDLNQLTGYKKPASRELKVTFFPDLNGDVVAIDYKHYTP
SFKKGAKLLHKPIVWHVNNATNKATYKPNTWCIRCLWSTKPVETSNSF
DVLKSEDAQGMDNLACEDLKPVSEEVVENPTIQKDVLECNVKTTEVVG
DIILKPANNSLKITEEVGHTDLMAAYVDNSSLTIKKPNELSRVLGLKTLA
THGLAAVNSVPWDTIANYAKPFLNKVVSTTTNIVTRCLNRVCTNYMPYF
FTLLLQLCTFTRSTNSRIKASMPTTIAKNTVKSVGKFCLEASFNYLKSPN
FSKLINIIIWFLLLSVCLGSLIYSTAALGVLMSNLGMPSYCTGYREGYLN
STNVTIATYCTGSIPCSVCLSGLDSLDTYPSLETIQITISSFKWDLTAFG
LVAEWFLAYILFTRFFYVLGLAAIMQLFFSYFAVHFISNSWLMWLIINLV
QMAPISAMVRMYIFFASFYYVWKSYVHVVDGCNSSTCMMCYKRNRATRVE
CTTIVNGVRRSFYVYANGGKGFCKLHNWNCVNCDTFCAGSTFISDEVAR
DLSLQFKRPINPTDQSSYIVDSVTVKNGSIHLYFDKAGQKTYERHSLSHF
VNLDNLRANNTKGSLPINVIVFDGKSKCEESSAKSASVYYSQLMCQPILL
LDQALVSDVGDSAEVAVKMFDAYVNTFSSTFNVPMEKLKTLVATAEAE
LAKNVSLDNVLSTFISAARQGFVDSDVETKDVVECLKLSHQSDIEVTGDS
CNNYMLTYNKVENMTPRDLGACIDCSARHINAQVAKSHNIALIWNVKD
FMSLSEQLRKQIRSAAKKNNLPFKLTCATTRQVVNVVTTKIALKGG

Nsp4 amino acid sequence is provided below as SEQ ID NO:23:

KIVNNWLKQLIKVTLVFLFVAAIFYLITPVHVMSKHTDFSSEIIGYKAID
GGVTRDIASTDTCFANKHADFDTWFSQRGGSYTNDKACPLIAAVITREVG
FVVPGLPGTILRTTNGDFLHFLPRVFSAVGNICYTPSKLIEYTDFATSAC
VLAAECTIFKDASGKPVPYCYDTNVLEGSVAYESLRPDTRYVLMDGSIIQ
FPNTYLEGSVRVVTTFDSEYCRHGTCERSEAGVCVSTSGRWVLNNDYYR
SLPGVFCGVDAVNLLTNMFTPLIQPIGALDISASIVAGGIVAIVVTCLAY
YFMRFRRAFGEYSHVVAFNTLLFLMSFTVLCLTPVYSFLPGVYSVIYLYL
TFYLTNDVSFLAHIQWMVMFTPLVPFWITIAYIICISTKHFYWFFSNYLK
RRVVFNGVSFSTFEEAALCTFLLNKEMYLKLRSDVLLPLTQYNRYLALYN
KYKYFSGAMDTTSYREAACCHLAKALNDFSNSGSDVLYQPPQTSITSAVL
Q

Nsp5 amino acid sequence is provided below as SEQ ID NO:24:

SGFRKMAFPSGKVEGCMVQVTCGTTTLNGLWLDDVVYCPRHVICTSED
MLNPNYEDLLIRKSNHNFLVQAGNVQLRVIGHSMQNCVLKLKVDTANP
KTPKYKFVRIQSGQTFSVLACYNGSPSGVYQCAMRPNFTIKGSFLNGSC
GSVGFNIDYDCVSFCYMHHMELPTGVHAGTDLEGNFYGPFVDRQTAQAA
GTDTTITVNVLAWLYAAVINGDRWFLNRFTTTLNDFNLVAMKYNYEPL
TQDHVDILGPLSAQTGIAVLDMCASLKELLQNGMNGRTILGSALLEDEF
TPFDVVRQCSGVTFQ

Nsp6 amino acid sequence is provided below as SEQ ID NO:25:

SAVKRTIKGTHHWLLLTILTSLLVLVQSTQWSLFFFLYENAFLPFAMGI
IAMSAFAMMFVKHKHAFLCLFLLPSLATVAYFNMVYMPASWVMRIMTWL
DMVDTSLSGFKLKDCVMYASAVVLLILMTARTVYDDGARRVWTLMNV
LTLVYKVYYGNALDQAISMWALIISVTSNYSGVVTTVMFLARGIVFMCV
EYCPIFFITGNTLQCIMLVYCFLGYFCTCYFGLFCLLNRYFRLTLGVYD
YLVSTQEFRYMNSQGLLPPKNSIDAFKLNIKLLGVGGKPCIKVATVQ

Nsp7 amino acid sequence is provided below as SEO ID NO: 26:

SKMSDVKCTSVVLLSVLQQLRVESSSKLWAQCVQLHNDILLAKDTTEA
FEKMVSLLSVLLSMQGAVDINKLCEEMLDNRATLQ

Nsp8 amino acid sequence is provided below as SEQ ID NO:27:

AIASEFSSLPSYAAFATAQEAYEQAVANGDSEVVLKKLKKSLNVAKS
EFDRDAAMQRKLEKMADQAMTQMYKQARSEDKRAKVTSAMQTMLFTM
LRKLDNDALNNIINNARDGCVPLNIIPLTTAAKLMVVIPDYNTYKNTC
DGTTFTYASALWEIQQVVDADSKIVQLSEISMDNSPNLAWPLIVTALR
ANSAVKLQ

Nsp9 amino acid sequence is provided below as SEQ ID NO:28:

NNELSPVALRQMSCAAGTTQTACTDDNALAYYNTTKGGRFVLALLSDL
QDLKWARFPKSDGTGTIYTELEPPCRFVTDTPKGPKVKYLYFIKGLNN
LNRGMVLGSLAATVRLQ

Nsp10 amino acid sequence is provided below as SEQ ID NO:29:

AGNATEVPANSTVLSFCAFAVDAAKAYKDYLASGGQPITNCVKMLCTH
TGTGQAITVTPEANMDQESFGGASCCLYCRCHIDHPNPKGFCDLKGKY
VQIPTTCANDPVGFTLKNTVCTVCGMWKGYGCSCDQLREPMLQ

Nsp11 amino acid sequence is provided below as SEQ ID NO:30:

SADAQSFLNGFAV

Nsp12 amino acid sequence is provided below as SEQ ID NO:31:

SADAQSFLNRVCGVSAARLTPCGTGTSTDVVYRAFDIYNDKVAGFAKFL
KTNCCRFQEKDEDDNLIDSYFVVKRHTFSNYQHEETIYNLLKDCPAVAK
HDFFKFRIDGDMVPHISRQRLTKYTMADLVYALRHFDEGNCDTLKEILV
TYNCCDDDYFNKKDWYDFVENPDILRVYANLGERVRQALLKTVQFCD
AMRNAGIVGVLTLDNQDLNGNWYDFGDFIQTTPGSGVPVVDSYYSLLM
PILTLTRALTAESHVDTDLTKPYIKWDLLKYDFTEERLKLFDRYFKYWD
QTYHPNCVNCLDDRCILHCANFNVLFSTVFPLTSFGPLVRKIFVDGVPF
VVSTGYHFRELGVVHNQDVNLHSSRLSFKELLVYAADPAMHAASGNLLL
DKRTTCFSVAALTNNVAFQTVKPGNFNKDFYDFVVSKGFFKEGSSVELK
HFFFAQDGNAAISDYDYYRYNLPTMCDIRQLLFVVEVVDKYFDCYDGG
CINANQVIVNNLDKSAGFPFNKWGKARLYYDSMSYEDQDALFAYTKRN
VIPTITQMNLKYAISAKNRARTVAGVSICSTMTNRQFHQKLLKSIAATR
GATVVIGTSKFYGGWHNMLKTVYSDVENPHLMGWDYPKCDRAMPNML
RIMASLVLARKHTTCCSLSHRFYRLANECAQVLSEMVMCGGSLYVKPG
GTSSGDATTAYANSVFNICQAVTANVNALLSTDGNKIADKYVRNLQHR
LYECLYRNRDVDTDFVNEFYAYLRKHFSMMILSDDAVVCFNSTYASQG
LVASIKNFKSVLYYQNNVFMSEAKCWTETDLTKGPHEFCSQHTMLVKQ
GDDYVYLPYPDPSRILGAGCFVDDIVKTDGTLMIERFVSLAIDAYPLTK
HPNQEYADVFHLYLQYIRKLHDELTGHMLDMYSVMLINDNTSRYWEPEF
YEAMYTPHTVLQ

Nsp13 amino acid sequence is provided below as SEQ ID NO:32:

AVGACVLCNSQTSLRCGACIRRPFLCCKCCYDHVISTSHKLVLSVNPYV
CNAPGCDVTDVTQLYLGGMSYYCKSHKPPISFPLCANGQVFGLYKNTC
VGSDNVTDFNAIATCDWTNAGDYILANTCTERLKLFAAETLKATEETFK
LSYGIATVREVLSDRELHLSWEVGKPRPPLNRNYVFTGYRVTKNSKVQI
GEYTFEKGDYGDAVVYRGTTTYKLNVGDYFVLTSHTVMPLSAPTLVPQ
EHYVRITGLYPTLNISDEFSSNVANYQKVGMQKYSTLQGPPGTGKSHFA
IGLALYYPSARIVYTACSHAAVDALCEKALKYLPIDKCSRIIPARARVE
CFDKFKVNSTLEQYVFCTVNALPETTADIVVFDEISMATNYDLSVVNAR
LRAKHYVYIGDPAQLPAPRTLLTKGTLEPEYFNSVCRLMKTIGPDMFLG
TCRRCPAEIVDTVSALVYDNKLKAHKDKSAQCFKMFYKGVITHDVSSAI
NRPQIGVVREFLTRNPAWRKAVFISPYNSQNAVASKILGLPTQTVDSSQ
GSEYDYVIFTQTTETAHSCNVNRFNVAITRAKVGILCIMSDRDLYDKLQ
FTSLEIPRRNVATLQ

Nsp14 amino acid sequence is provided below as SEQ ID NO:33:

AENVTGLFKDCSKVITGLHPTQAPTHLSVDTKFKTEGLCVDIPGIPKDM
TYRRLISMMGFKMNYQVNGYPNMFITREEAIRHVRAWIGFDVEGCHATR
EAVGTNLPLQLGFSTGVNLVAVPTGYVDTPNNTDFSRVSAKPPPGDQFK
HLIPLMYKGLPWNVVRIKIVQMLSDTLKNLSDRVVFVLWAHGFELTSM
KYFVKIGPERTCCLCDRRATCFSTASDTYACWHHSIGFDYVYNPFMIDV
QQWGFTGNLQSNHDLYCQVHGNAHVASCDAIMTRCLAVHECFVKRVD
WTIEYPIIGDELKINAACRKVQHMVVKAALLADKFPVLHDIGNPKAIKC
VPQADVEWKFYDAQPCSDKAYKIEELFYSYATHSDKFTDGVCLFWNCN
VDRYPANSIVCRFDTRVLSNLNLPGCDGGSLYVNKHAFHTPAFDKSAFV
NLKQLPFFYYSDSPCESHGKQVVSDIDYVPLKSATCITRQNLGGAVCRH
HANEYRLYLDAYNMMISAGFSLWVYKQFDTYNLWNTFTRLQ

Nsp15 amino acid sequence is provided below as SEQ ID NO:34:

SLENVAFNVVNKGHFDGQQGEVPVSIINNTVYTKVDGVDVELFENKTT
LPVNVAFELWAKRNIKPVPEVKILNNLGVDIAANTVIWDYKRDAPAHI
STIGVCSMTDIAKKPTETICAPLTVFFDGRVDGQVDLFRNARNGVLIT
EGSVKGLQPSVGPKQASLNGVTLIGEAVKTQFNYYKKVDGVVQQLPET
YFTQSRNLQEFKPRSQMEIDFLELAMDEFIERYKLEGYAFEHIVYGDF
SHSQLGGLHLLIGLAKRFKESPFELEDFIPMDSTVKNYFITDAQTGSS
KCVCSVIDLLLDDFVEIIKSQDLSVVSKVVKVTIDYTEISFMLWCKDG
HVETFYPKLQ

Nsp16 amino acid sequence is provided below as SEQ ID NO:35:

SSQAWQPGVAMPNLYKMQRMLLEKCDLQNYGDSATLPKGIMMNVAK
YTQLCQYLNTLTLAVPYNMRVIHFGAGSDKGVAPGTAVLRQWLPTGTL
LVDSDLNDFVSDADSTLIGDCATVHTANKWDLIISDMYDPKTKNVTKE
NDSKEGFFTYICGFIQQKLALGGSVAIKITEHSWNADLYKLMGHFAWW
TAFVTNVNASSSEAFLIGCNYLGKPREQIDGYVMHANYIFWRNTNPIQ
LSSYSLFDMSKFPLKLRGTAVMSLKEGQINDMILSLLSKGRLIIRENN
RVVISSDVLVNN

S amino acid sequence is provided below as SEQ ID NO:36:

MFVFLVLLPLVSSQCVNLTTRTQLPPAYTNSFTRGVYYPDKVFRSSVL
HSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVY
YHKNNKSWMESEFRVYSSANNCTFEYVSQPFLMDLEGKQGNFKNLREF
VFKNIDGYFKIYSKHTPINLVRDLPQGFSALEPLVDLPIGINITRFQT
LLALHRSYLTPGDSSSGWTAGAAAYYVGYLQPRTFLLKYNENGTITDA
VDCALDPLSETKCTLKSFTVEKGIYQTSNFRVQPTESIVRFPNITNLC
PFGEVFNATRFASVYAWNRKRISNCVADYSVLYNSASFSTFKCYGVSP
TKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPC
NGVEGENCYFPLQSYGFQPTNGVGYQPYRVVVLSFELLHAPATVCGPK
KSTNLVKNKCVNFNFNGLTGTGVLTESNKKFLPFQQFGRDIADTTDAV
RDPQTLEILDITPCSFGGVSVITPGTNTSNQVAVLYQGVNCTEVPVAI
HADQLTPTWRVYSTGSNVFQTRAGCLIGAEHVNNSYECDIPIGAGICA
SYQTQTNSPRRARSVASQSIIAYTMSLGAENSVAYSNNSIAIPTNFTI
SVTTEILPVSMTKTSVDCTMYICGDSTECSNLLLQYGSFCTQLNRALT
GIAVEQDKNTQEVFAQVKQIYKTPPIKDFGGFNFSQILPDPSKPSKRS
FIEDLLFNKVTLADAGFIKQYGDCLGDIAARDLICAQKFNGLTVLPPL
LTDEMIAQYTSALLAGTITSGWTFGAGAALQIPFAMQMAYRFNGIGVT
QNVLYENQKLIANQFNSAIGKIQDSLSSTASALGKLQDVVNQNAQALN
TLVKQLSSNFGAISSVLNDILSRLDKVEAEVQIDRLITGRLQSLQTYV
TQQLIRAAEIRASANLAATKMSECVLGQSKRVDFCGKGYHLMSFPQSA
PHGVVFLHVTYVPAQEKNFTTAPAICHDGKAHFPREGVFVSNGTHWFV
TQRNFYEPQIITTDNTFVSGNCDVVIGIVNNTVYDPLQPELDSFKEEL
DKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCC
SCGSCCKFDEDDSEPVLKGVKLHYT

ORF3a amino acid sequence is provided below as SEO ID NO. 37.

MDLFMRIFTIGTVTLKQGEIKDATPSDFVRATATIPIQASLPFGWLIV
GVALLAVFQSASKIITLKKRWQLALSKGVHFVCNLLLLFVTVYSHLLL
VAAGLEAPFLYLYALVYFLQSINFVRIIMRLWLCWKCRSKNPLLYDAN
YFLCWHTNCYDYCIPYNSVTSSIVITSGDGTTSPISEHDYQIGGYTEK
WESGVKDCVVLHSYFTSDYYQLYSTQLSTDTGVEHVTFFIYNKIVDEP
EEHVQIHTIDGSSGVVNPVMEPIYDEPTTTTSVPL

E amino acid sequence is provided below as SEQ ID NO:38:

MYSFVSEETGTLIVNSVLLFLAFVVFLLVTLAILTALRLCAYCCNIVN
VSLVKPSFYVYSRVKNLNSSRVPDLLV

M amino acid sequence is provided below as SEQ ID NO:39:

MADSNGTITVEELKKLLEQWNLVIGFLFLTWICLLQFAYANRNRFLYI
IKLIFLWLLWPVTLACFVLAAVYRINWITGGIAIAMACLVGLMWLSYF
IASFRLFARTRSMWSFNPETNILLNVPLHGTILTRPLLESELVIGAVI
LRGHLRIAGHHLGRCDIKDLPKEITVATSRTLSYYKLGASQRVAGDSG
FAAYSRYRIGNYKLNTDHSSSSDNIALLVQ

ORF6 amino acid sequence is provided below as SEQ ID NO:40:

MFHLVDFQVTIAEILLIIMRTFKVSIWNLDYIINLIIKNLSKSLTENK
YSQLDEEQPMEID

ORF7a amino acid sequence is provided below as SEO ID NO: 41:

MKIILFLALITLATCELYHYQECVRGTTVLLKEPCSSGTYEGNSPFHP
LADNKFALTCFSTQFAFACPDGVKHVYQLRARSVSPKLFIRQEEVQEL
YSPIFLIVAAIVFITLCFTLKRKTE

ORF7b amino acid sequence is provided below as SEQ ID NO:42:

MIELSLIDFYLCFLAFLLFLVLIMLIIFWFSLELQDHNETCHA

ORF8 amino acid sequence is provided below as SEQ ID NO:43:

MKFLVFLGIITTVAAFHQECSLQSCTQHQPYVVDDPCPIHFYSKWYIRV
GARKSAPLIELCVDEAGSKSPIQYIDIGNYTVSCLPFTINCQEPKLGSL
VVRCSFYEDFLEYHDVRVVLDFI

N amino acid sequence is provided below as SEQ ID NO:44:

MSDNGPQNQRNAPRITFGGPSDSTGSNQNGERSGARSKQRRPQGLPNNT
ASWFTALTQHGKEDLKFPRGQGVPINTNSSPDDQIGYYRRATRRIRGGD
GKMKDLSPRWYFYYLGTGPEAGLPYGANKDGIIWVATEGALNTPKDHI
GTRNLANNAAIVLQLPQGTTLPKGFYAEGSRGGSQASSRSSSRSRNSSR
NSTPGSSKRTSPARMAGNGGDAALALLLLDRLNQLESKMSGKGQQQQGQ
TVTKKSAAEASKKPRQKRTATKAYNVTQAFGRRGPEQTQGNFGDQELI
RQGTDYKHWPQIAQFAPSASAFFGMSRIGMEVTPSGTWLTYTGAIKLDD
KDPNFKDQVILLNKHIDAYKTFPPTEPKKDKKKKADETQALPQRQKKQQ
TVTLLPAADLDDFSKQLQQSMSSADSTQA

ORF10 amino acid sequence is provided below as SEQ ID NO:45:

MGYINVFAFPFTIYSLLLCRMNSRNYIAQVDVVNFNLT

or a polypeptide having at least 80%, 82%, 84%, 85%, 87%, 89%, 90%, 92%, 94%, 95%, 96%, 97%, 98% or 99% amino acid sequence identity to any of Nsp1, Nsp2, Nsp3, Nsp4, Nsp5, Nsp6, Nsp7, Nsp8, Nsp9, Nsp10, Nsp11, Nsp12, Nsp13, Nsp14, Nsp15, Nsp16, S, E, M, N, ORF3a, ORF6, ORF7a, ORF7b, ORF8, and ORF10.

In one embodiment, a cold-adapted coronavirus has a mutation in one or more of genes for Nsp1, Nsp2, Nsp3, Nsp6, Nsp13, Nsp14, Nsp15, ORF6, ORF7b, ORF8, or any combination thereof. In one embodiment, a cold-adapted coronavirus comprise nucleic acids encoding a polypeptide for Nsp1, Nsp2, Nsp3, Nsp6, Nsp13, Nsp14, Nsp15, or a polypeptide encoded by ORF6, with at least 80%, 82%, 84%, 85%, 87%, 89%, 90%, 92%, 94%, 95%, 96%, 97%, 98% or 99% amino acid sequence identity to one of SEQ ID Nos. 20-22, 25, 32-34, or 40.

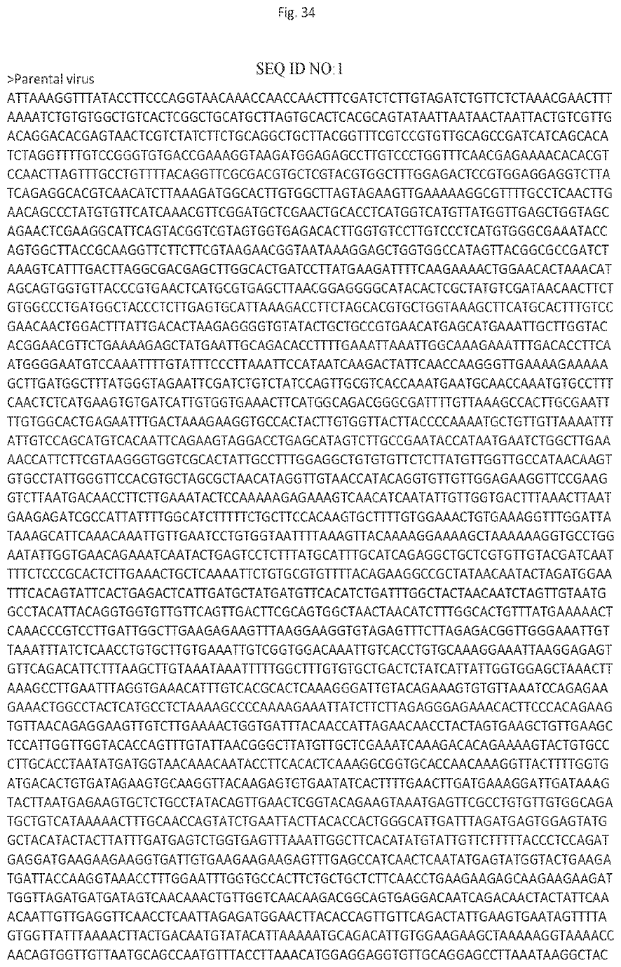

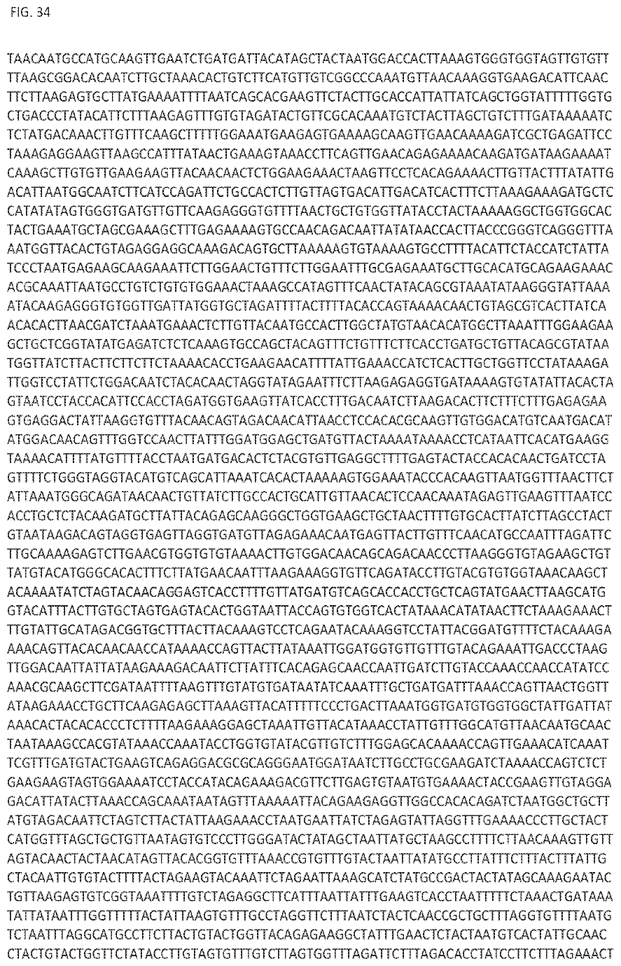

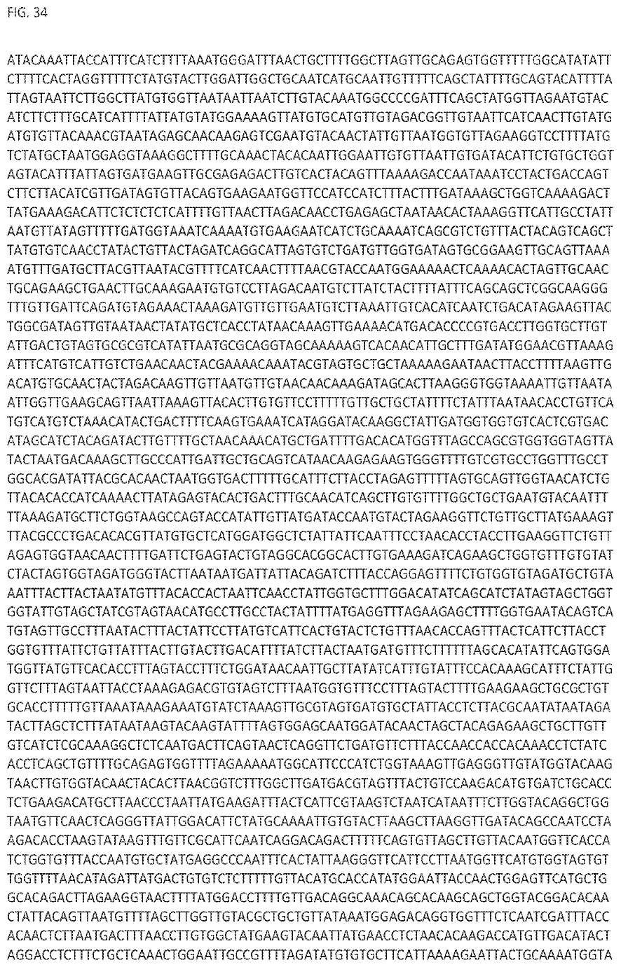

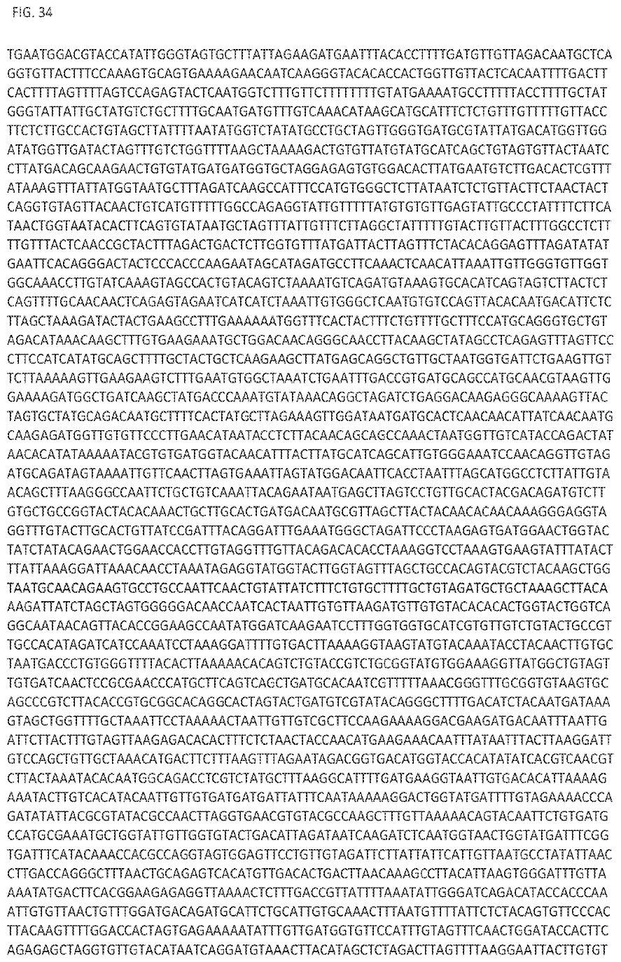

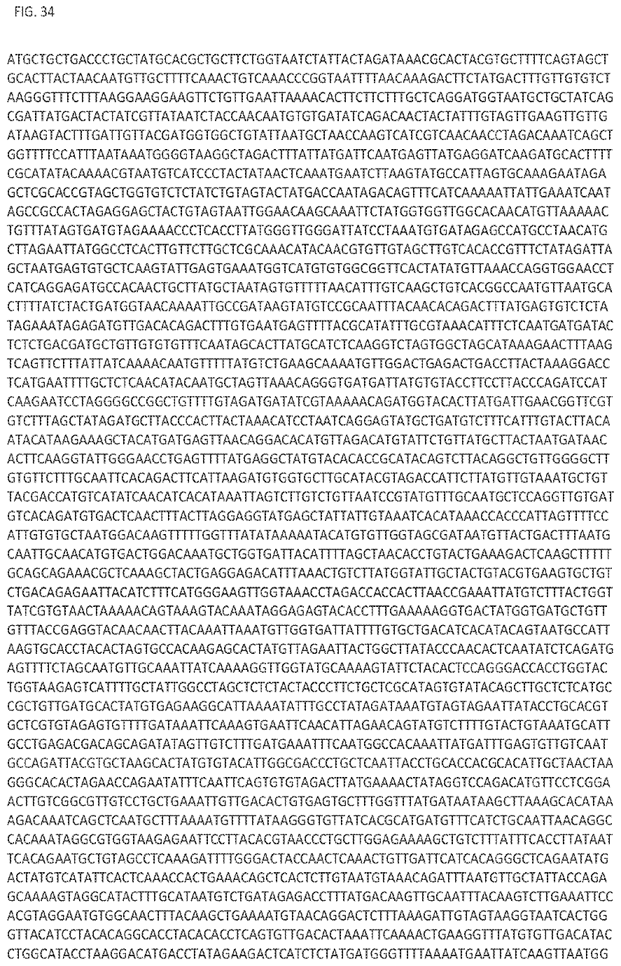

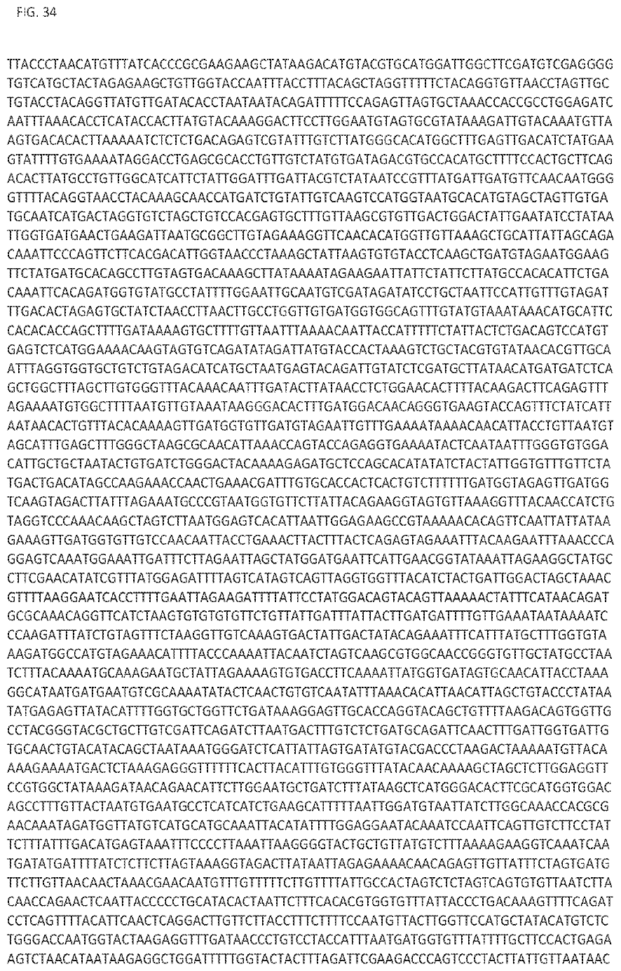

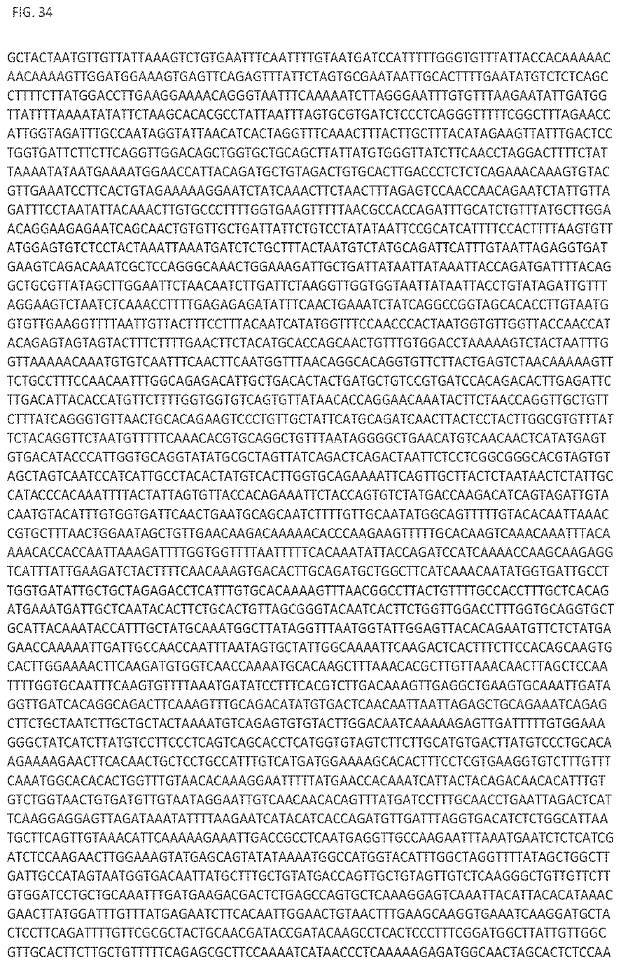

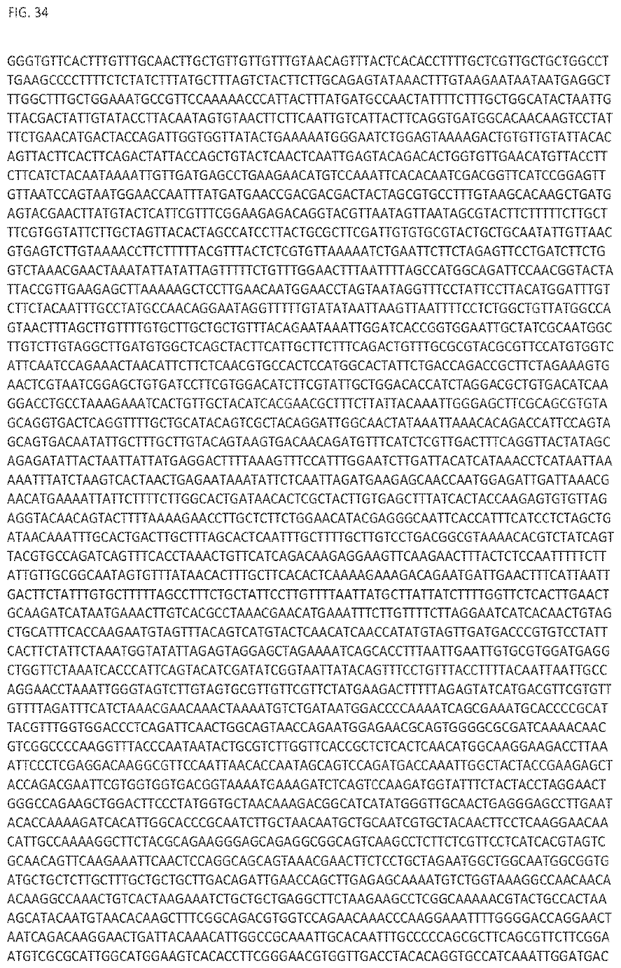

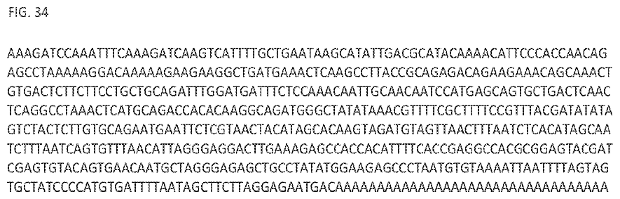

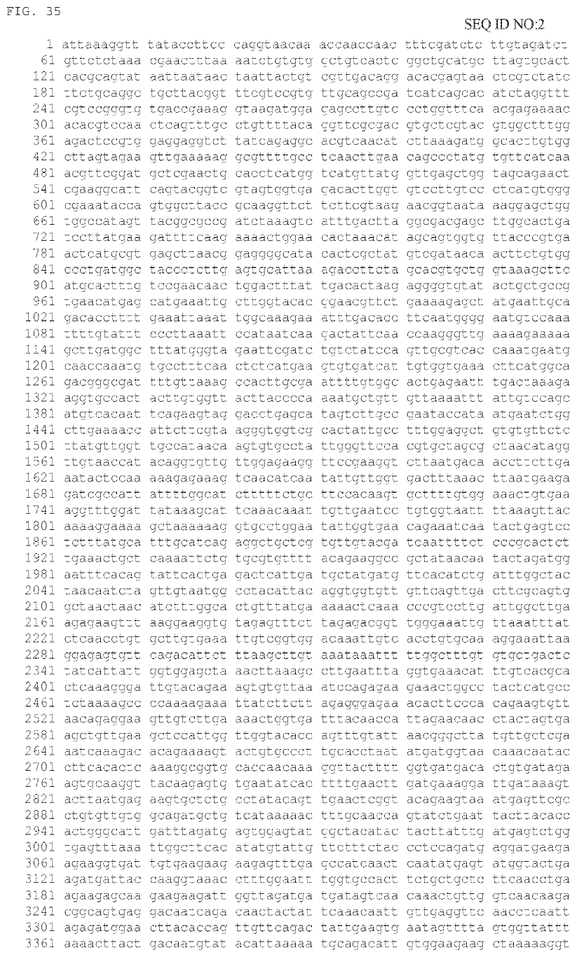

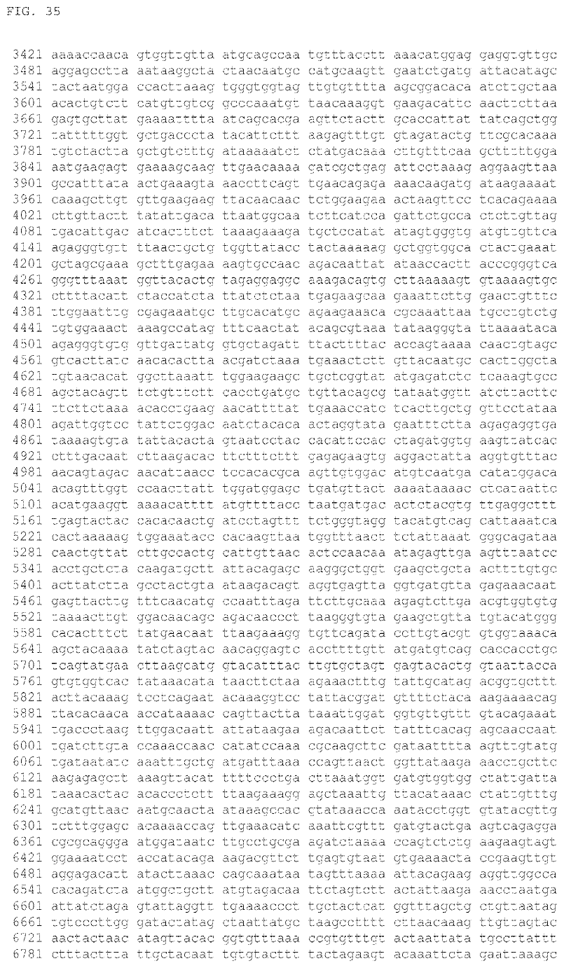

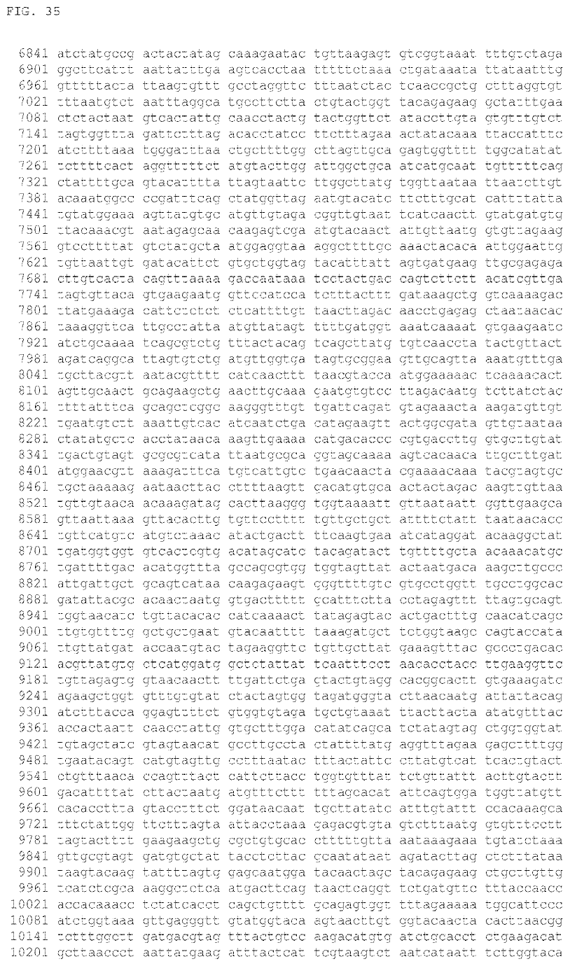

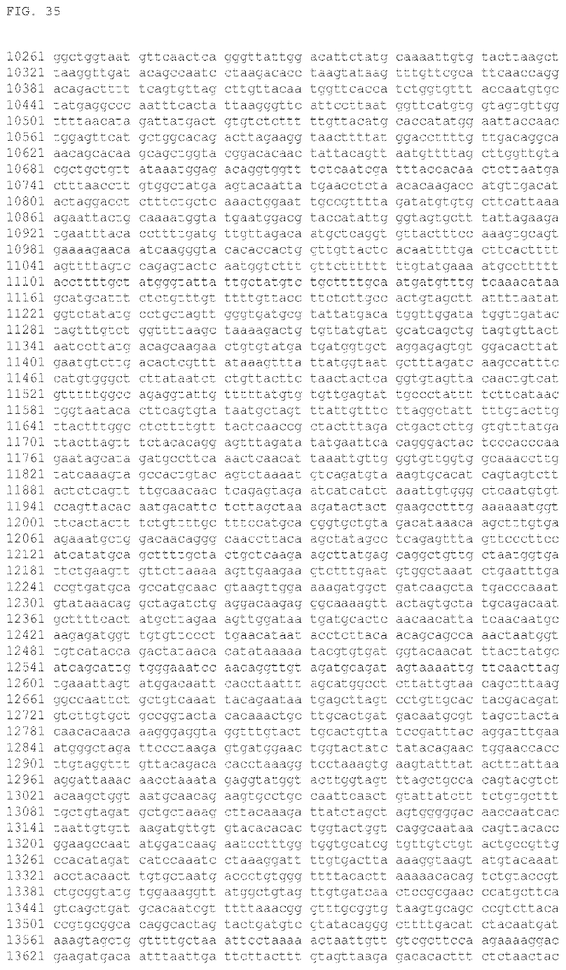

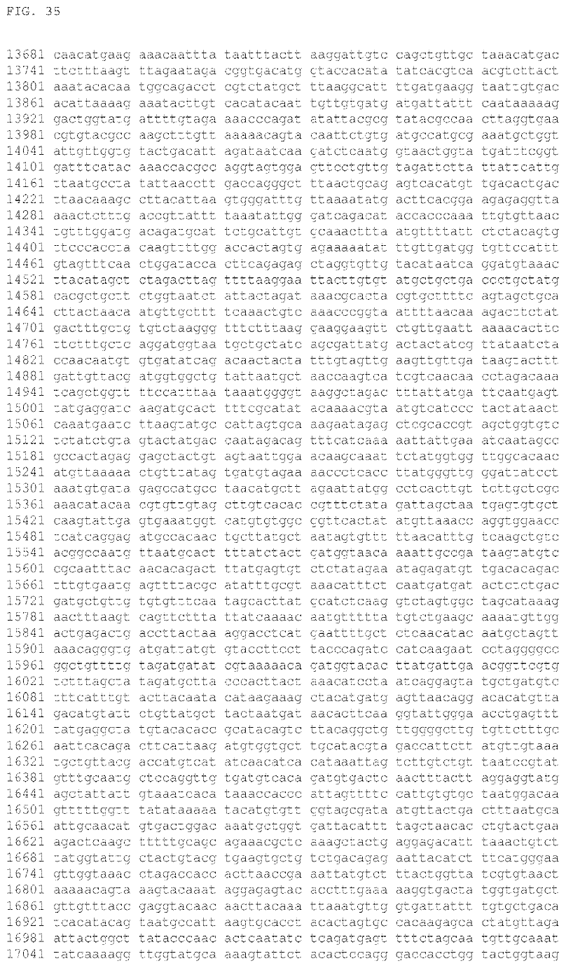

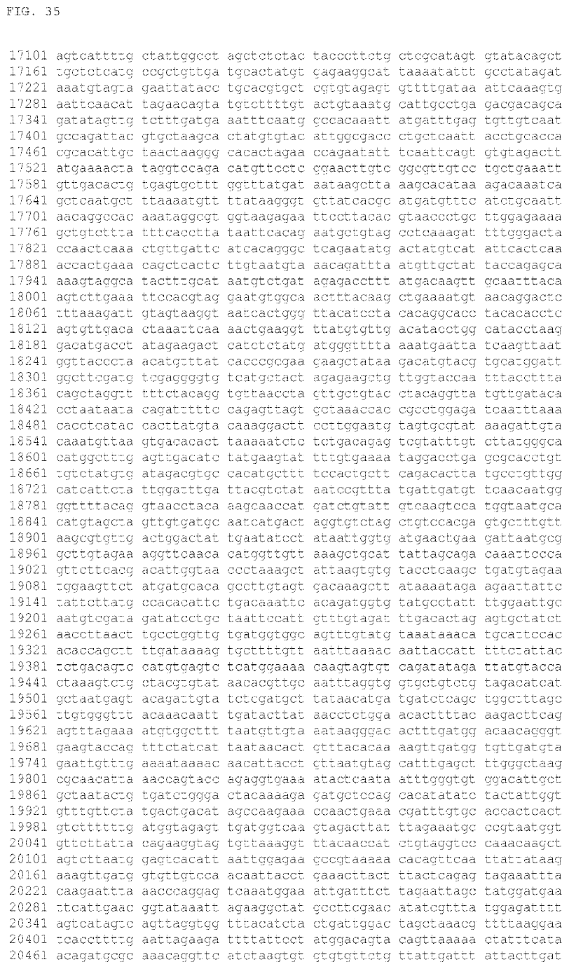

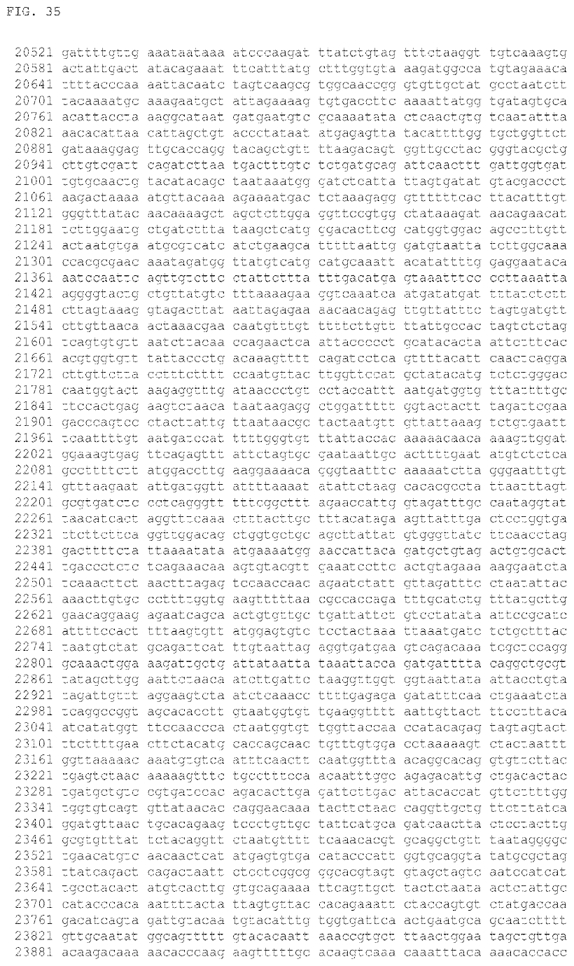

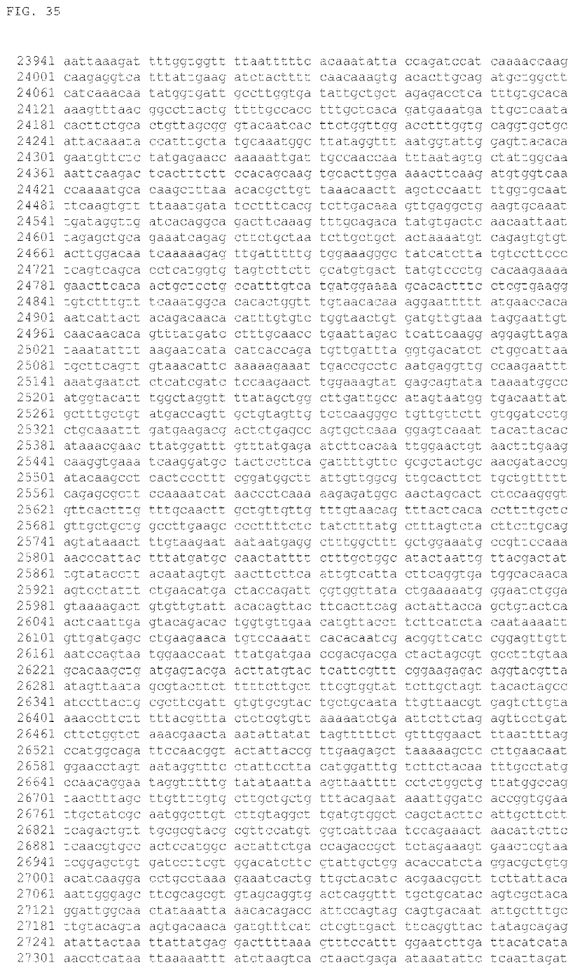

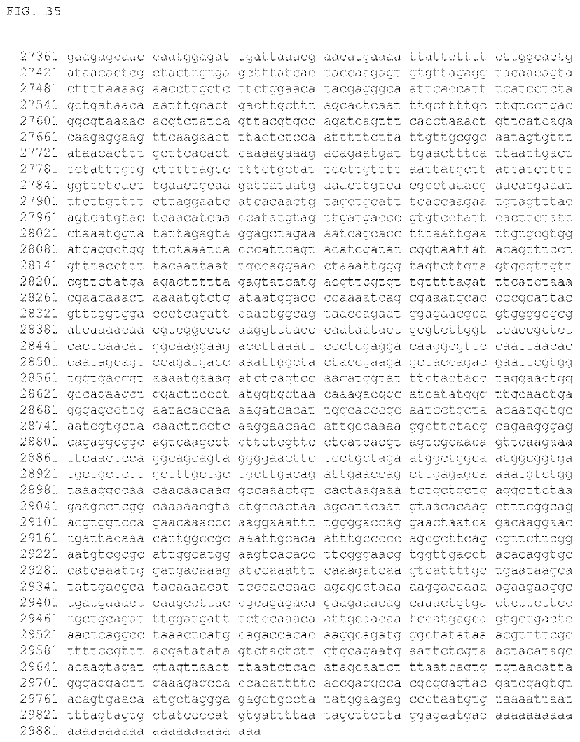

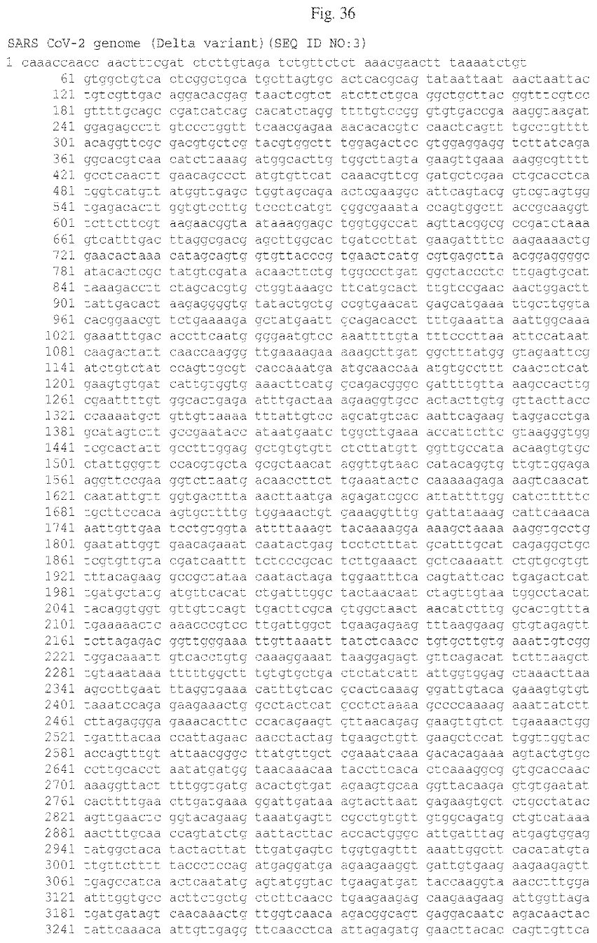

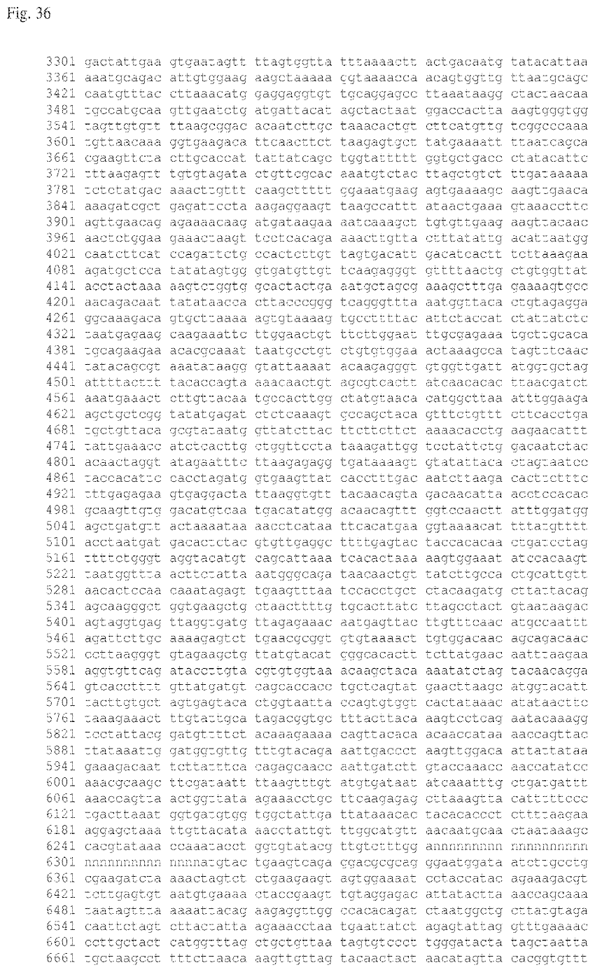

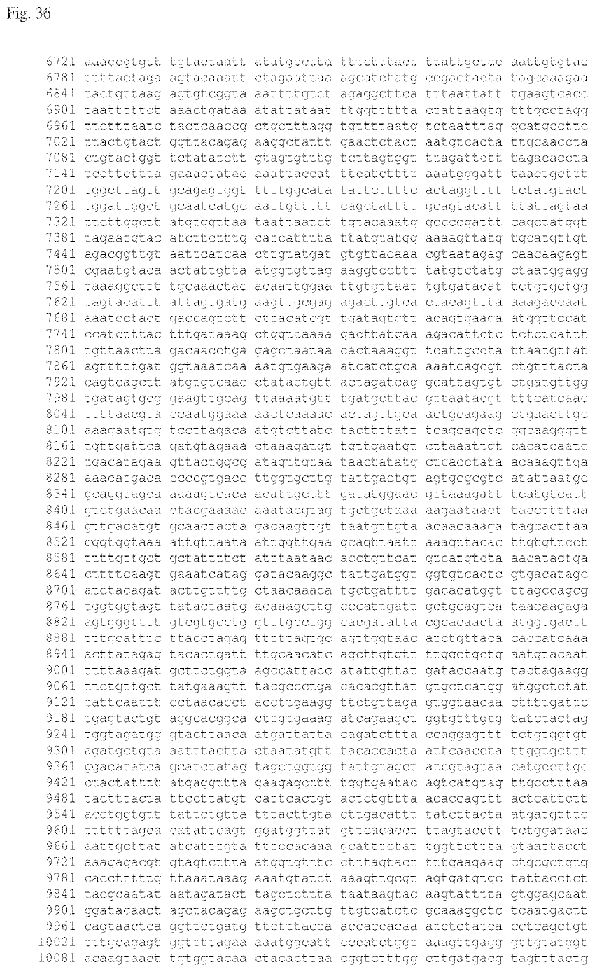

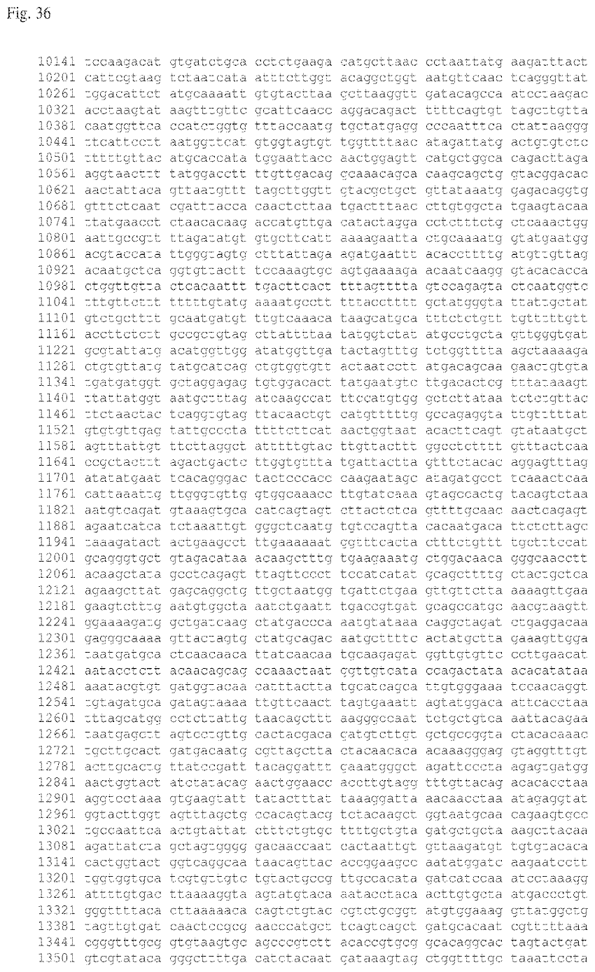

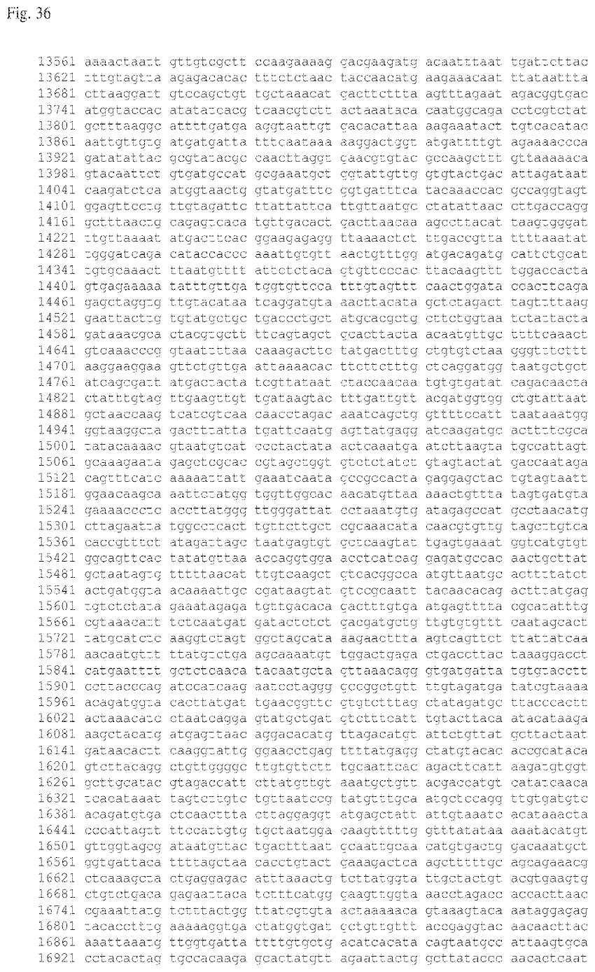

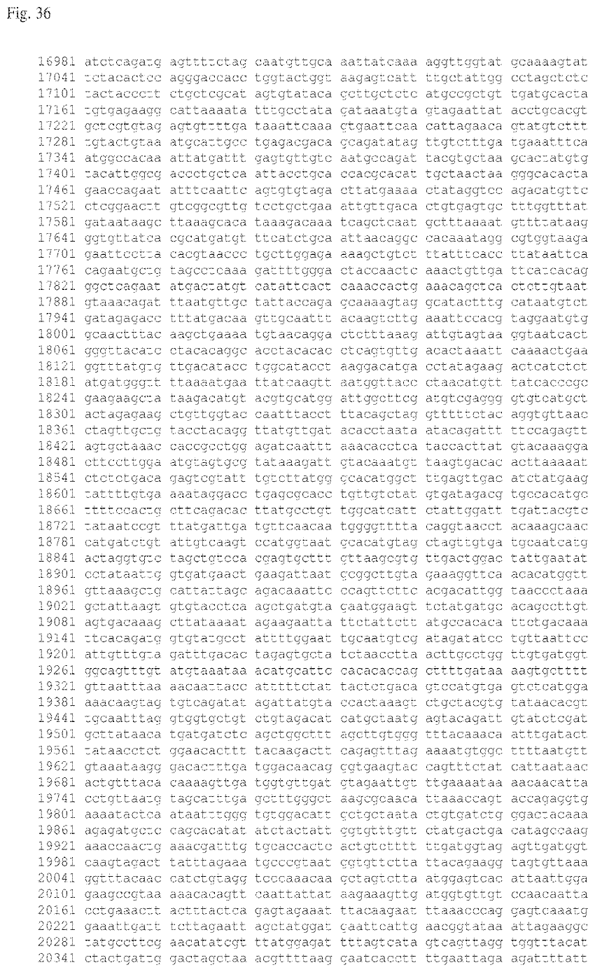

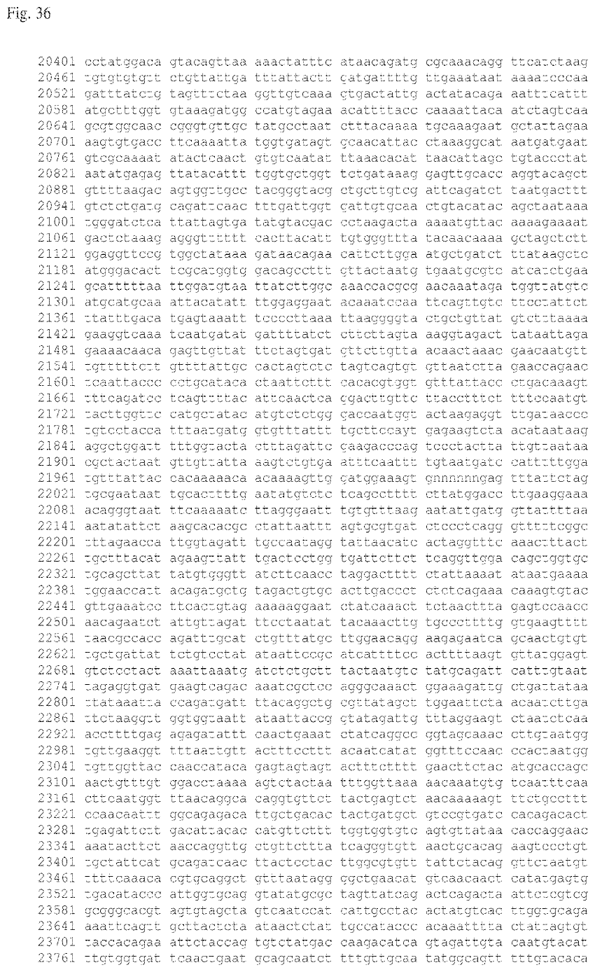

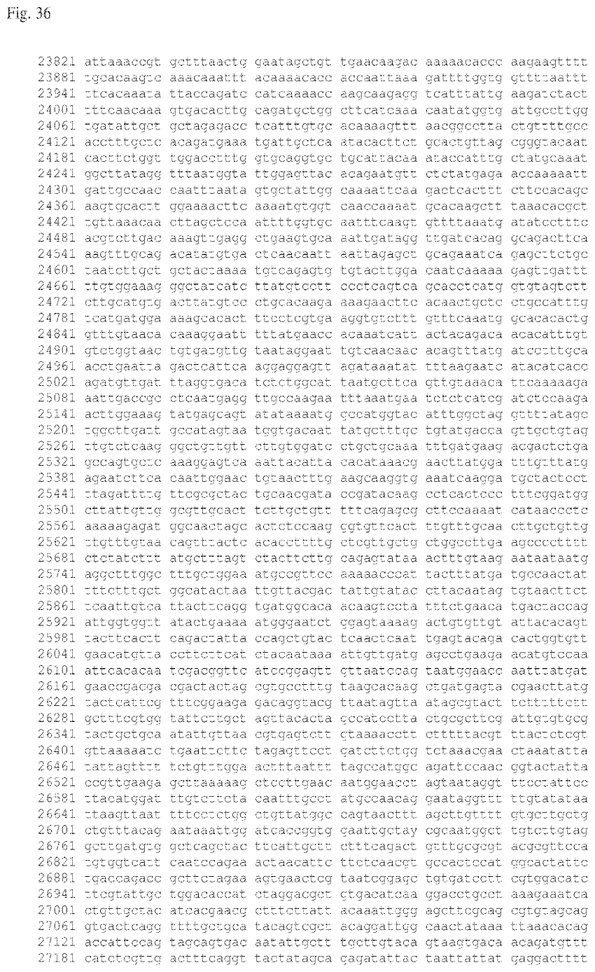

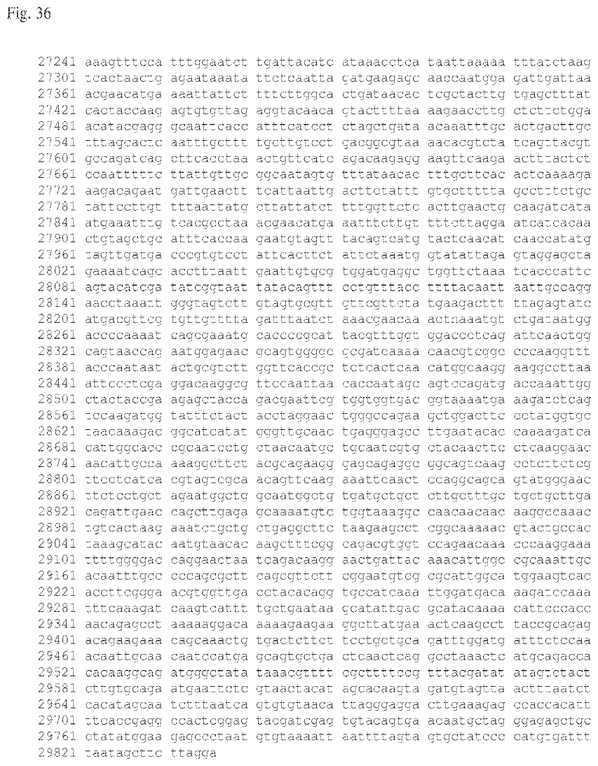

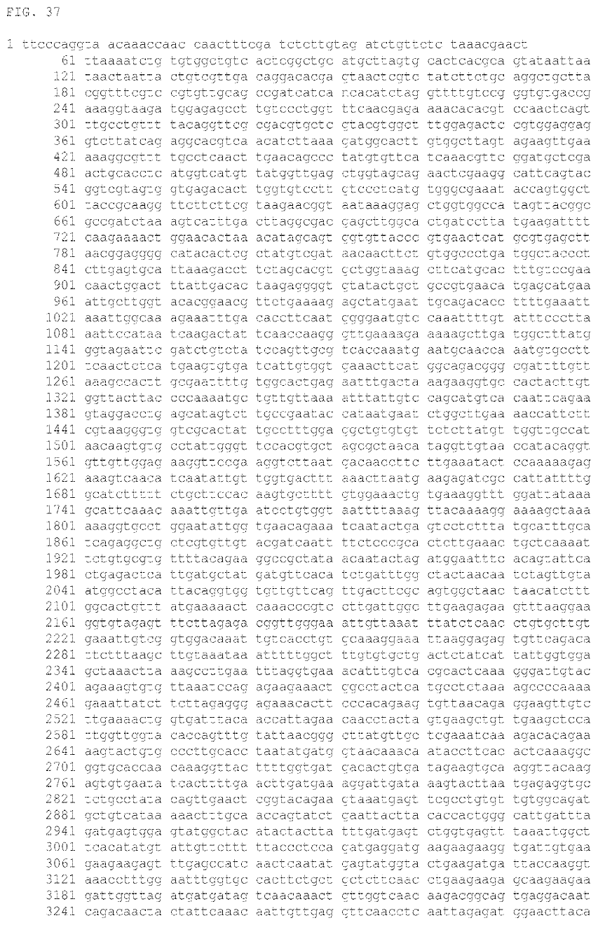

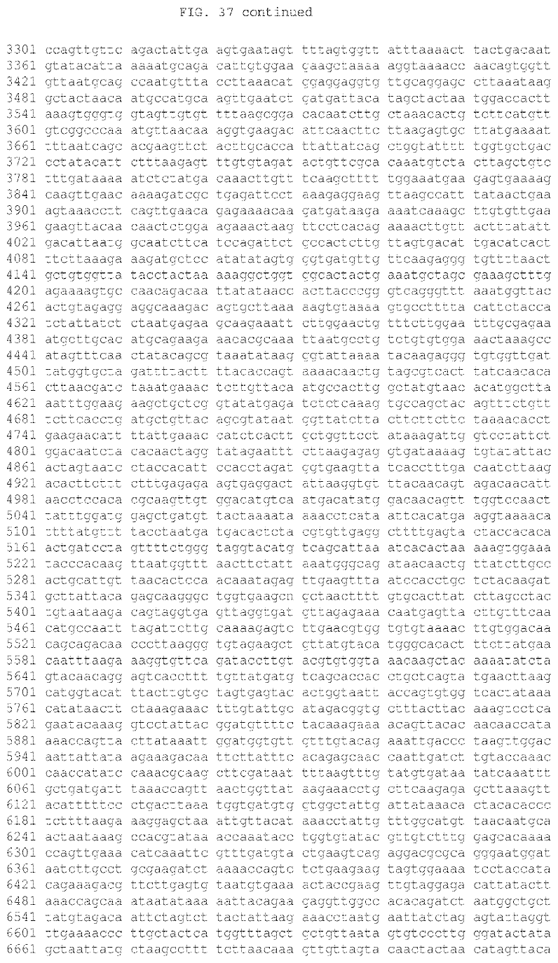

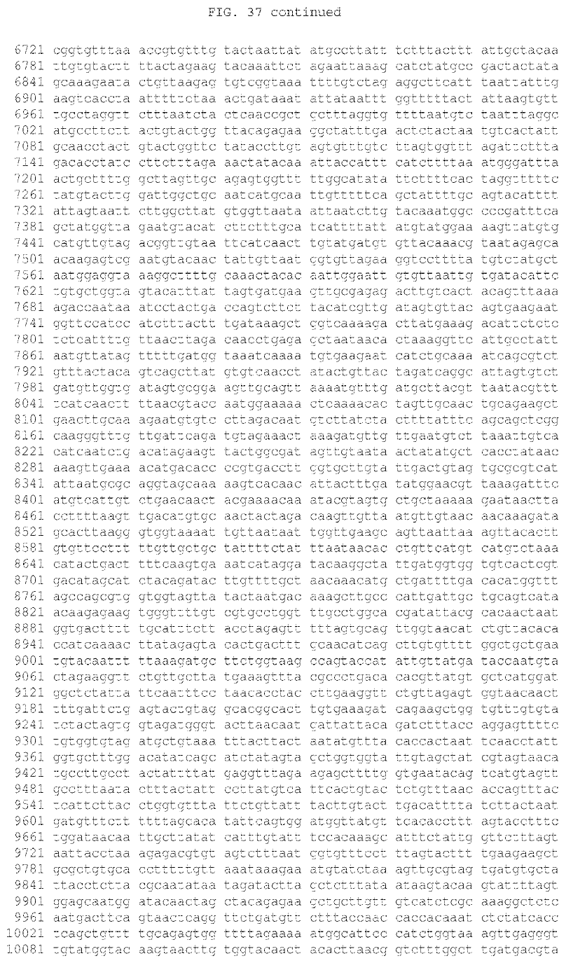

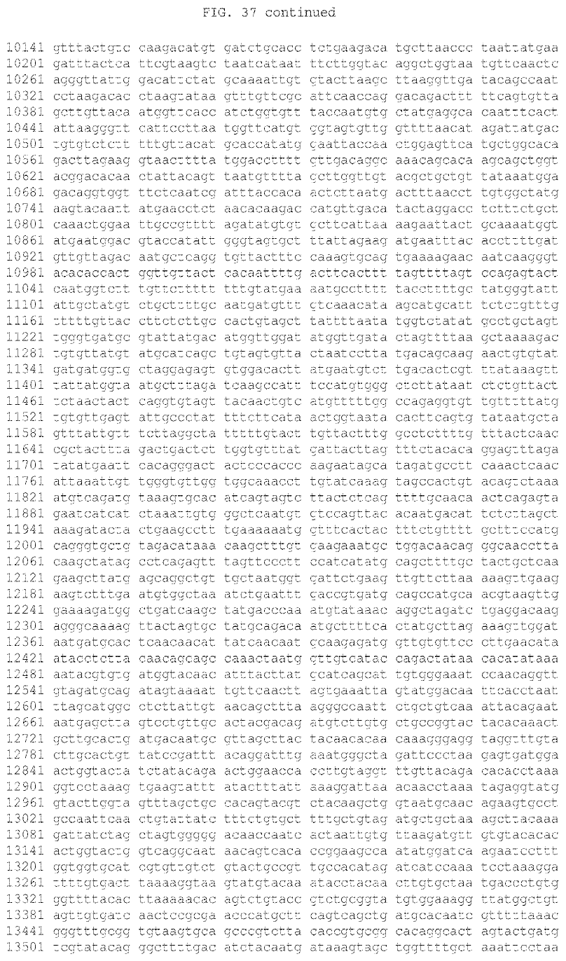

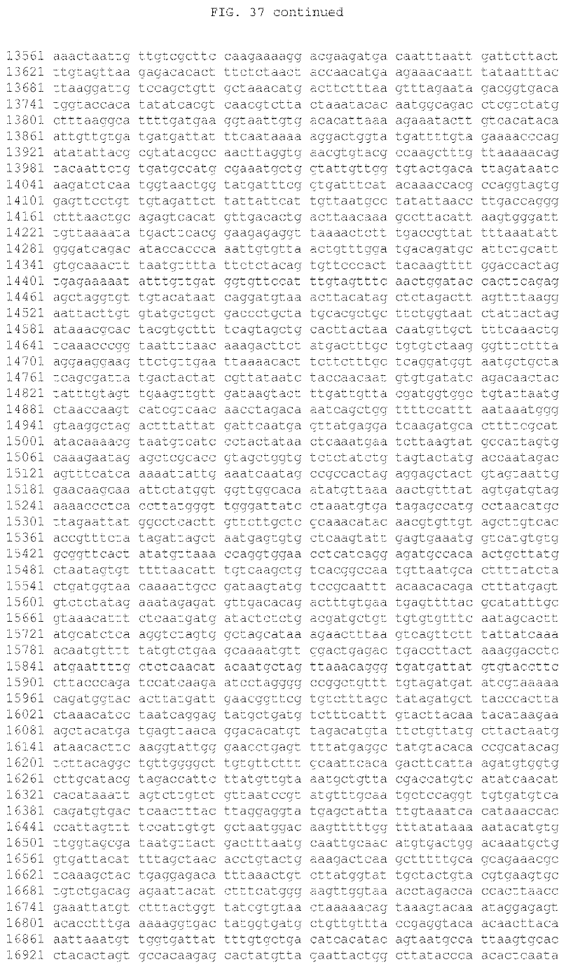

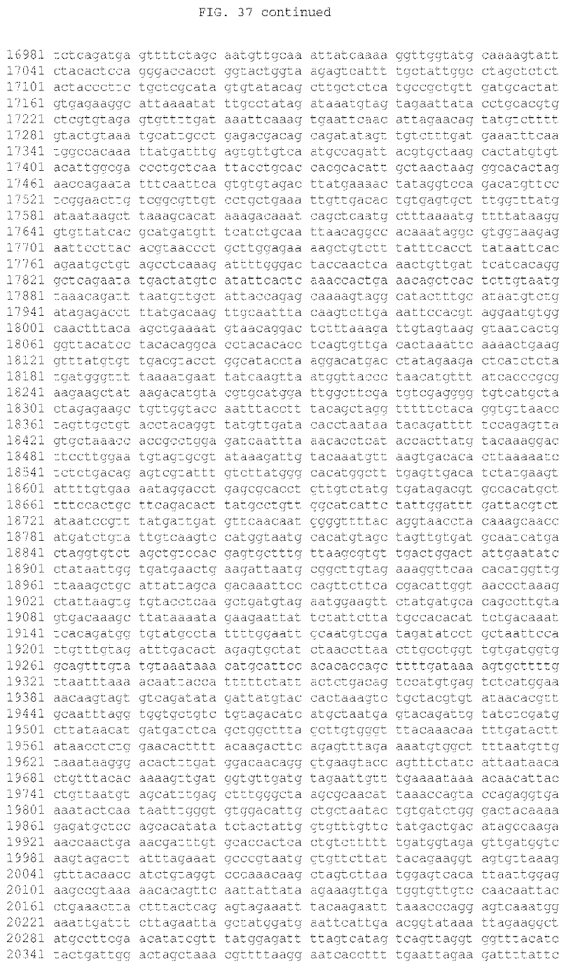

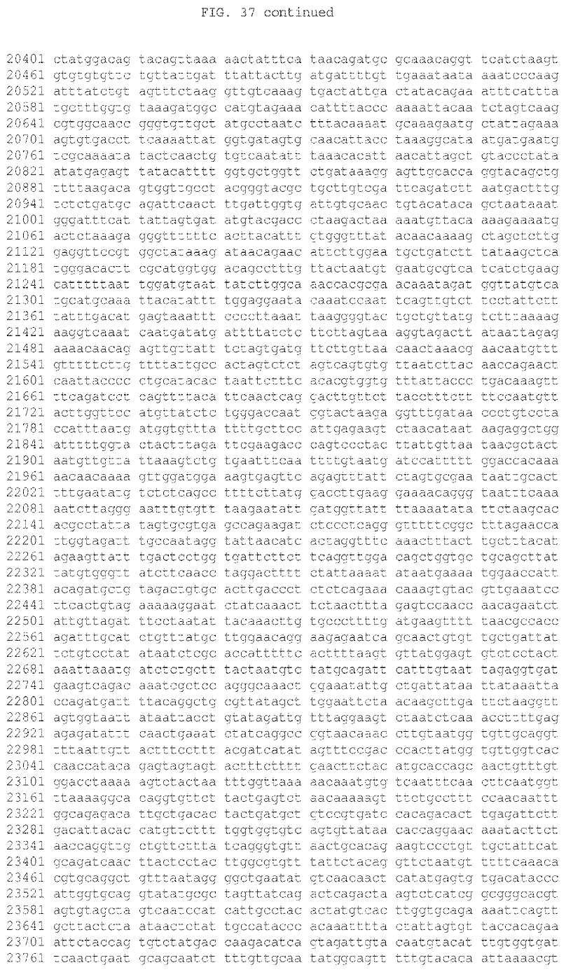

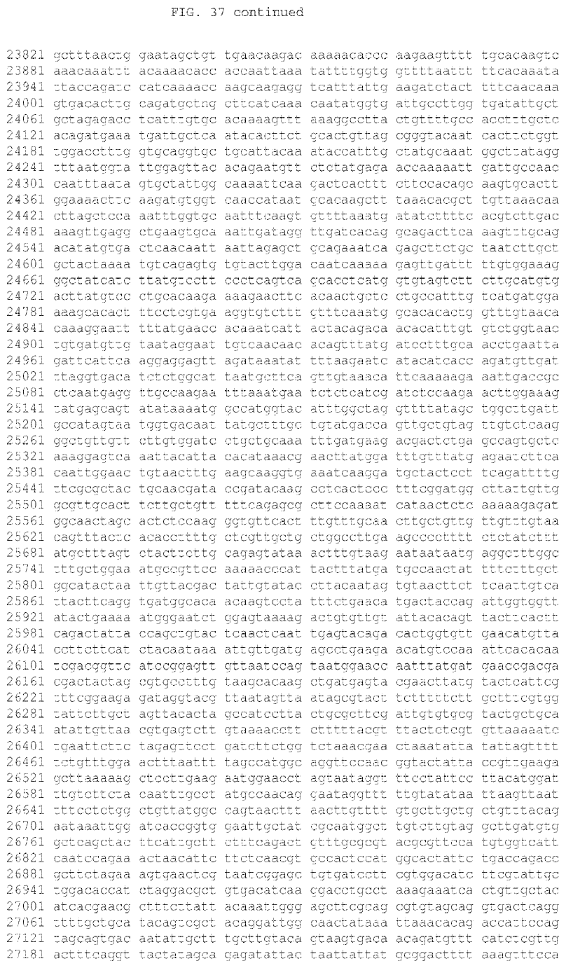

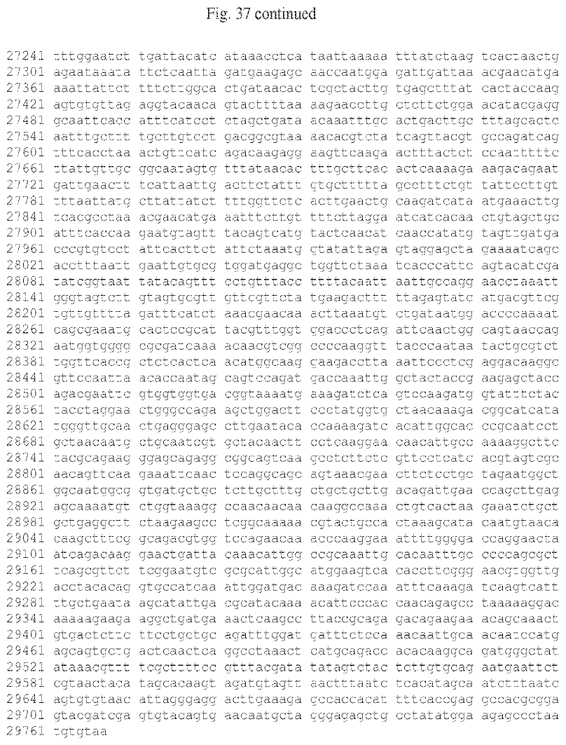

An exemplary cold-adapted coronavirus has a genome with one or more alterations in nucleotide sequences for one or more of Nsp1, Nsp2, Nsp3, Nsp4, Nsp5, Nsp6, Nsp7, Nsp8, Nsp9, Nsp10, Nsp11, Nsp12, Nsp13, Nsp14, Nsp15, Nsp16, S, E, M, N, or any combination thereof, which alteration may be a deletion of one or more nucleotides, a substitution of one or more nucleotides, an insertion of one or more nucleotides, or any combination thereof, relative to a genome having SEQ ID NO:1 (FIG. 34), SEQ ID NO:2 (FIG. 35), SEQ ID NO:3, or SEQ ID NO: 4, or a nucleic acid sequence with at least 80%, 82%, 84%, 85%, 87%, 89%, 90%, 92%, 94%, 95%, 96%, 97%, 98% or 99% nucleic acid sequence identity thereto.

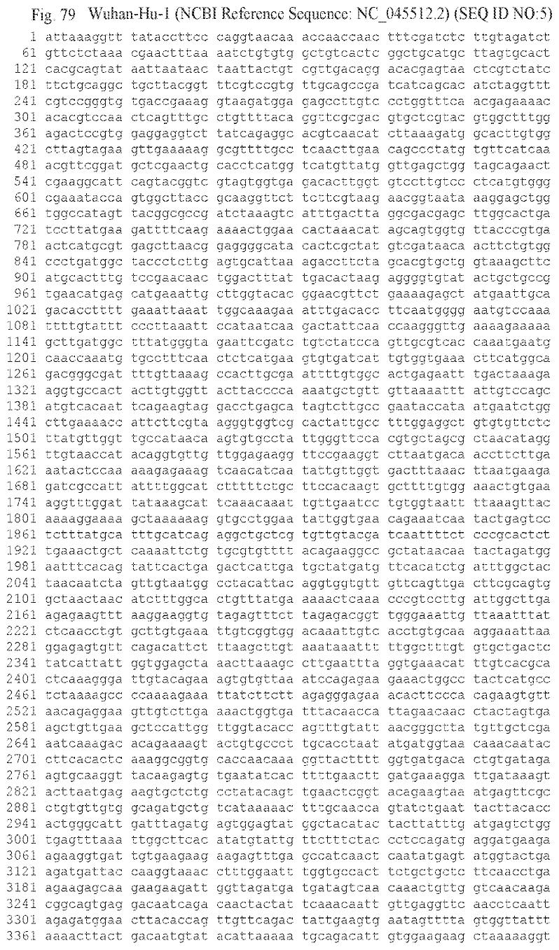

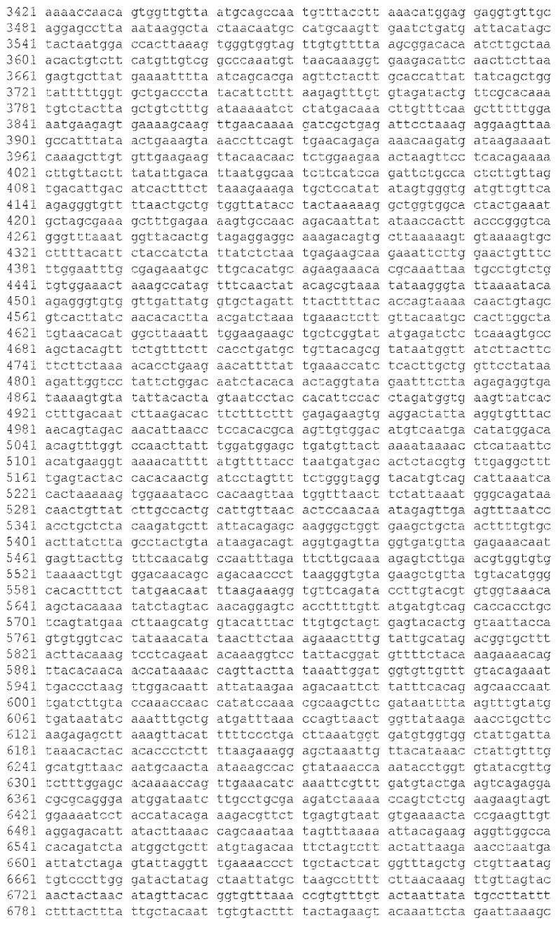

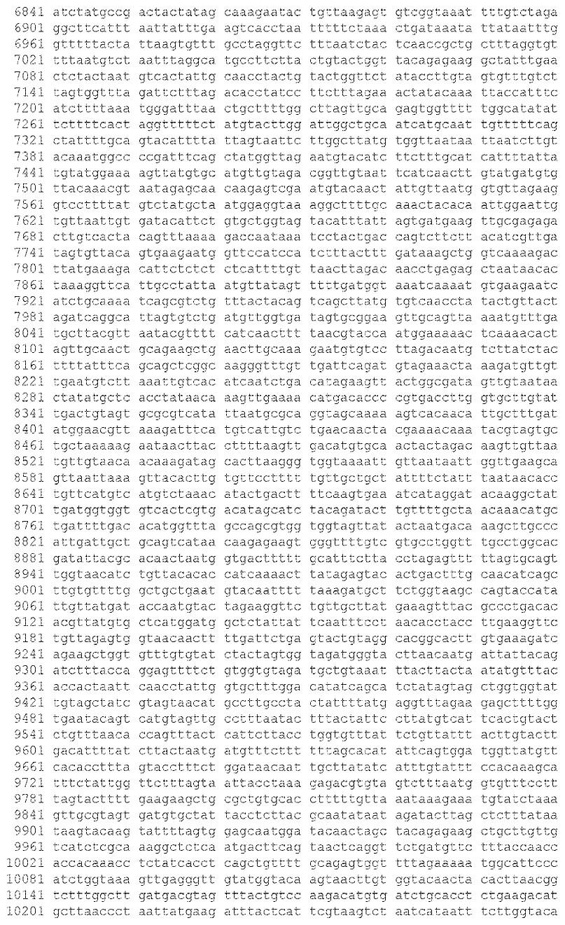

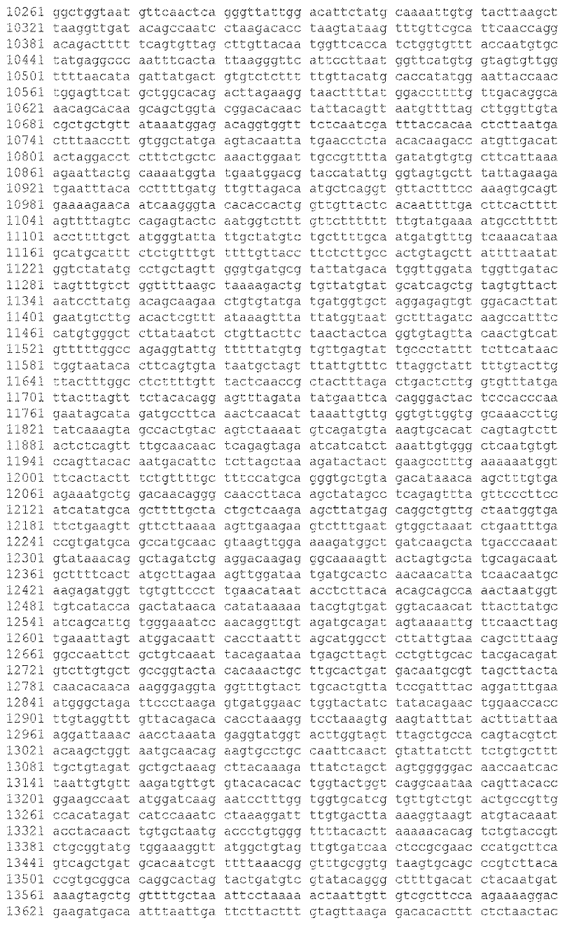

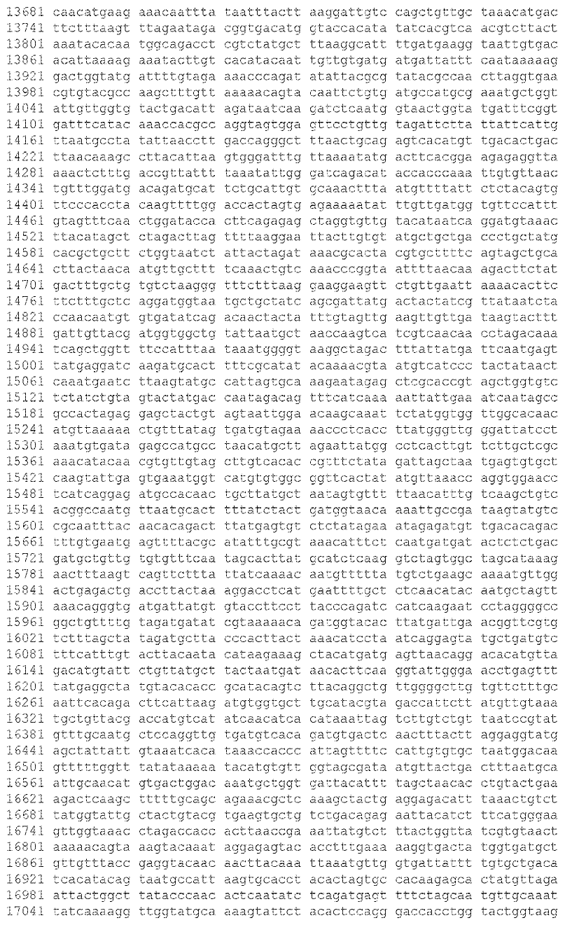

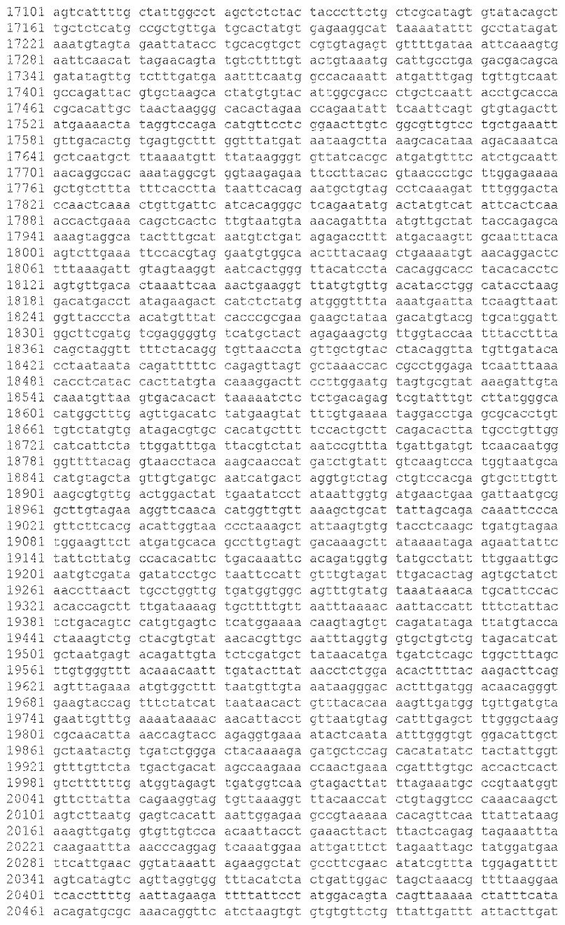

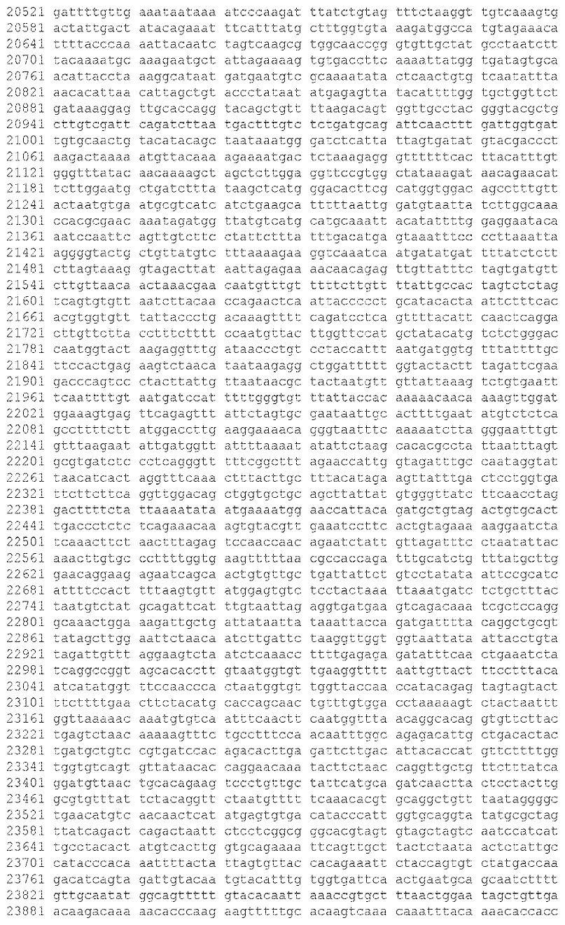

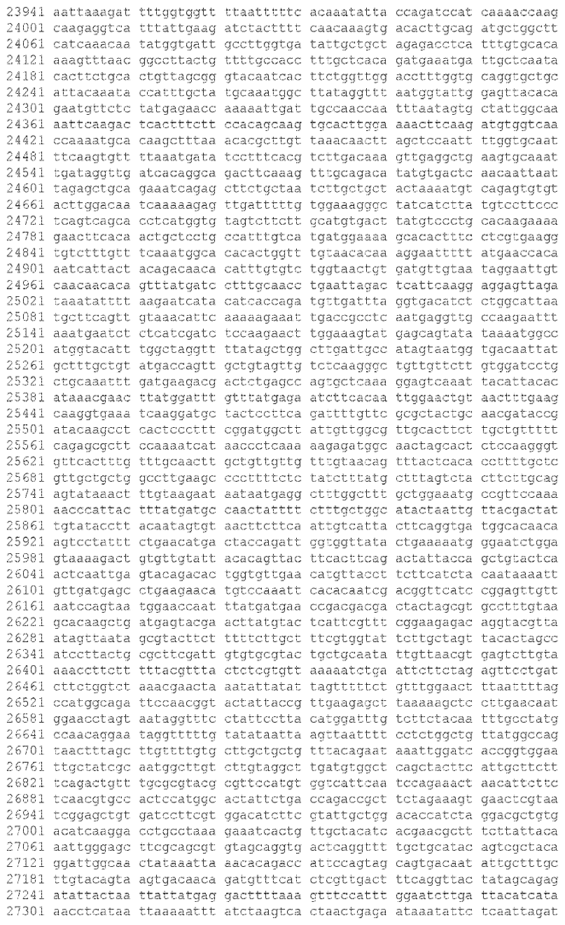

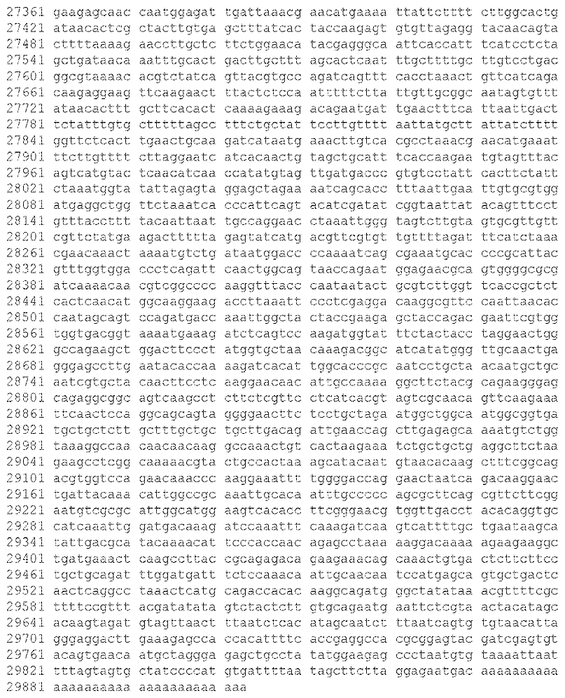

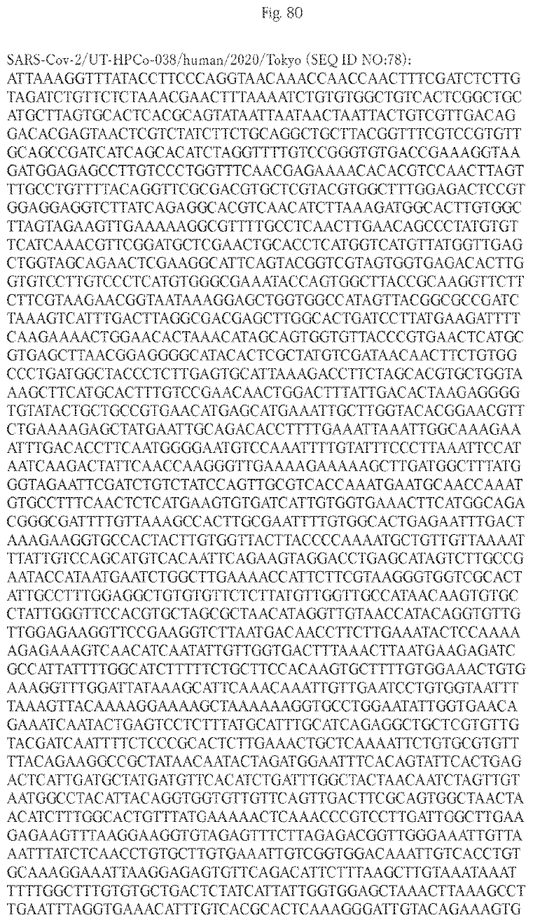

An exemplary cold-adapted coronavirus has one or more alterations in one or more of Nsp1, Nsp2, Nsp3, Nsp4, Nsp5, Nsp6, Nsp7, Nsp8, Nsp9, Nsp10, Nsp11, Nsp12, Nsp13, Nsp14, Nsp15, Nsp16, S, E, M or N relative to proteins encoded by severe acute respiratory syndrome coronavirus 2 isolate Wuhan-Hu-1 (NCBI Reference Sequence: NC_045512.2) (SEQ ID NO: 5; FIG. 79), such as a genome that encodes the following polypeptides.

ORF1ab amino acid sequence is provided below as (SEQ ID NO:51):

MESLVPGFNEKTHVQLSLPVLQVRDVLVRGFGDSVEEVLSEARQHLKD
GTCGLVEVEKGVLPQLEQPYVFIKRSDARTAPHGHVMVELVAELEGIQY
GRSGE
TLGVLVPHVGEIPVAYRKVLLRKNGNKGAGGHSYGADLKSFDLGDELG
TDPYEDFQENWNTKHSSGVTRELMRELNGGAYTRYVDNNFCGPDGYPL
ECIKDLLARAGKASCTLSEQLDFIDTKRGVYCCREHEHEIAWYTERSEKS
YELQTPFEIKLAKKFDTFNGECPNFVFPLNSIIKTIQPRVEKKKLDGFMG
RIRSVYPVASPNECNQMCLSTLMKCDHCGETSWQTGDFVKATCEFCGTE
NLTKEGATTCGYLPQNAVVKIYCPACHNSEVGPEHSLAEYHNESGLKTI
LRKGGRTIAFGGCVFSYVGCHNKCAYWVPRASANIGCNHTGVVGEGSE
GLNDNLLEILQKEKVNINIVGDFKLNEEIAIILASFSASTSAFVETVKGL
DYKAFKQIVESCGNFKVTKGKAKKGAWNIGEQKSILSPLYAFASEAARVV
RSIFSRTLETAQNSVRVLQKAAITILDGISQYSLRLIDAMMFTSDLATNN
LVVMAYITGGVVQLTSQWLTNIFGTVYEKLKPVLDWLEEKFKEGVEFLRD
GWEIVKFISTCACEIVGGQIVTCAKEIKESVQTFFKLVNKFLALCADSII
IGGAKLKALNLGETFVTHSKGLYRKCVKSREETGLLMPLKAPKEIIFLEG
ETLPTEVLTEEVVLKTGDLQPLEQPTSEAVEAPLVGTPVCINGLMLLEIK
DTEKYCALAPNMMVTNNTFTLKGGAPTKVTFGDDTVIEVQGYKSVNITFE
LDERIDKVLNEKCSAYTVELGTEVNEFACVVADAVIKTLQPVSELLTPLG
IDLDEWSMATYYLFDESGEFKLASHMYCSFYPPDEDEEEGDCEEEEFEPS
TQYEYGTEDDYQGKPLEFGATSAALQPEEEQEEDWLDDDSQQTVGQQD
GSEDNQTTTIQTIVEVQPQLEMELTPVVQTIEVNSFSG
YLKLTDNVYIKNADIVEEAKKVKPTVVVNAANVYLKHGGGVAGALNK
ATNNAMQVESDDYIATNGPLKVGGSCVLSGHNLAKHCLHVVGPNVNK
GEDIQLLKSAYENFNQHEVLLAPLLSAGIFGADPIHSLRVCVDTVRTNVY
LAVFDKNLYDKLVSSFLEMKSEKQVEQKIAEIPKEEVKPFITESKPSVEQ
RKQDDKKIKACVEEVTTTLEETKFLTENLLLYIDINGNLHPDSATLVSDI
DITFLKKDAPYIVGDVVQEGVLTAVVIPTKKAGGTTEMLAKALRKVPTDN
YITTYPGQGLNGYTVEEAKTVLKKCKSAFYILPSIISNEKQEILGTVSWN
LREMLAHAEETRKLMPVCVETKAIVSTIQRKYKGIKIQEGVVDYGARFYF
YTSKTTVASLINTLNDLNETLVTMPLGYVTHGLNLEEAARYMRSLKVPA
TVSVSSPDAVTAYNGYLTSSSKTPEEHFIETISLAGSYKDWSYSGQSTQL
GIEFLKRGDKSVYYTSNPTTFHLDGEVITFDNLKTLLSLREVRTIKVFTT
VDNINLHTQVVDMSMTYGQQFGPTYLDGADVTKIKPHNSHEGKTFYVLP
NDDTLRVEAFEYYHTTDPSFLGRYMSALNHTKKWKYPQVNGLTSIKWA
DNNCYLATALLTLQQIELKFNPPALQDAYYRARAGEAANFCALILAYCN
KTVGELGDVRETMSYLFQHANLDSCKRVLNVVCKTCGQQQTTLKGVE
AVMYMGTLSYEQFKKGVQIPCTCGKQATKYLVQQESPFVMMSAPPAQ
YELKHGTFTCASEYTGNYQCGHYKHITSKETLYCIDGALLTKSSEYKGPI
TDVFYKENSYTTTIKPVTYKLDGVVCTEIDPKLDNYYKKDNSYFTEQPID
LVPNQPYPNASFDNFKFVCDNIKFADDLNQLTGYKKPASRELKVTFFPD
LNGDVVAIDYKHYTPSFKKGAKLLHKPIVWHVNNATNKATYKPNTWCI
RCLWSTKPVETSNSFDVLKSEDAQGMDNLACEDLKPVSEEVVENPTIQK
DVLECNVKTTEVVGDIILKPANNSLKITEEVGHTDLMAAYVDNSSLTIKK
PNELSRVLGLKTLATHGLAAVNSVPWDTIANYAKPFLNKVVSTTTNIVT
RCLNRVCTNYMPYFFTLLLQLCTFTRSTNSRIKASMPTTIAKNTVKSVGK
FCLEASFNYLKSPNFSKLINIIIWFLLLSVCLGSLIYSTAALGVLMSNLG
MPSYCTGYREGYLNSTNVTIATYCTGSIPCSVCLSGLDSLDTYPSLETIQ
ITISSFKWDLTAFGLVAEWFLAYILFTRFFYVLGLAAIMQLFFSYFAVHF
ISNSWLMWLIINLVQMAPISAMVRMYIFFASFYYVWKSYVHVVDGCNSST
CMMCYKRNRATRVECTTIVNGVRRSFYVYANGGKGFCKLHNWNCVNCDT
FCAGSTFISDEVARDLSLQFKRPINPTDQSSYIVDSVTVKNGSIHLYFDK
AGQKTYERHSLSHFVNLDNLRANNTKGSLPINVIVFDGKSKCEESSAKSA
SVYYSQLMCQPILLLDQALVSDVGDSAEVAVKMFDAYVNTFSSTFNVPM
EKLKTLVATAEAELAKNVSLDNVLSTFISAARQGFVDSDVETKDVVECL
KLSHQSDIEVTGDSCNNYMLTYNKVENMTPRDLGACIDCSARHINAQV
AKSHNIALIWNVKDFMSLSEQLRKQIRSAAKKNNLPFKLTCATTRQVVN
VVTTKIALKGGKIVNNWLKQLIKVTLVFLFVAAIFYLITPVHVMSKHTDF
SSEIIGYKAIDGGVTRDIASTDTCFANKHADFDTWFSQRGGSYTNDKACP
LIAAVITREVGFVVPGLPGTILRTTNGDFLHFLPRVFSAVGNICYTPSKL
IEYTDFATSACVLAAECTIFKDASGKPVPYCYDTNVLEGSVAYESLRPDT
RYVLMDGSIIQFPNTYLEGSVRVVTTFDSEYCRHGTCERSEAGVCVSTSG
RWVLNNDYYRSLPGVFCGVDAVNLLTNMFTPLIQPIGALDISASIVAGGI
VAIVVTCLAYYFMRFRRAFGEYSHVVAFNTLLFLMSFTVLCLTPVYSFLP
GVYSVIYLYLTFYLTNDVSFLAHIQWMVMFTPLVPFWITIAYIICISTKH
FYWFFSNYLKRRVVFNGVSFSTFEEAALCTFLLNKEMYLKLRSDVLLPLT
QYNRYLALYNKYKYFSGAMDTTSYREAACCHLAKALNDESNSGSDVLY
QPPQTSITSAVLQSGFRKMAFPSGKVEGCMVQVTCGTTTLNG
LWLDDVVYCPRHVICTSEDMLNPNYEDLLIRKSNHNFLVQAGNVQLRVI
GHSMQNCVLKLKVDTANPKTPKYKFVRIQPGQTFSVLACYNGSPSGVY
QCAMRPNFTIKGSFLNGSCGSVGFNIDYDCVSFCYMHHMELPTGVHAGT
DLEGNFYGPFVDRQTAQAAGTDTTITVNVLAWLYAAVINGDRWFLNRF
TTTLNDFNLVAMKYNYEPLTQDHVDILGPLSAQTGIAVLDMCASLKELL
QNGMNGRTILGSALLEDEFTPFDVVRQCSGVTFQSAVKRTIKGTHHWLL
LTILTSLLVLVQSTQWSLFFFLYENAFLPFAMGIIAMSAFAMMFVKHKHA
FLCLFLLPSLATVAYFNMVYMPASWVMRIMTWLDMVDTSLSGFKLKDC
VMYASAVVLLILMTARTVYDDGARRVWTLMNVLTLVYKVYYGNALD
QAISMWALIISVTSNYSGVVTTVMFLARGIVFMCVEYCPIFFITGNTLQC
IMLVYCFLGYFCTCYFGLFCLLNRYFRLTLGVYDYLVSTQEFRYMNSQG
LLPPKNSIDAFKLNIKLLGVGGKPCIKVATVQSKMSDVKCTSVVLLSVLQ
QLRVESSSKLWAQCVQLHNDILLAKDTTEAFEKMVSLLSVLLSMQGAV
DINKLCEEMLDNRATLQAIASEFSSLPSYAAFATAQEAYEQAVANGDSE
VVLKKLKKSLNVAKSEFDRDAAMQRKLEKMADQAMTQMYKQARSED
KRAKVTSAMQTMLFTMLRKLDNDALNNIINNARDGCVPLNIIPLTTAAK
LMVVIPDYNTYKNTCDGTTFTYASALWEIQQVVDADSKIVQLSEISMDN
SPNLAWPLIVTALRANSAVKLQNNELSPVALRQMSCAAGTTQTACTDD
NALAYYNTTKGGRFVLALLSDLQDLKWARFPKSDGTGTIYTELEPPCRF
VTDTPKGPKVKYLYFIKGLNNLNRGMVLGSLAATVRLQAGNATEVPAN
STVLSFCAFAVDAAKAYKDYLASGGQPITNCVKMLCTHTGTGQAITVTP
EANMDQESFGGASCCLYCRCHIDHPNPKGFCDLKGKYVQIPTTCANDPV
GFTLKNTVCTVCGMWKGYGCSCDQLREPMLQSADAQSFLNRVCGVSA
ARLTPCGTGTSTDVVYRAFDIYNDKVAGFAKFLKTNCCRFQEKDEDDN
LIDSYFVVKRHTFSNYQHEETIYNLLKDCPAVAKHDFFKFRIDGDMVPHI
SRQRLTKYTMADLVYALRHFDEGNCDTLKEILVTYNCCDDDYFNKKD
WYDFVENPDILRVYANLGERVRQALLKTVQFCDAMRNAGIVGVLTLDN
QDLNGNWYDFGDFIQTTPGSGVPVVDSYYSLLMPILTLTRALTAESHVD
TDLTKPYIKWDLLKYDFTEERLKLFDRYFKYWDQTYHPNCVNCLDDRC
ILHCANFNVLFSTVFPPTSFGPLVRKIFVDGVPFVVSTGYHFRELGVVHN
QDVNLHSSRLSFKELLVYAADPAMHAASGNLLLDKRTTCFSVAALTNN
VAFQTVKPGNFNKDFYDFAVSKGFFKEGSSVELKHFFFAQDGNAAISDY
DYYRYNLPTMCDIRQLLFVVEVVDKYFDCYDGGCINANQVIVNNLDKS
AGFPFNKWGKARLYYDSMSYEDQDALFAYTKRNVIPTITQMNLKYAIS
AKNRARTVAGVSICSTMTNRQFHQKLLKSIAATRGATVVIGTSKFYGGW
HNMLKTVYSDVENPHLMGWDYPKCDRAMPNMLRIMASLVLARKHTTC
CSLSHRFYRLANECAQVLSEMVMCGGSLYVKPGGTSSGDATTAYANSV
FNICQAVTANVNALLSTDGNKIADKYVRNLQHRLYECLYRNRDVDTDF
VNEFYAYLRKHFSMMILSDDAVVCFNSTYASQGLVASIKNFKSVLYYQN
NVFMSEAKCWTETDLTKGPHEFCSQHTMLVKQGDDYVYLPYPDPSRIL
GAGCFVDDIVKTDGTLMIERFVSLAIDAYPLTKHPNQEYADVFHLYLQYI
RKLHDELTGHMLDMYSVMLTNDNTSRYWEPEFYEAMYTPHTVLQAVG
ACVLCNSQTSLRCGACIRRPFLCCKCCYDHVISTSHKLVLSVNPYVCNAP
GCDVTDVTQLYLGGMSYYCKSHKPPISFPLCANGQVFGLYKNTCVGSD
NVTDFNAIATCDWTNAGDYILANTCTERLKLFAAETLKATEETFKLSYGI
ATVREVLSDRELHLSWEVGKPRPPLNRNYVFTGYRVTKNSKVQIGEYTF
EKGDYGDAVVYRGTTTYKLNVGDYFVLTSHTVMPLSAPTLVPQEHYVR
ITGLYPTLNISDEFSSNVANYQKVGMQKYSTLQGPPGTGKSHFAIGLALY
YPSARIVYTACSHAAVDALCEKALKYLPIDKCSRIIPARARVECFDKFKV
NSTLEQYVFCTVNALPETTADIVVFDEISMATNYDLSVVNARLRAKHYV
YIGDPAQLPAPRTLLTKGTLEPEYFNSVCRLMKTIGPDMFLGTCRRCPAE
IVDTVSALVYDNKLKAHKDKSAQCFKMFYKGVITHDVSSAINRPQIGVV
REFLTRNPAWRKAVFISPYNSQNAVASKILGLPTQTVDSSQGSEYDYVIF
TQTTETAHSCNVNRFNVAITRAKVGILCIMSDRDLYDKLQFTSLEIPRRN
VATLQAENVTGLFKDCSKVITGLHPTQAPTHLSVDTKFKTEGLCVDIPGI
PKDMTYRRLISMMGFKMNYQVNGYPNMFITREEAIRHVRAWIGFDVEG
CHATREAVGTNLPLQLGFSTGVNLVAVPTGYVDTPNNTDFSRVSAKPPP
GDQFKHLIPLMYKGLPWNVVRIKIVQMLSDTLKNLSDRVVFVLWAHGF
ELTSMKYFVKIGPERTCCLCDRRATCFSTASDTYACWHHSIGFDYVYNP
FMIDVQQWGFTGNLQSNHDLYCQVHGNAHVASCDAIMTRCLAVHECF
VKRVDWTIEYPIIGDELKINAACRKVQHMVVKAALLADKFPVLHDIGNP
KAIKCVPQADVEWKFYDAQPCSDKAYKIEELFYSYATHSDKFTDGVCLF
WNCNVDRYPANSIVCRFDTRVLSNLNLPGCDGGSLYVNKHAFHTPAFD
KSAFVNLKQLPFFYYSDSPCESHGKQVVSDIDYVPLKSATCITRQNLGGA
VCRHHANEYRLYLDAYNMMISAGFSLWVYKQFDTYNLWNTFTRLQSL
ENVAFNVVNKGHFDGQQGEVPVSIINNTVYTKVDGVDVELFENKTTLPV
NVAFELWAKRNIKPVPEVKILNNLGVDIAANTVIWDYKRDAPAHISTIGV
CSMTDIAKKPTETICAPLTVFFDGRVDGQVDLFRNARNGVLITEGSVKGL
QPSVGPKQASLNGVTLIGEAVKTQFNYYKKVDGVVQQLPETYFTQSRNL
QEFKPRSQMEIDFLELAMDEFIERYKLEGYAFEHIVYGDFSHSQLGGLHL
LIGLAKRFKESPFELEDFIPMDSTVKNYFITDAQTGSSKCVCSVIDLLLD
DFVEIIKSQDLSVVSKVVKVTIDYTEISFMLWCKDGHVETFYPKLQSSQA
WQPGVAMPNLYKMQRMLLEKCDLQNYGDSATLPKGIMMNVAKYTQLC
QYLNTLTLAVPYNMRVIHFGAGSDKGVAPGTAVLRQWLPTGTLLVDSD
LNDFVSDADSTLIGDCATVHTANKWDLIISDMYDPKTKNVTKENDSKEG
FFTYICGFIQQKLALGGSVAIKITEHSWNADLYKLMGHFAWWTAFVTNV
NASSSEAFLIGCNYLGKPREQIDGYVMHANYIFWRNTNPIQLSSYSLFDM
SKFPLKLRGTAVMSLKEGQINDMILSLLSKGRLIIRENNRVVISSDVLVN
N

a polypeptide having residues 266 to 805 of ORF1ab (nsp1) is provided below as SEQ ID NO:52:

MESLVPGFNEKTHVQLSLPVLQVRDVLVRGFGDSVEEVLSEARQHLKD
GTCGLVEVEKGVLPQLEQPYVFIKRSDARTAPHGHVMVELVAELEGIQY
GRSGETLGVLVPHVGEIPVAYRKVLLRKNGNKGAGGHSYGADLKSFDL
GDELGTDPYEDFQENWNTKHSSGVTRELMRELNGG

a polypeptide having residues 806 to 2719 of ORF1ab (nsp2) is provided below as SEQ ID NO:53:

AYTRYVDNNFCGPDGYPLECIKDLLARAGKASCTLSEQLDFIDTKRGV
YCCREHEHEIAWYTERSEKSYELQTPFEIKLAKKFDTFNGECPNFVFP
LNSIIKTIQPRVEKKKLDGFMGRIRSVYPVASPNECNQMCLSTLMKCD
SHCGETWQTGDFVKATCEFCGTENLTKEGATTCGYLPQNAVVKIYCPA
CHNSEVGPEHSLAEYHNESGLKTILRKGGRTIAFGGCVFSYVGCHNKC
AYWVPRASANIGCNHTGVVGEGSEGLNDNLLEILQKEKVNINIVGDFK
LNEEIAIILASFSASTSAFVETVKGLDYKAFKQIVESCGNFKVTKGKA
KKGAWNIGEQKSILSPLYAFASEAARVVRSIFSRTLETAQNSVRVLQK
AAITILDGISQYSLRLIDAMMFTSDLATNNLVVMAYITGGVVQLTSQW
LTNIFGTVYEKLKPVLDWLEEKFKEGVEFLRDGWEIVKFISTCACEIV
GGQIVTCAKEIKESVQTFFKLVNKFLALCADSIIIGGAKLKALNLGET
FVTHSKGLYRKCVKSREETGLLMPLKAPKEIIFLEGETLPTEVLTEEV
VLKTGDLQPLEQPTSEAVEAPLVGTPVCINGLMLLEIKDTEKYCALAP
NMMVTNNTFTLKGG

a polypeptide having residues 2720 to 554 of ORF1ab (nsp3) is provided below as SEQ ID NO:54:

APTKVTFGDDTVIEVQGYKSVNITFELDERIDKVLNEKCSAYTVELGT
EVNEFACVVADAVIKTLQPVSELLTPLGIDLDEWSMATYYLFDESGEF
KLASHMYCSFYPPDEDEEEGDCEEEEFEPSTQYEYGTEDDYQGKPLEF
GATSAALQPEEEQEEDWLDDDSQQTVGQQDGSEDNQTTTIQTIVEVQP
QLEMELTPVVQTIEVNSFSGYLKLTDNVYIKNADIVEEAKKVKPTVVV
NAANVYLKHGGGVAGALNKATNNAMQVESDDYIATNGPLKVGGSCVLS
GHNLAKHCLHVVGPNVNKGEDIQLLKSAYENFNQHEVLLAPLLSAGIF
GADPIHSLRVCVDTVRTNVYLAVFDKNLYDKLVSSFLEMKSEKQVEQK
IAEIPKEEVKPFITESKPSVEQRKQDDKKIKACVEEVTTTLEETKFLT
ENLLLYIDINGNLHPDSATLVSDIDITFLKKDAPYIVGDVVQEGVLTA
VVIPTKKAGGTTEMLAKALRKVPTDNYITTYPGQGLNGYTVEEAKTVL
KKCKSAFYILPSIISNEKQEILGTVSWNLREMLAHAEETRKLMPVCVE
TKAIVSTIQRKYKGIKIQEGVVDYGARFYFYTSKTTVASLINTLNDLN
ETLVTMPLGYVTHGLNLEEAARYMRSLKVPATVSVSSPDAVTAYNGYL
TSSSKTPEEHFIETISLAGSYKDWSYSGQSTQLGIEFLKRGDKSVYYT
SNPTTFHLDGEVITFDNLKTLLSLREVRTIKVFTTVDNINLHTQVVDM
SMTYGQQFGPTYLDGADVTKIKPHNSHEGKTFYVLPNDDTLRVEAFEY
YHTTDPSFLGRYMSALNHTKKWKYPQVNGLTSIKWADNNCYLATALLT
LQQIELKFNPPALQDAYYRARAGEAANFCALILAYCNKTVGELGDVRE
TMSYLFQHANLDSCKRVLNVVCKTCGQQQTTLKGVEAVMYMGTLSYEQ
FKKGVQIPCTCGKQATKYLVQQESPFVMMSAPPAQYELKHGTFTCASE
YTGNYQCGHYKHITSKETLYCIDGALLTKSSEYKGPITDVFYKENSYT
TTIKPVTYKLDGVVCTEIDPKLDNYYKKDNSYFTEQPIDLVPNQPYPN
ASFDNFKFVCDNIKFADDLNQLTGYKKPASRELKVTFFPDLNGDVVAI
DYKHYTPSFKKGAKLLHKPIVWHVNNATNKATYKPNTWCIRCLWSTKP
VETSNSFDVLKSEDAQGMDNLACEDLKPVSEEVVENPTIQKDVLECNV
KTTEVVGDIILKPANNSLKITEEVGHTDLMAAYVDNSSLTIKKPNELS
RVLGLKTLATHGLAAVNSVPWDTIANYAKPFLNKVVSTTTNIVTRCLN
RVCTNYMPYFFTLLLQLCTFTRSTNSRIKASMPTTIAKNTVKSVGKFC
LEASFNYLKSPNFSKLINIIIWFLLLSVCLGSLIYSTAALGVLMSNLG
MPSYCTGYREGYLNSTNVTIATYCTGSIPCSVCLSGLDSLDTYPSLET
IQITISSFKWDLTAFGLVAEWFLAYILFTRFFYVLGLAAIMQLFFSYF
AVHFISNSWLMWLIINLVQMAPISAMVRMYIFFASFYYVWKSYVHVVD
GCNSSTCMMCYKRNRATRVECTTIVNGVRRSFYVYANGGKGFCKLHNW
NCVNCDTFCAGSTFISDEVARDLSLQFKRPINPTDQSSYIVDSVTVKN
GSIHLYFDKAGQKTYERHSLSHFVNLDNLRANNTKGSLPINVIVFDGK
SKCEESSAKSASVYYSQLMCQPILLLDQALVSDVGDSAEVAVKMFDAY
VNTFSSTFNVPMEKLKTLVATAEAELAKNVSLDNVLSTFISAARQGFV
DSDVETKDVVECLKLSHQSDIEVTGDSCNNYMLTYNKVENMTPRDLGA
CIDCSARHINAQVAKSHNIALIWNVKDFMSLSEQLRKQIRSAAKKNNL
PFKLTCATTRQVVNVVTTKIALKGG

a polypeptide having residues 8555 to 10054 of ORF1ab (nsp4) is provided below as SEQ ID NO:55:

KIVNNWLKQLIKVTLVFLFVAAIFYLITPVHVMSKHTDFSSEIIGYKA
IDGGVTRDIASTDTCFANKHADFDTWFSQRGGSYTNDKACPLIAAVIT
REVGFVVPGLPGTILRTTNGDFLHFLPRVFSAVGNICYTPSKLIEYTD
FATSACVLAAECTIFKDASGKPVPYCYDTNVLEGSVAYESLRPDTRYV
LMDGSIIQFPNTYLEGSVRVVTTFDSEYCRHGTCERSEAGVCVSTSGR
WVLNNDYYRSLPGVFCGVDAVNLLTNMFTPLIQPIGALDISASIVAGG
IVAIVVTCLAYYFMRFRRAFGEYSHVVAFNTLLFLMSFTVLCLTPVYS
FLPGVYSVIYLYLTFYLTNDVSFLAHIQWMVMFTPLVPFWITIAYIIC
ISTKHFYWFFSNYLKRRVVFNGVSFSTFEEAALCTFLLNKEMYLKLRS
DVLLPLTQYNRYLALYNKYKYFSGAMDTTSYREAACCHLAKALNDFSN
SGSDVLYQPPQTSITSAVLQ

a polypeptide having residues 10055 to 10972 of ORF1ab (nsp5A_3CLpro and nsp5B_3CLpro) is provided below as SEQ ID NO:56:

SGFRKMAFPSGKVEGCMVQVTCGTTTLNGLWLDDVVYCPRHVICTSED
MLNPNYEDLLIRKSNHNFLVQAGNVQLRVIGHSMQNCVLKLKVDTANP
KTPKYKFVRIQPGQTFSVLACYNGSPSGVYQCAMRPNFTIKGSFLNGS
SCGVGFNIDYDCVSFCYMHHMELPTGVHAGTDLEGNFYGPFVDRQTAQ
AAGTDTTITVNVLAWLYAAVINGDRWFLNRFTTTLNDFNLVAMKYNYE
PLTQDHVDILGPLSAQTGIAVLDMCASLKELLQNGMNGRTILGSALLE
DEFTPFDVVRQCSGVTFQ

a polypeptide having residues 10973 to 11842 of ORF 1ab (nsp6) is provided below as SEQ ID NO:57:

SAVKRTIKGTHHWLLLTILTSLLVLVQSTQWSLFFFLYENAFLPFAMG
IIAMSAFAMMFVKHKHAFLCLFLLPSLATVAYFNMVYMPASWVMRIMT
WLDMVDTSLSGFKLKDCVMYASAVVLLILMTARTVYDDGARRVWTLMN
VLTLVYKVYYGNALDQAISMWALIISVTSNYSGVVTTVMFLARGIVFM
CVEYCPIFFITGNTLQCIMLVYCFLGYFCTCYFGLFCLLNRYFRLTLG
VYDYLVSTQEFRYMNSQGLLPPKNSIDAFKLNIKLLGVGGKPCIKVAT
VQ

a polypeptide having residues 11843 to 12091 of ORF 1ab (nsp7) is provided below SEQ ID NO:58:

SKMSDVKCTSVVLLSVLQQLRVESSSKLWAQCVQLHNDILLAKDTTEA
FEKMVSLLSVLLSMQGAVDINKLCEEMLDNRATLQ

a polypeptide having residues 12092 to 12685 of PRF 1ab (nsp8) is provided below as SEQ ID NO:59:

AIASEFSSLPSYAAFATAQEAYEQAVANGDSEVVLKKLKKSLNVAKSE
FDRDAAMQRKLEKMADQAMTQMYKQARSEDKRAKVTSAMQTMLFTM
LRKLDNDALNNIINNARDGCVPLNIIPLTTAAKLMVVIPDYNTYKNTC
DCTTFTYASALWEIQQVVDADSKIVQLSEISMDNSPNLAWPLIVTALR
ANSAVKLQ

a polypeptide having residues 12686 to 13024 of ORF 1ab (nsp9) is provided below as SEQ ID NO:60:

NNELSPVALRQMSCAAGTTQTACTDDNALAYYNTTKGGRFVLALLSDL
QDLKWARFPKSDGTGTIYTELEPPCRFVTDTPKGPKVKYLYFIKGLNN
LNRGMVLGSLAATVRLQ

a polypeptide having residues 13025 to 13441 of ORF1ab (nsp10) is provided below as SEQ ID NO:61:

AGNATEVPANSTVLSFCAFAVDAAKAYKDYLASGGQPITNCVKMLCTH
TGTGQAITVTPEANMDQESFGGASCCLYCRCHIDHPNPKGFCDLKGKY
VQIPTTCANDPVGFTLKNTVCTVCGMWKGYGCSCDQLREPMLQ

a polypeptide having residues 13442 to 13480 of ORF1ab (nsp11) is provided below as SEQ ID NO:62:

SADAQSFLNGFAV

a polypeptide having residues 13442 to 13468 and 13468 to 16236 of ORF1ab (nsp12; RNA-dependent RNA polymerase) is provided below as SEQ ID NO: 63:

SADAQSFLNRVCGVSAARLTPCGTGTSTDVVYRAFDIYNDKVAGFAK
FLKTNCCRFQEKDEDDNLIDSYFVVKRHTFSNYQHEETIYNLLKDCP
AVAKHDFFKFRIDGDMVPHISRQRLTKYTMADLVYALRHFDEGNCDT
LKEILVTYNCCDDDYFNKKDWYDFVENPDILRVYANLGERVRQALLK
TVQFCDAMRNAGIVGVLTLDNQDLNGNWYDFGDFIQTTPGSGVPVVD
SYYSLLMPILTLTRALTAESHVDTDLTKPYIKWDLLKYDFTEERLKL
FDRYFKYWDQTYHPNCVNCLDDRCILHCANFNVLFSTVFPPTSFGPL
VRKIFVDGVPFVVSTGYHFRELGVVHNQDVNLHSSRLSFKELLVYAA
DPAMHAASGNLLLDKRTTCFSVAALTNNVAFQTVKPGNFNKDFYDFA
VSKGFFKEGSSVELKHFFFAQDGNAAISDYDYYRYNLPTMCDIRQLL
FVVEVVDKYFDCYDGGCINANQVIVNNLDKSAGFPFNKWGKARLYYD
SMSYEDQDALFAYTKRNVIPTITQMNLKYAISAKNRARTVAGVSICS
TMTNRQFHQKLLKSIAATRGATVVIGTSKFYGGWHNMLKTVYSDVEN
PHLMGWDYPKCDRAMPNMLRIMASLVLARKHTTCCSLSHRFYRLANE
CAQVLSEMVMCGGSLYVKPGGTSSGDATTAYANSVFNICQAVTANVN
ALLSTDGNKIADKYVRNLQHRLYECLYRNRDVDTDFVNEFYAYLRKH
FSMMILSDDAVVCFNSTYASQGLVASIKNFKSVLYYQNNVFMSEAKC
WTETDLTKGPHEFCSQHTMLVKQGDDYVYLPYPDPSRILGAGCFVDD
IVKTDGTLMIERFVSLAIDAYPLTKHPNQEYADVFHLYLQYIRKLHD
ELTGHMLDMYSVMLINDNTSRYWEPEFYEAMYTPHTVLQ

a polypeptide having residues 16237 to 18039 (nsp13; helicase) is provided below as SEQ ID NO:64:

AVGACVLCNSQTSLRCGACIRRPFLCCKCCYDHVISTSHKLVLSVNP
YVCNAPGCDVTDVTQLYLGGMSYYCKSHKPPISFPLCANGQVFGLYK
NTCVGSDNVTDFNAIATCDWTNAGDYILANTCTERLKLFAAETLKAT
EETFKLSYGIATVREVLSDRELHLSWEVGKPRPPLNRNYVFTGYRVT
KNSKVQIGEYTFEKGDYGDAVVYRGTTTYKLNVGDYFVLTSHTVMPL
SAPTLVPQEHYVRITGLYPTLNISDEFSSNVANYQKVGMQKYSTLQG
PPGTGKSHFAIGLALYYPSARIVYTACSHAAVDALCEKALKYLPIDK
CSRIIPARARVECFDKFKVNSTLEQYVFCTVNALPETTADIVVFDEI
SMATNYDLSVVNARLRAKHYVYIGDPAQLPAPRTLLTKGTLEPEYFN
SVCRLMKTIGPDMFLGTCRRCPAEIVDTVSALVYDNKLKAHKDKSAQ
CFKMFYKGVITHDVSSAINRPQIGVVREFLTRNPAWRKAVFISPYNS
QNAVASKILGLPTQTVDSSQGSEYDYVIFTQTTETAHSCNVNRFNVA
ITRAKVGILCIMSDRDLYDKLQFTSLEIPRRNVATLQ

a polypeptide having residues 18040 to 19620 (nsp14A2 and nsp14; 3′-to-5′ exonuclease) is provided below as SEQ ID NO:65:

AENVTGLFKDCSKVITGLHPTQAPTHLSVDTKFKTEGLCVDIPGIPK
DMTYRRLISMMGFKMNYQVNGYPNMFITREEAIRHVRAWIGFDVEGC
HATREAVGTNLPLQLGFSTGVNLVAVPTGYVDTPNNTDFSRVSAKPP
PGDQFKHLIPLMYKGLPWNVVRIKIVQMLSDTLKNLSDRVVFVLWAH
GFELTSMKYFVKIGPERTCCLCDRRATCFSTASDTYACWHHSIGFDY
VYNPFMIDVQWGFTGNLQSNHDLYCQVHGNAHVASCDAIMTRCLAVH
ECFVKRVDWTIEYPIIGDELKINAACRKVQHMVVKAALLADKFPVLH
DIGNPKAIKCVPQADVEWKFYDAQPCSDKAYKIEELFYSYATHSDKF
DGVCLFWNCNVDRYPANSIVCRFDTRVLSNLNLPGCDGGSLYVNKHA
FHTPAFDKSAFVNLKQLPFFYYSDSPCESHGKQVVSDIDYVPLKSAT
CITRQNLGGAVCRHHANEYRLYLDAYNMMISAGFSLWVYKQFDTYNL
WNTFTRLQ

a polypeptide having residues 19621 to 20658 (nsp15A1 and nsp15B; endoRNAse) is provided below as SEQ ID NO:66:

SLENVAFNVVNKGHFDGQQGEVPVSIINNTVYTKVDGVDVELFENKTT
LPVNVAFELWAKRNIKPVPEVKILNNLGVDIAANTVIWDYKRDAPAHI
STIGVCSMTDIAKKPTETICAPLTVFFDGRVDGQVDLFRNARNGVLIT
EGSVKGLQPSVGPKQASLNGVTLIGEAVKTQFNYYKKVDGVVQQLPET
YFTQSRNLQEFKPRSQMEIDFLELAMDEFIERYKLEGYAFEHIVYGDF
SHSQLGGLHLLIGLAKRFKESPFELEDFIPMDSTVKNYFITDAQTGSS
KCVCSVIDLLLDDFVEIIKSQDLSVVSKVVKVTIDYTEISFMLWCKDG
HVETFYPKLQ

a polypeptide having residues 20659 to 21552 (nsp16; 2′-O-ribose methyltransferase) is provided below as SEQ ID NO:67:

SSQAWQPGVAMPNLYKMQRMLLEKCDLQNYGDSATLPKGIMMNVAKYT
QLCQYLNTLTLAVPYNMRVIHFGAGSDKGVAPGTAVLRQWLPTGTLLV
DSDLNDFVSDADSTLIGDCATVHTANKWDLIISDMYDPKTKNVTKEND
SKEGFFTYICGFIQQKLALGGSVAIKITEHSWNADLYKLMGHFAWWTA
FVTNVNASSSEAFLIGCNYLGKPREQIDGYVMHANYIFWRNTNPIQLS
SYSLFDMSKFPLKLRGTAVMSLKEGQINDMILSLLSKGRLIIRENNRV
VISSDVLVNN

S amino acid sequence is provided below as (SEQ ID NO:68):

MFVFLVLLPLVSSQCVNLTTRTQLPPAYTNSFTRGVYYPDKVFRSSV
LHSTQDLFLPFFSNVTWFHAIHVSGTNGTKRFDNPVLPFNDGVYFAS
TEKSNIIRGWIFGTTLDSKTQSLLIVNNATNVVIKVCEFQFCNDPFL
GVYYHKNNKSWMESEFRVYSSANNCTFEYVSQPFLMDLEGKQGNFKN
LREFVFKNIDGYFKIYSKHTPINLVRDLPQGFSALEPLVDLPIGINI
TRFQTLLALHRSYLTPGDSSSGWTAGAAAYYVGYLQPRTFLLKYNEN
GTITDAVDCALDPLSETKCTLKSFTVEKGIYQTSNFRVQPTESIVRF
PNITNLCPFGEVFNATRFASVYAWNRKRISNCVADYSVLYNSASFST
FKCYGVSPTKLNDLCFTNVYADSFVIRGDEVRQIAPGQTGKIADYNY
KLPDDFTGCVIAWNSNNLDSKVGGNYNYLYRLFRKSNLKPFERDIST
EIYQAGSTPCNGVEGENCYFPLQSYGFQPTNGVGYQPYRVVVLSFEL
LHAPATVCGPKKSTNLVKNKCVNFNFNGLTGTGVLTESNKKFLPFQQ
FGRDIADTTDAVRDPQTLEILDITPCSFGGVSVITPGTNTSNQVAVL
YQDVNCTEVPVAIHADQLTPTWRVYSTGSNVFQTRAGCLIGAEHVNN
SYECDIPIGAGICASYQTQTNSPRRARSVASQSIIAYTMSLGAENSV
AYSNNSIAIPTNFTISVTTEILPVSMTKTSVDCTMYICGDSTECSNL
LLQYGSFCTQLNRALTGIAVEQDKNTQEVFAQVKQIYKTPPIKDFGG
FNFSQILPDPSKPSKRSFIEDLLFNKVTLADAGFIKQYGDCLGDIAA
RDLICAQKFNGLTVLPPLLTDEMIAQYTSALLAGTITSGWTFGAGAA
LQIPFAMQMAYRFNGIGVTQNVLYENQKLIANQFNSAIGKIQDSLSS
TASALGKLQDVVNQNAQALNTLVKQLSSNFGAISSVLNDILSRLDKV
EAEVQIDRLITGRLQSLQTYVTQQLIRAAEIRASANLAATKMSECVL
GQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDV
VIGIVNNTVYDPLQPELDSFKEELDKYFKNHTSPDVDLGDISGINAS
VVNIQKEIDRLNEVAKNLNESLIDLQELGKYEQYIKWPWYIWLGFIA
GLIAIVMVTIMLCCMTSCCSCLKGCCSCGSCCKFDEDDSEPVLKGVK
LHYT

ORF3a amino acid sequence is provided below as (SEQ ID NO:69):

MDLFMRIFTIGTVTLKQGEIKDATPSDFVRATATIPIQASLPFGWLI
VGVALLAVFQSASKIITLKKRWQLALSKGVHFVCNLLLLFVTVYSHL
LLVAAGLEAPFLYLYALVYFLQSINFVRIIMRLWLCWKCRSKNPLLY
DANYFLCWHTNCYDYCIPYNSVTSSIVITSGDGTTSPISEHDYQIGG
YTEKWESGVKDCVVLHSYFTSDYYQLYSTQLSTDTGVEHVTFFIYNK
IVDEPEEHVQIHTIDGSSGVVNPVMEPIYDEPTTTTSVPL

E amino acid sequence is provided below as (SEQ ID NO:70):

MYSFVSEETGTLIVNSVLLFLAFVVFLLVTLAILTALRLCAYCCNIVNVSL
VKPSFYVYSRVKNLNSSRVPDLLV

M amino acid sequence is provided below as (SEQ ID NO:71):

MADSNGTITVEELKKLLEQWNLVIGFLFLTWICLLQFAYANRNRFLYIIK
LIFLWLLWPVTLACFVLAAVYRINWITGGIAIAMACLVGLMWLSYFIASF
RLFARTRSMWSFNPETNILLNVPLHGTILTRPLLESELVIGAVILRGHLR
IAGHHLGRCDIKDLPKEITVATSRTLSYYKLGASQRVAGDSGFAAYSRYR
IGNYKLNTDHSSSSDNIALLVQ

ORF6 amino acid sequence is provided below as (SEQ ID NO:72):

MFHLVDFQVTIAEILLIIMRTFKVSIWNLDYIINLIIKNLSKSLTENKY
SQLDEEQPMEID

ORF7a amino acid sequence is provided below as (SEQ ID NO:73):

MKIILFLALITLATCELYHYQECVRGTTVLLKEPCSSGTYEGNSPFHPL
ADNKFALTCFSTQFAFACPDGVKHVYQLRARSVSPKLFIRQEEVQELYS
PIFLIVAAIVFITLCFTLKRKTE

ORF7b amino acid sequence is provided below as (SEQ ID NO:74):

MIELSLIDFYLCFLAFLLFLVLIMLIIFWFSLELQDHNETCHA

ORF8 amino acid sequence is provided below as (SEQ ID NO:75):

MKFLVFLGIITTVAAFHQECSLQSCTQHQPYVVDDPCPIHFYSKWYIRV
GARKSAPLIELCVDEAGSKSPIQYIDIGNYTVSCLPFTINCQEPKLGSL
VVRCSFYEDFLEYHDVRVVLDFI

N amino acid sequence is provided below as (SEQ ID NO:76):

MSDNGPQNQRNAPRITFGGPSDSTGSNQNGERSGARSKQRRPQGLPNNT
ASWFTALTQHGKEDLKFPRGQGVPINTNSSPDDQIGYYRRATRRIRGGD
GKMKDLSPRWYFYYLGTGPEAGLPYGANKDGIIWVATEGALNTPKDHIG
TRNPANNAAIVLQLPQGTTLPKGFYAEGSRGGSQASSRSSSRSRNSSRN
STPGSSRGTSPARMAGNGGDAALALLLLDRLNQLESKMSGKGQQQQGQT
VTKKSAAEASKKPRQKRTATKAYNVTQAFGRRGPEQTQGNFGDQELIRQ
GTDYKHWPQIAQFAPSASAFFGMSRIGMEVTPSGTWLTYTGAIKLDDKD
PNFKDQVILLNKHIDAYKTFPPTEPKKDKKKKADETQALPQRQKKQQTV
TLLPAADLDDFSKQLQQSMSSADSTQA

ORF10 amino acid sequence is provided below as (SEQ ID NO:77):

MGYINVFAFPFTIYSLLLCRMNSRNYIAQVDVVNFNLT

or a polypeptide having at least 80%, 82%, 84%, 85%, 87%, 89%, 90%, 92%, 94%, 95%, 96%, 97%, 98% or 99& % amino acid sequence identity thereto.

In one embodiment, a cold-adapted coronavirus has a mutation in one or more of genes for Nsp1, Nsp2, Nsp3, Nsp6, Nsp13, Nsp14, Nsp15, ORF6, ORF7b, ORF8, or any combination thereof. In one embodiment, a cold-adapted coronavirus has a Nsp1, Nsp2, Nsp3, Nsp6, Nsp13, Nsp14, Nsp15, polypeptide encoded by ORF6, with at least 80%, 82%, 84%, 85%, 87%, 89%, 90%, 92%, 94%, 95%, 96%, 97%, 98% or 99& % amino acid sequence identity to one of SEQ ID Nos. 20-22, 25, 33-34, 72, 74, or 75.

The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a given gene or protein will possess a relatively high degree of sequence identity when aligned using standard methods. Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237-244, 1988; Higgins and Sharp, CABIOS 5:151-153, 1989; Corpet et al., Nucleic Acids' Research 16:10881-10890, 1988; and Pearson and Lipman, Proc. Natl. Acad. Sci. U.S.A. 85:2444, 1988. Altschul et al., Nature Genet. 6:119-129, 1994. The NCBI Basic Local Alignment Search Tool (BLAST™) (Altschul et al., J. Mol. Biol. 215:403-410, 1990) is available from several sources, including the National Center for Biotechnology Information (NCBI, Bethesda, MD) and on the Internet, for use in connection with the sequence analysis programs blastp, blastn, blastx, tblastn and tblastx.

Sequence identity between nucleic acid sequences, or between amino acid sequences, can be determined by comparing an alignment of the sequences. When an equivalent position in the compared sequences is occupied by the same nucleotide, or amino acid, then the molecules are identical at that position. Scoring an alignment as a percentage of identity is a function of the number of identical nucleotides or amino acids at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include MatGat (Campanella et al., 2003, BMC Bioinformatics 4:29; program available from bitincka.com/ledion/matgat), Gap (Needleman & Wunsch, 1970, J. Mol. Biol. 48:443-453), FASTA (Altschul et al., 1990, J. Mol. Biol. 215:403-410; program available from ebi.ac.uk/fasta), Clustal W 2.0 and X 2.0 (Larkin et al., 2007, Bioinformatics 23:2947-2948; program available from ebi.uk/tools/clustalw2) and EMBOSS Pairwise Alignment Algorithms (Needleman & Wunsch, 1970, supra; Kruskal, 1983, In: Time warps, string edits and macromolecules: the theory and practice of sequence comparison, Sankoff & Kruskal (eds), pp 1-44, Addison Wesley; programs available from ebi.ac.uk/tools/emboss/align. All programs may be run using default parameters.

For example, sequence comparisons may be undertaken using the “needle” method of the EMBOSS Pairwise Alignment Algorithms, which determines an optimum alignment (including gaps) of two sequences when considered over their entire length and provides a percentage identity score. Default parameters for amino acid sequence comparisons (“Protein Molecule” option) may be Gap Extend penalty: 0.5, Gap Open penalty: 10.0, Matrix: Blosum 62.

The sequence comparison may be performed over the full length of the reference sequence.

Sequences described herein include reference to an amino acid sequence comprising an amino acid residue “at a position corresponding to an amino acid residue position” of another sequence. Such corresponding positions may be identified, for example, from an alignment of the sequences using a sequence alignment method described herein, or another sequence alignment method known to the person of ordinary skill in the art.

The subject for the vaccine may be any animal, including a human and non-human animals. Non-human animals include all vertebrates, e.g., mammals and non-mammals, such as non-human primates, sheep, dogs, cats, cows, horses, chickens, amphibians, and reptiles, e.g., mammals, such as humans or non-human primates, sheep, dogs, cats, cows and horses. The subject may also be livestock such as, cattle, swine, sheep, poultry, and horses, or pets, such as dogs and cats.

The methods described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, children, and infants.

Examples of human ethnic populations include Caucasians, Asians, Hispanics, Africans, African Americans, Native Americans, Semites, and Pacific Islanders. The methods may be more appropriate for some ethnic populations such as Caucasians, especially northern European populations, as well as Asian populations.

The term subject also includes subjects of any genotype or phenotype as long as they are in need of the compositions, as described above. In addition, the subject can have the genotype or phenotype for any hair color, eye color, skin color or any combination thereof.

The term subject includes a subject of any body height, body weight, or any organ or body part size or shape.

The invention will be further described by the following non-limiting examples.

### EXAMPLES

The invention will be described by the following non-limiting examples.

### Example 1: Identification of Mutations Associated with Attenuation and Temperature-Sensitive, Cold-Adaption of Live Attenuated Coronavirus

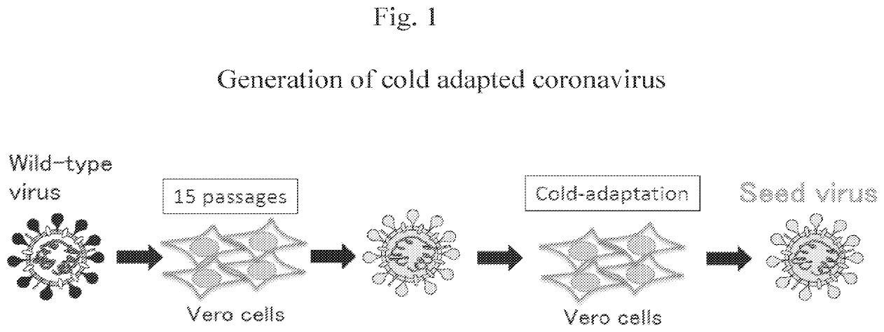

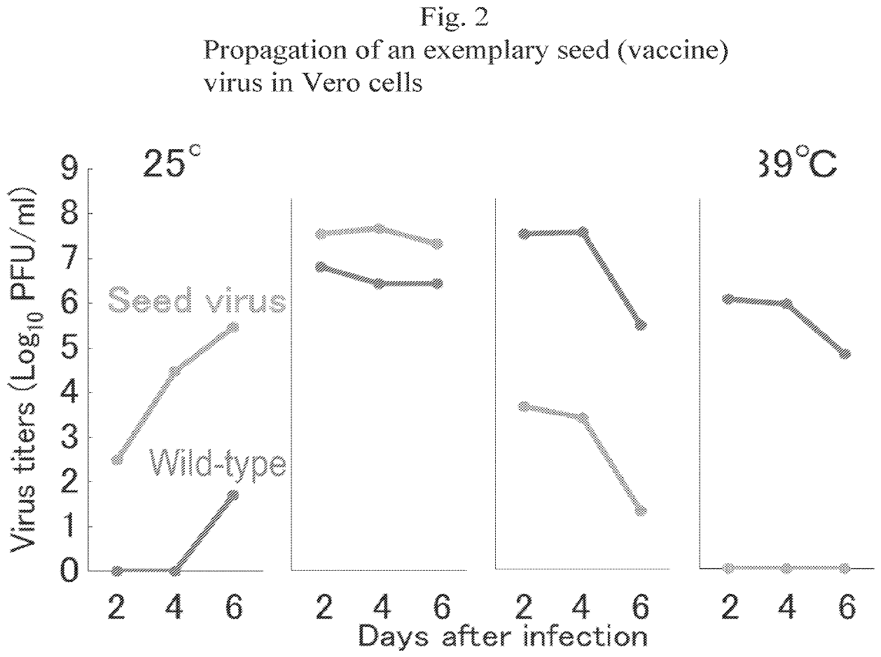

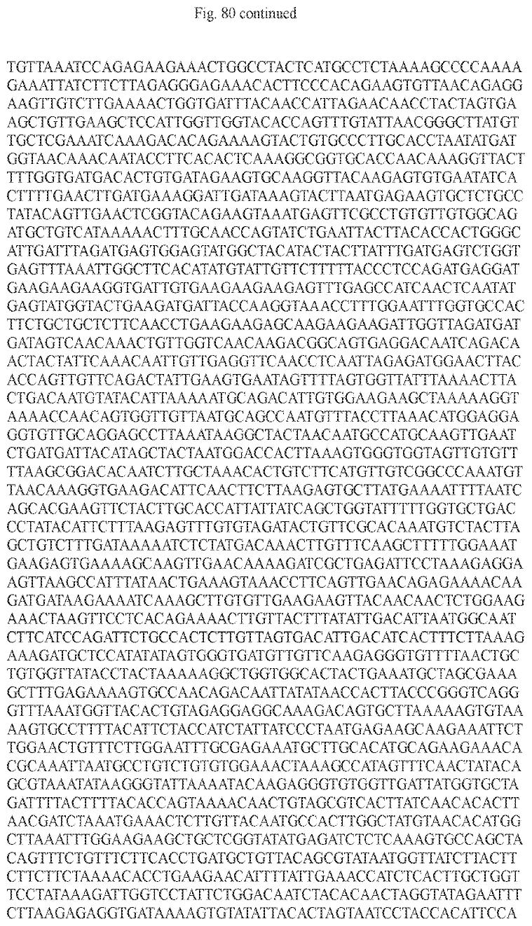

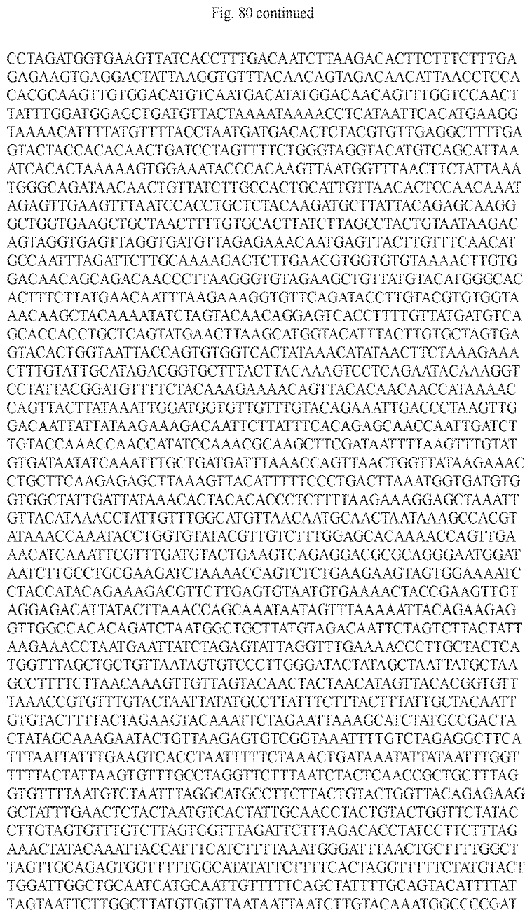

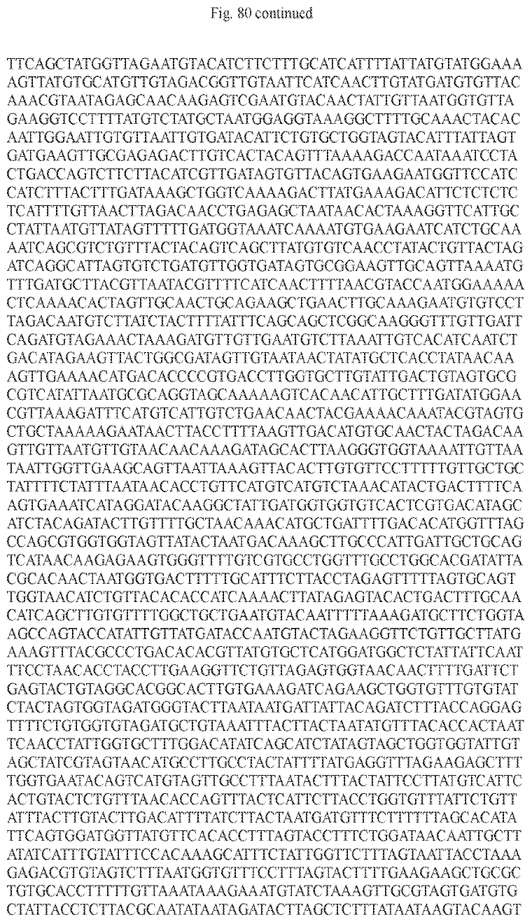

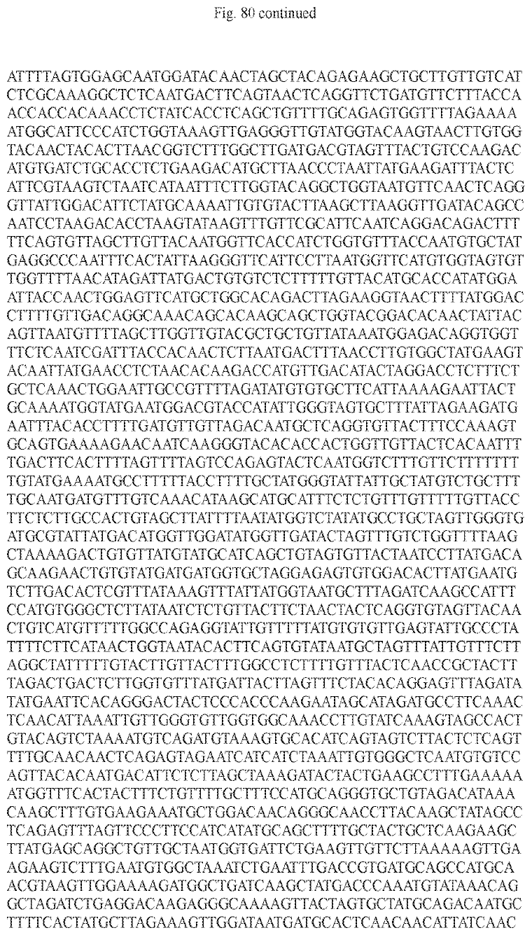

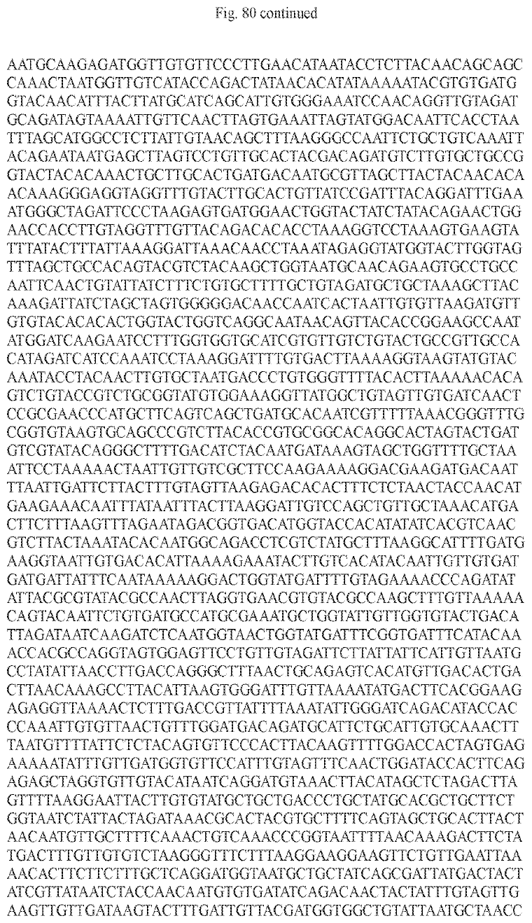

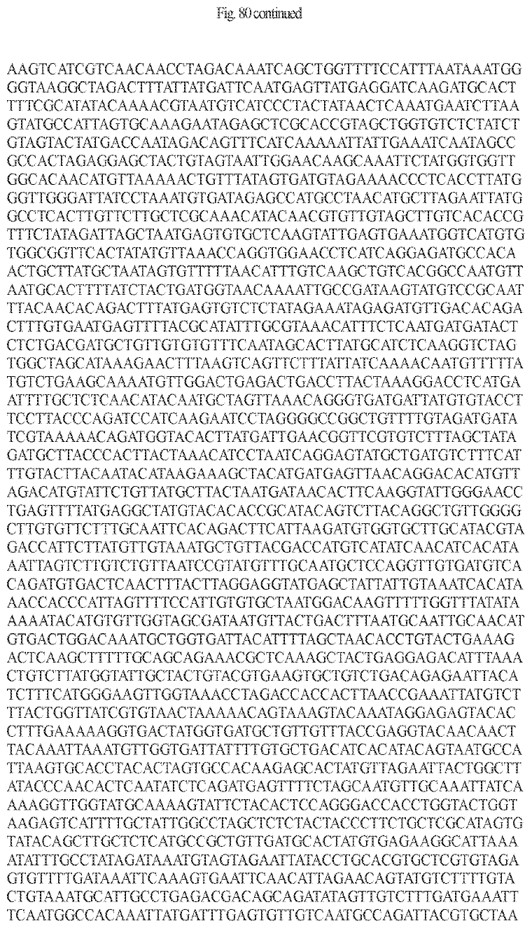

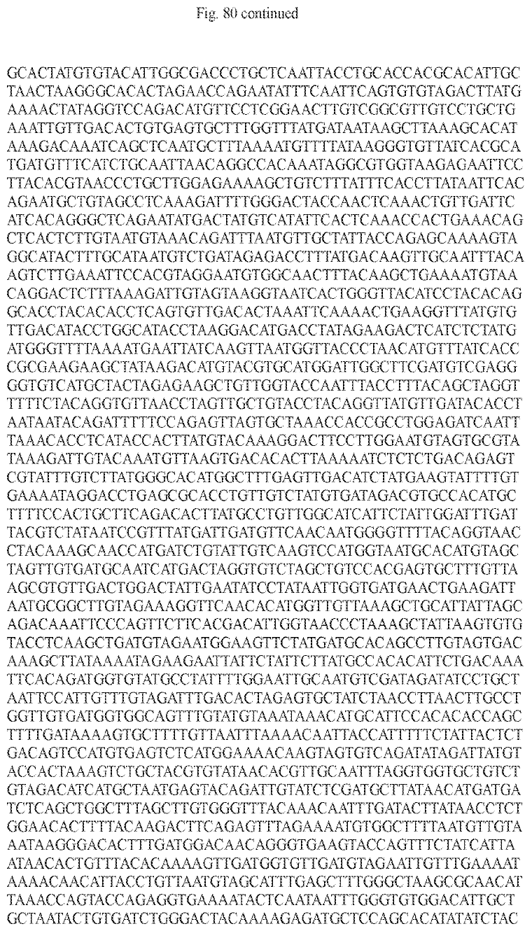

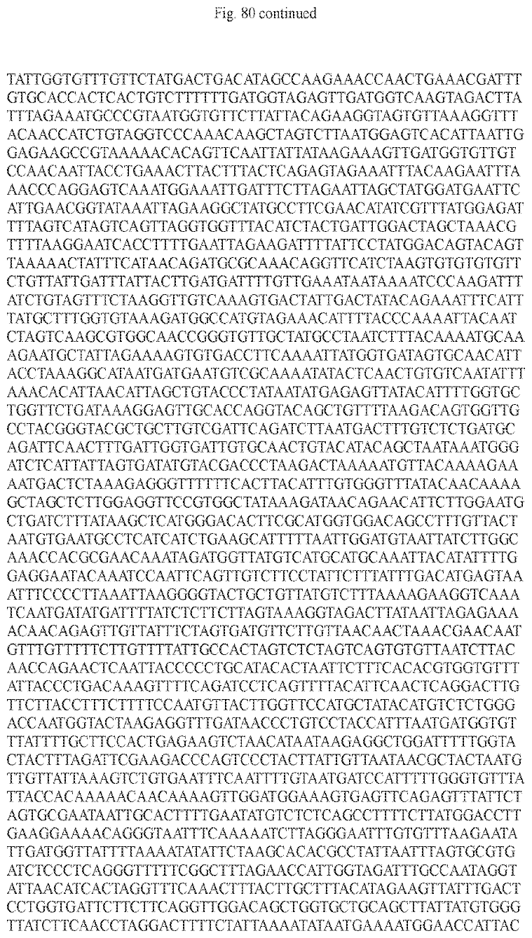

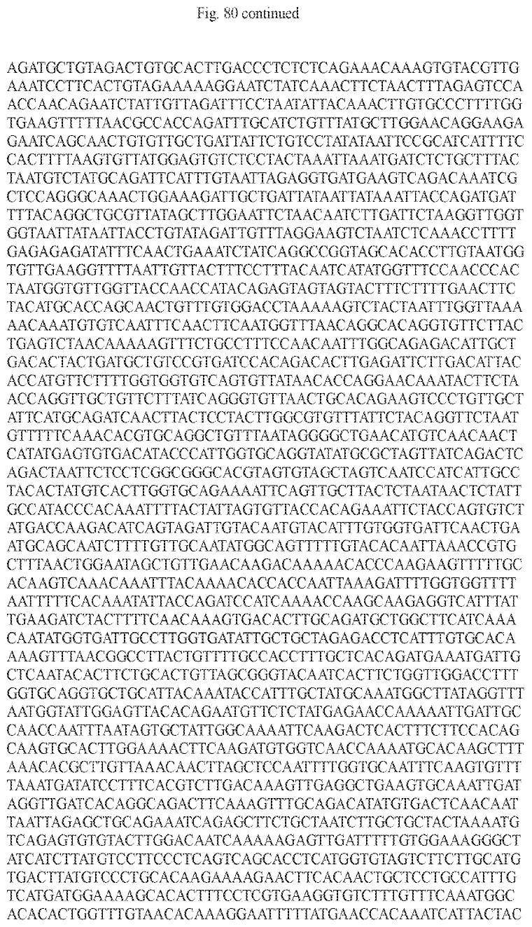

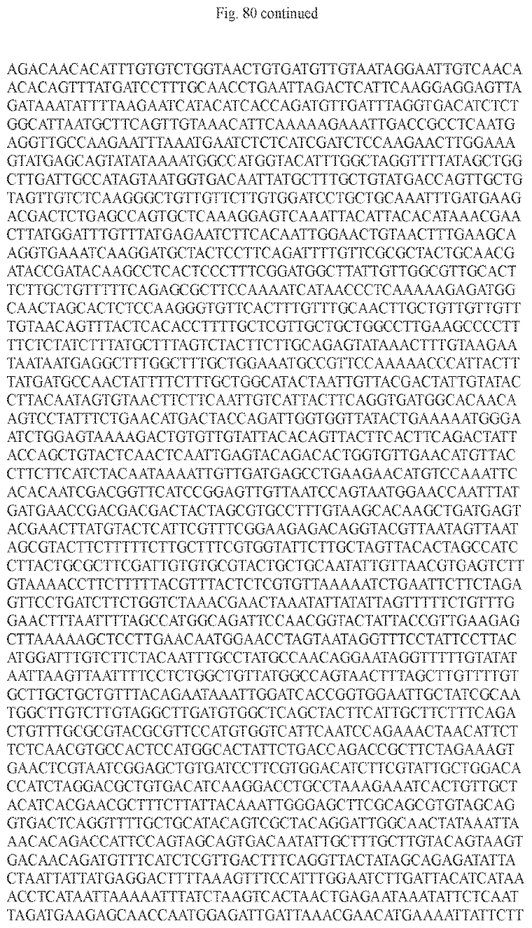

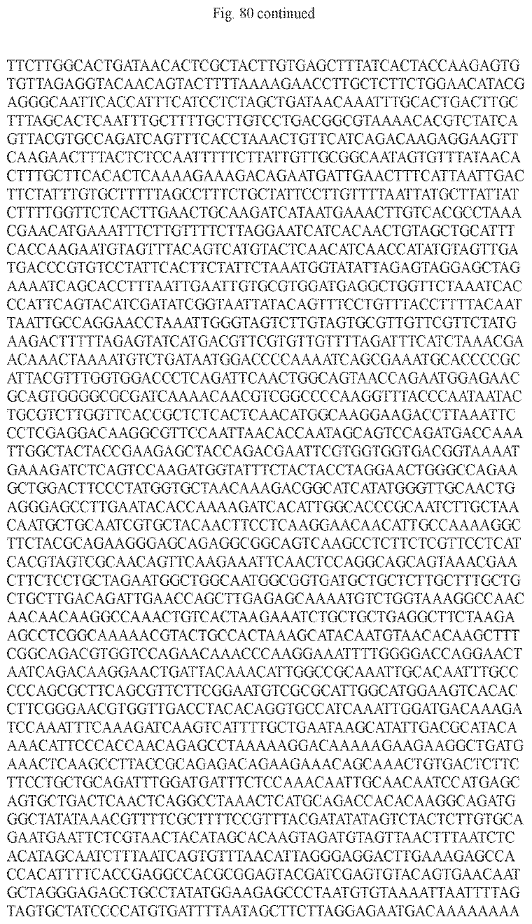

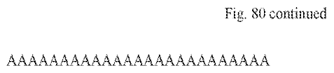

A clinical isolate of wild-type SARS-Cov-2/UT-HPCo038/human/2020/Tokyo (FIG. 80, SEQ ID NO:78) was passaged 15 times at 37° C. in Vero cells. After cloning the virus by plaque purification, cold adaptation was initiated at progressively lower temperatures from 35° C. to 25° C. with the last 3 passages at 25° C. (FIGS. 1 and 2). When viruses grew well in Vero cells at 25° C., plaque purification was performed again to obtain cold-adapted coronavirus. The cold-adapted coronavirus replicated well at 25° C. and 32° C. but failed to propagate at 37° C. and 39° C. compared to the wild-type virus.

The cells are cultured at 30° C. for 1 day before infection. To demonstrate attenuation, golden Syrian hamsters were infected with the parent virus (before passaging) and the cold-adapted virus after 15 passages. Syrian hamsters are highly susceptible to SARS-CoV-2 infection and present with pathological phenotypes similar to those of infected humans; therefore, Syrian hamsters are a model to evaluate the attenuation of cold-adapted SARS-CoV-2.

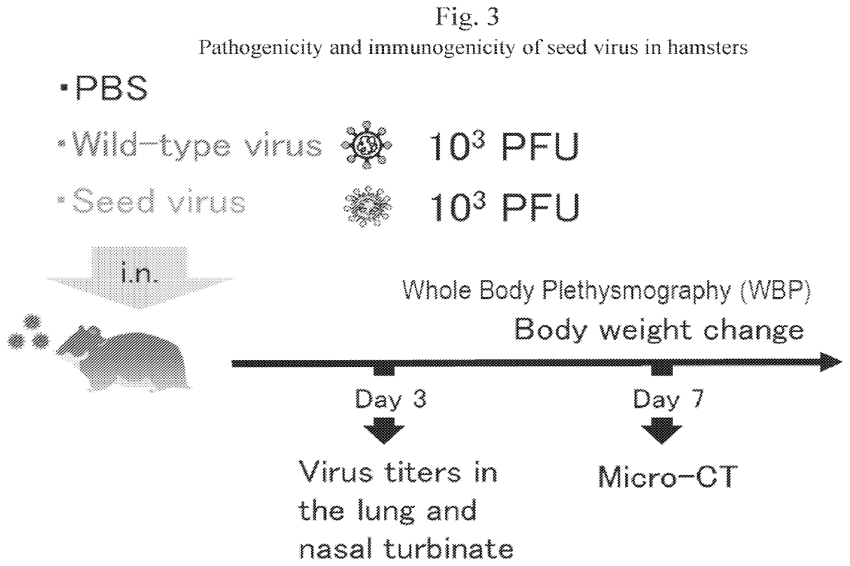

To test the pathogenicity of the cold-adapted coronavirus, hamsters were intranasally inoculated with 10³ PFU of cold-adapted coronavirus (FIG. 3). Body weight and respiratory function were monitored for 8-9 days post-infection. Lung and nasal titers were measured 3 days after infection. Micro-CT analysis was performed at 7 days post-infection.

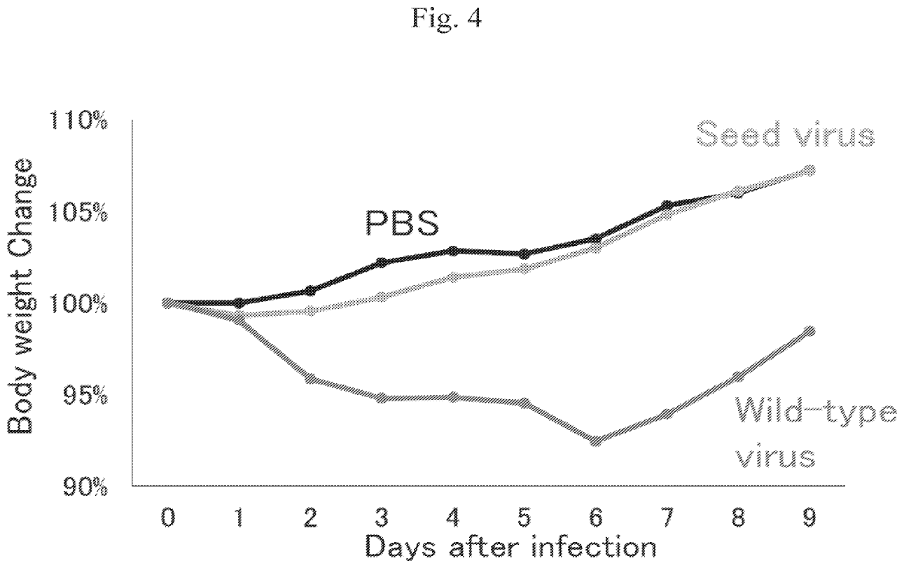

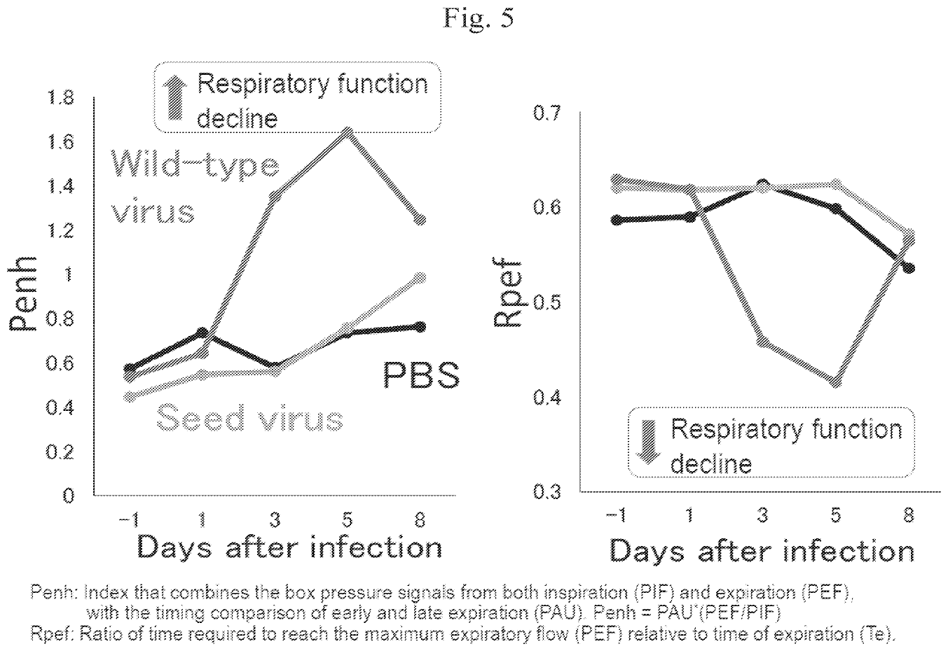

The body weight of hamsters infected with cold-adapted coronavirus gradually increased, similar to PBS controls, whereas that of hamsters infected with wild-type virus was significantly reduced (FIG. 4). Respiratory function was tested by whole-body plethysmography (WBP) before infection and at 1-, 3-, 5-, and 8-days post infection (dpi). Whole body plethysmography (WBP) showed that the cold-adapted coronavirus did not cause a decrease in respiratory function based on Penh and Rpef (FIG. 5). Penh: Index that combines the box pressure signals from both inspiration (PIF) and expiration (PEF), with the timing comparison of early and late expiration (PAU). Penh=PAU*(PEF/PIF). Rpef: Ratio of time required to reach the maximum expiratory flow (PEF) relative to time of expiration (Te).

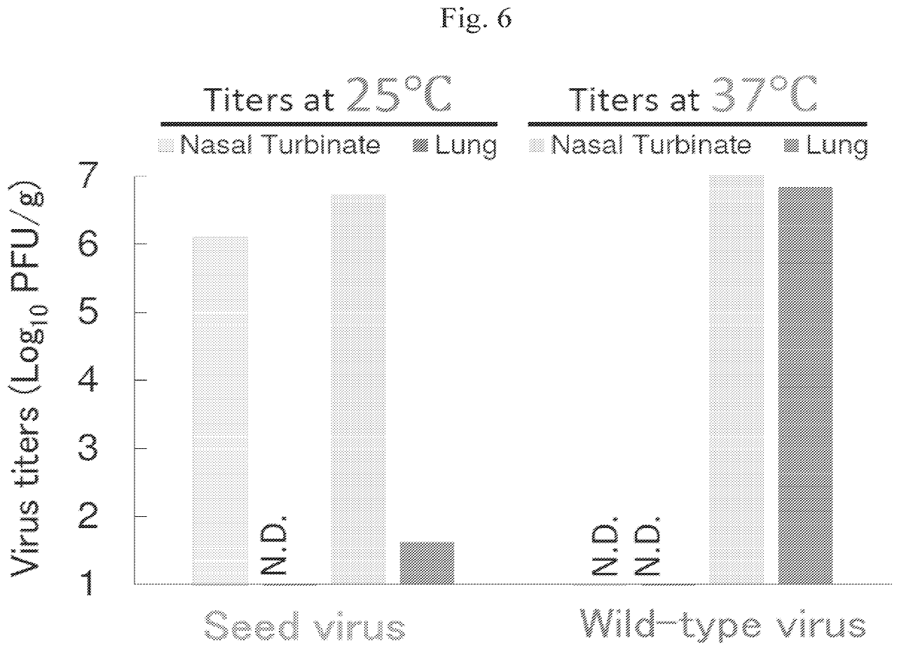

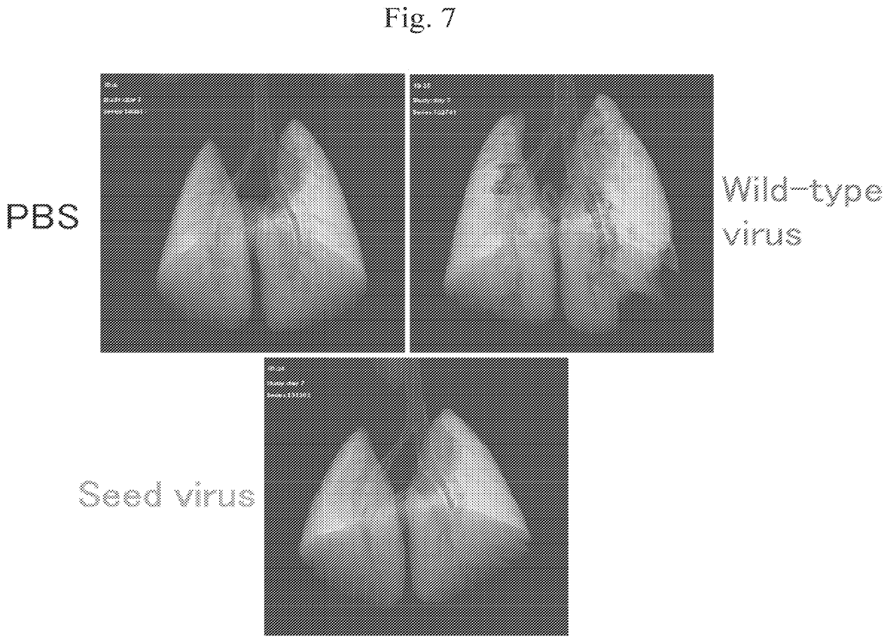

Hamsters infected with wild-type virus exhibited a decline in respiratory function relative to the cold-adapted coronavirus. Four days after infection, the amount of virus in the lungs was determined. Virus titers in the lungs of hamsters infected with cold-adapted coronavirus were significantly reduced compared with those of hamsters infected with wild-type virus; however, virus titers in the nasal turbinates were similar for cold-adapted coronavirus- and wild-type virus-infected hamsters (FIG. 6). Computed tomography (CT) scans of the lungs of hamsters administered the phosphate buffered saline control, cold-adapted coronavirus, and wild-type virus were performed (FIG. 7). No significant inflammatory findings were detected by micro-CT analysis in the lungs of hamsters infected with the cold-adapted coronavirus. Wild-type virus caused emphysema and extensive inflammation in the lungs of infected hamsters.

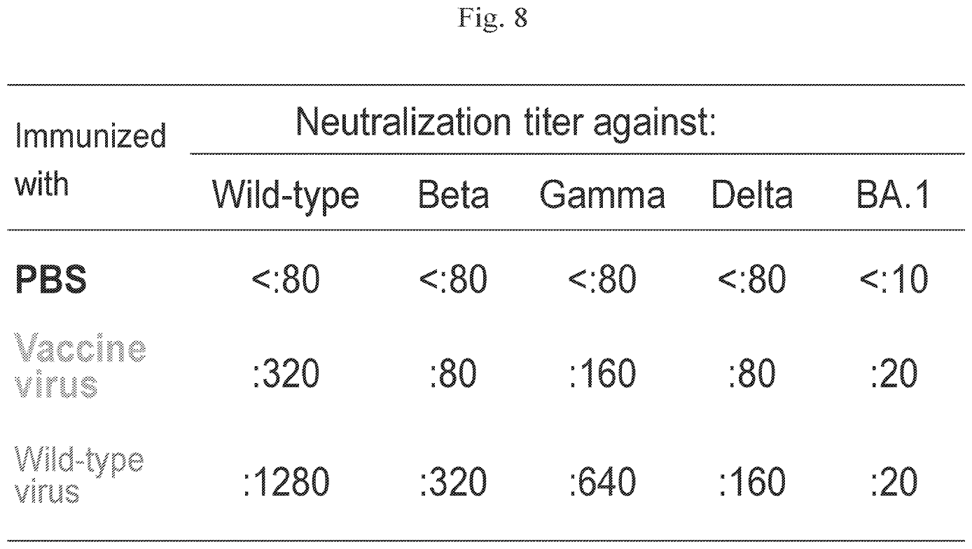

Serological assays were performed on the cold-adapted coronavirus and wild-type inoculated hamsters for detecting neutralizing antibodies against SARS-CoV-2 (FIG. 8). Both seed and wild-type virus induced neutralizing antibodies against wild-type, beta, gamma, delta and BA. 1 variants in infected hamsters.

Given the attenuation in hamsters, this cold-adapted version of SARS-CoV-2 may be used as a vaccine candidate.

ORF1ab-Y1090N was the most important mutation for attenuation in the hamster. ORF1ab-M85del also contributed to the attenuation.

In one embodiment, the cold-adapted virus has at least one of Nsp1-M85del, nsp3 (PLpro)-Y272N, nsp6-L260F, S-V1094F.

TABLE 1

| | | | | nsp3 | nsp3 | | nsp13 |
|---|---|---|---|---|---|---|---|
| | nsp1 | nsp2 | nsp2 | (PLpro) | (PLpro) | nsp6 | (Helicase) |
| Nucleotide | del517- | c1342t | g1495t | t3533a | c4301t | c11750t | c16575t |
| change | 519 | | | | | | |
| compared | | | | | | | |
| with | | | | | | | |
| parental | | | | | | | |
| virus | | | | | | | |
| Amino acid | [symbol] M85 | — | — | Y272N | L528F | L260F | — |
| change | | | | | | | |
| | nsp14(3′-5′ | nsp15 | | | | | ORF7b- |
| | exonuclease) | (endoRNase) | S | S | S | ORF6 | ORF8 |
| Nucleotide | g19117t | a20318c | t21752a | a22320g | g24842t | del27209- | del27816- |
| change | | | | | | 27266 | 28244 |
| compared | | | | | | | |
| with | | | | | | | |
| parental | | | | | | | |
| virus | | | | | | | |
| Amino acid | A360S | E233A | W64R | D253G | V1094F | C-terminus | Deletion of |
| change | | | | | | deleted | ORF8 and |
| | | | | | | ORF6 | ORF7b |

### Example 2: Challenge of Vaccine Virus Immunized Hamsters with Wild-Type Wuhan-Like (SEQ ID NO: 5) Virus

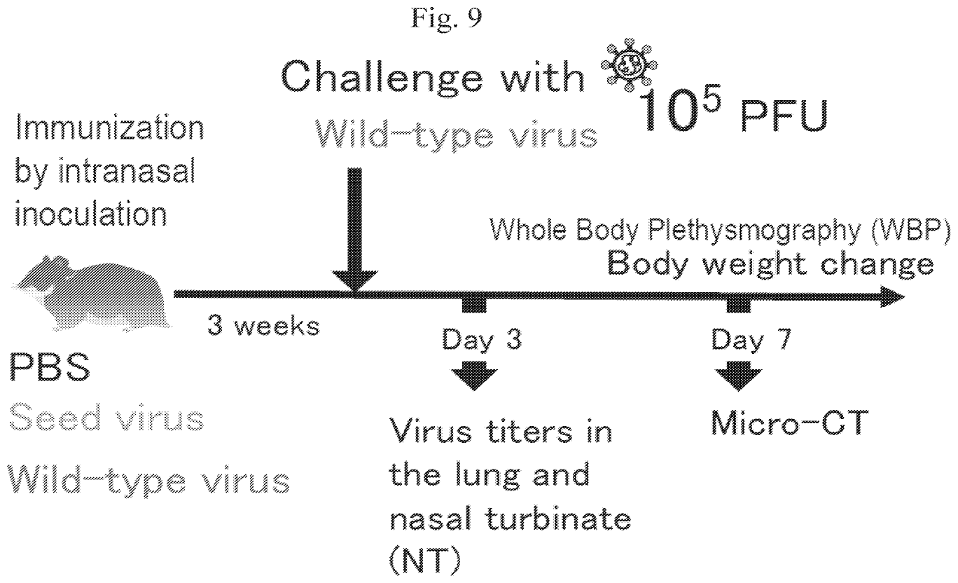

The cold-adapted coronavirus produced as described in Example 1 from the wild-type SARS-Cov-2/UT-HPCo038/human/2020/Tokyo (FIG. 80, SEQ ID NO: 78) was used to immunize hamsters by intranasal inoculation. The immunized hamsters were intranasally challenged with wild-type virus at 10⁵ PFU (FIG. 9). Body weight and respiratory function were monitored for 7 days post-infection. Lung and nasal titers were measured 3 days after infection. Micro-CT analysis was performed at 7 days post-infection.

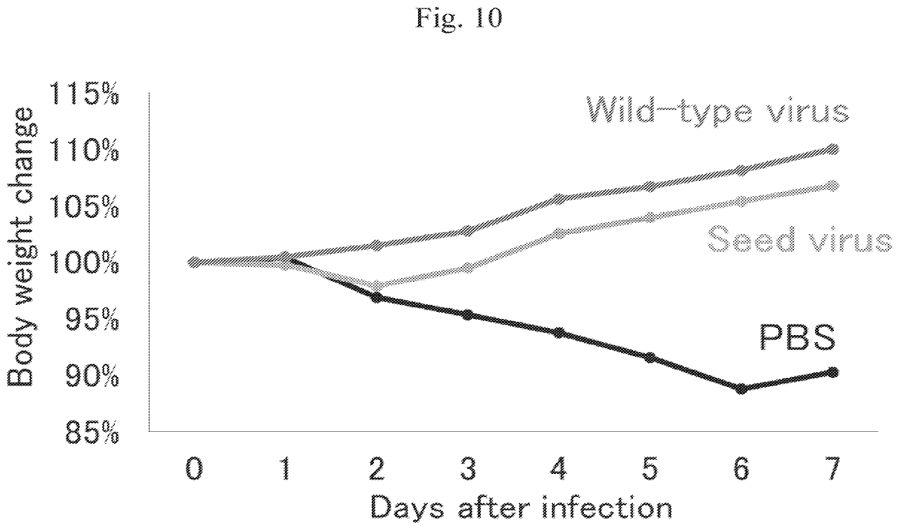

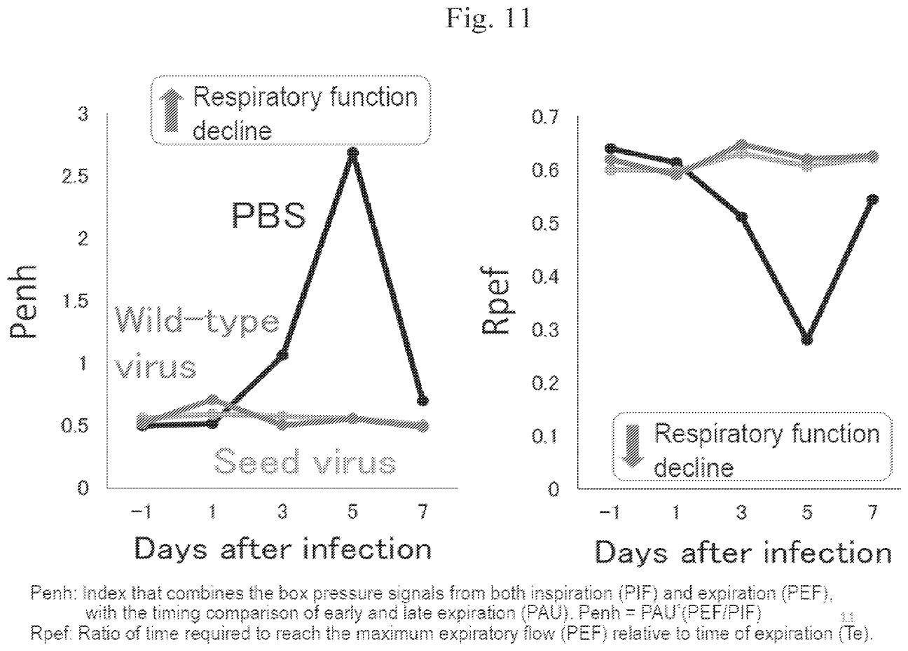

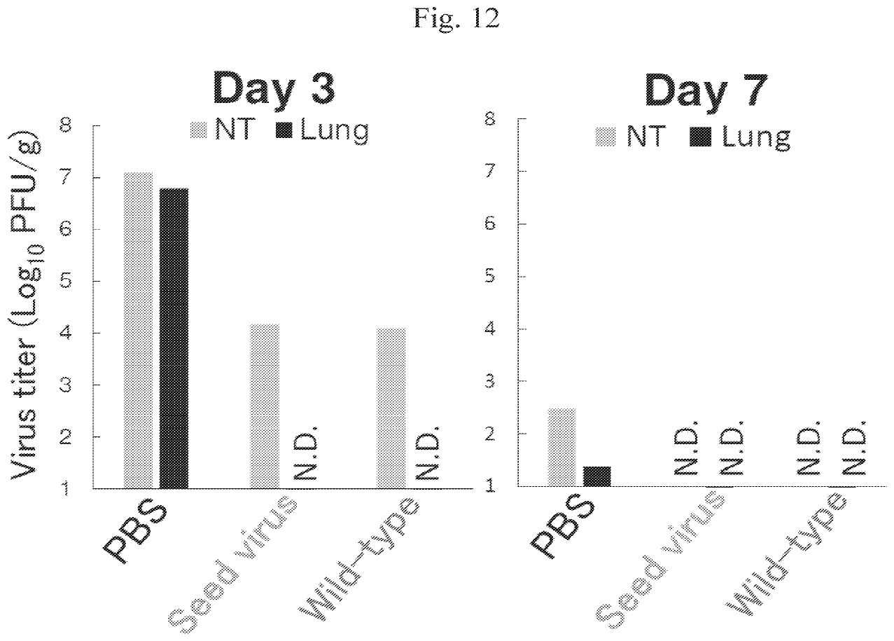

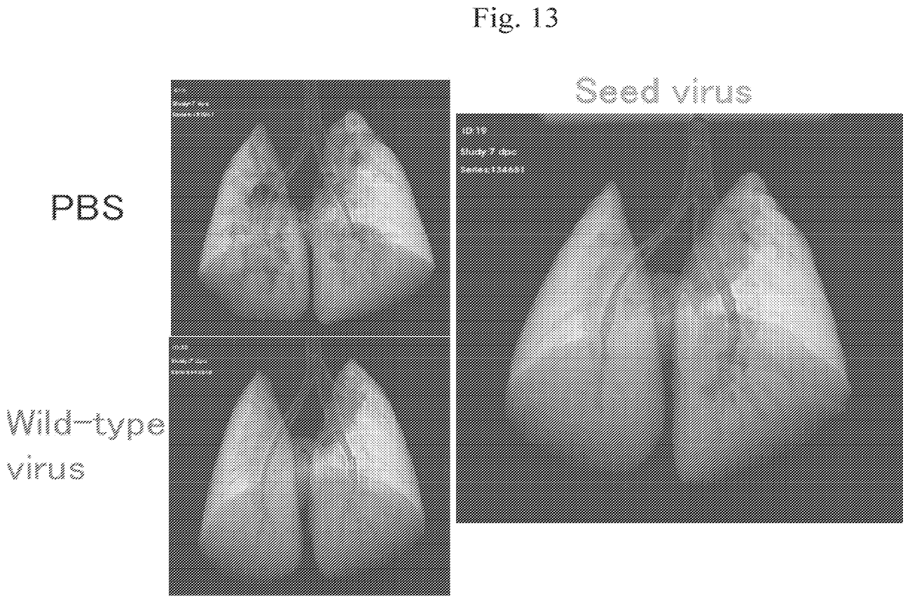

Body weight loss of cold-adapted coronavirus-immunized hamsters was limited, whereas PBS-administrated hamsters showed significant body weight loss after challenge infection (FIG. 10). Whole body plethysmography (WBP) showed that cold-adapted coronavirus-immunized hamsters had no reduction in respiratory function in terms of either Penh or Rpef. In contrast, respiratory function was impaired in PBS-treated hamsters (FIG. 11). Virus titers in the lungs and nasal turbinates of cold-adapted coronavirus-immunized hamsters were significantly reduced compared with those of the PBS group at 3 and 7 days post-infection (FIG. 12). In hamsters immunized with wild-type or vaccine (seed) virus, virus titers were detected in nasal turbinates at about 99.9% less than in unimmunized hamsters and undetected in lung on day 3. By day 7, virus was undetectable in the immunized hamsters. CT scans of lungs of each group are shown in FIG. 13, which showed no significant inflammatory findings as detected by micro-CT analysis in the lungs of cold-adapted coronavirus-immunized hamsters. Extensive inflammation in the lungs of PBS-administrated hamsters was observed.

### Example 3: Challenge of Vaccine Virus Immunized Hamsters with Wild-Type Delta Virus

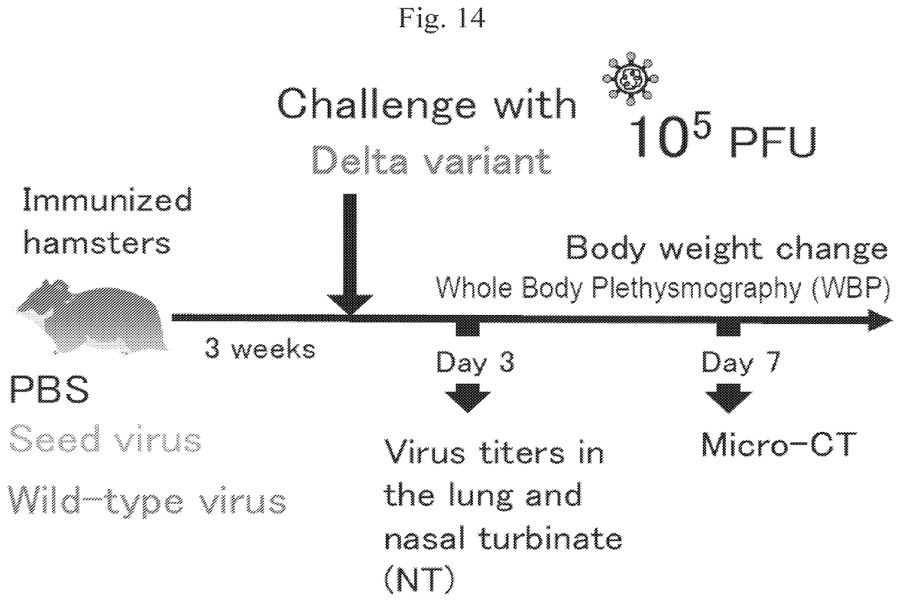

The cold-adapted coronavirus produced as described in Example 1 from the wild-type SARS-Cov-2/UT-HPCo038/human/2020/Tokyo (FIG. 80, SEQ ID NO: 78) was used to immunize hamsters by intranasal inoculation (FIG. 14). Hamsters immunized with the cold-adapted coronavirus were intranasally challenged with wild-type virus at 10⁵ PFU. Body weight and respiratory function were monitored for 7 days post-infection. Lung and nasal titers were measured 3 days after infection. Micro-CT analysis was performed at 7 days post-infection.

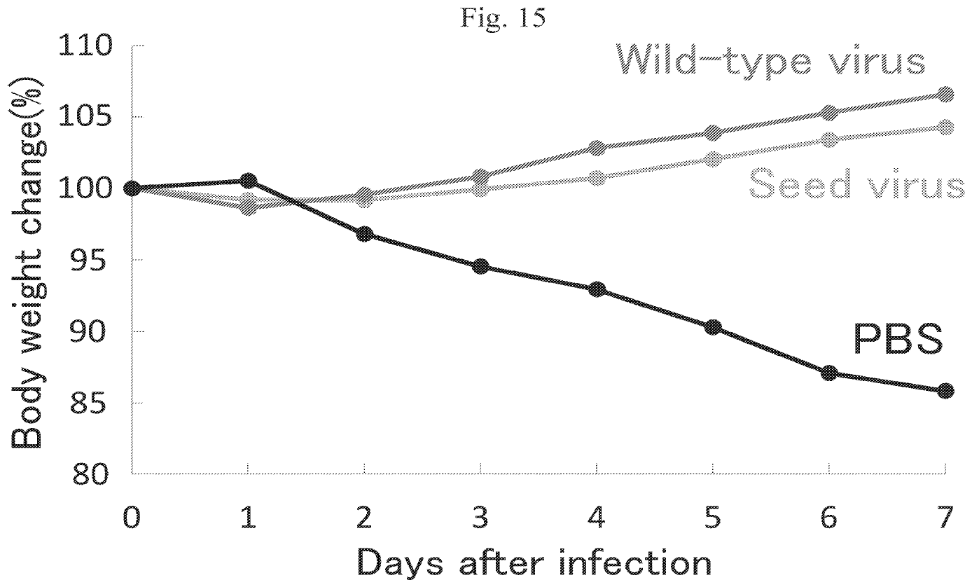

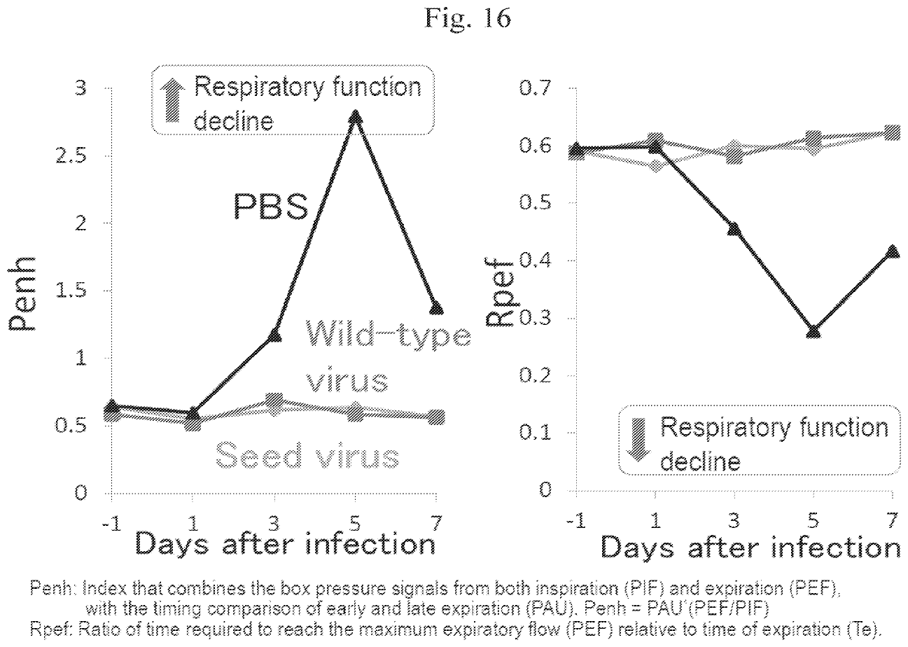

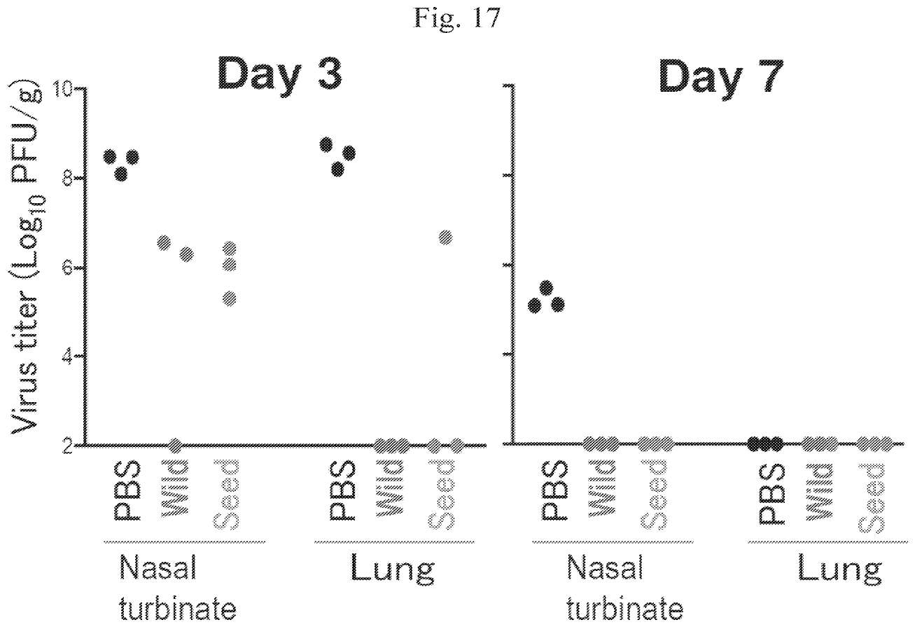

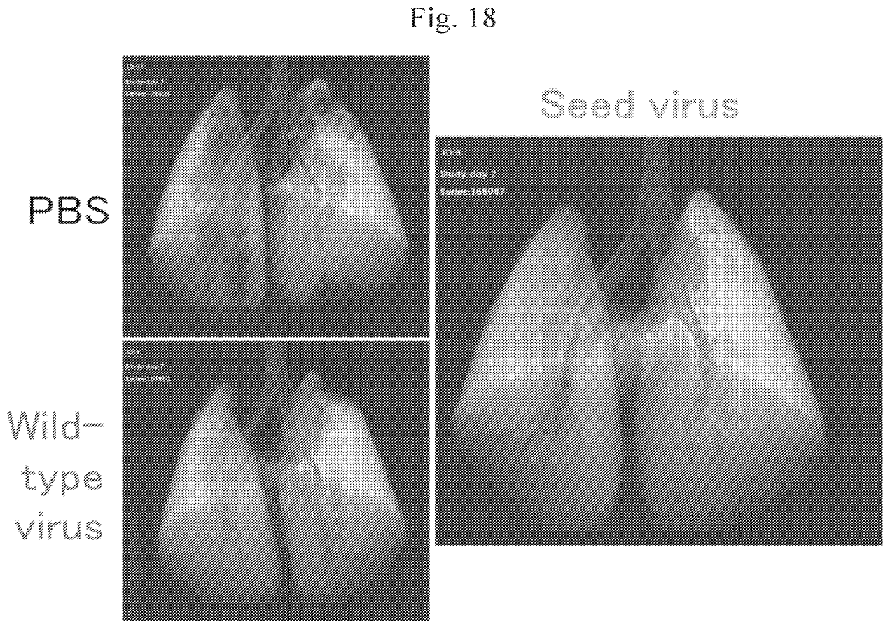

Body weight loss of cold-adapted coronavirus-immunized hamsters was limited, whereas PBS-administrated hamsters showed significant body weight loss after challenge infection (FIG. 15). Whole body plethysmography (WBP) showed that cold-adapted coronavirus-immunized hamsters had no reduction in respiratory function in terms of either Penh or Rpef. In contrast, respiratory function was impaired in PBS-treated hamsters (FIG. 16). Virus titers in the lungs and nasal turbinates of cold-adapted coronavirus-immunized hamsters were significantly reduced compared with those of the PBS group at 3 and 7 days after infection (FIG. 17). In hamsters immunized with wild-type or vaccine (seed) virus, virus titers were detected in NT at about 99% less than in unimmunized hamsters and undetected in lung on day 3 with the exception of one hamster. By day 7, virus was undetectable in the immunized hamsters in either NT or lung. CT scans of lungs of each group are shown in FIG. 18, which showed no significant inflammatory findings were detected by micro-CT analysis in the lungs of cold-adapted coronavirus-immunized hamsters. Extensive inflammation in the lungs of PBS-administrated hamsters was observed.

### Example 4: Passage of Cold-Adapted Coronavirus Through Hamster Nasal Turbinate

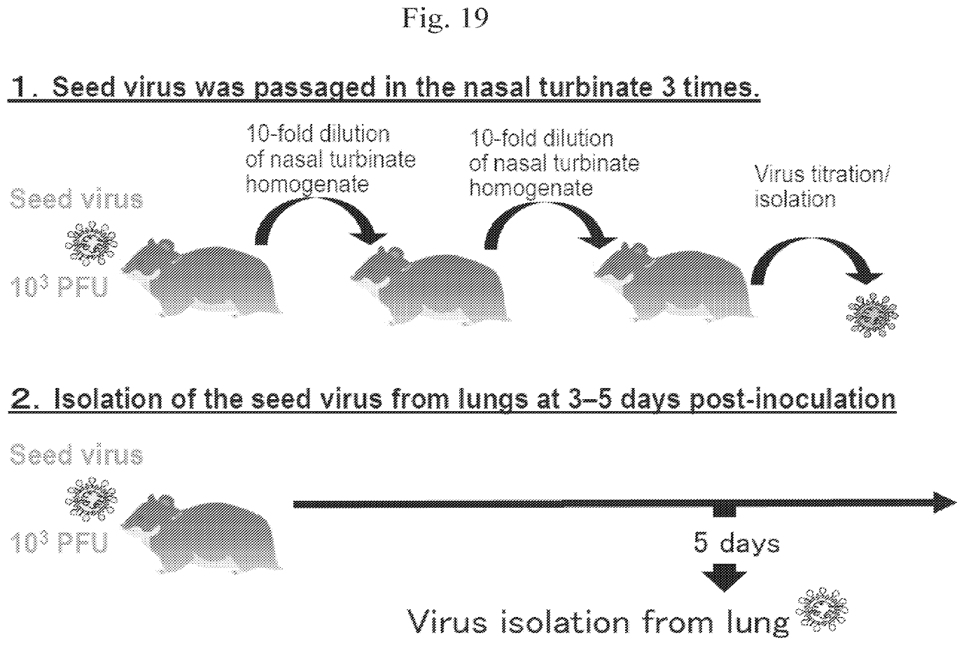

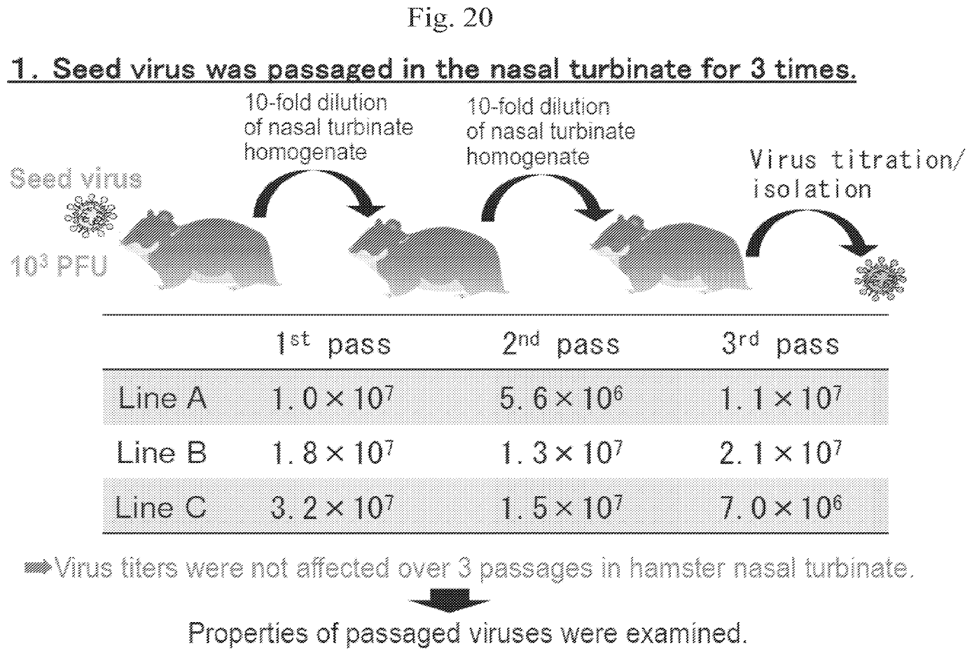

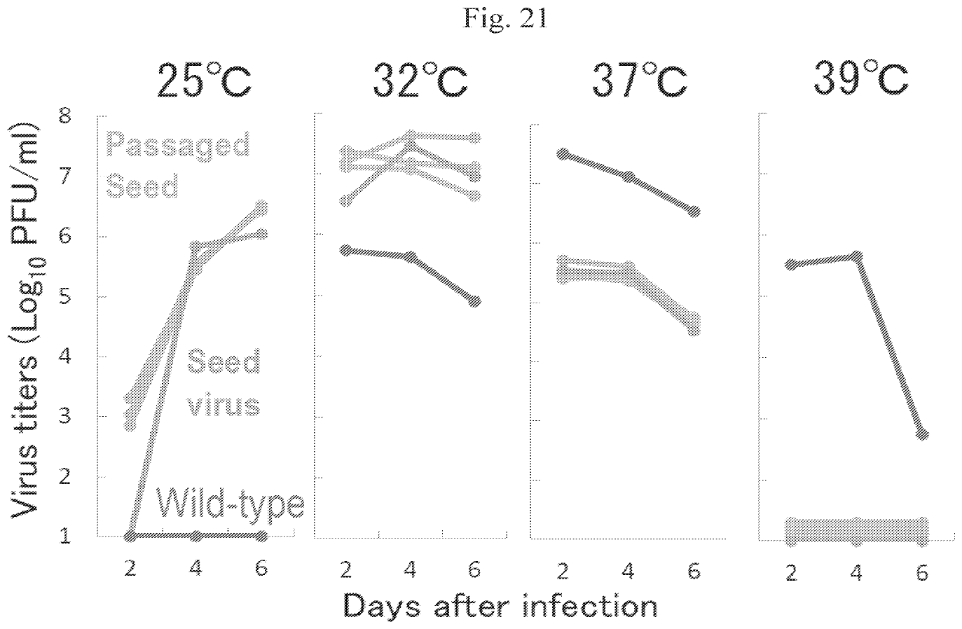

To test the in vivo stability of the attenuated cold-adapted coronavirus, the cold-adapted coronavirus was passaged in the nasal turbinates 3 times or it was isolated from the lungs of hamsters 3-5 days after infection and then tested for cold adaptation, temperature sensitivity, and pathogenicity in hamsters (FIG. 19). Three lineages (Line A, Line B, and Line C) of passaged virus were isolated from lungs at days 3-, 4-, and 5-days post infection. When the cold-adapted coronavirus was passaged in triplicates in the nasal turbinate of hamsters, the virus titers in the nasal turbinate did not change during the 3 passages (FIG. 20). Three lines of cold-adapted coronaviruses passaged 3 times in the nasal turbinates showed similar growth kinetics at 25, 32, 37, and 39° C. to those of the unpassaged cold-adapted coronavirus (FIG. 21).

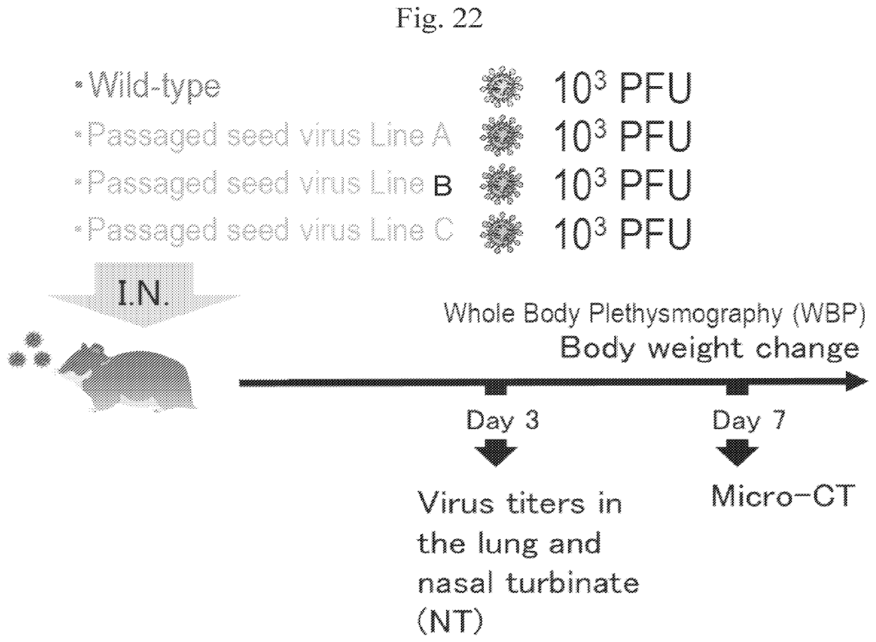

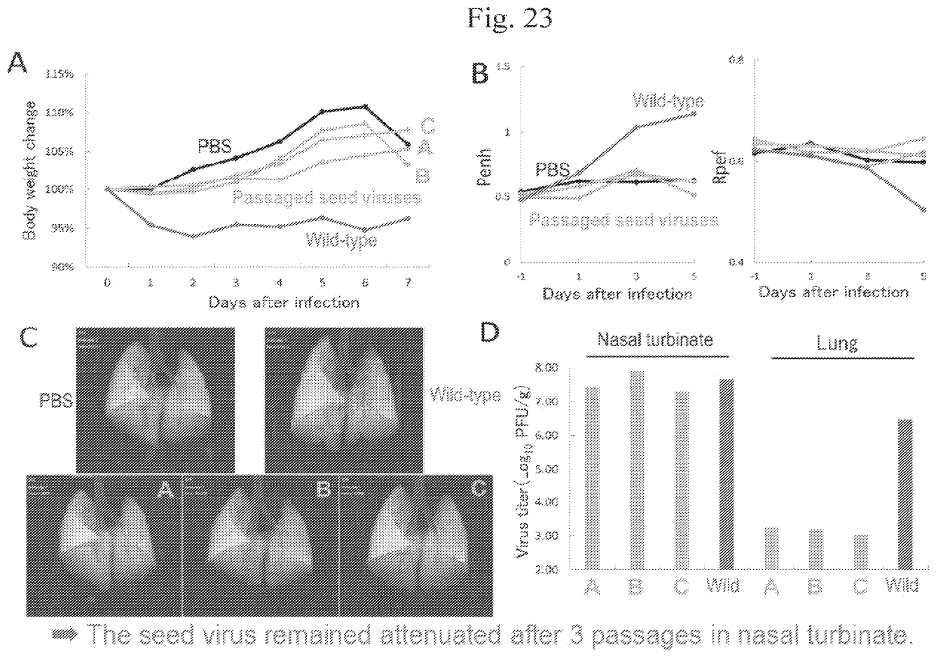

To test the pathogenicity of Line A, Line B, or Line C of passaged virus, hamsters were intranasally inoculated with 10³ PFU of passaged viruses. Body weight and respiratory function were monitored for 7 days post-infection. Lung and nasal titers were measured 3 days after infection. Micro-CT analysis was performed at 7 days post-infection (FIG. 22). Body weight change, respiratory function for Penh and Rpef, micro-CT analysis, and virus titers in the nasal turbinates and lungs of hamsters showed that the three lines of passaged virus maintained the attenuated phenotype in the hamsters (FIGS. 23A and 23B). CT scans of lungs of each group are shown in FIG. 23C. Virus titer in nasal turbinates (NT) and lungs is shown in FIG. 23D. The vaccine virus is shown to remain attenuated after 3 passages in NT.

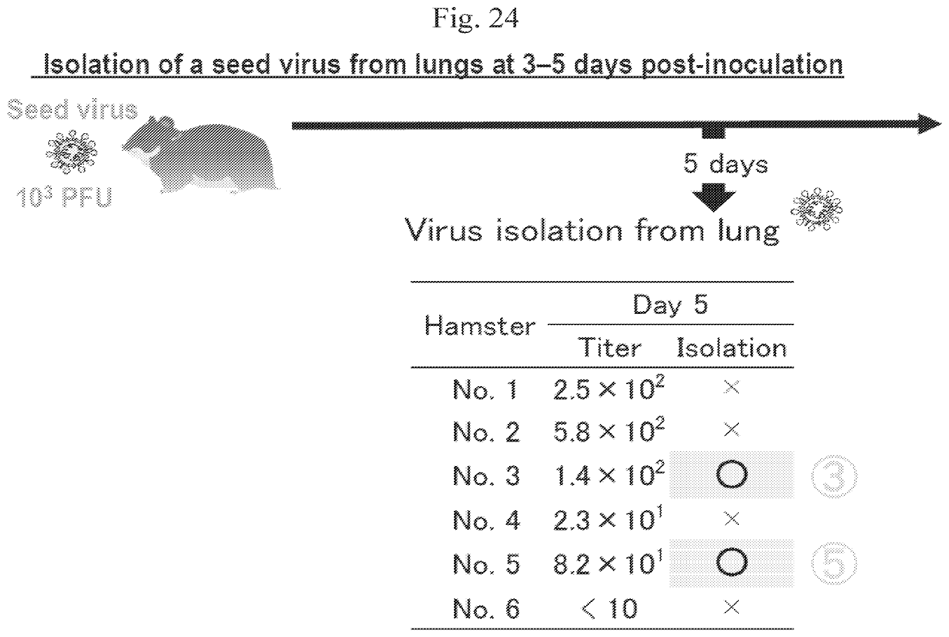

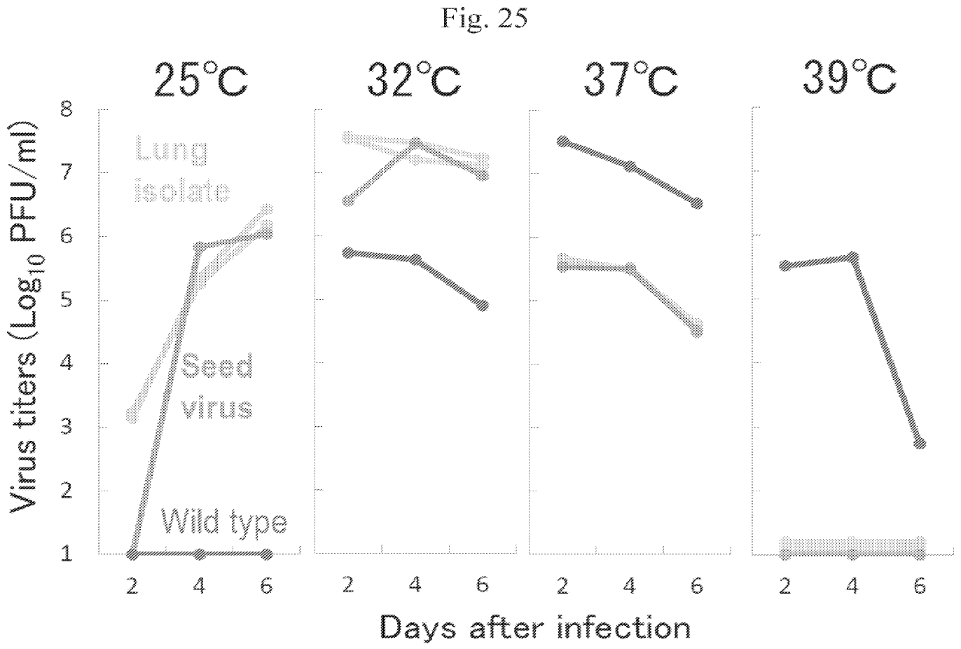

At 5 days post-infection, cold-adapted coronavirus was detected in the lungs of hamsters. Among these 5 samples, cold-adapted coronavirus was isolated from two samples (#3 and #5) (FIG. 24). The two viruses (#3 and #5) showed similar growth kinetics at 25, 32, 37, and 39° C. to those of the cold-adapted coronavirus (FIG. 25).

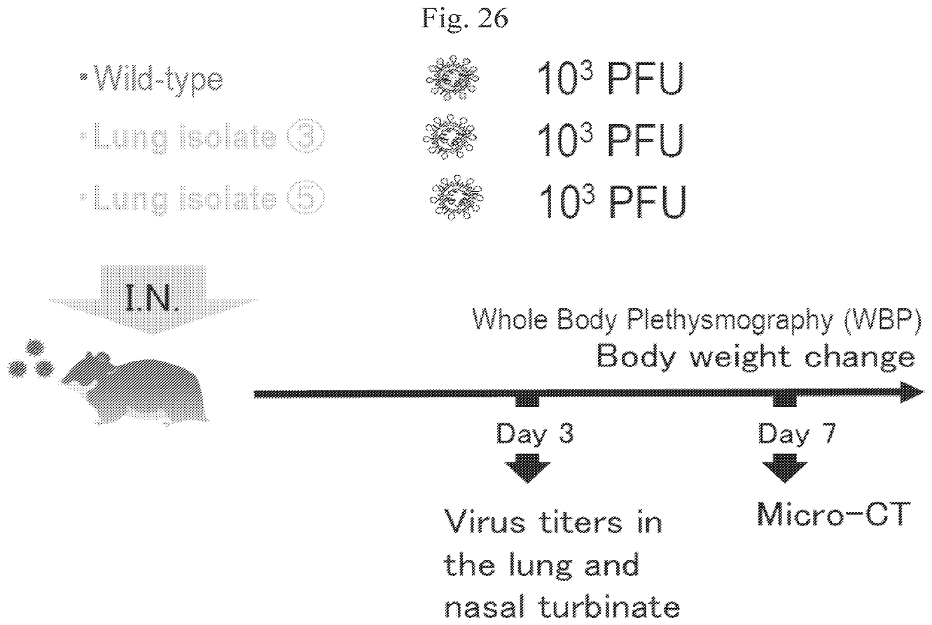

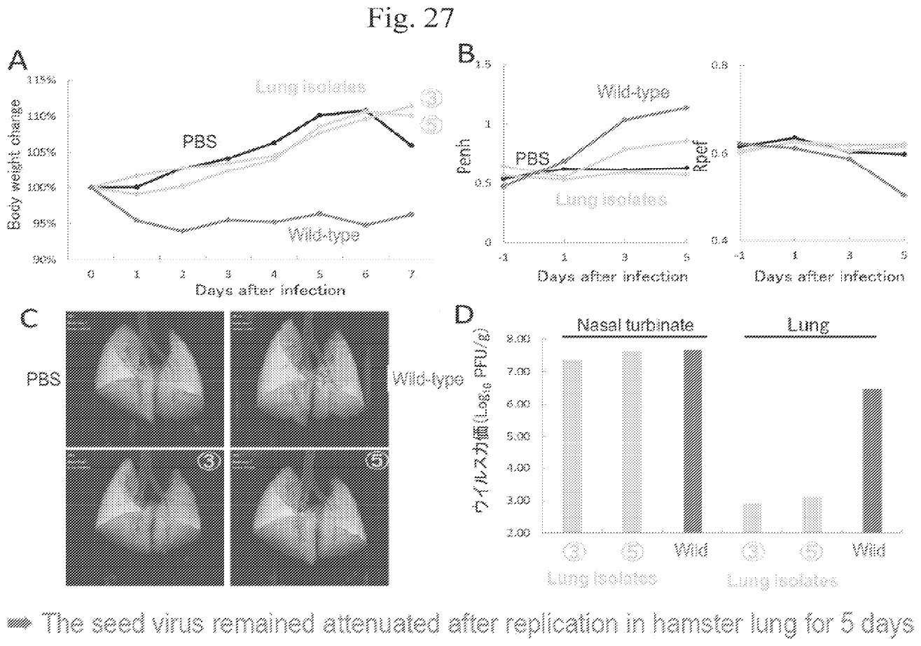

To test the pathogenicity of the two isolated viruses, hamsters were intranasally inoculated with 10³ PFU of the viruses. Body weight and respiratory function were monitored for 7 days post-infection. Lung and nasal titers were measured 3 days after infection. Micro-CT analysis was performed at 7 days post-infection (FIG. 26). Body weight and respiratory function were monitored for 7 days post infection. Lung and nasal titers were measured 3 days after infection. Micro-CT analysis was performed at 7 days post infection. As shown in FIGS. 27A-27D, body weight change (FIG. 27A), respiratory function for Penh and Rpef (FIG. 27B), micro-CT analysis (FIG. 27C), and virus titers in the nasal turbinates and lungs of hamsters (FIG. 27D) showed that the two isolated viruses maintained the attenuated phenotype in the hamsters.

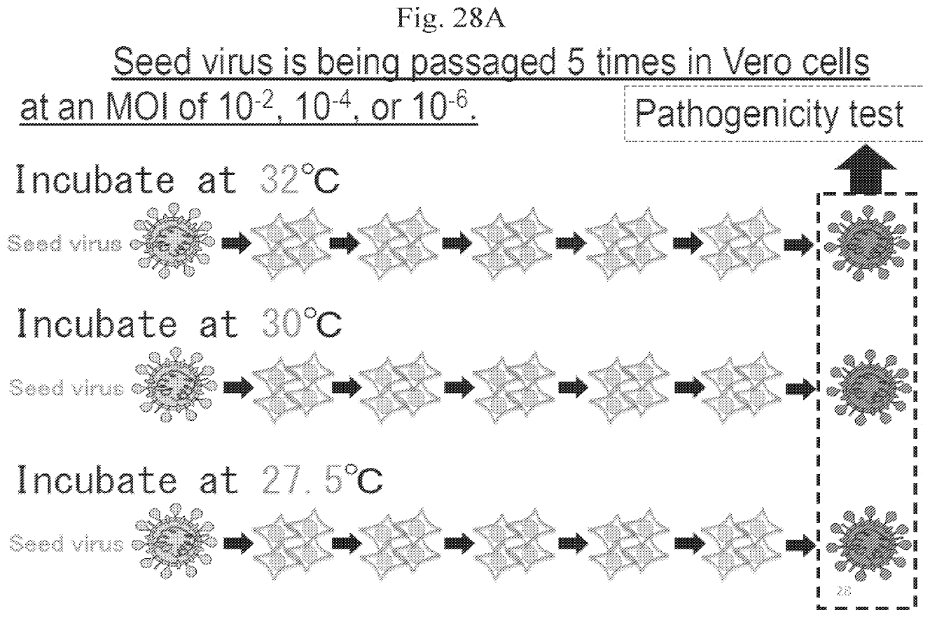

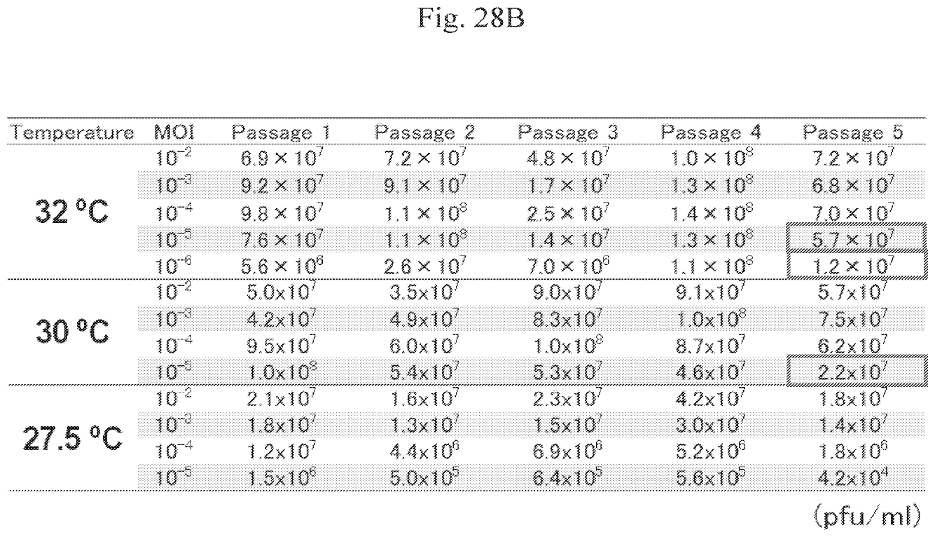

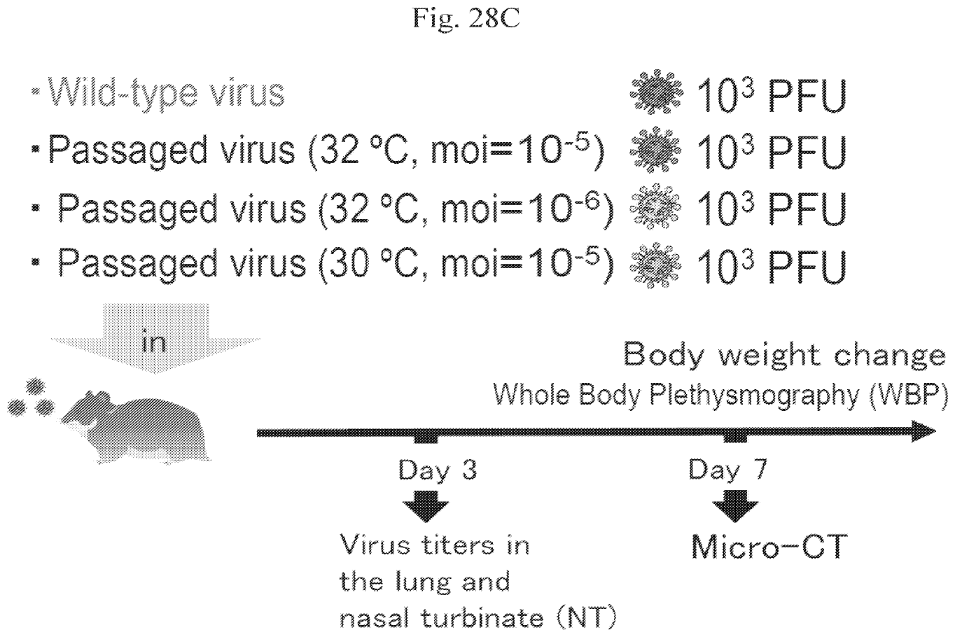

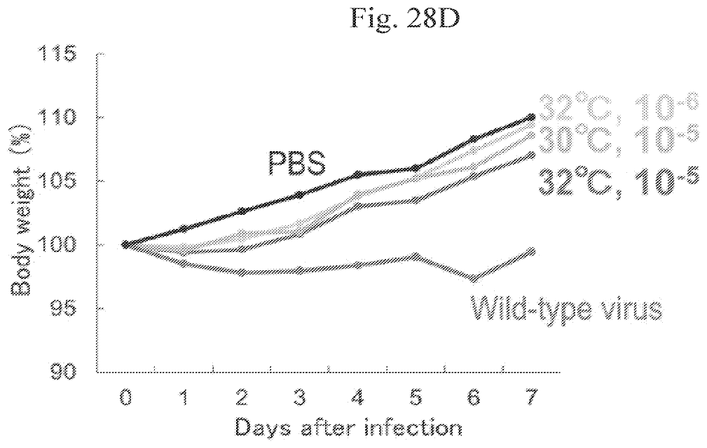

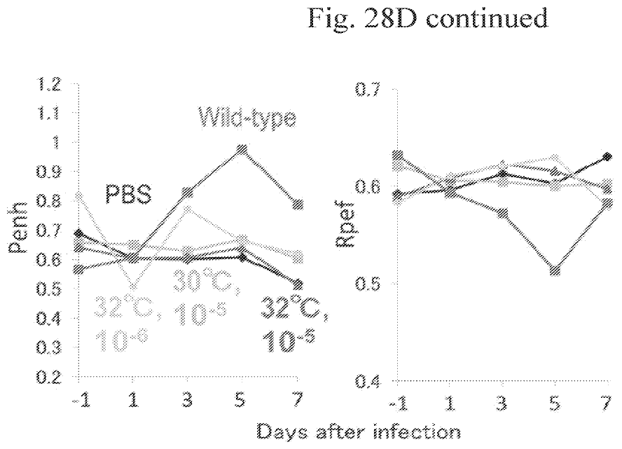

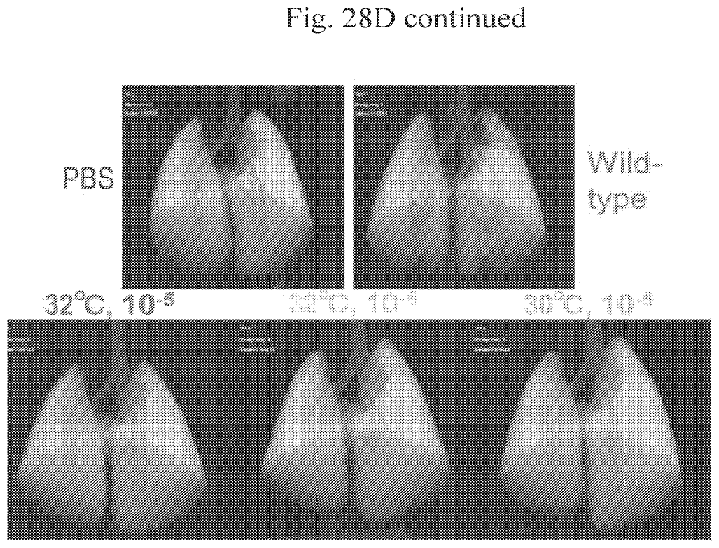

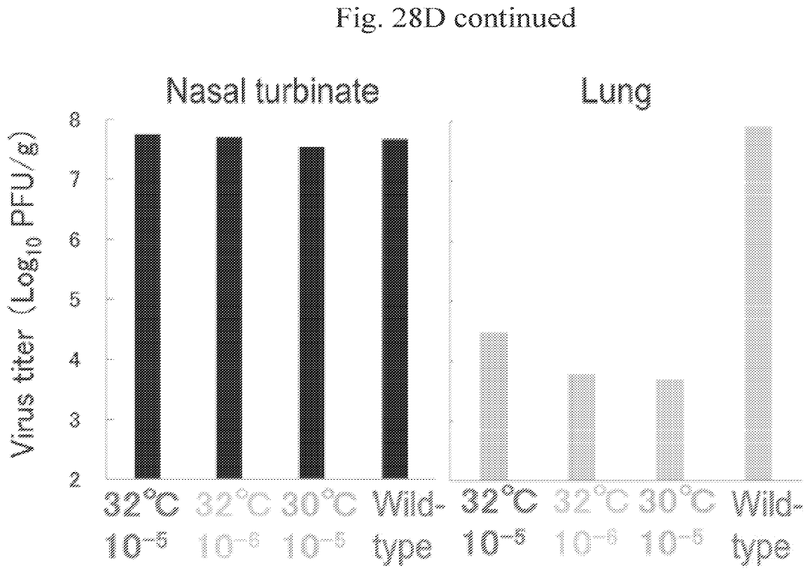

To test the in vitro stability of the cold-adapted coronavirus for the attenuation property, the cold-adapted coronavirus was passaged in Vero cells 5 times at 32, 30, and 27.5° C. at a multiplicity of infection (MOI) of 10⁻², 10⁻³, 10⁻⁴, 10⁻⁵, or 10⁻⁶ (FIG. 28A). When the cold-adapted coronavirus was passaged in +Vero cells at 32, 30, and 27.5° C., the virus titers ranged between ~10⁷ and ~10⁸ pfu/ml, except for the low MOI condition (10⁻⁴ and 10⁻⁵) at 27.5° C. Three samples passaged at 30° C. and 32° C. at a lower MOI condition (surrounded by brown squares) were tested for cold adaptation, temperature sensitivity, and pathogenicity in hamsters (FIG. 28B). To test the pathogenicity of the 3 passaged viruses, hamsters were intranasally inoculated with 10³ PFU of passaged viruses. Body weight and respiratory function were monitored for 7 days post-infection. Lung and nasal titers were measured 3 days after infection. Micro-CT analysis was performed at 7 days post-infection (FIG. 28C). Body weight change, respiratory function for Penh and Rpef, micro-CT analysis, and virus titers in the nasal turbinates and lungs of the hamsters showed that the three passaged viruses maintained the attenuated phenotype in the hamsters (FIG. 28D).

### Example 5: Determining the Amino Acid Changes that Contribute to Attenuation

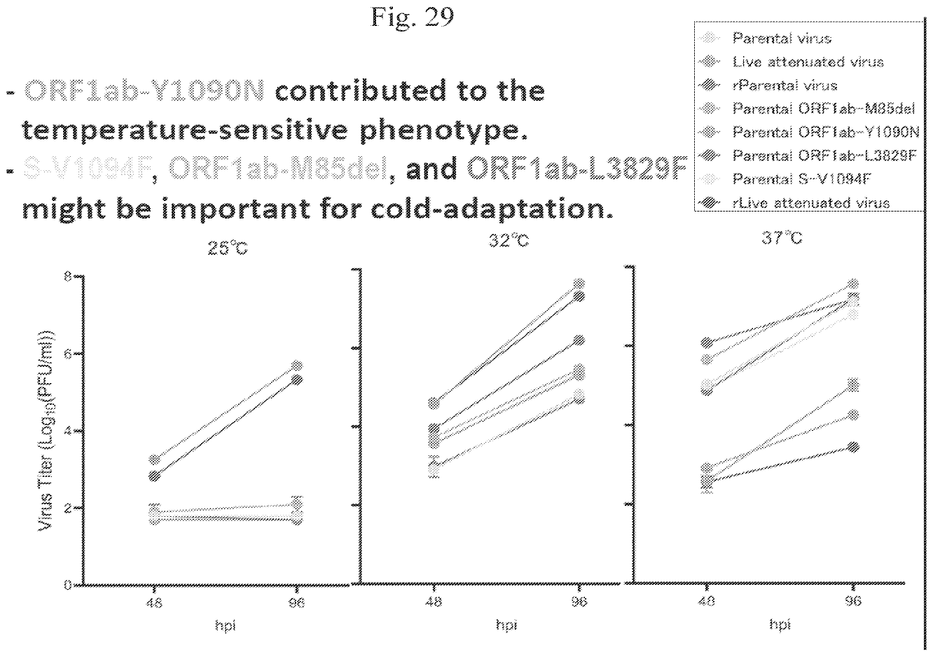

To determine which amino acid changes contribute to the attenuation, cold-adaptation, and temperature-sensitivity, recombinant wild-type viruses possessing a candidate substitution (ORF1ab-M85del, ORF1ab-Y1090N, ORF1ab-L3829F, and S-V1094F) were generated by reverse genetics. ORF1ab-Y1090N contributed to the temperature sensitivity. ORF1ab-M85del, ORF1ab-Y1090N, ORF1ab-L3829F, and S-V1094F were important for cold-adaptation (FIG. 29).

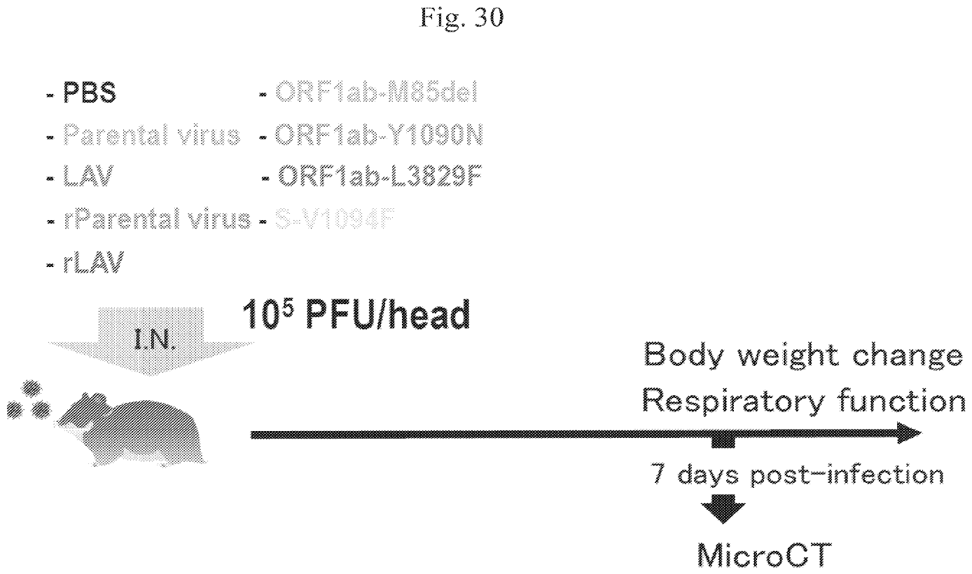

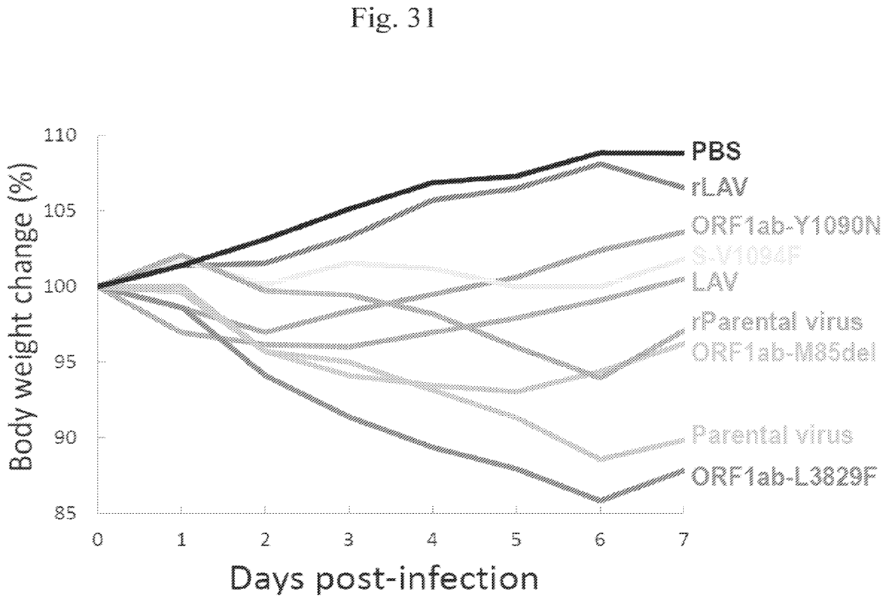

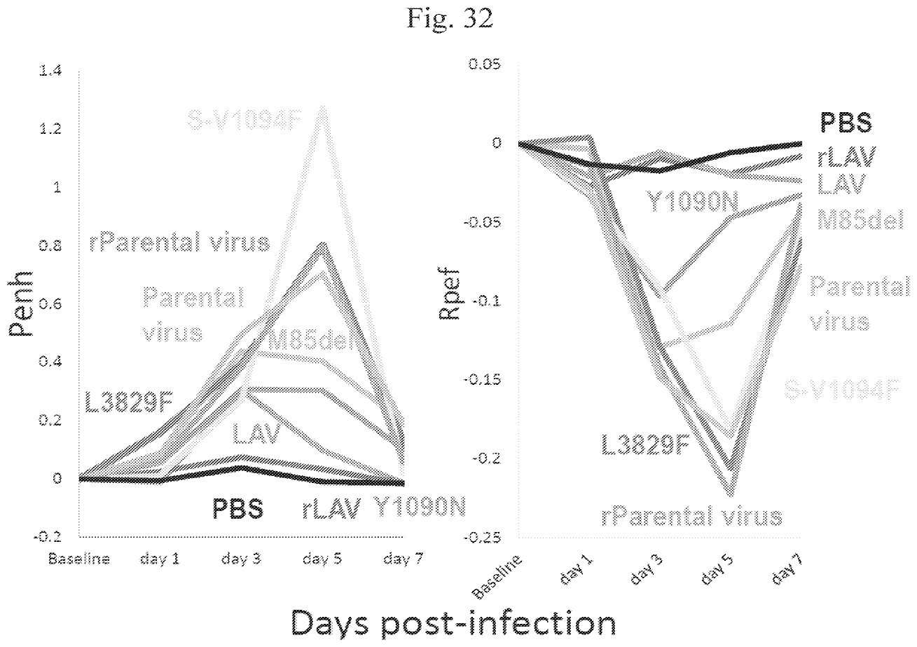

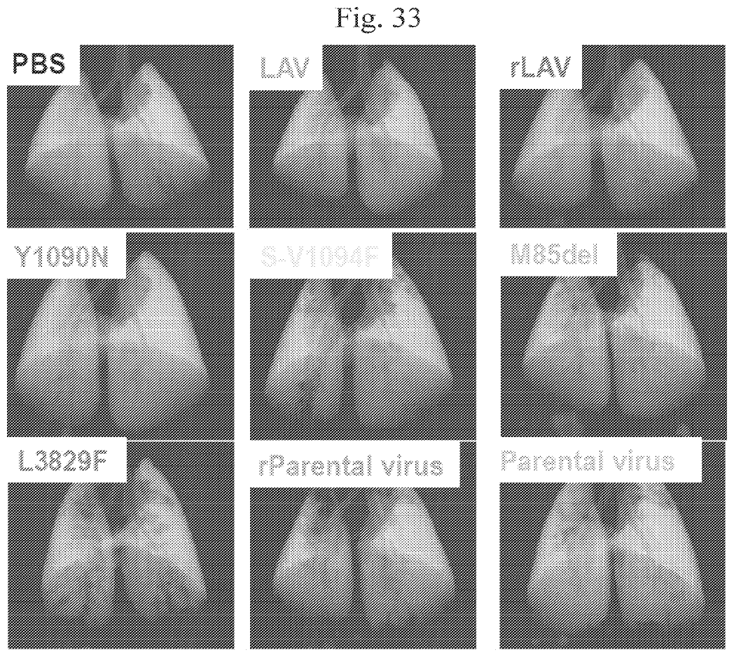

To test the pathogenicity of recombinant viruses, hamsters were intranasally inoculated with 10⁵ PFU. Body weight and respiratory function were monitored for 7 days post infection. Micro-CT analysis was performed at 7 days post infection (FIG. 30). ORF1ab-Y1090N and S-V1094F suppressed body weight loss of infected hamsters (FIG. 31). In the assessment of respiratory function, ORF1ab-M85del and ORF1ab-Y1090N contributed to attenuation in hamsters (FIG. 32). Micro-CT analysis showed that ORF1ab-M85del and ORF1ab-Y1090N caused attenuation in hamsters (FIG. 33).

### Example 6: Determining the Amino Acid Changes that Contribute to Attenuation

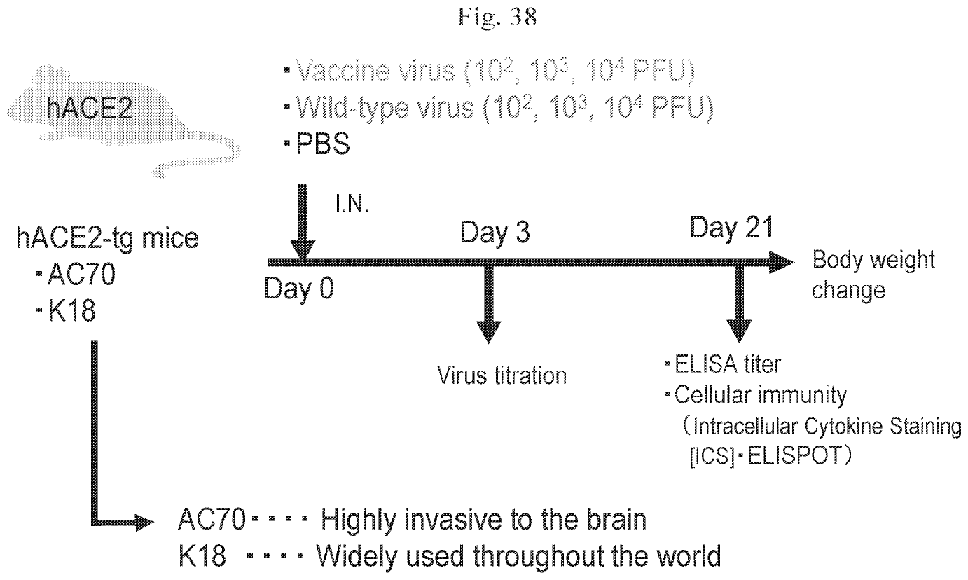

To test the attenuation of the cold-adapted coronavirus and induction of antibodies and cellular immunity, human ACE2-transgenic mice (strains AC70 and K18) were infected with the vaccine cold-adapted coronavirus at 10, 10², 10³, or 10⁴ PFU (FIG. 38). Body weight change was measured for 2 weeks and virus titers in the nasal turbinate and lungs were determined at 3 days post-infection. At 3 weeks after infection, antibody titers and cellular immunity were detected by ELISA using the receptor-binding domain (RBD) of spike protein and ectodomain of spike protein, intracellular cytokine staining, and ELISPOT.

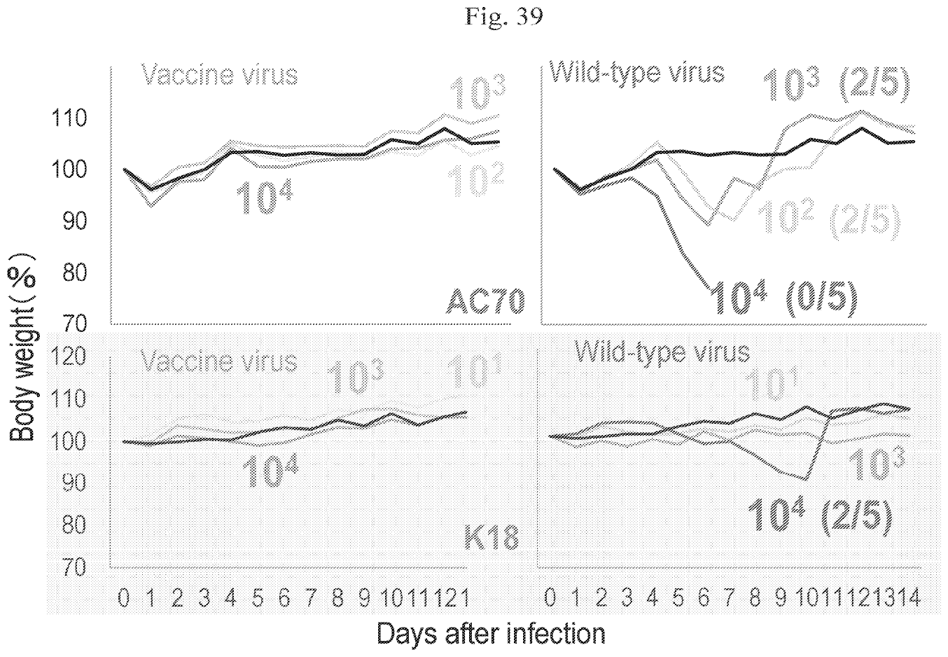

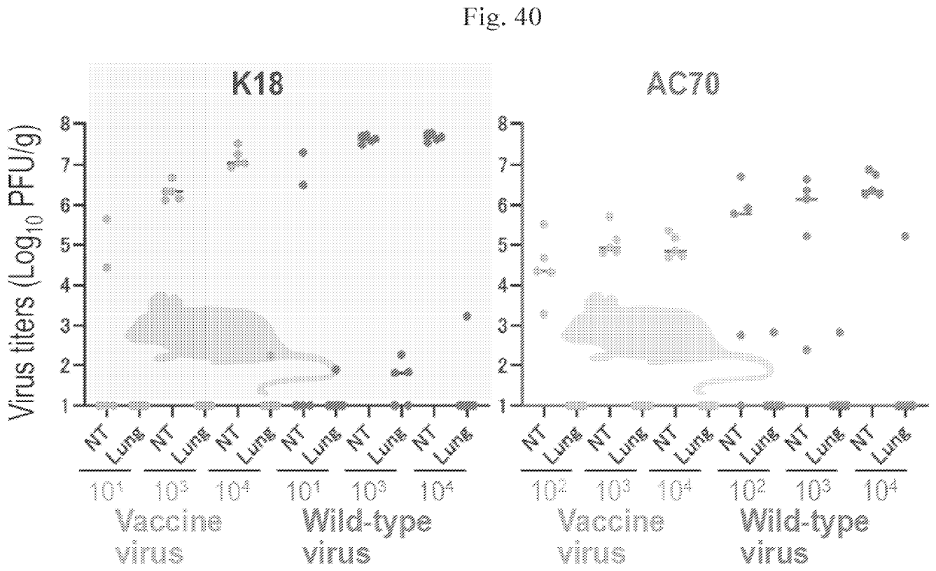

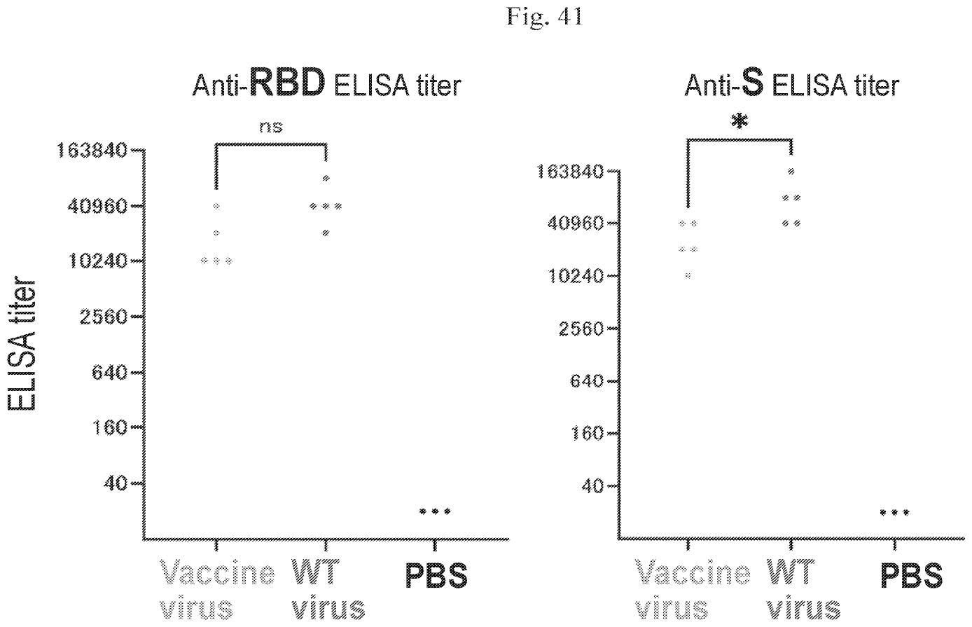

AC70 and K18 mice did not lose body weight or die after infection with the vaccine cold-adapted coronavirus at 10², 10³, or 10⁴ PFU. Wild-type virus caused body weight loss and the death of all or some of the AC70 and K18 mice at 10², 10³, or 10⁴ PFU infection (FIG. 39). Vaccine cold-adapted coronavirus was not detected in the lungs of AC70 or K18 mice, whereas wild-type virus was detected in the lungs of both strains. In the nasal turbinate, virus titers of the vaccine cold-adapted coronavirus were similar to or lower than those of wild-type virus (FIG. 40). Vaccine cold-adapted coronavirus induced antibodies against the receptor binding domain (RBD) and S protein at levels similar to those induced by wild-type virus (FIG. 41).

### Example 7: Th1 dependence of induced antibodies IgG1 and IgG2c

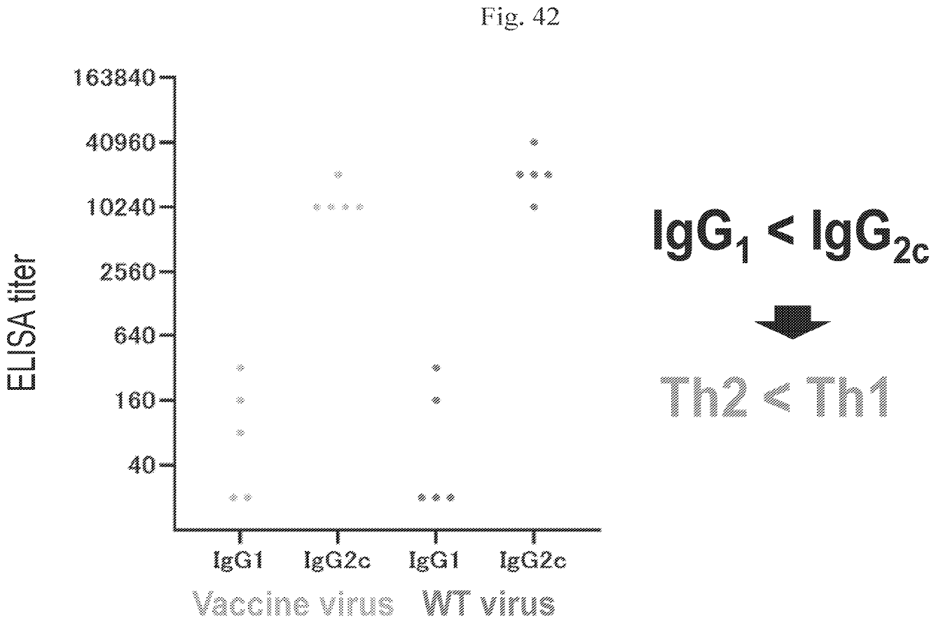

To evaluate the Th1/Th2 balance, the induced antibodies IgG1 and IgG2c were measured by ELISA. A high IgG2c antibody titer was detected, confirming that the immune response was Th1 dependent (FIG. 42).

### Example 8: Cellular Immunity

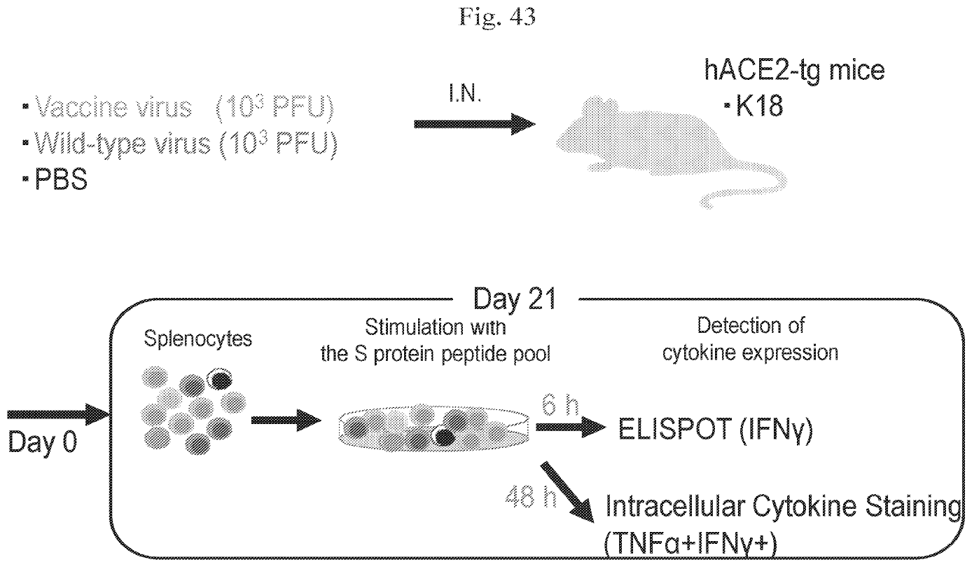

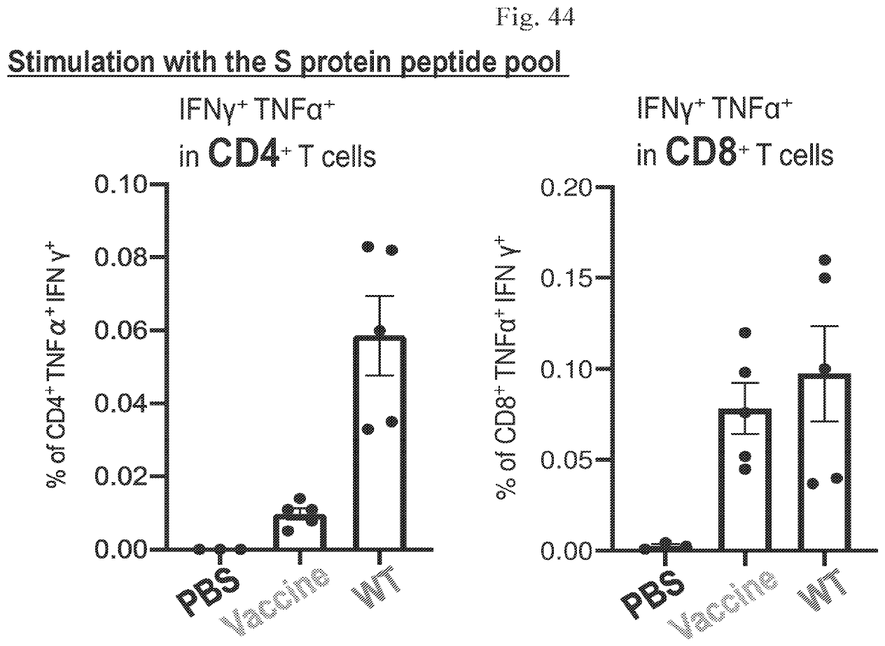

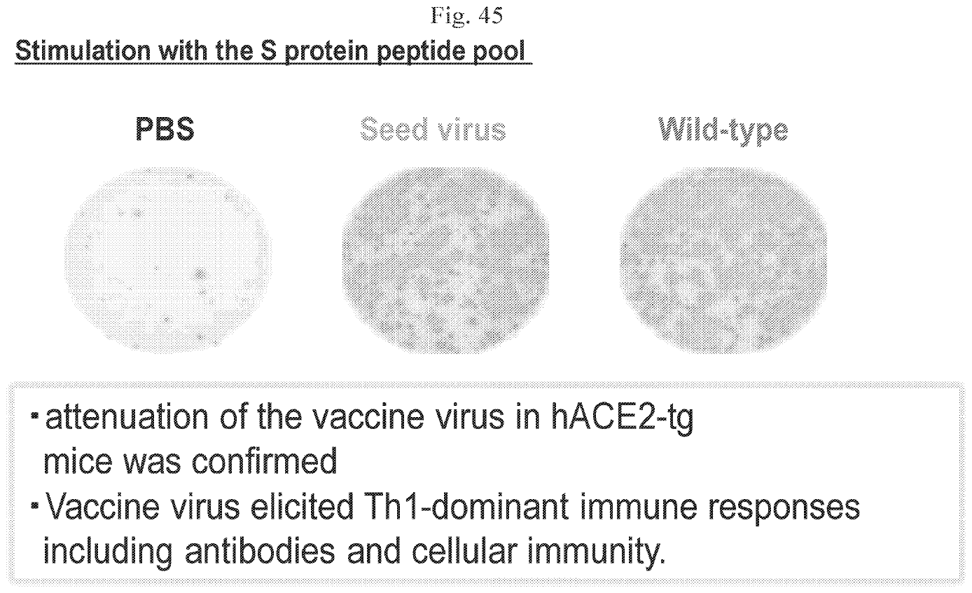

To evaluate the cellular immunity, splenocytes collected from K18-mice infected with the vaccine cold-adapted coronavirus were stimulated with the spike protein peptide pool and ELISPOT and intracellular cytokine straining were performed at 3 weeks after infection (FIG. 43). IFNγ- and TNFα-positive CD4 and CD8 T cells were detected in the splenocytes of vaccine cold-adapted coronavirus (FIG. 44). Similarly, ELISPOT showed that cellular immunity against the spike protein was induced in K18-mice after vaccine cold-adapted coronavirus infection (FIG. 45).

### Example 9: Challenge of Cold-Adapted Coronavirus Immunized Hamsters with Wild Type Omicron Variant BA.1 Virus

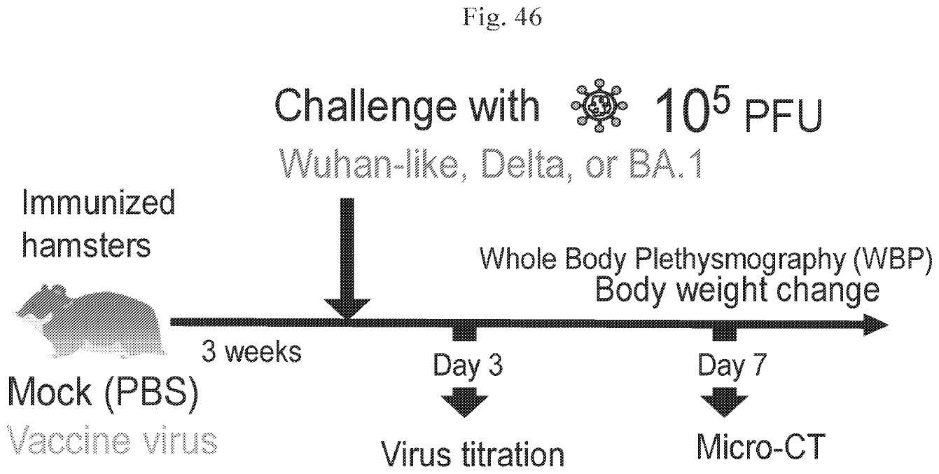

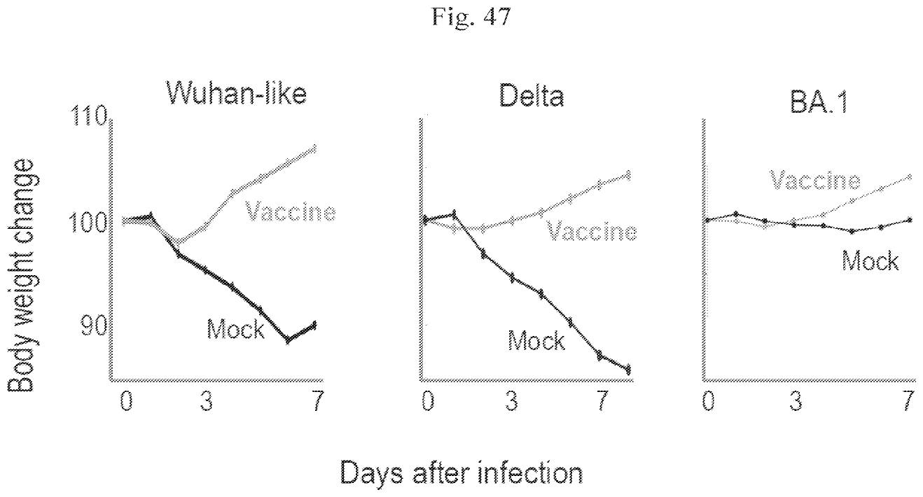

Hamsters immunized with the cold-adapted coronavirus were intranasally challenged with wild-type omicron variant BA.1 virus at 10⁵ PFU (FIG. 46). Body weight and respiratory function were monitored for 7 days post-infection. Lung and nasal titers were measured 3 days after infection. Micro-CT analysis was performed at 7 days post-infection. Body weight loss of cold-adapted coronavirus-immunized hamsters was limited after BA.1 infection (FIG. 47).

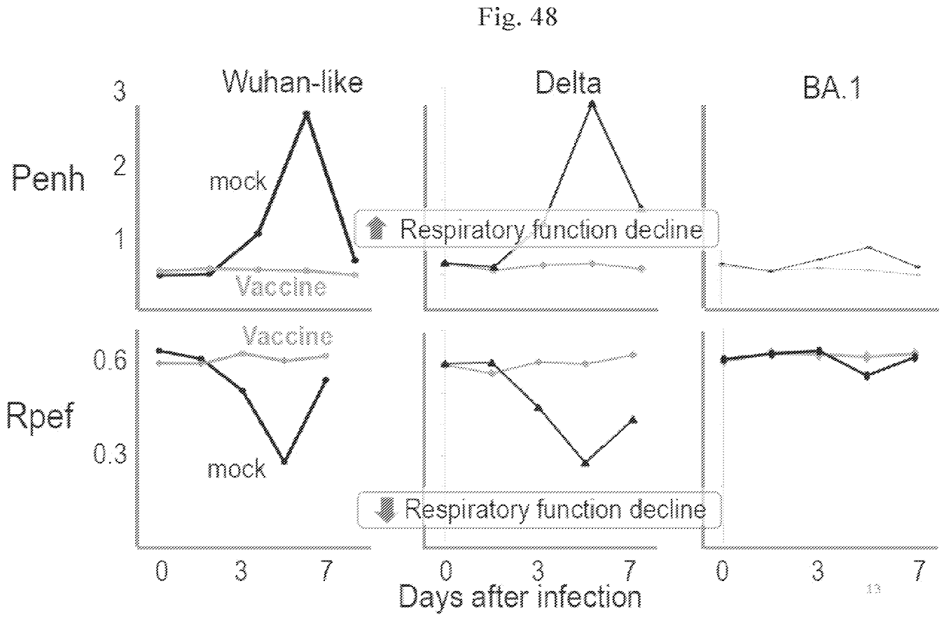

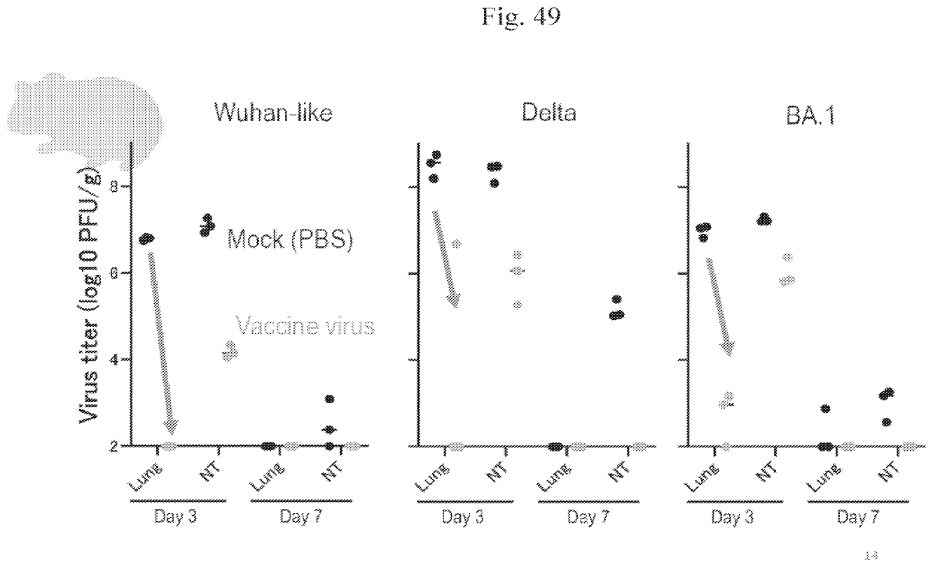

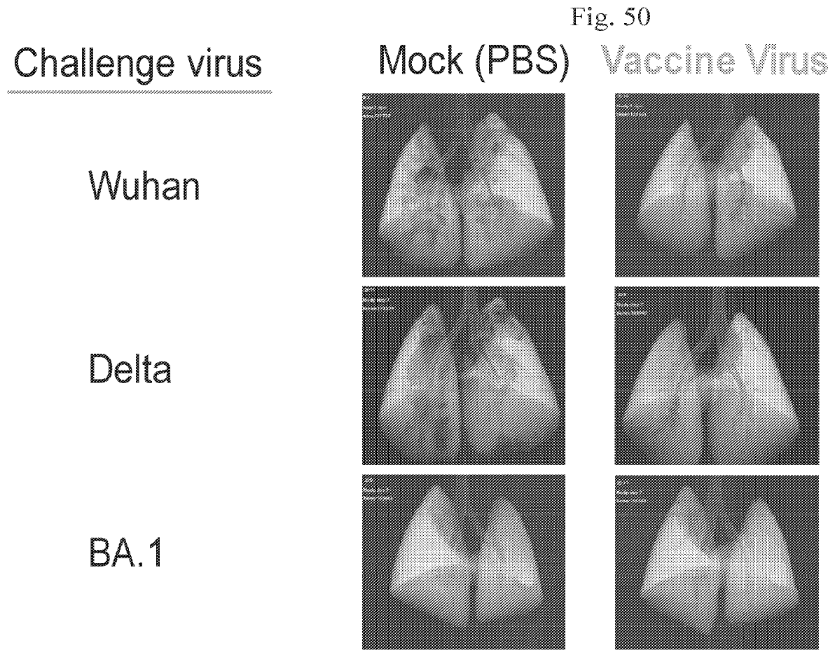

Whole body plethysmography (WBP) showed that cold-adapted coronavirus-immunized hamsters challenged with BA.1 showed no reduction in respiratory function in terms of either Penh or Rpef (FIG. 48). Virus titers in the lungs and nasal turbinates of cold-adapted coronavirus-immunized hamsters after challenge infection were significantly reduced compared with those of the PBS group at 3 days after infection (FIG. 49). No significant inflammatory findings were detected by micro-CT analysis in the lungs of cold-adapted coronavirus-immunized and PBS-administrated hamsters after BA.1 infection (FIG. 50).

### Example 10: Determining the Amino Acid Changes that Contribute to Attenuation in Cold-Adapted Coronaviruses Possessing an Amino Acid Substitution

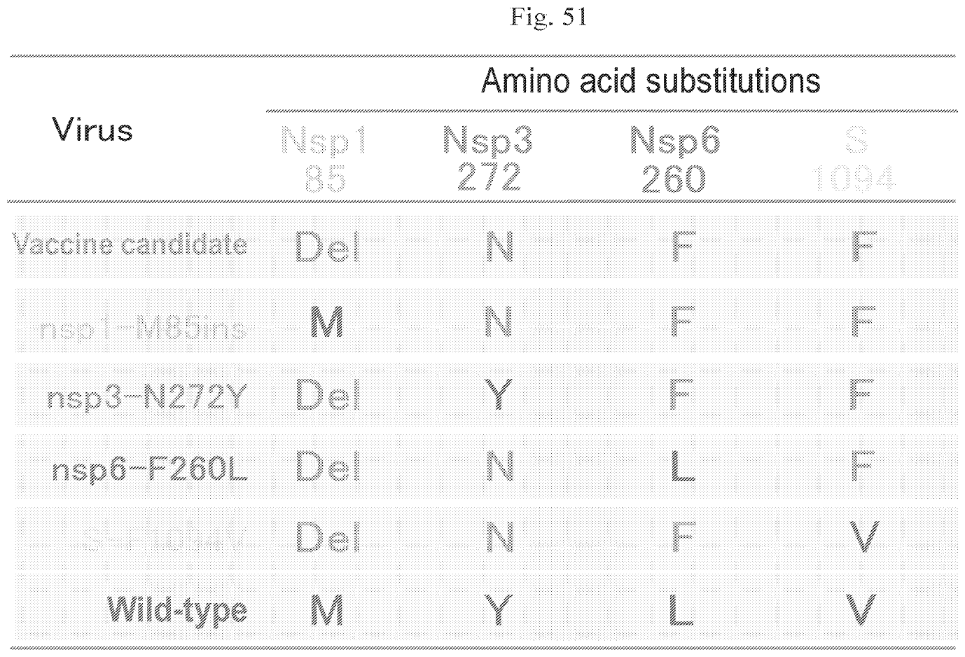

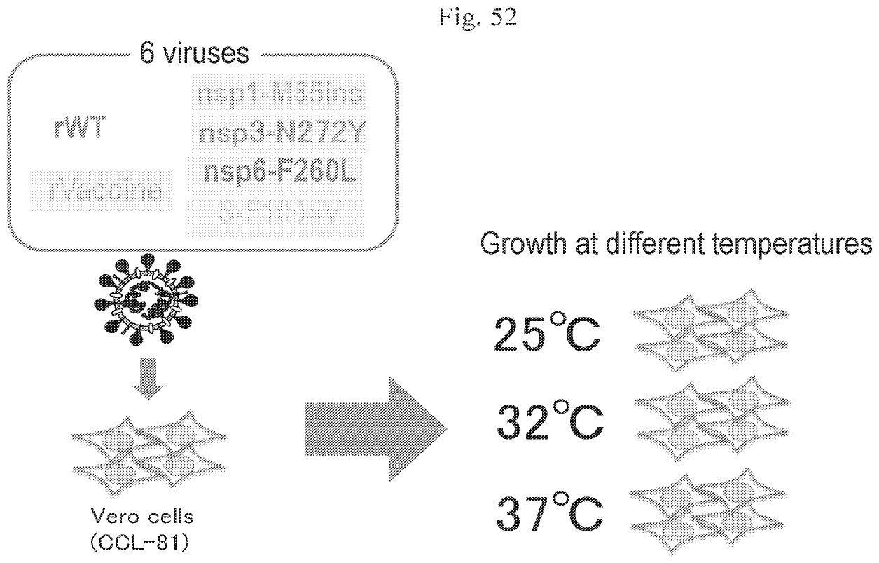

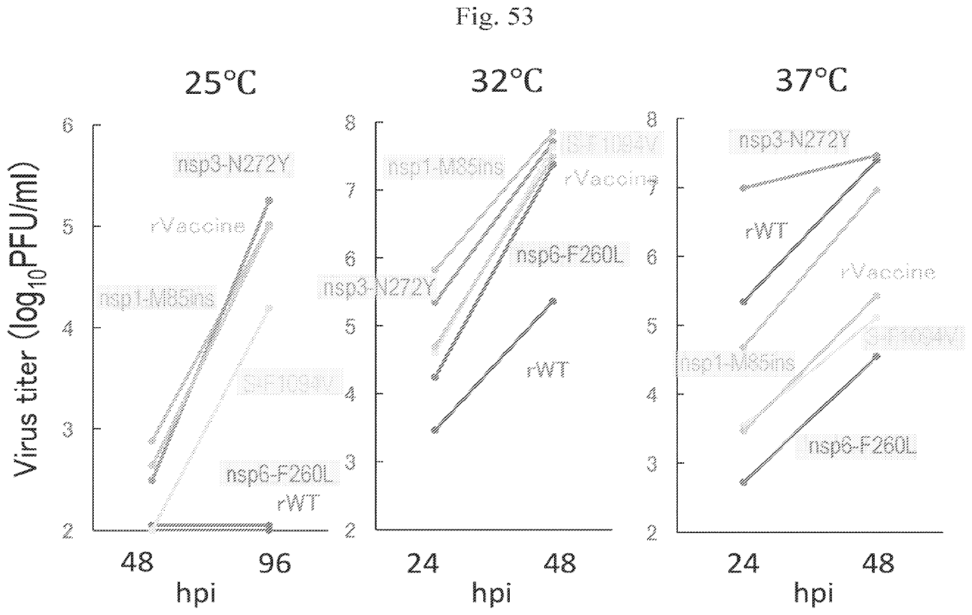

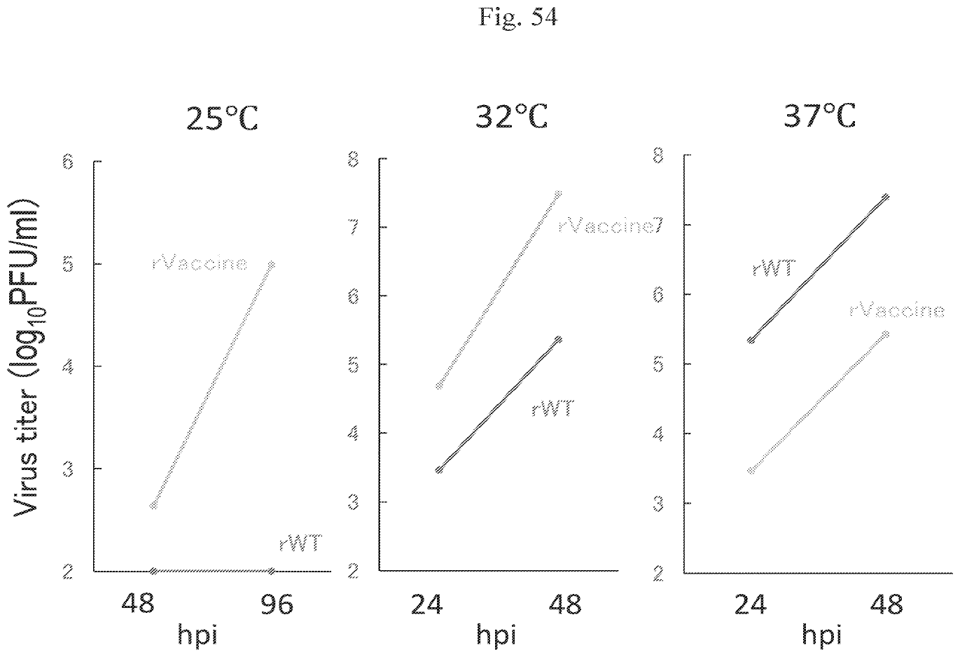

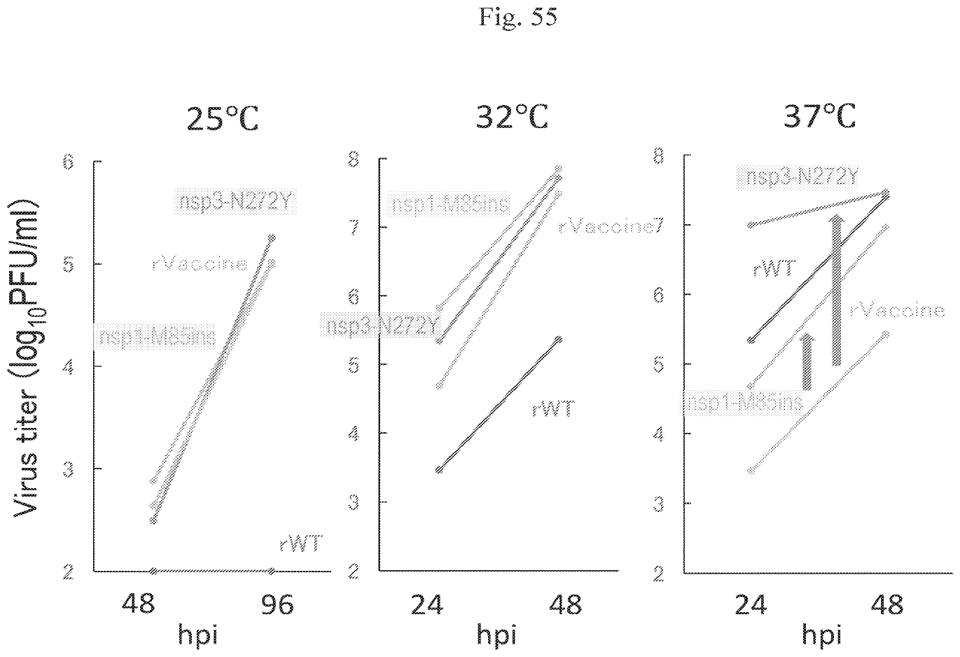

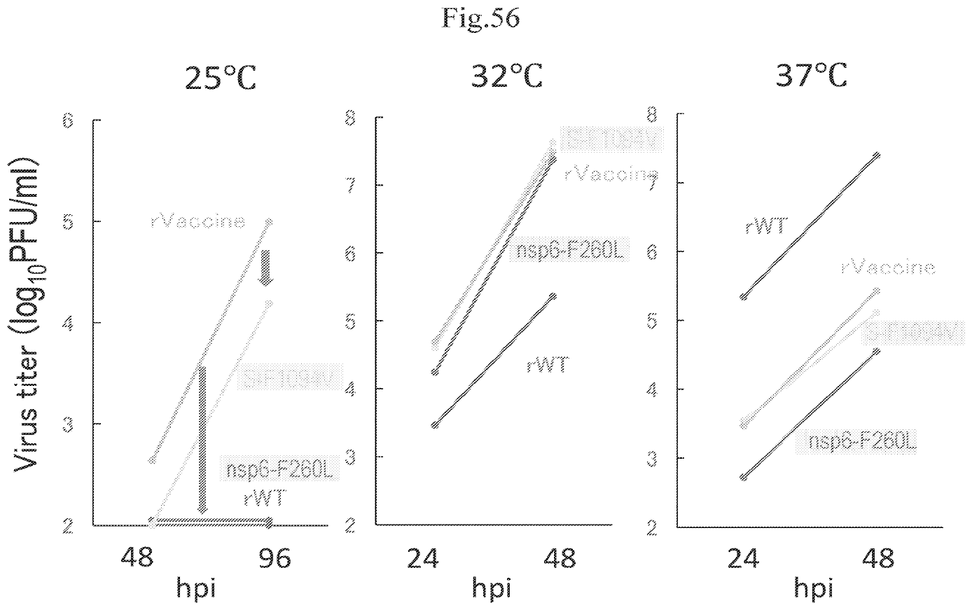

To determine which amino acid changes contribute to the attenuation, cold-adaptation, and temperature sensitivity, recombinant vaccine cold-adapted coronaviruses possessing a substitution (nsp1-M85ins, nsp3-N272Y, nsp6-F260L, and S-F1094V) were generated by reverse genetics (FIG. 51). These recombinant viruses were inoculated into Vero cells and virus growth was examined at 25, 32, and 37° C. (FIGS. 52 and 53). The growth of the reverse engineered vaccine cold-adapted coronavirus and wild type virus at 25, 32, and 37° C. is shown in FIG. 54. The mutant cold-adapted coronaviruses nsp1-M85ins and nsp3-N272Y correlate with temperature sensitivity (FIG. 55). The mutant cold-adapted coronaviruses with nsp6-F260L and S-F1094V mutations correlate with cold-adaptation (FIG. 56).

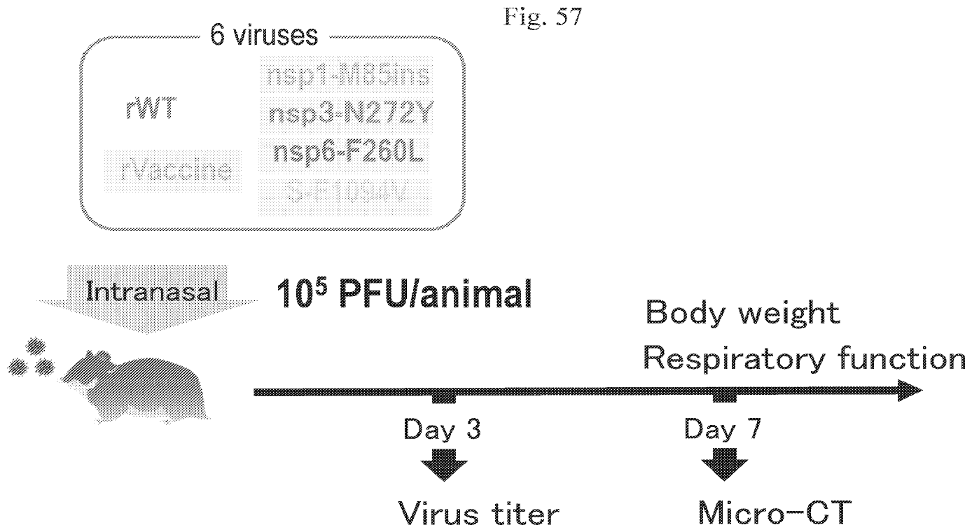

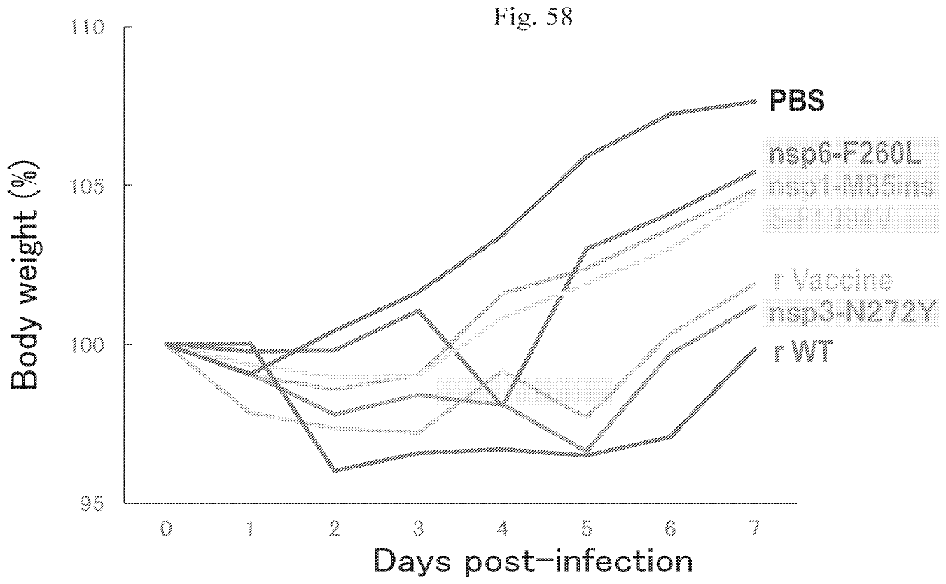

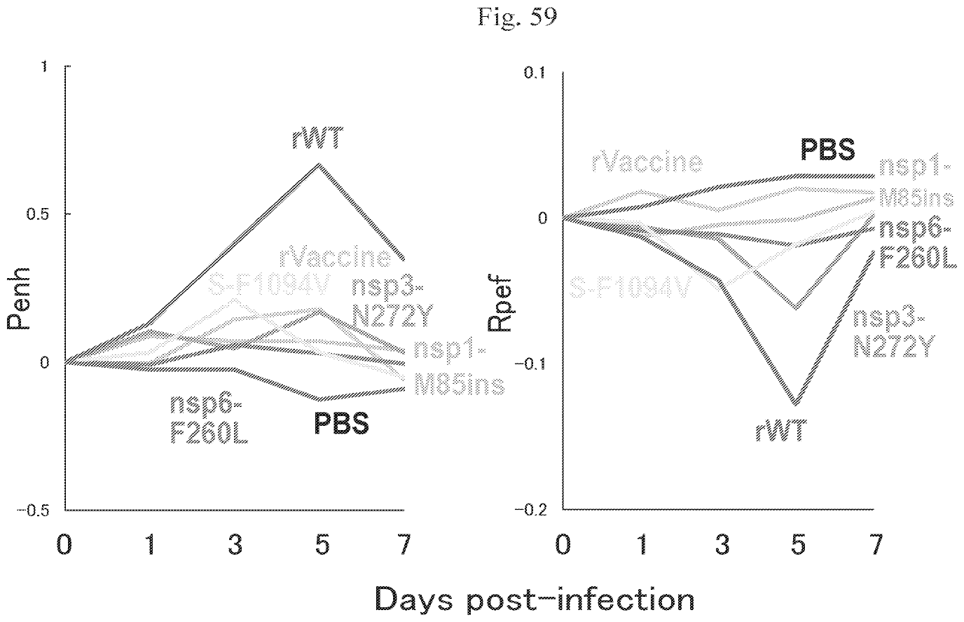

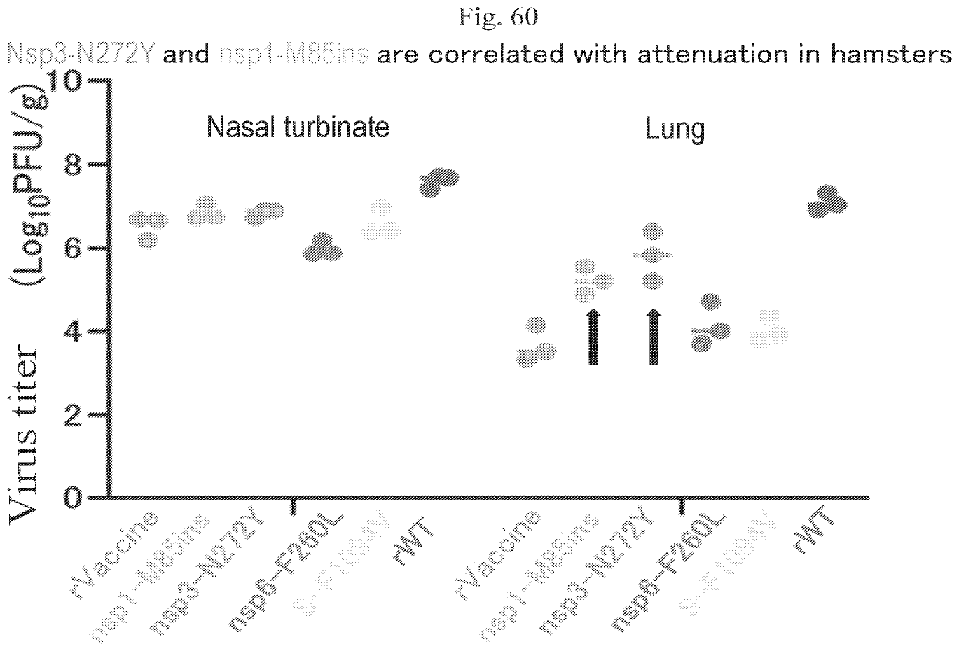

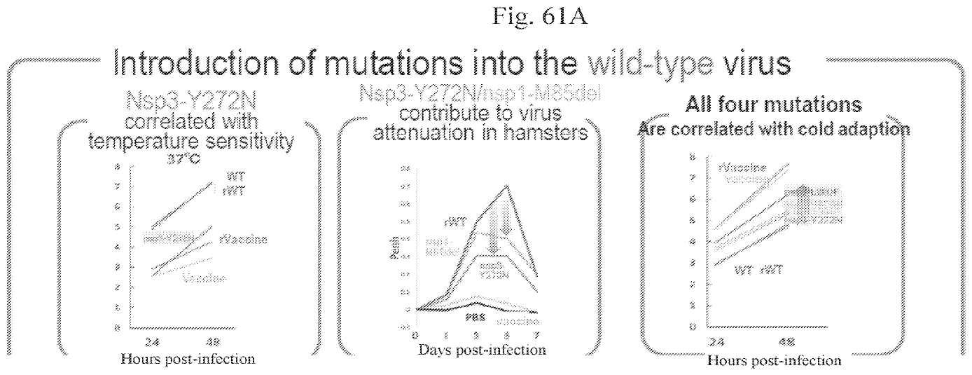

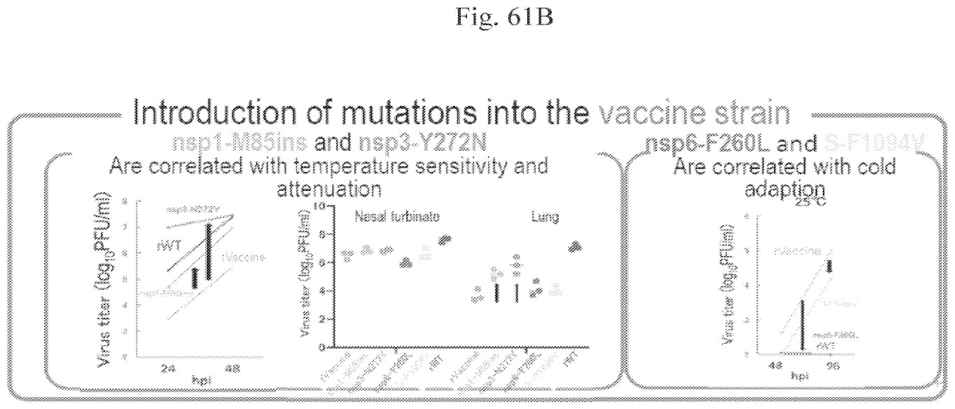

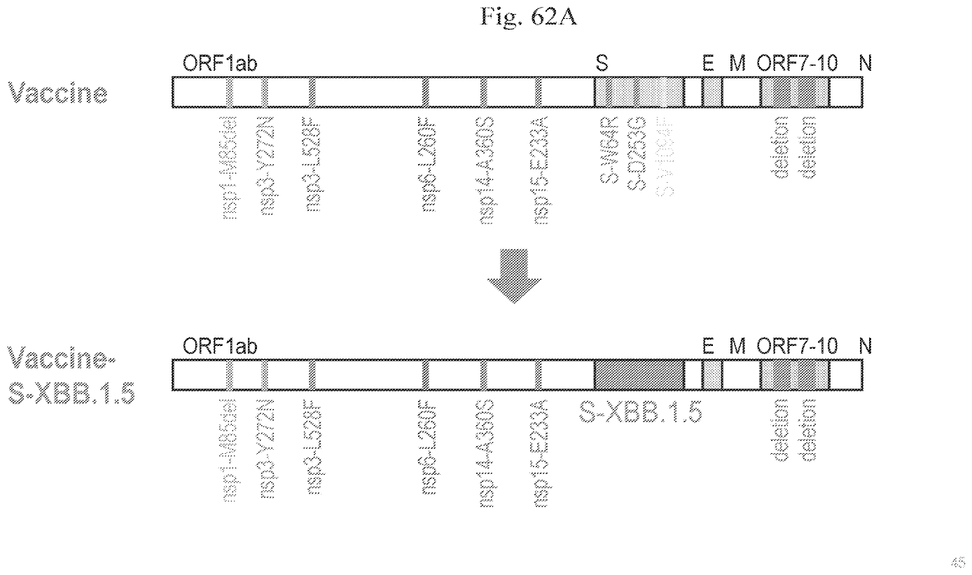

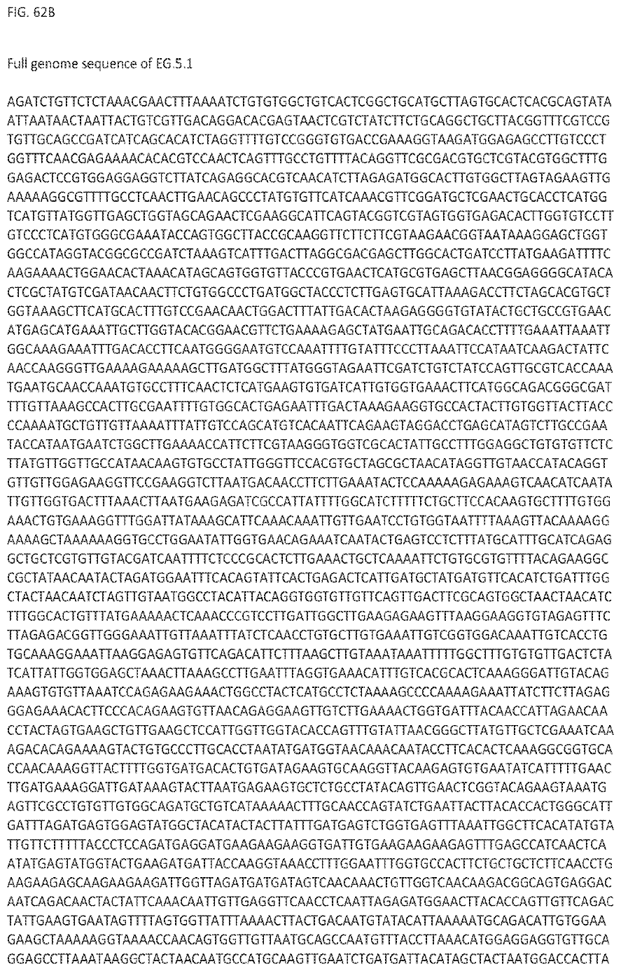

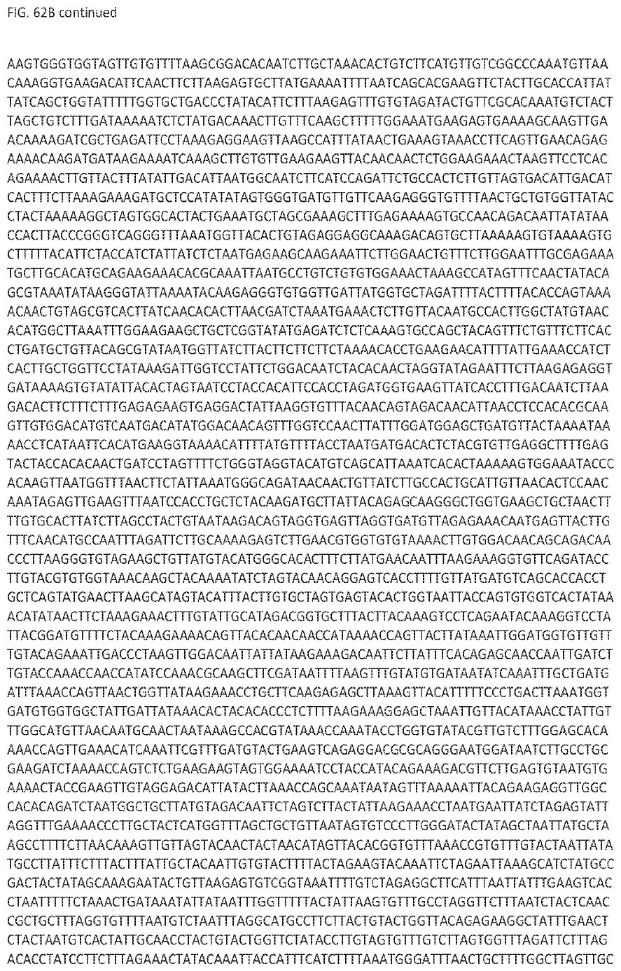

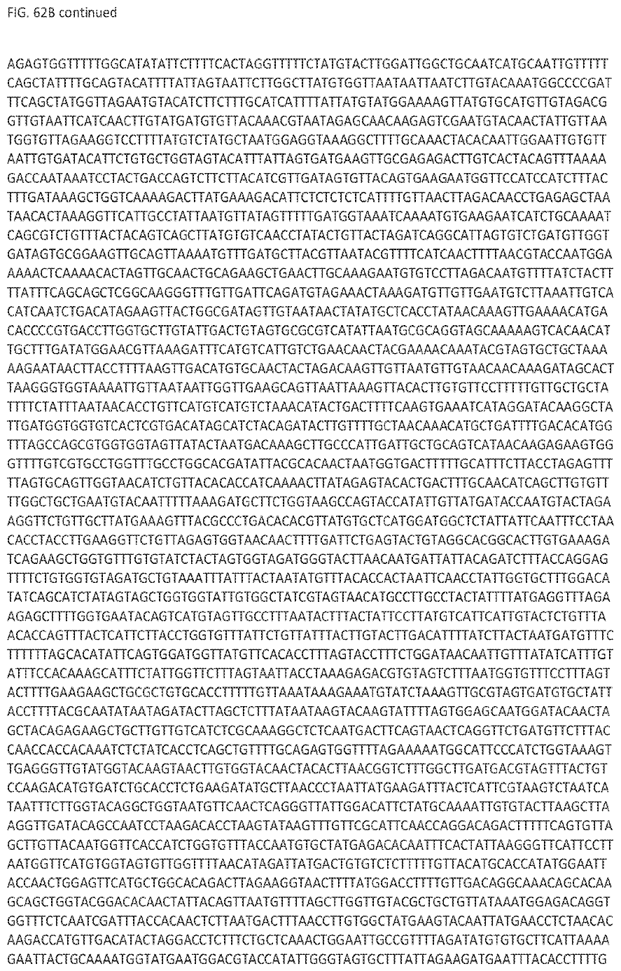

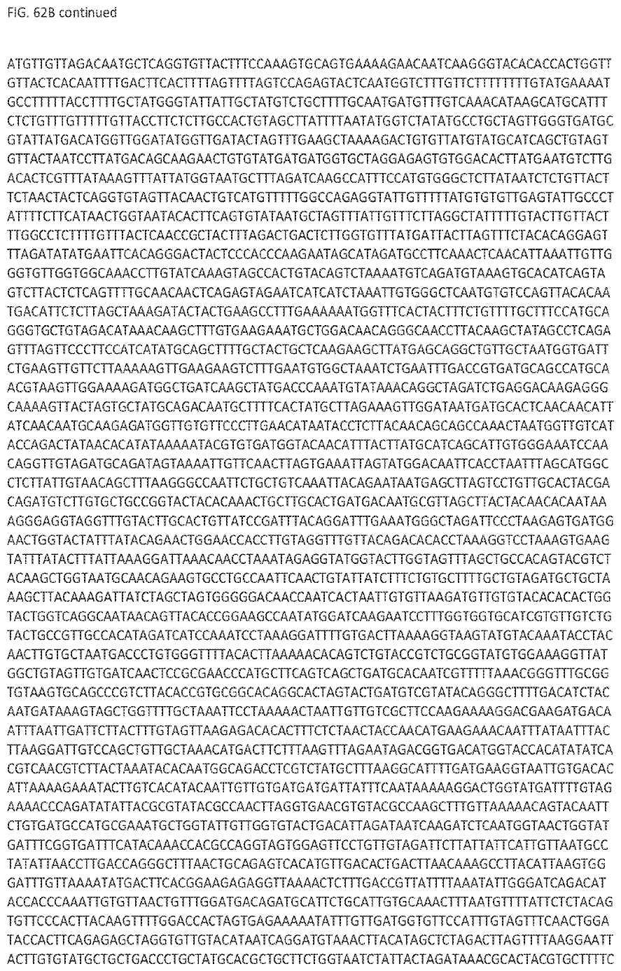

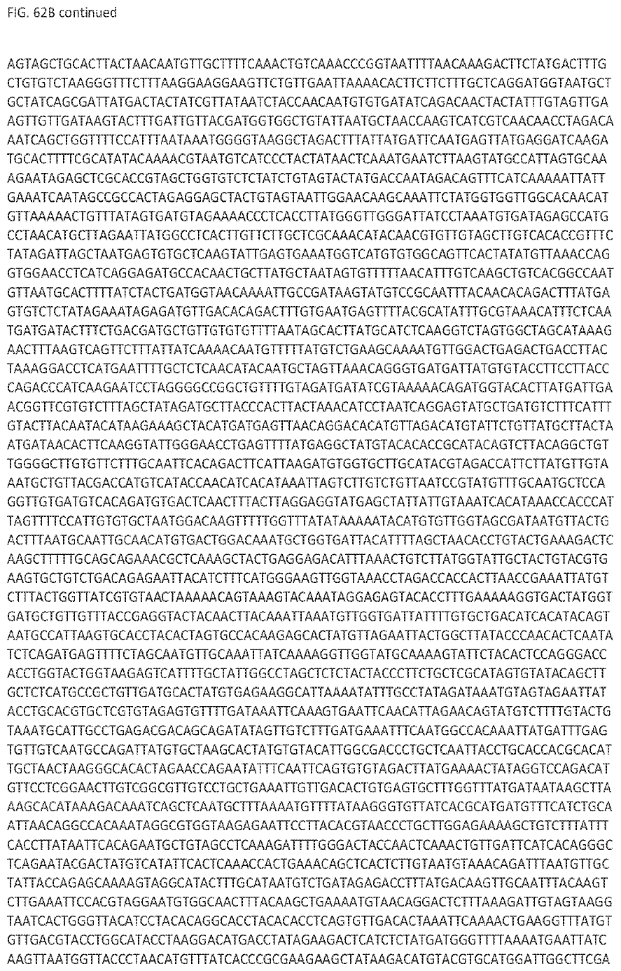

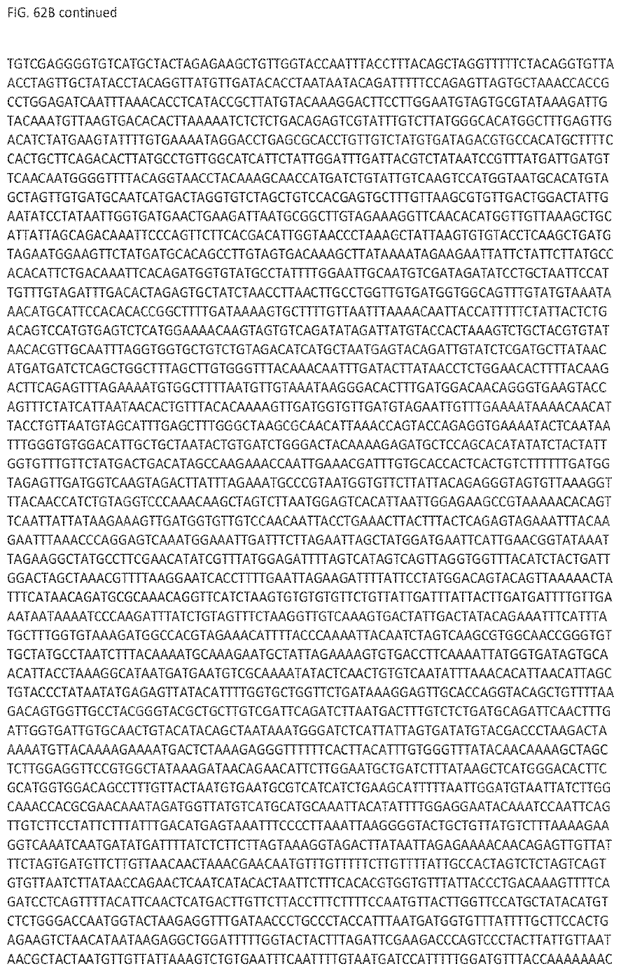

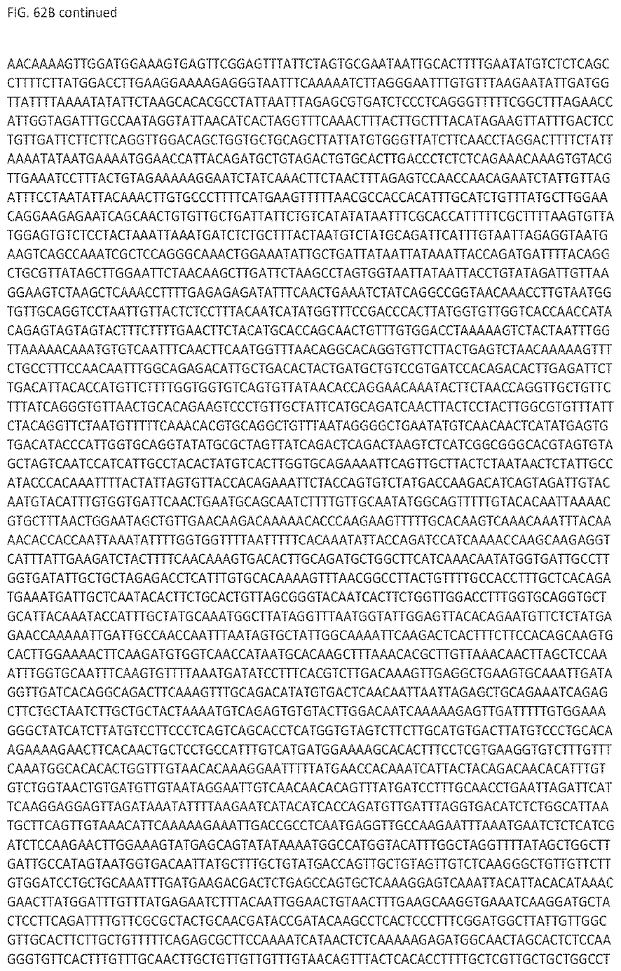

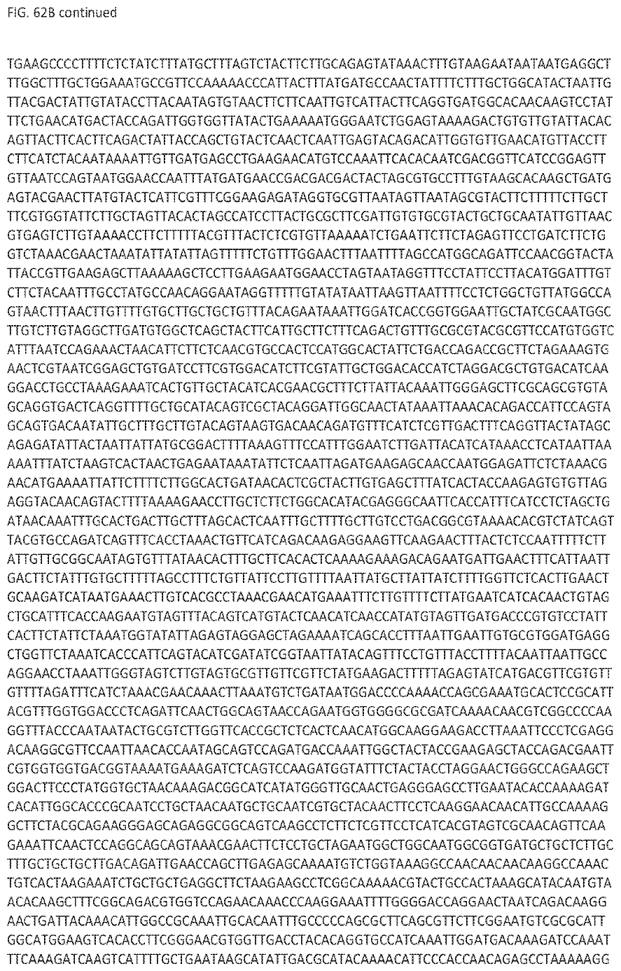

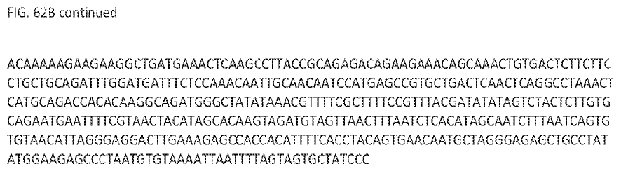

To test the pathogenicity of the recombinant viruses, hamsters were intranasally inoculated with 10⁵ PFU (FIG. 57). Body weight (FIG. 58) and respiratory function (FIG. 59) were monitored for 7 days post-infection. Virus titers were determined at 3 days post-infection (FIG. 60). The mutant cold-adapted coronaviruses nsp1-M85ins and nsp3-N272Y correlate with attenuation. All four substitutions (nsp1-M85ins, nsp3-N272Y, nsp6-F260L, and S-F1094V) contributed to the attenuation of the cold-adapted coronavirus (FIGS. 61A-61B).

### Example 11

To investigate whether the vaccine cold-adapted coronavirus works as a vaccine backbone, the S gene of the vaccine cold-adapted coronavirus was replaced with the S gene of XBB.1.5 (FIG. 62A). The genome of the vaccine cold-adapted coronavirus with the substitution of the S gene from XBB.1.5 is shown in FIG. 62B.

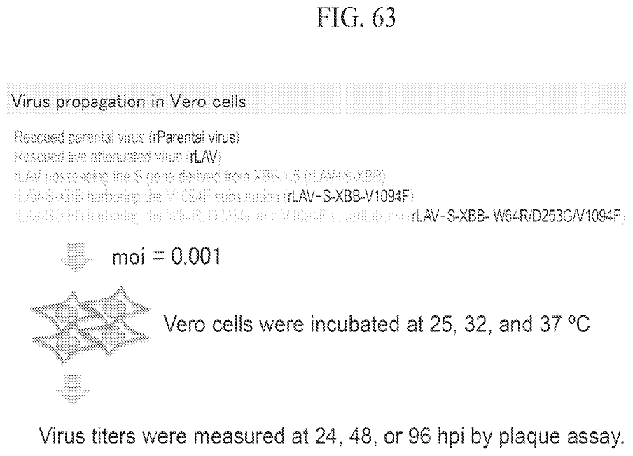

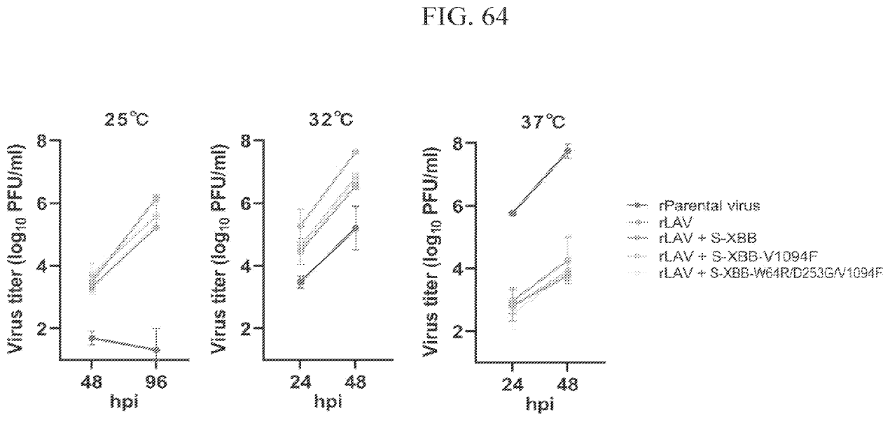

In addition to the virus shown in FIG. 62A, two vaccine cold-adapted coronaviruses possessing the S gene of XBB.1.5 harboring the V1094F substitution or the W64R, D253G, and V1094F substitutions were prepared by reverse genetics and their replication in Vero cells was examined at 25, 32, and 37° C. (FIG. 63). The three viruses possessing the XBB. 1.5 S gene showed similar growth kinetics to those of the vaccine cold-adapted coronavirus (LAV) (FIG. 64).

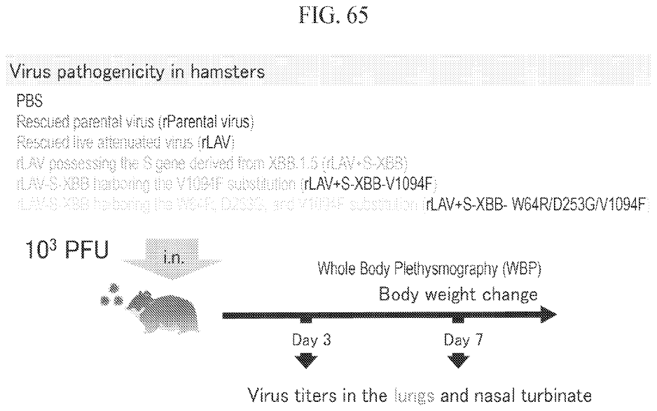

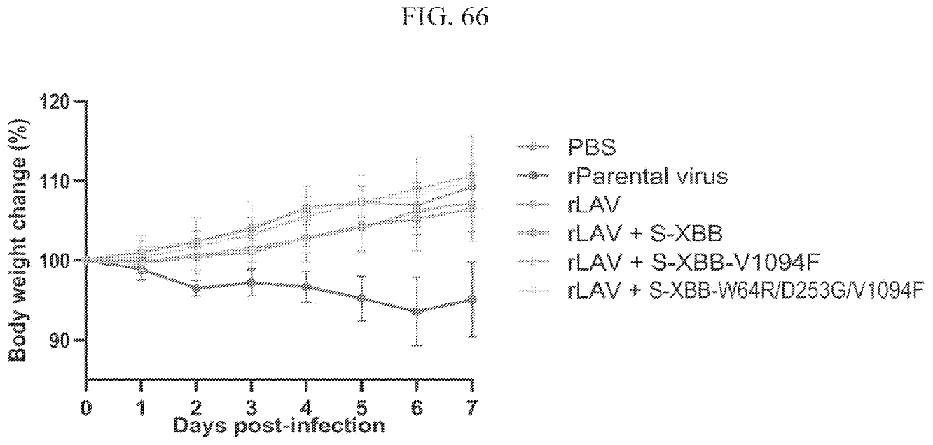

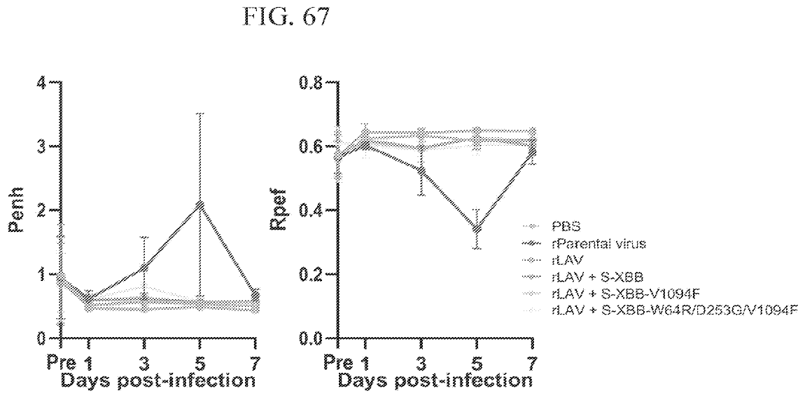

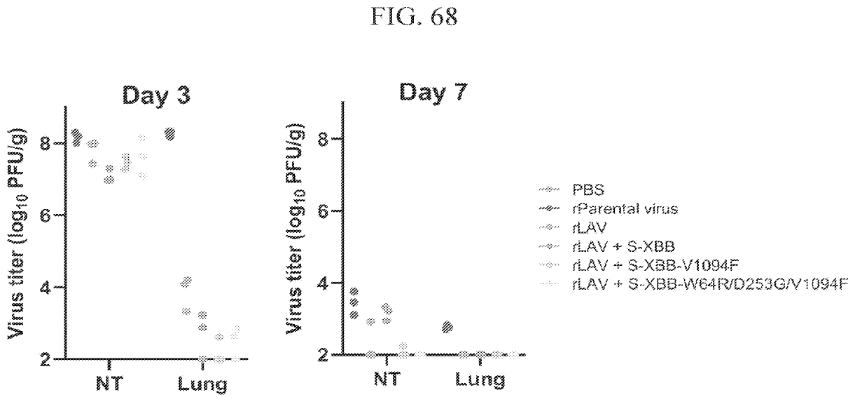

To test the pathogenicity of the three viruses possessing the XBB.1.5 S gene, hamsters were intranasally inoculated with 10³ PFU (FIG. 65). Body weight and respiratory function were monitored for 7 days post-infection. Virus titers were determined at 3 and 7 days post-infection. The three viruses possessing the XBB.1.5 S gene did not cause body weight loss (FIG. 66). The three viruses possessing the XBB.1.5 S gene impaired respiratory functions (FIG. 67). Virus titers in the lungs of hamsters infected with the three viruses possessing the XBB.1.5 S gene were similar to those of the vaccine cold-adapted coronavirus (LAV) (FIG. 68).

### Example 12

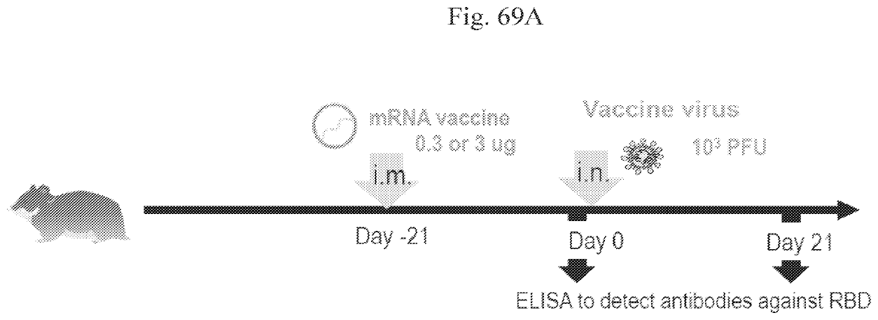

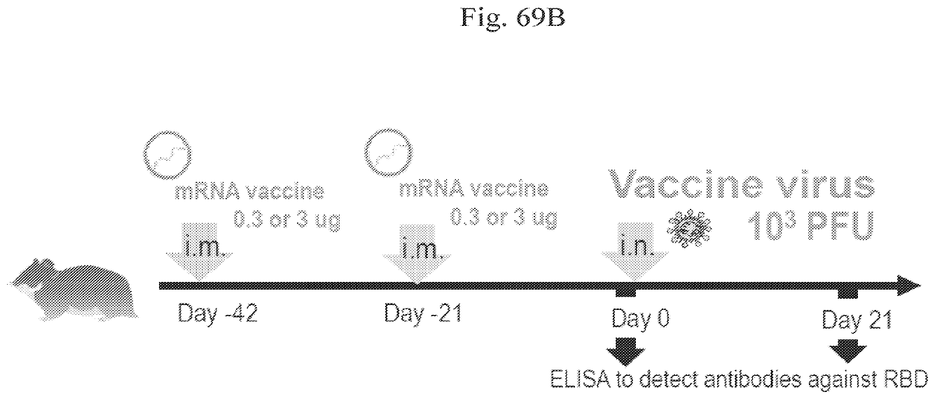

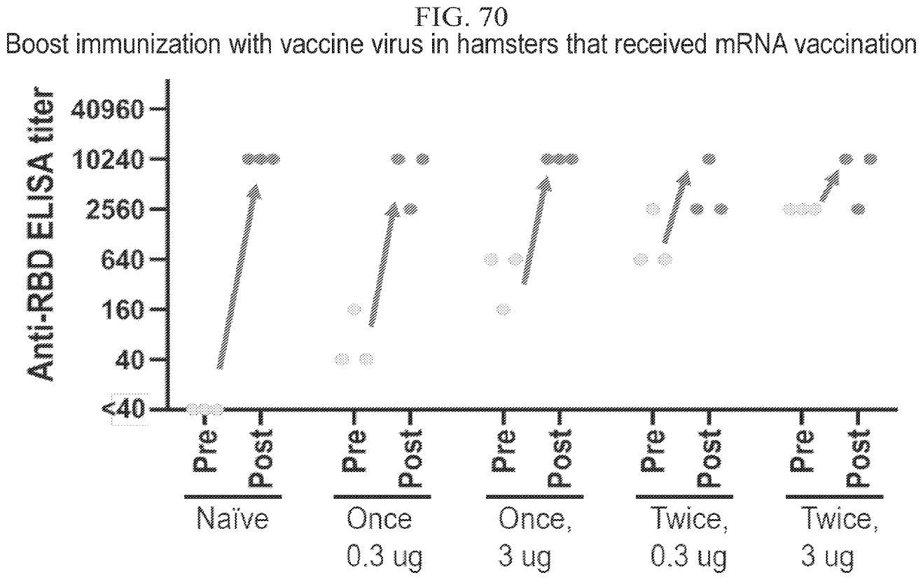

To test the boost immunization capability of the vaccine cold-adapted coronavirus, hamsters were immunized with the mRNA vaccine once (FIG. 69A) or twice (FIG. 69B) at 0.3 or 3 μg per shot before infection with the vaccine cold-adapted coronavirus. Antibody titers against the RBD were measured pre- and post-boost immunization with the vaccine cold-adapted coronavirus. Antibody titers increased after the boost immunization with the vaccine cold-adapted coronavirus, even when antibodies to the RBD were present prior to the boost immunization (FIG. 70).

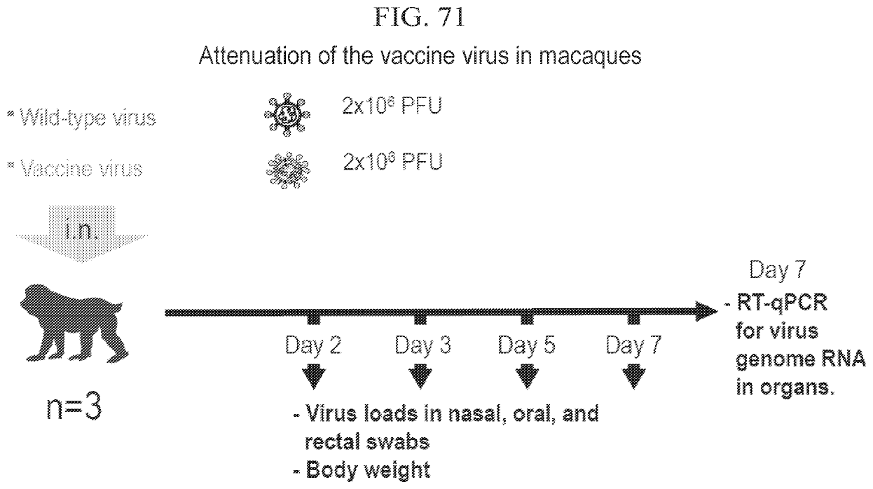

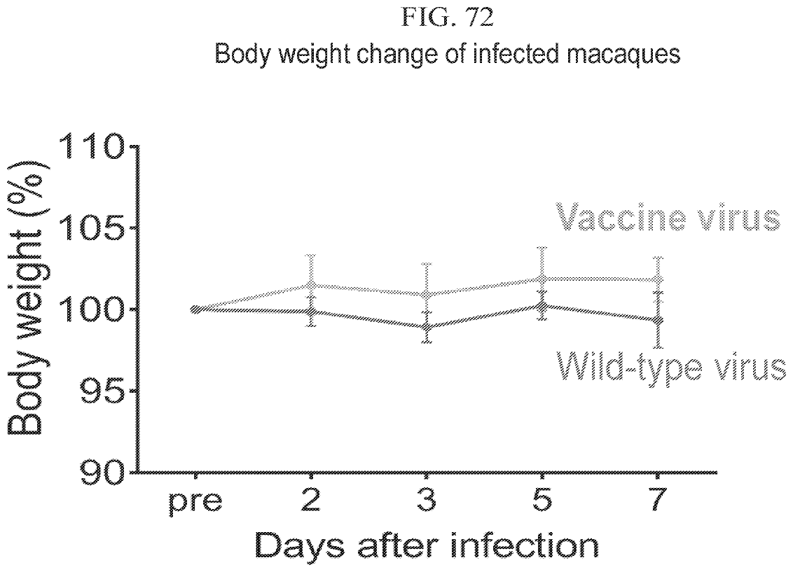

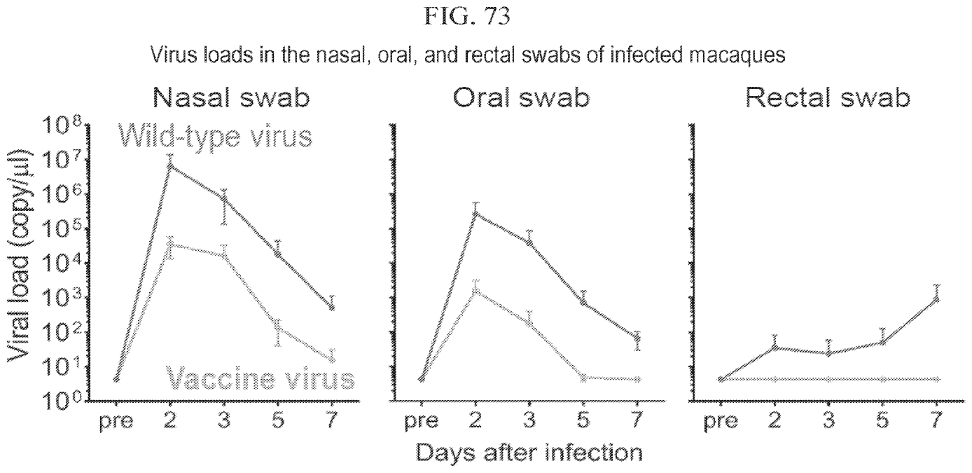

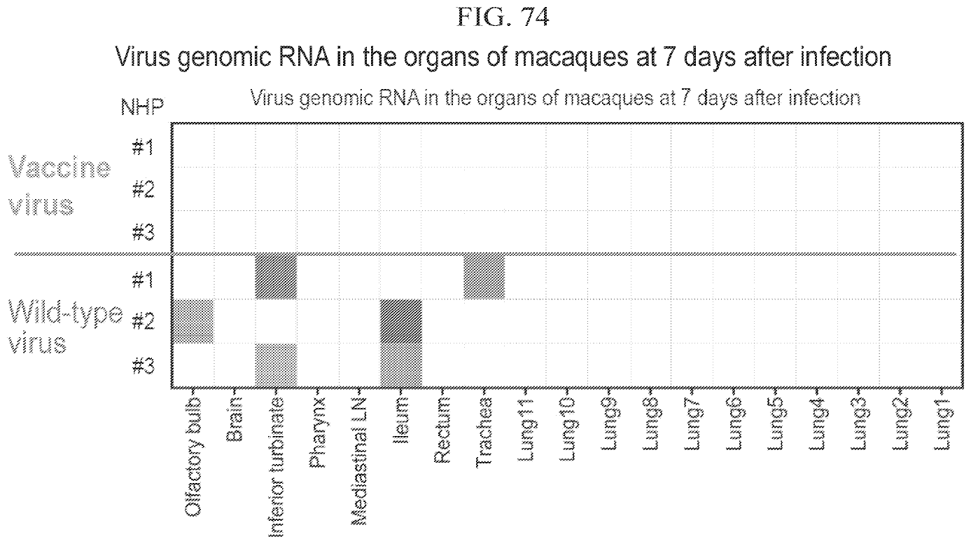

To test attenuation in non-human primates, macaques were inoculated with the vaccine cold-adapted coronavirus and virus loads in nasal, oral, and rectal swabs and body weight were assessed. At 7 days post-infection, virus RNA titers in organs were determined by RT-qPCR (FIG. 71). Neither the wild-type nor the vaccine cold-adapted coronavirus caused body weight loss (FIG. 72). Vaccine cold-adapted coronavirus loads in the nasal, oral, and rectal swabs were lower than the wild-type virus loads (FIG. 73). Vaccine cold-adapted coronavirus RNA was not detected in any organ tested, whereas wild-type virus RNA was detected in the olfactory bulb, turbinates, ileums, and trachea (FIG. 74).

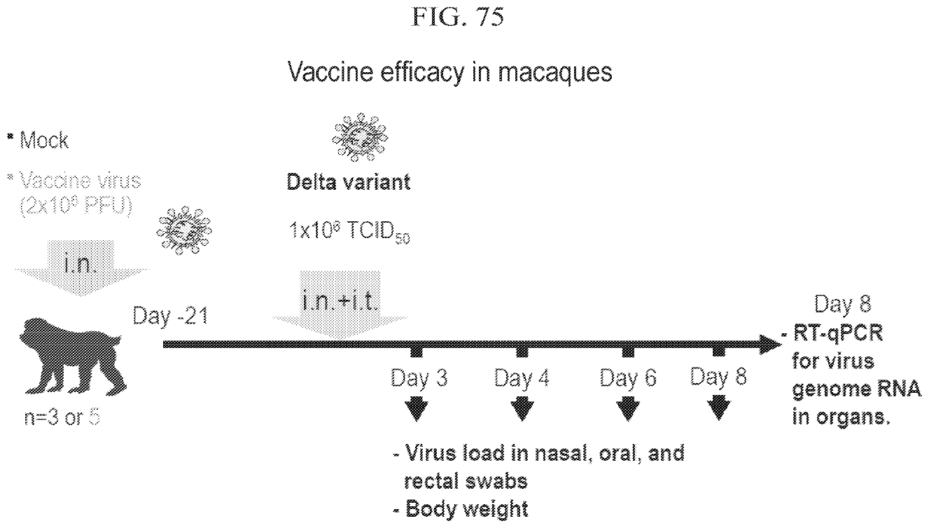

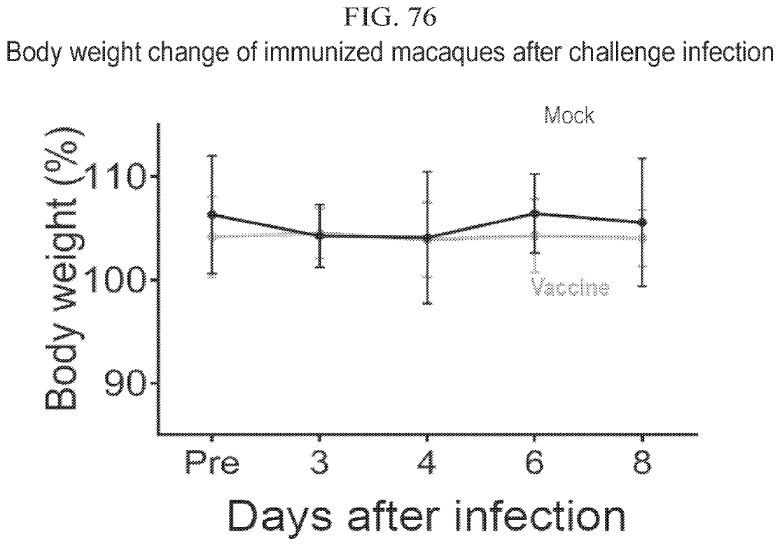

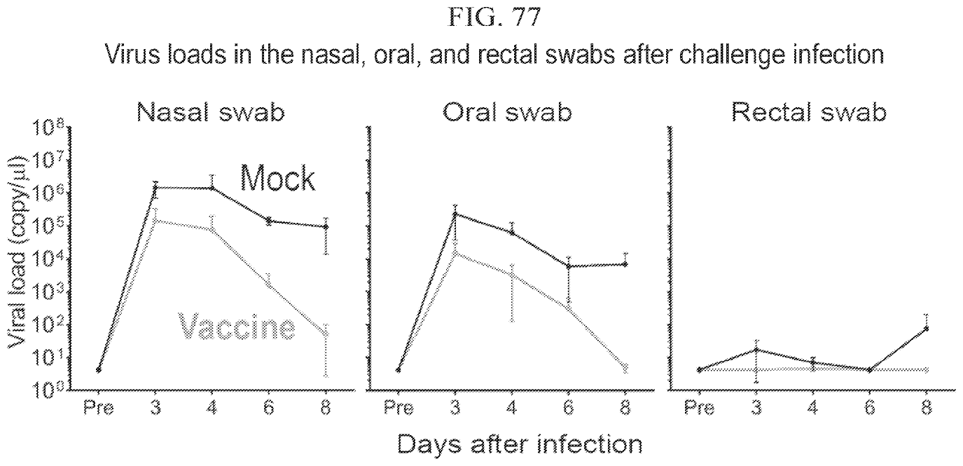

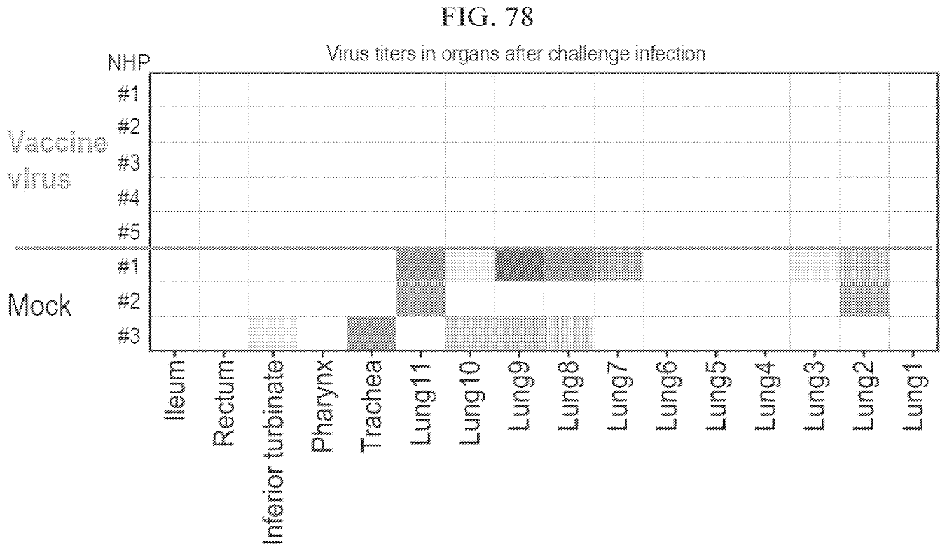

Macaques immunized with the vaccine cold-adapted coronavirus were intranasally and intratracheally challenged with a delta variant at 10⁶ TCID₅₀ (FIG. 75). Virus loads in nasal, oral, and rectal swabs and body weight were assessed. At 8 days post-infection, virus RNA titers in organs were determined by RT-qPCR. The delta variant did not cause body weight loss in either the immunized or mock macaques (FIG. 76). The virus loads of the challenge virus in the nasal, oral, and rectal swabs of the immunized macaques were lower than those of the mock animals (FIG. 77). Challenge virus RNA levels in the organs of the immunized macaques were significantly lower than those in the organs of the mock animals (FIG. 78).

### Statements

1. An isolated cold-adapted SARS-CoV-2 virus that has one or more mutations in one or more open reading frames that encode a non-structural protein for Nsp1, Nsp2, Nsp3, Nsp6, Nsp13, Nsp14, Nsp15, S, ORF6, ORF7b, ORF8, or any combination thereof, relative to a parental virus that is not cold-adapted.

2. The isolated cold-adapted SARS-CoV-2 virus of statement 1, wherein the one or more mutations includes:
a deletion of a codon for the amino acid at position 85 in Nsp1;
a nucleotide substitution in the gene for Nsp2 at position 1341 or 1495, or
   both, and wherein the substitution is optionally a silent substitution;
an amino acid substitution in Nsp3 at position 272 or 528, or both; or
a combination thereof.

3. The isolated cold-adapted SARS-CoV-2 virus of statement 2, wherein the amino acid substitution in Nsp3 comprises:
an amino acid substitution at position 272 that is not Y;
an amino acid substitution at position 272 is N, D, E or Q;
an amino acid substitution at position 528 is not L;
an amino acid substitution at position 528 is F, W or Y; or
a combination thereof.

4. The isolated cold-adapted coronavirus of any one of statements 1 to 3, wherein the one or more mutations include an amino acid substitution in Nsp6 at position 260, wherein the amino acid substitution is not L or the amino acid substitution is F, W or Y.

5. The isolated cold-adapted coronavirus of any one of statements 1 to 4, wherein the one or more mutations include a nucleotide substitution in the gene for Nsp13 at position 16575, which is optionally a silent substitution, or wherein the one or more mutations include an amino acid substitution in Nsp14 at position 360, or a combination thereof.

6. The isolated cold-adapted coronavirus of statement 7, wherein the amino acid substitution in Nsp14 at position 360 is not A or the amino acid substitution is S, T, L, I or M.

7. The isolated cold-adapted coronavirus of any one of statements 1 to 6, wherein the one or more mutations include an amino acid substitution in Nsp15 at position 233, wherein the amino acid at position 233 is not E or the amino acid at position 233 is A, I, L, G, S, or T.

8. The isolated cold-adapted coronavirus of any one of statements 1 to 7, wherein the one or more mutations include an amino acid substitution in S at position 64, 235, 1094, or a combination thereof, wherein the amino acid substitution in S at position 64 is not W, position 235 is not D, or position 1094 is not V or wherein the amino acid substitution in S at position 64 is R, H or K; position 235 is G, A, L, I, T or S; or position 1094 is F, Y or W.

9. The isolated cold-adapted coronavirus of any one of statements 1 to 8, wherein the one or more mutations include a deletion of the C-terminus of a polypeptide encoded by ORF6, which deletion optionally inhibits interferon antagonism.

10. The isolated cold-adapted coronavirus of any one of statements 1 to 9, wherein the one or more mutations include a deletion of ORF7b and/or ORF8 genes, which optionally results in a deletion in nucleotides from position 27816 to position 28244 in a nucleic acid sequence of the parental virus, which yields a portion of ORF7b protein or the inability to encode a protein from ORF8 or both.

11. A method of making a cold-adapted SARS-CoV-2 virus comprising modifying a genome thereof to include one or more mutations in one or more open reading frames that encode for a non-structural protein for Nsp1, Nsp2, Nsp3, Nsp6, Nsp13, Nsp14, Nsp15, S, ORF6, ORF7b, ORF8, or any combination thereof, relative to a parental virus that is not cold-adapted.

12. The method of statement 11, wherein the one or more mutations include: a deletion of a codon for the amino acid at position 85 in Nsp1;
a nucleotide substitution in the gene for Nsp2 at position 1341 or 1495, or both;
an amino acid substitution in Nsp3 at position 272 or 528, or both; or a combination thereof.

13. The method of statement 12, wherein the amino acid substitution in Nsp3 at position 272 is not Y or the amino acid substitution is N, D, E or Q; the amino acid substitution in Nsp3 at position 528 is not L or the amino acid substitution is F, W or Y; or a combination thereof.

14. The method of any one of statements 11 to 13, wherein the one or more mutations include an amino acid substitution in Nsp6 at position 260 is not L or the amino acid substitution is F, W or Y; the amino acid substitution in Nsp14 at position 360 is not A or the amino acid substitution is S, T, L, I or M; the amino acid substitution in Nsp15 at position 233 is not E or the substitution is A, I, L, G, S, or T; or a combination thereof.

15. The method of any one of statements 11 to 14, wherein the one or more mutations include a nucleotide substitution in the gene for Nsp13 at position 16575.

16. The method of any one of statements 11 to 15, wherein the one or more mutations include an amino acid substitution in S at position 64, 235, 1094, or a combination thereof, wherein the amino acid substitution at position 64 is not W, position 235 is not D, or position 1094 is not V, or a combination thereof, or wherein the amino acid substitution at position 64 is R, H or K, position 235 is G, A, L, I, T or S, position 1094 is F, Y or W, or a combination thereof.

17. The method of any one of statements 11 to 16, wherein the one or more mutations include a deletion of the C-terminus of polypeptide encoded by ORF6, a deletion of ORF7b, a deletion of ORF8, or a combination thereof.

18. A method to immunize an animal, comprising administering to the animal a composition comprising the cold-adapted coronavirus of any one of statements 1 to 10.

19. The method of statement 18, wherein the animal is a mammal.

20. The method of statement 22, wherein the mammal is a human.

21. The method of any one of statements 18-20, wherein the composition is injected, systemically administered, or intranasally administered.

22. A pharmaceutical composition comprising the cold-adapted coronavirus of any one of statements 1 to 10.

23. The pharmaceutical composition of statement 22 further comprising a pharmaceutically acceptable carrier, a different immunogen, a different virus, or a combination thereof.

24. The pharmaceutical composition of statement 23, wherein the different virus is not a coronavirus or the different virus is an influenza virus or both.

The specific methods, devices and compositions described herein are representative of example embodiments and not intended as limitations on the scope of the technology. Other objects, aspects, and embodiments will occur to those skilled in the art upon consideration of this specification and are encompassed within the spirit of the technology as defined by the scope of the claims. It will be readily apparent to one skilled in the art that varying substitutions and modifications can be made to the technology disclosed herein without departing from the scope and spirit of the technology.

The technology illustratively described herein suitably can be practiced in the absence of any element or elements, or limitation or limitations, which is not specifically disclosed herein as essential. The methods and processes illustratively described herein suitably can be practiced in differing orders of steps, and the methods and processes are not necessarily restricted to the orders of steps indicated herein or in the claims.

Under no circumstances can the patent be interpreted to be limited to the specific examples or embodiments or methods specifically disclosed herein. Under no circumstances can the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the technology as claimed. Thus, it will be understood that although the present technology has been specifically disclosed by example embodiments and optional features, modification and variation of the concepts herein disclosed can be resorted to by those skilled in the art, and that such modifications and variations are considered to be within the scope of this technology as defined by the appended claims and statements of the technology.

The technology has been described broadly and generically herein. Each of the narrower species and subgeneric groupings falling within the generic disclosure also form part of the technology. This includes the generic description of the technology with a proviso or negative limitation removing any subject matter from the genus, regardless of whether or not the excised material is specifically recited herein. In addition, where features or aspects of the technology are described in terms of Markush groups, those skilled in the art will recognize that the technology is also thereby described in terms of any individual member or subgroup of members of the Markush group.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. (canceled)

2. The method of claim 13 a) wherein one plasmid has eight transcription cassettes for vRNA production.

3. The method of claim 2 wherein one plasmid has one transcription cassette for mRNA production and another plasmid has three transcription cassettes for mRNA production.

4. The method of claim 13 a) wherein one plasmid has six transcription cassettes for vRNA production and another plasmid has two transcription cassettes for vRNA production.

5. The method of claim 4 wherein one plasmid has four transcription cassettes for mRNA production.

6. The method of claim 13 a) wherein the HA is a type A HA.

7. The) The method of claim 13 a) wherein the HA is a type B HA.

8. (canceled)

9. The method of claim 13 b) further comprising four transcription cassettes for mRNA production.

10. The method of claim 9 wherein one plasmid has one transcription cassette for mRNA production and another plasmid has three transcription cassettes for mRNA production.

11. The method of claim 9 wherein one plasmid has the four transcription cassettes for mRNA production.

12. (canceled)

13. A method to prepare influenza virus, comprising:
contacting a cell with a set of plasmids in an amount effective to yield infectious influenza virus, wherein the set of plasmids is
a) a set of three plasmids for influenza virus production together having eight transcription cassettes for influenza virus vRNA production and four transcription cassettes for influenza virus mRNA production, wherein the transcription cassettes for vRNA production include a transcription cassette comprising a promoter operably linked to an influenza virus PA cDNA linked to a transcription termination sequence, a transcription cassette comprising a promoter operably linked to an influenza virus PB1 cDNA linked to a transcription termination sequence, a transcription cassette comprising a promoter operably linked to an influenza virus PB2 cDNA linked to a transcription termination sequence, a transcription cassette comprising a promoter operably linked to an influenza virus HA cDNA linked to a transcription termination sequence, a transcription cassette comprising a promoter operably linked to an influenza virus NP cDNA linked to a transcription termination sequence, a transcription cassette comprising a promoter operably linked to an influenza virus NA cDNA linked to a transcription termination sequence, a transcription cassette comprising a promoter operably linked to an influenza virus M cDNA linked to a transcription termination sequence, and a transcription cassette comprising a promoter operably linked to an influenza virus NS cDNA linked to a transcription termination sequence; wherein the transcription cassettes for mRNA production include a transcription cassette comprising a PolII promoter operably linked to a DNA coding region for influenza virus PA linked to a PolII transcription termination sequence, a transcription cassette comprising a PolII promoter operably linked to a DNA coding region for influenza virus PB1 linked to a PolII transcription termination sequence, a transcription cassette comprising a PolII promoter operably linked to a DNA coding region for influenza virus PB2 linked to a PolII transcription termination sequence, and a transcription cassette comprising a PolII promoter operably linked to a DNA coding region for influenza virus NP linked to a PolII transcription termination sequence, and wherein transfection of a cell with the composition yields titers of at least 1×10² TCID₅₀/mL;
b) a set of two plasmids for influenza virus vRNA production together comprising eight transcription cassettes for vRNA production, which transcription cassettes comprise a transcription cassette comprising a Poll promoter operably linked to an influenza virus PA cDNA linked to a Poll transcription termination sequence, a transcription cassette comprising a Poll promoter operably linked to an influenza virus PB1 cDNA linked to a Poll transcription termination sequence, a transcription cassette comprising a Poll promoter operably linked to an influenza virus PB2 cDNA linked to a Poll transcription termination sequence, a transcription cassette comprising a Poll promoter operably linked to an influenza virus HA cDNA linked to a Poll transcription termination sequence, a transcription cassette comprising a Poll promoter operably linked to an influenza virus NP cDNA linked to a Poll transcription termination sequence, a transcription cassette comprising a Poll promoter operably linked to an influenza virus NA cDNA linked to a Poll transcription termination sequence, a transcription cassette comprising a Poll promoter operably linked to an influenza virus M cDNA linked to a Poll transcription termination sequence, and/or a transcription cassette comprising a Poll promoter operably linked to an influenza virus NS cDNA linked to a Poll transcription termination sequence, wherein the composition includes at least six transcription cassettes for vRNA, transfection of a cell with the composition yields titers of at least 1×10² TCID₅₀/mL; or
c) a set of plasmids comprising one or two plasmids having eight transcription cassettes for influenza virus vRNA production and four plasmids each having a transcription cassette for influenza virus mRNA production for a different influenza virus protein, wherein the transcription cassettes for vRNA production include a transcription cassette comprising a Poll promoter operably linked to an influenza virus PA cDNA linked to a Poll transcription termination sequence, a transcription cassette comprising a Poll promoter operably linked to an influenza virus PB1 cDNA linked to a Poll transcription termination sequence, a transcription cassette comprising a Poll promoter operably linked to an influenza virus PB2 cDNA linked to a Poll transcription termination sequence, a transcription cassette comprising a Poll promoter operably linked to an influenza virus HA cDNA linked to a Poll transcription termination sequence, a transcription cassette comprising a Poll promoter operably linked to an influenza virus NP cDNA linked to a Poll transcription termination sequence, a transcription cassette comprising a Poll promoter operably linked to an influenza virus NA cDNA linked to a Poll transcription termination sequence, a transcription cassette comprising a Poll promoter operably linked to an influenza virus M cDNA linked to a Poll transcription termination sequence, and a transcription cassette comprising a Poll promoter operably linked to an influenza virus NS cDNA linked to a Poll transcription termination sequence; wherein the transcription cassettes for mRNA production include a transcription cassette comprising a PolII promoter operably linked to a DNA coding region for influenza virus PA linked to a PolII transcription termination sequence, a transcription cassette comprising a PolII promoter operably linked to a DNA coding region for influenza virus PB1 linked to a PolII transcription termination sequence, a transcription cassette comprising a PolII promoter operably linked to a DNA coding region for influenza virus PB2 linked to a PolII transcription termination sequence, and a transcription cassette comprising a PolII promoter operably linked to a DNA coding region for influenza virus NP linked to a PolII transcription termination sequence.

14. (canceled)

15. A method to prepare influenza virus, comprising contacting a cell with at least two plasmids which include transcription cassettes for vRNA production including a transcription cassette comprising a Poll promoter operably linked to an influenza virus PA cDNA linked to a Poll transcription termination sequence, a transcription cassette comprising a Poll promoter operably linked to an influenza virus PB1 cDNA linked to a Poll transcription termination sequence, a transcription cassette comprising a Poll promoter operably linked to an influenza virus PB2 cDNA linked to a Poll transcription termination sequence, a transcription cassette comprising a Poll promoter operably linked to an influenza virus HA cDNA linked to a Poll transcription termination sequence, a transcription cassette comprising a Poll promoter operably linked to an influenza virus NP cDNA linked to a Poll transcription termination sequence, a transcription cassette comprising a Poll promoter operably linked to an influenza virus NA cDNA linked to a Poll transcription termination sequence, a transcription cassette comprising a Poll promoter operably linked to an influenza virus M cDNA linked to a Poll transcription termination sequence, and a transcription cassette comprising a Poll promoter operably linked to an influenza virus NS cDNA linked to a Poll transcription termination sequence, wherein the cell is contacted with eight transcription cassettes for vRNA, wherein the cell expresses influenza virus PA, PB1, PB2 and NP and wherein the cell produces titers of at least 1×10² TCID₅₀/mL.

16. The method of claim 15 wherein the influenza virus PA, PB1, PB2 and NP are expressed from four plasmids for mRNA production each having a transcription cassette for mRNA production of one of PA, PB1, PB2 or NP.

17. A method to prepare influenza virus, comprising contacting a cell with four plasmids for mRNA production and eight transcription cassettes for influenza virus vRNA production, wherein the eight transcription cassettes for vRNA production include a transcription cassette comprising a Poll promoter operably linked to an influenza virus PA cDNA linked to a Poll transcription termination sequence, a transcription cassette comprising a Pol promoter operably linked to an influenza virus PB1 cDNA linked to a Poll transcription termination sequence, a transcription cassette comprising a Poll promoter operably linked to an influenza virus PB2 cDNA linked to a Poll transcription termination sequence, a transcription cassette comprising a Poll promoter operably linked to an influenza virus HA cDNA linked to a Poll transcription termination sequence, a transcription cassette comprising a Poll promoter operably linked to an influenza virus NP cDNA linked to a Poll transcription termination sequence, a transcription cassette comprising a Poll promoter operably linked to an influenza virus NA cDNA linked to a Poll transcription termination sequence, a transcription cassette comprising a Poll promoter operably linked to an influenza virus M cDNA linked to a Poll transcription termination sequence, and a transcription cassette comprising a Poll promoter operably linked to an influenza virus NS cDNA linked to a Poll transcription termination sequence, wherein the four plasmids for mRNA production each having a different transcription cassettes for mRNA production of an influenza virus protein selected from PA, PB1, PB2 and NP, and wherein the cell produces titers of at least 1×10² TCID 50/mL

18. The method of claim 15 or 17 further comprising a transcription cassette comprising a promoter linked to 5′ influenza virus sequences comprising 5′ influenza virus noncoding sequences linked to a DNA of interest linked to 3′ influenza virus sequences comprising 3′ influenza virus noncoding sequences linked to a transcription termination sequence.

19. The method of claim 15 or 17 further comprising isolating the virus.

20. A plasmid for vRNA production comprising a transcription cassette comprising a Poll promoter operably linked to an influenza virus HA cDNA linked to a Poll transcription termination sequence and a transcription cassette comprising a Poll promoter operably linked to an influenza virus NA cDNA linked to a Poll transcription termination sequence, wherein transfection of a cell with the plasmid and transcription cassettes for vRNA production of PA, PB1, PB2, NP, M and NS yields titers of at least 1×10² TCID₅₀/mL.

21. A plasmid for influenza virus production comprising a transcription cassette comprising a Poll promoter operably linked to an influenza virus HA cDNA linked to a Poll transcription termination sequence, a transcription cassette comprising a Poll promoter operably linked to an influenza virus NA cDNA linked to a Poll transcription termination sequence, a transcription cassette comprising a Poll promoter operably linked to an influenza virus M cDNA linked to a Poll transcription termination sequence, a transcription cassette comprising a Poll promoter operably linked to an influenza virus NS cDNA linked to a Poll transcription termination sequence, a transcription cassette comprising a Poll promoter and a Poll transcription termination sequence and a PolII promoter and a PolII transcription termination sequence each operably linked to a DNA segment for influenza virus PA, a transcription cassette comprising a Poll promoter and a Poll transcription termination sequence and a PolII promoter and PolII transcription termination sequence each operably linked to a DNA segment for influenza virus PB1, a transcription cassette comprising a Poll promoter and a Poll transcription termination sequence and a PolII promoter and a PolII transcription termination sequence each operably linked to a DNA segment encoding influenza virus PB2, and a transcription cassette comprising a Poll promoter and a Poll transcription termination sequence and a PolII promoter and a PolII transcription termination sequence each operably linked to a DNA segment for influenza virus NP.

22. A method to prepare influenza virus, comprising:
contacting a cell with the plasmid of claim 21 in an amount effective to yield infectious influenza virus.


SEQUENCE LISTING (78 sequences)

SEQ ID NO: 1 - DNA, 29903 bp, Coronavirus 2
  source 1..29903: mol_type = genomic DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggttttgtccgggtgtgaccgaaaggtaagatggagagccttgtccctggtttcaacgagaaaacacacgtccaacttagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttttaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatccctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaataatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaatcaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacttacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgttgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcctcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcttgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaaacgaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtttaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 2 - DNA, 29903 bp, Coronavirus 2
  source 1..29903: mol_type = genomic DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 3 - DNA, 29836 bp, Coronavirus 2
  source 1..29836: mol_type = genomic DNA
caaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgttttgcagccgatcatcagcacatctaggttttgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttttaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaagtctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatatccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgcggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggannnnnnnnnnnnnnnnnnnnnnnnnnnnnnnnnatgtactgaagtcagaggacgcgcagggaatggataatcttgcctgcgaagatctaaaactagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctatatcttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgattttgcaacatcagcttgtgttttggctgctgaatgtacaatttttaaagatgcttctggtaagccattaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaatctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccgctgtagcttattttaatatggtctatatgcctgctagttgggtgatgcgtattatgacatggttggatatggttgatactagtttgtctggttttaagctaaaagactgtgttatgtatgcatcagctgtggtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacttacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcagttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaactacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgttaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttagaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccaytgagaagtctaacataataagaggctggatttttggtactactttagattcgaagacccagtccctacttattgttaataacgctactaatgttgttattaaagtctgtgaatttcaattttgtaatgatccatttttggatgtttattaccacaaaaacaacaaaagttggatggaaagtgnnnnnngagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagattgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcgtcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaaaatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactcctttagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctaycgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagcttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaatttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttaatctaaacgaacaaactnaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaaggccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtatgggaacttctcctgctagaatggctggcaatggctgtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggcttatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccactcggagtacgatcgagtgtacagtgaacaatgctagggagagctgcctatatggaagagccctaatgtgtaaaattaattttagtagtgctatccccatgtgattttaatagcttcttagga

SEQ ID NO: 4 - DNA, 29767 bp, Coronavirus 2
  source 1..29767: mol_type = genomic DNA
ttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcancacatctaggttttgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaggtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttttaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagcngctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatataaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattactttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaatctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcacaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtgttgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagtcacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacttacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaatatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacgtacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatttcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgttatctctgggaccaatggtactaagaggtttgataaccctgtcctaccatttaatgatggtgtttattttgcttccattgagaagtctaacataataagaggctggatttttggtactactttagattcgaagacccagtccctacttattgttaataacgctactaatgttgttattaaagtctgtgaatttcaattttgtaatgatccatttttggaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattatagtgcgtgagccagaagatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttgatgaagtttttaacgccaccagatttgcatctgtttatgcttggaacaggaagagaatcagcaactgtgttgctgattattctgtcctatataatctcgcaccatttttcacttttaagtgttatggagtgtctcctactaaattaaatgatctctgctttactaatgtctatgcagattcatttgtaattagaggtgatgaagtcagacaaatcgctccagggcaaactggaaatattgctgattataattataaattaccagatgattttacaggctgcgttatagcttggaattctaacaagcttgattctaaggttagtggtaattataattacctgtatagattgtttaggaagtctaatctcaaaccttttgagagagatatttcaactgaaatctatcaggccggtaacaaaccttgtaatggtgttgcaggttttaattgttactttcctttacgatcatatagtttccgacccacttatggtgttggtcaccaaccatacagagtagtagtactttcttttgaacttctacatgcaccagcaactgtttgtggacctaaaaagtctactaatttggttaaaaacaaatgtgtcaatttcaacttcaatggtttaaaaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaatatgtcaacaactcatatgagtgtgacatacccattggtgcaggtatatgcgctagttatcagactcagactaagtctcatcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaacgtgctttaactggaatagctgttgaacaagacaaaaacacccaagaagtttttgcacaagtcaaacaaatttacaaaacaccaccaattaaatattttggtggttttaatttttcacaaatattaccagatccatcaaaaccaagcaagaggtcatttattgaagatctacttttcaacaaagtgacacttgcagatgctngcttcatcaaacaatatggtgattgccttggtgatattgctgctagagacctcatttgtgcacaaaagtttaaaggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccataatgcacaagctttaaacacgcttgttaaacaacttagctccaaatttggtgcaatttcaagtgttttaaatgatatcttttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagattcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataactctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagataggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcaggttccaacggtactattaccgttgaagagcttaaaaagctccttgaagaatggaacctagtaataggtttcctattccttacatggatttgtcttctacaatttgcctatgccaacaggaataggtttttgtatataattaagttaattttcctctggctgttatggccagtaactttaacttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgcggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgttattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaacttaaatgtctgataatggaccccaaaatcagcgaaatgcactccgcattacgtttggtggaccctcagattcaactggcagtaaccagaatggtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaaacgaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaa

SEQ ID NO: 5 - DNA, 29903 bp, Coronavirus 2; identical to SEQ ID NO: 2
  source 1..29903: mol_type = genomic DNA

SEQ ID NO: 6 - DNA, 29727 bp, synthetic construct
  source 1..29727: mol_type = other DNA
agatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggttttgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttagagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccataggtacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgttgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcatttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttttaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctagtggcactactgaaatgctagcgaaagctttgagaaaagtgccaacagacaattatataaccacttacccgggtcagggtttaaatggttacactgtagaggaggcaaagacagtgcttaaaaagtgtaaaagtgctttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatagtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgttttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttatttactaatatgtttacaccactaattcaacctattggtgctttggacatatcagcatctatagtagctggtggtattgtggctatcgtagtaacatgccttgcctactattttatgaggtttagaagagcttttggtgaatacagtcatgtagttgcctttaatactttactattccttatgtcattcattgtactctgtttaacaccagtttactcattcttacctggtgtttattctgttatttacttgtacttgacattttatcttactaatgatgtttcttttttagcacatattcagtggatggttatgttcacacctttagtacctttctggataacaattgtttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattaccttttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaatctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagatatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgagacacaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaataaagggaggtaggtttgtacttgcactgttatccgatttacaggatttgaaatgggctagattccctaagagtgatggaactggtactatttatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacttacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcagttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactttctgacgatgctgttgtgtgttttaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagacccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcataccaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtactacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattatgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatacgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacgtacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctatacctacaggttatgttgatacacctaataatacagatttttccagagttagtgctaaaccaccgcctggagatcaatttaaacacctcataccgcttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccggcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaattgaaacgatttgtgcaccactcactgtcttttttgatggtagagttgatggtcaagtagacttatttagaaatgcccgtaatggtgttcttattacagagggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccacgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttataaccagaactcaatcatacactaattctttcacacgtggtgtttattaccctgacaaagttttcagatcctcagttttacattcaactcatgacttgttcttacctttcttttccaatgttacttggttccatgctatacatgtctctgggaccaatggtactaagaggtttgataaccctgccctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttggatgtttaccaaaaaaacaacaaaagttggatggaaagtgagttcggagtttattctagtgcgaataattgcacttttgaatatgtctctcagccttttcttatggaccttgaaggaaaagagggtaatttcaaaaatcttagggaatttgtgtttaagaatattgatggttattttaaaatatattctaagcacacgcctattaatttagagcgtgatctccctcagggtttttcggctttagaaccattggtagatttgccaataggtattaacatcactaggtttcaaactttacttgctttacatagaagttatttgactcctgttgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatcctttactgtagaaaaaggaatctatcaaacttctaactttagagtccaaccaacagaatctattgttagatttcctaatattacaaacttgtgcccttttcatgaagtttttaacgccaccacatttgcatctgtttatgcttggaacaggaagagaatcagcaactgtgttgctgattattctgtcatatataatttcgcaccatttttcgcttttaagtgttatggagtgtctcctactaaattaaatgatctctgctttactaatgtctatgcagattcatttgtaattagaggtaatgaagtcagccaaatcgctccagggcaaactggaaatattgctgattataattataaattaccagatgattttacaggctgcgttatagcttggaattctaacaagcttgattctaagcctagtggtaattataattacctgtatagattgttaaggaagtctaagctcaaaccttttgagagagatatttcaactgaaatctatcaggccggtaacaaaccttgtaatggtgttgcaggtcctaattgttactctcctttacaatcatatggtttccgacccacttatggtgttggtcaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaatatgtcaacaactcatatgagtgtgacatacccattggtgcaggtatatgcgctagttatcagactcagactaagtctcatcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaacgtgctttaactggaatagctgttgaacaagacaaaaacacccaagaagtttttgcacaagtcaaacaaatttacaaaacaccaccaattaaatattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccataatgcacaagctttaaacacgcttgttaaacaacttagctccaaatttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagattcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatctttacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataactctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacattggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagataggtgcgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaagaatggaacctagtaataggtttcctattccttacatggatttgtcttctacaatttgcctatgccaacaggaataggtttttgtatataattaagttaattttcctctggctgttatggccagtaactttaacttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcatttaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgcggacttttaaagtttccatttggaatcttgattacatcataaacctcataattaaaaatttatctaagtcactaactgagaataaatattctcaattagatgaagagcaaccaatggagattctctaaacgaacatgaaaattattcttttcttggcactgataacactcgctacttgtgagctttatcactaccaagagtgtgttagaggtacaacagtacttttaaaagaaccttgctcttctggcacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgttattccttgttttaattatgcttattatcttttggttctcacttgaactgcaagatcataatgaaacttgtcacgcctaaacgaacatgaaatttcttgttttcttatgaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaacttaaatgtctgataatggaccccaaaaccagcgaaatgcactccgcattacgtttggtggaccctcagattcaactggcagtaaccagaatggtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaaacgaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagccgtgctgactcaactcaggcctaaactcatgcagaccacacaaggcagatgggctatataaacgttttcgcttttccgtttacgatatatagtctactcttgtgcagaatgaattttcgtaactacatagcacaagtagatgtagttaactttaatctcacatagcaatctttaatcagtgtgtaacattagggaggacttgaaagagccaccacattttcacctacagtgaacaatgctagggagagctgcctatatggaagagccctaatgtgtaaaattaattttagtagtgctatccc

SEQ ID NO: 7-19 - skipped (13 entries)

SEQ ID NO: 20 - protein, 180 aa, Coronavirus 2
MESLVPGFNEKTHVQLSLPVLQVRDVLVRGFGDSVEEVLSEARQHLKDGTCGLVEVEKGVLPQLEQPYVFIKRSDARTAPHGHVMVELVAELEGIQYGRSGETLGVLVPHVGEIPVAYRKVLLRKNGNKGAGGHSYGADLKSFDLGDELGTDPYEDFQENWNTKHSSGVTRELMRELNGG

SEQ ID NO: 21 - protein, 638 aa, Coronavirus 2
AYTRYVDNNFCGPDGYPLECIKDLLARAGKASCTLSEQLDFIDTKRGVYCCREHEHEIAWYTERSEKSYELQTPFEIKLAKKFDTFNGECPNFVFPLNSIIKTIQPRVEKKKLDGFMGRIRSVYPVASPNECNQMCLSTLMKCDHCGETSWQTGDFVKATCEFCGTENLTKEGATTCGYLPQNAVVKIYCPACHNSEVGPEHSLAEYHNESGLKTILRKGGRTIAFGGCVFSYVGCHNKCAYWVPRASANIGCNHTGVVGEGSEGLNDNLLEILQKEKVNINIVGDFKLNEEIAIILASFSASTSAFVETVKGLDYKAFKQIVESCGNFKVTKGKAKKGAWNIGEQKSILSPLYAFASEAARVVRSIFSRTLETAQNSVRVLQKAAITILDGISQYSLRLIDAMMFTSDLATNNLVVMAYITGGVVQLTSQWLTNIFGTVYEKLKPVLDWLEEKFKEGVEFLRDGWEIVKFISTCACEIVGGQIVTCAKEIKESVQTFFKLVNKFLALCADSIIIGGAKLKALNLGETFVTHSKGLYRKCVKSREETGLLMPLKAPKEIIFLEGETLPTEVLTEEVVLKTGDLQPLEQPTSEAVEAPLVGTPVCINGLMLLEIKDTEKYCALAPNMMVTNNTFTLKGG

SEQ ID NO: 22 - protein, 1945 aa, Coronavirus 2
APTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIIPNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGG

SEQ ID NO: 23 - protein, 500 aa, Coronavirus 2
KIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYSFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQ

SEQ ID NO: 24 - protein, 306 aa, Coronavirus 2
SGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQSGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQ

SEQ ID NO: 25 - protein, 290 aa, Coronavirus 2
SAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQ

SEQ ID NO: 26 - protein, 83 aa, Coronavirus 2
SKMSDVKCTSVVLLSVLQQLRVESSSKLWAQCVQLHNDILLAKDTTEAFEKMVSLLSVLLSMQGAVDINKLCEEMLDNRATLQ

SEQ ID NO: 27 - protein, 198 aa, Coronavirus 2
AIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQ

SEQ ID NO: 28 - protein, 113 aa, Coronavirus 2
NNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQ

SEQ ID NO: 29 - protein, 139 aa, Coronavirus 2
AGNATEVPANSTVLSFCAFAVDAAKAYKDYLASGGQPITNCVKMLCTHTGTGQAITVTPEANMDQESFGGASCCLYCRCHIDHPNPKGFCDLKGKYVQIPTTCANDPVGFTLKNTVCTVCGMWKGYGCSCDQLREPMLQ

SEQ ID NO: 30 - protein, 13 aa, Coronavirus 2
SADAQSFLNGFAV

SEQ ID NO: 31 - protein, 932 aa, Coronavirus 2
SADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPLTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFVVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 32 - protein, 601 aa, Coronavirus 2
AVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKVGILCIMSDRDLYDKLQFTSLEIPRRNVATLQ

SEQ ID NO: 33 - protein, 527 aa, Coronavirus 2
AENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQ

SEQ ID NO: 34 - protein, 346 aa, Coronavirus 2
SLENVAFNVVNKGHFDGQQGEVPVSIINNTVYTKVDGVDVELFENKTTLPVNVAFELWAKRNIKPVPEVKILNNLGVDIAANTVIWDYKRDAPAHISTIGVCSMTDIAKKPTETICAPLTVFFDGRVDGQVDLFRNARNGVLITEGSVKGLQPSVGPKQASLNGVTLIGEAVKTQFNYYKKVDGVVQQLPETYFTQSRNLQEFKPRSQMEIDFLELAMDEFIERYKLEGYAFEHIVYGDFSHSQLGGLHLLIGLAKRFKESPFELEDFIPMDSTVKNYFITDAQTGSSKCVCSVIDLLLDDFVEIIKSQDLSVVSKVVKVTIDYTEISFMLWCKDGHVETFYPKLQ

SEQ ID NO: 35 - protein, 298 aa, Coronavirus 2
SSQAWQPGVAMPNLYKMQRMLLEKCDLQNYGDSATLPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPKTKNVTKENDSKEGFFTYICGFIQQKLALGGSVAIKITEHSWNADLYKLMGHFAWWTAFVTNVNASSSEAFLIGCNYLGKPREQIDGYVMHANYIFWRNTNPIQLSSYSLFDMSKFPLKLRGTAVMSLKEGQINDMILSLLSKGRLIIRENNRVVISSDVLVNN

SEQ ID NO: 36 - protein, 1273 aa, Coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 37 - protein, 275 aa, Coronavirus 2
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 38 - protein, 75 aa, Coronavirus 2
MYSFVSEETGTLIVNSVLLFLAFVVFLLVTLAILTALRLCAYCCNIVNVSLVKPSFYVYSRVKNLNSSRVPDLLV

SEQ ID NO: 39 - protein, 222 aa, Coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 40 - protein, 61 aa, Coronavirus 2
MFHLVDFQVTIAEILLIIMRTFKVSIWNLDYIINLIIKNLSKSLTENKYSQLDEEQPMEID

SEQ ID NO: 41 - protein, 121 aa, Coronavirus 2
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 42 - protein, 43 aa, Coronavirus 2
MIELSLIDFYLCFLAFLLFLVLIMLIIFWFSLELQDHNETCHA

SEQ ID NO: 43 - protein, 121 aa, Coronavirus 2
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 44 - protein, 419 aa, Coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNLANNAAIVLQLPQGTTLPKGFYAEGSRGGSQASSRSSSRSRNSSRNSTPGSSKRTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 45 - protein, 38 aa, Coronavirus 2
MGYINVFAFPFTIYSLLLCRMNSRNYIAQVDVVNFNLT

SEQ ID NO: 46-50 - skipped (5 entries)

SEQ ID NO: 51 - protein, 7096 aa, synthetic construct [sequence omitted]

SEQ ID NO: 52 - protein, 180 aa, synthetic construct; identical to SEQ ID NO: 20

SEQ ID NO: 53 - protein, 638 aa, synthetic construct; identical to SEQ ID NO: 21

SEQ ID NO: 54 - protein, 1945 aa, synthetic construct
APTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGG

SEQ ID NO: 55 - protein, 500 aa, synthetic construct; identical to SEQ ID NO: 23

SEQ ID NO: 56 - protein, 306 aa, synthetic construct [sequence omitted]

SEQ ID NO: 57 - protein, 290 aa, synthetic construct; identical to SEQ ID NO: 25

SEQ ID NO: 58 - protein, 83 aa, synthetic construct; identical to SEQ ID NO: 26

SEQ ID NO: 59 - protein, 198 aa, synthetic construct; identical to SEQ ID NO: 27

SEQ ID NO: 60 - protein, 113 aa, synthetic construct; identical to SEQ ID NO: 28

SEQ ID NO: 61 - protein, 139 aa, synthetic construct; identical to SEQ ID NO: 29

SEQ ID NO: 62 - protein, 13 aa, synthetic construct
SADAQSFLNGFAV

SEQ ID NO: 63 - protein, 932 aa, synthetic construct
SADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 64 - protein, 601 aa, synthetic construct; identical to SEQ ID NO: 32

SEQ ID NO: 65 - protein, 527 aa, synthetic construct; identical to SEQ ID NO: 33

SEQ ID NO: 66 - protein, 346 aa, synthetic construct; identical to SEQ ID NO: 34

SEQ ID NO: 67 - protein, 298 aa, synthetic construct; identical to SEQ ID NO: 35

SEQ ID NO: 68 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 69 - protein, 275 aa, synthetic construct; identical to SEQ ID NO: 37

SEQ ID NO: 70 - protein, 75 aa, synthetic construct; identical to SEQ ID NO: 38

SEQ ID NO: 71 - protein, 222 aa, synthetic construct; identical to SEQ ID NO: 39

SEQ ID NO: 72 - protein, 61 aa, synthetic construct; identical to SEQ ID NO: 40

SEQ ID NO: 73 - protein, 121 aa, synthetic construct; identical to SEQ ID NO: 41

SEQ ID NO: 74 - protein, 43 aa, synthetic construct; identical to SEQ ID NO: 42

SEQ ID NO: 75 - protein, 121 aa, synthetic construct; identical to SEQ ID NO: 43

SEQ ID NO: 76 - protein, 419 aa, synthetic construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 77 - protein, 38 aa, synthetic construct; identical to SEQ ID NO: 45

SEQ ID NO: 78 - DNA, 29903 bp, Coronavirus 2; identical to SEQ ID NO: 1
  source 1..29903: mol_type = genomic DNA